(19) United States
(12) Patent Application Publication
(10) Pub. No.: US 2023/0167097 A1
(43) Pub. Date: Jun. 1, 2023

(54) HETEROCYCLIC COMPOUND HAVING CYANO-SUBSTITUTION

(71) Applicant: BEIJING SUMMER SPROUT TECHNOLOGY CO., LTD., Beijing (CN)

(72) Inventors: Feng Li, Beijing (CN); Yang Wang, Beijing (CN); Jianfei Yao, Beijing (CN); Gang Yang, Beijing (CN); Chi Yuen Raymond Kwong, Beijing (CN); Chuanjun Xia, Beijing (CN)

(73) Assignee: BEIJING SUMMER SPROUT TECHNOLOGY CO., LTD., Beijing (CN)

(21) Appl. No.: 17/989,201

(22) Filed: Nov. 17, 2022

(30) Foreign Application Priority Data
Nov. 20, 2021 (CN) 202111381241.1
Sep. 21, 2022 (CN) 202211147926.4

(51) Int. Cl. C07D 405/04; C07D 405/14; C07D 409/14; C07D 491/048; H10K 85/30; H10K 50/13; H10K 85/60

(52) U.S. Cl. CPC C07D 405/04; C07D 405/14; C07D 409/14; C07D 491/048; H01L 51/0085; H01L 51/0087; H01L 51/5044; H01L 51/0071; C07B 2200/05

(57) ABSTRACT

Provided is a heterocyclic compound having cyano-substitution. The compound has a structure represented by Formula 1. These novel compounds are applicable to electroluminescent devices and can provide better device performance, especially improved device efficiency such as power efficiency, current efficiency and external quantum efficiency. Further provided are an organic electroluminescent device comprising the compound and a compound composition comprising the compound.

### CROSS-REFERENCE TO RELATED APPLICATION(S)

This application claims priority to Chinese Patent Application No. 202111381241.1 filed on Nov. 20, 2021 and Chinese Patent Application No. 202211147926.4 filed on Sep. 21, 2022, the disclosure of which are incorporated herein by reference in their entireties.

### TECHNICAL FIELD

The present disclosure relates to compounds for organic electronic devices such as organic light-emitting devices. In particular, the present disclosure relates to a heterocyclic compound with a cyano substitution, an organic electroluminescent device comprising the compound and a compound composition comprising the compound.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which includes an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may include multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently,

Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules. Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may include one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime.

WO2019132545A1 discloses an organic light-emitting device comprising a compound having the following structure:

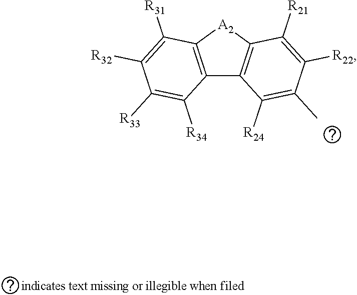

wherein X₂ is O or S; R₂₁, R₂₂, R₂₃ and R₂₄ are each -L₂₁-Ar₁ or hydrogen; R₃₁, R₃₂, R₃₃ and R₃₄ are each -L₂₂-Ar₂ or hydrogen; Ar₁ has a structure represented as follows:

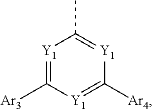

and at least one of Y₁ is selected from N; Ar₂ is selected from any one of the following structures:

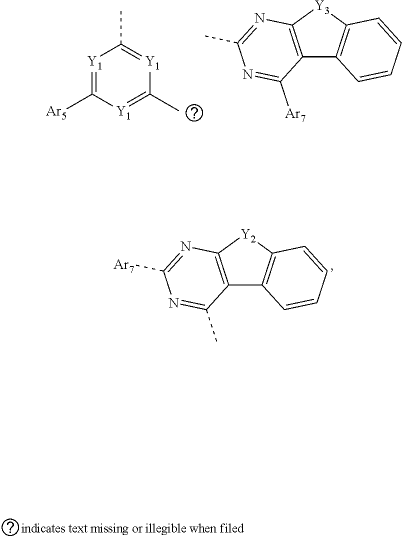

and at least one of Y₂ is selected from N. This application discloses compounds having the following specific structures:

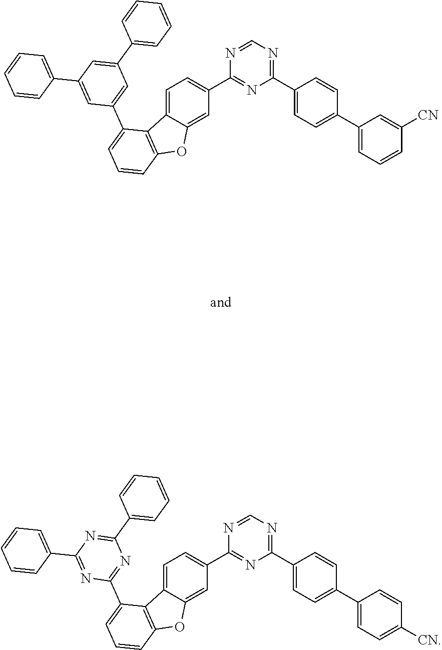 [SMILES: N#Cc1cccc(-c2ccc(-c3ncnc(-c4ccc5c(c4)oc4cccc(-c6cc(-c7ccccc7)cc(-c7ccccc7)c6)c45)n3)cc2)c1 ; N#Cc1ccc(-c2ccc(-c3ncnc(-c4ccc5c(c4)oc4cccc(-c6nc(-c7ccccc7)nc(-c7ccccc7)n6)c45)n3)cc2)cc1]

This application discloses and teaches a compound in which two benzene rings of dibenzofuran(thiophene) are both substituted by heteroaryl. However, this application does not disclose a compound having an aryl substituent on dibenzofuran(thiophene) and having a cyano substituent at a particular position, and does not disclose effects of the compound on device performance.

CN108250189A discloses an organic compound having the following structure and an organic light-emitting device comprising the compound:

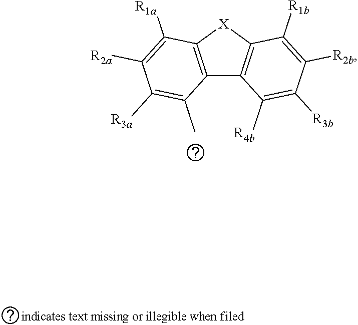

wherein X is O, S or SiR₅R₆; R₁ₐ to R₄ₐ are each independently L₁-HAr₁ or A₁, and at least one or more of R₁ₐ to _{R4a} are L₁-HAr₁; R_{1b} to R_{4b} are each independently L₂-HAr₂ or A₂, and at least one or more of R_{1b} to R_{4b} are L₂-HAr₂; HAr₁ and HAr₂ may each independently be

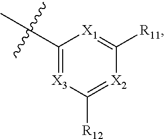

and at least two of X₁ to X₃ are selected from N. This application discloses compounds having the following specific structures:

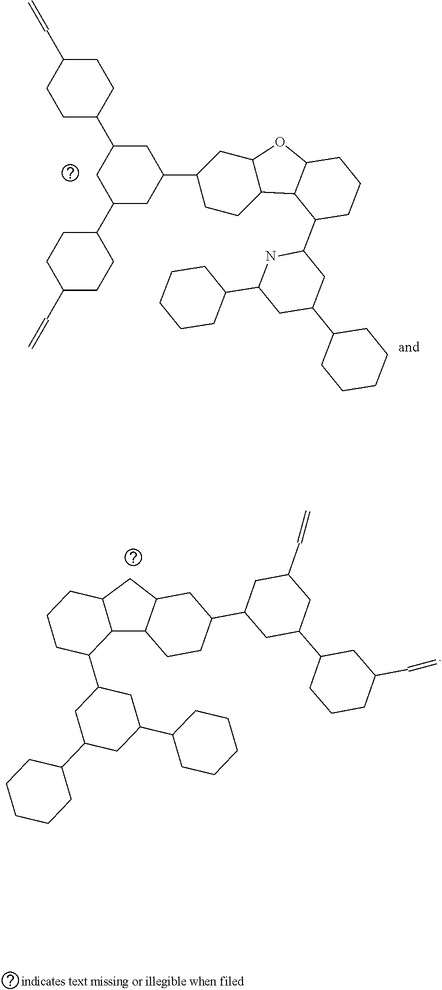

This application discloses and teaches a heterocyclic compound in which two phenyl of dibenzofuran(thiophene/silole) are both connected to heteroaryl and use of the heterocyclic compound in an organic electroluminescent device. However, this application does not disclose nor teach a compound having aryl and heteroaryl substituents at particular positions of two benzene rings of dibenzofuran(thiophene), respectively, and having a cyano substituent at a particular position, and does not disclose effects effect of the compound on device performance.

CN107619412A discloses an organic compound having the following structure and an organic light-emitting device comprising the compound:

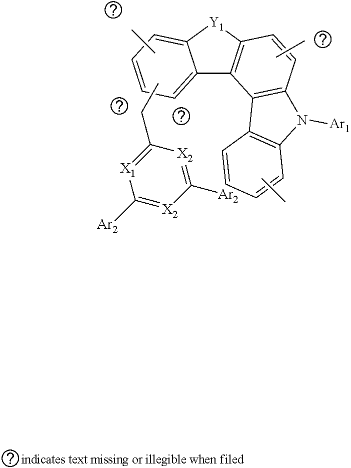

wherein Y₁ is O or S, X₁ to X₃ are each independently N or CR₁₁, and at least one of X₁ to X₃ is N. This application discloses compounds having the following specific structures:

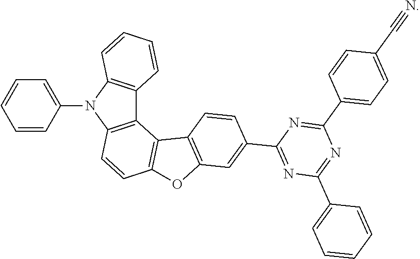 [SMILES: N#Cc1ccc(-c2nc(-c3ccccc3)nc(-c3ccc4c(c3)oc3ccc5c(c6ccccc6n5-c5ccccc5)c34)n2)cc1]

This application discloses and teaches a heterocyclic compound having an indole fused ring skeletal structure and use of the heterocyclic compound in an organic electroluminescent device. However, this application does not disclose nor teach a heterocyclic compound having a non-fused ring skeleton and having a cyano substitution at a particular position, and does not disclose effects of the heterocyclic compound on device performance.

### SUMMARY

The present disclosure aims to provide a series of heterocyclic compounds each having a cyano substitution to solve at least part of the preceding problems. These novel compounds each have a structure represented by Formula 1, may be applied to organic electroluminescent devices, and can provide better device performance, especially improved device efficiency.

According to an embodiment of the present disclosure, disclosed is a compound having a structure of Formula 1:

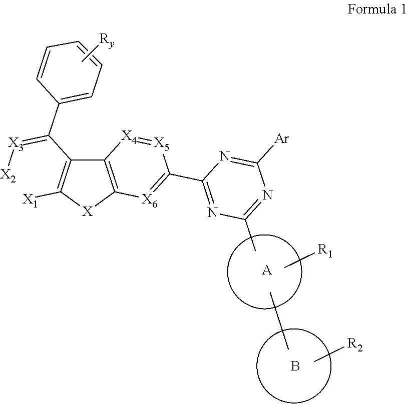

wherein

X is selected from O, S or Se;

X₁ to X₆ are, at each occurrence identically or differently, selected from CRₓ or N;

Ar is, at each occurrence identically or differently, selected from substituted or unsubstituted aryl having 6 to 30 carbon atoms, substituted or unsubstituted heteroaryl having 3 to 30 carbon atoms or a combination thereof;

the ring A and the ring B are, at each occurrence identically or differently, selected from an aromatic ring having 6 to 30 carbon atoms, a heteroaromatic ring having 3 to 30 carbon atoms or a combination thereof;

R_{y} and R₁ represent, at each occurrence identically or differently, mono-substitution, multiple substitutions or non-substitution;

R₂ represents, at each occurrence identically or differently, mono-substitution or multiple substitutions;

at least one of R₂ is selected from cyano;

Rₓ and R₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfanyl group, a sulfinyl group, a sulfonyl group, a phosphino group and combinations thereof;

R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, an isocyano group, a hydroxyl group, a sulfanyl group, a sulfinyl group, a sulfonyl group, a phosphino group and combinations thereof;

adjacent substituents Rₓ, R_{y} can be optionally joined to form a ring; and

adjacent substituents R₁, R₂ can be optionally joined to form a ring.

According to another embodiment of the present disclosure, disclosed is an organic electroluminescent device comprising an anode, a cathode and an organic layer disposed between the anode and the cathode, wherein at least one layer of the organic layer comprises the compound in the preceding embodiment.

According to another embodiment of the present disclosure, further disclosed is a compound composition comprising the compound in the preceding embodiment.

The present disclosure discloses a series of heterocyclic compounds each having a cyano substitution. These novel compounds may be applied to organic electroluminescent devices and can provide better device performance, especially improved device efficiency such as power efficiency, current efficiency and external quantum efficiency.

### BRIEF DESCRIPTION OF DRAWINGS

FIG. 1 is a schematic diagram of an organic light-emitting apparatus that may contain a compound and a compound composition disclosed herein.

FIG. 2 is a schematic diagram of another organic light-emitting apparatus that may contain a compound and a compound composition disclosed herein.

### DETAILED DESCRIPTION

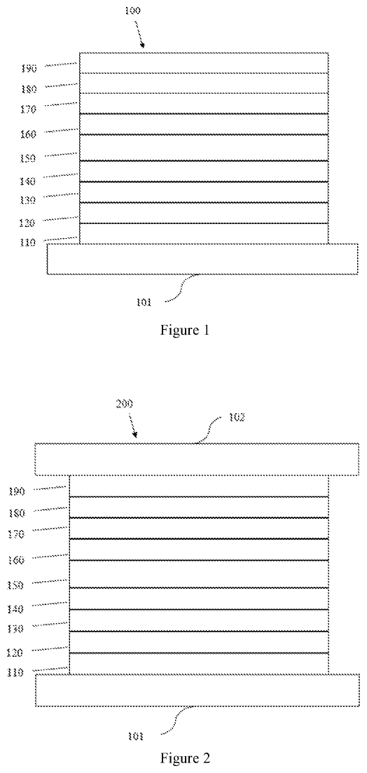

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent

Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}. Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butylmethylgermanylmethyl, triethylgermanylmethyl, triethylgermanylethyl, triisopropylgermanylmethyl, triisopropylgermanylethyl, trimethylsilylmethyl, trimethylsilylethyl, and trimethylsilylisopropyl, triisopropylsilylmethyl, triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl- 1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3 ,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur.

Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group.

Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more moieties selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl having 6 to 20 carbon atoms, unsubstituted alkylgermanyl having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen can also be replaced by their other stable isotopes. The replacement by other stable isotopes in the compounds may be preferred due to its enhancements of device efficiency and stability.

In the compounds mentioned in the present disclosure, multiple substitution refers to a range that includes a di-substitution, up to the maximum available substitution. When substitution in the compounds mentioned in the present disclosure represents multiple substitution (including di-, tri-, and tetra-substitutions, etc.), that means the substituent may exist at a plurality of available substitution positions on its linking structure, the substituents present at a plurality of available substitution positions may be the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fusedcyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

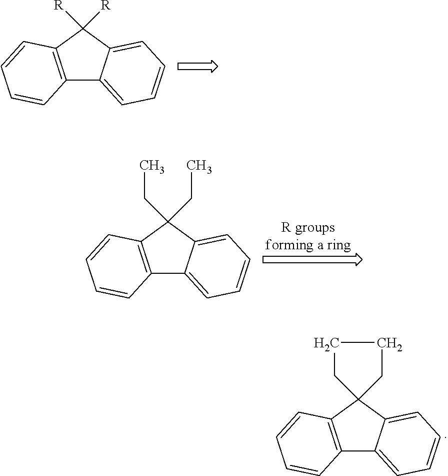

The expression that adjacent substituents can be optionally joined to form a ring is also intended to mean that two substituents bonded to carbon atoms which are directly bonded to each other are joined to each other via a chemical bond to form a ring, which can be exemplified by the following formula:

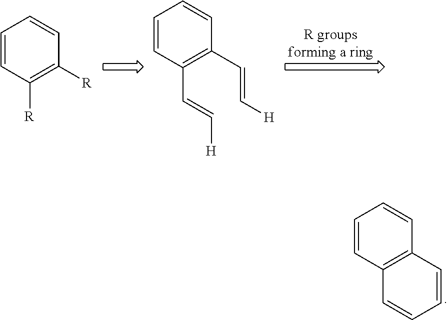

The expression that adjacent substituents can be optionally joined to form a ring is also intended to mean that two substituents bonded to a further distant carbon atom are joined to each other via a chemical bond to form a ring, which can be exemplified by the following formula:

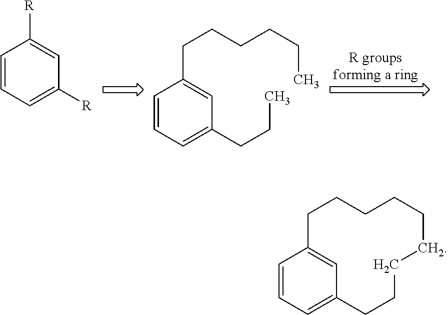

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

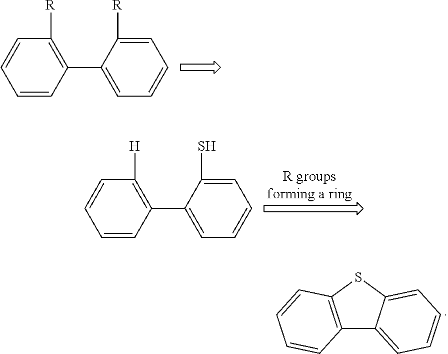

According to an embodiment of the present disclosure, disclosed is a compound having a structure of Formula 1:

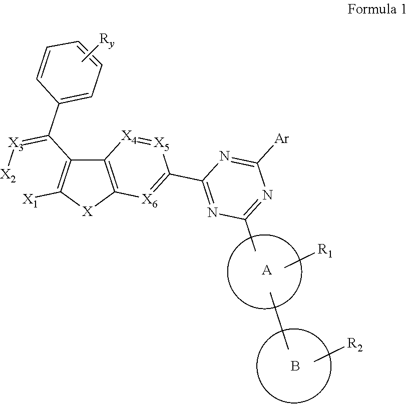

wherein

X is selected from O, S or Se;

X₁ to X₆ are, at each occurrence identically or differently, selected from CRₓ or N;

Ar is, at each occurrence identically or differently, selected from substituted or unsubstituted aryl having 6 to 30 carbon atoms, substituted or unsubstituted heteroaryl having 3 to 30 carbon atoms or a combination thereof;

the ring A and the ring B are, at each occurrence identically or differently, selected from an aromatic ring having 6 to 30 carbon atoms, a heteroaromatic ring having 3 to 30 carbon atoms or a combination thereof;

R_{y} and R₁ represent, at each occurrence identically or differently, mono-substitution, multiple substitutions or non-substitution;

R₂ represents, at each occurrence identically or differently, mono-substitution or multiple substitutions;

at least one of R₂ is selected from cyano;

Rₓ and R₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfanyl group, a sulfinyl group, a sulfonyl group, a phosphino group and combinations thereof;

R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, an isocyano group, a hydroxyl group, a sulfanyl group, a sulfinyl group, a sulfonyl group, a phosphino group and combinations thereof;

adjacent substituents Rₓ, R_{y} can be optionally joined to form a ring; and

adjacent substituents R₁, R₂ can be optionally joined to form a ring.

In this embodiment, the expression that “adjacent substituents Rₓ, R_{y} can be optionally joined to form a ring” is intended to mean that any one or more of groups of adjacent substituents, such as two substituents Rₓ, two substituents R_{y}, and substituents Rₓ and R_{y}, may be joined to form a ring. Obviously, it is possible that none of these substituents are joined to form a ring.

In this embodiment, the expression that “adjacent substituents R₁, R₂ can be optionally joined to form a ring” is intended to mean that any one or more of groups of adjacent substituents, such as two substituents R₁, two substituents R₂, and substituents R₁ and R₂, may be joined to form a ring. Obviously, it is possible that none of these substituents are joined to form a ring.

According to an embodiment of the present disclosure, adjacent substituents R_{y} are joined to form a carbocyclic ring; preferably, R_{y} are joined to form an aromatic ring.

According to an embodiment of the present disclosure, at least one of R₂ is selected from cyano, and the cyano substitution is located on the ring B at a meta or para position relative to the ring A. For example, when the ring B is selected from phenyl and at least one cyano substitution is located on the ring B at a meta position relative to the ring A, the compound has the following structure:

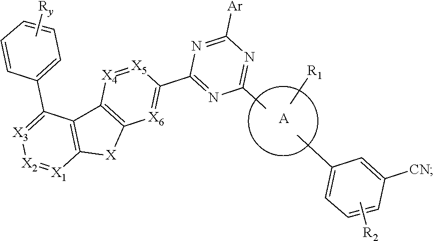

when the ring B is selected from phenyl and at least one cyano substitution is located on the ring B at a para position relative to the ring A, the compound has the following structure:

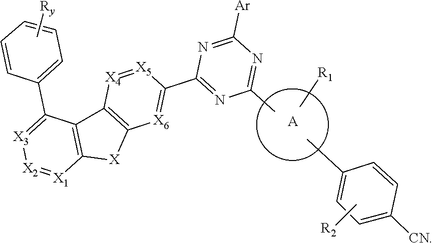

When the ring B is selected from other aryl or heteroaryl, the same is done.

According to an embodiment of the present disclosure, X is selected from O or S.

According to an embodiment of the present disclosure, X is selected from O.

According to an embodiment of the present disclosure, X₁ to X₆ are, at each occurrence identically or differently, selected from CRₓ.

According to an embodiment of the present disclosure, at least one of X₁ to X₆ is selected from N. For example, one of X₁ to X₆ is selected from N, or two of X₁ to X₆ are selected from N.

According to an embodiment of the present disclosure, Rₓ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, Rₓ is, at each occurrence identically or differently, selected from the group consisting of: hydrogen, deuterium, substituted or unsubstituted aryl having 6 to 30 carbon atoms, substituted or unsubstituted heteroaryl having 3 to 30 carbon atoms and combinations thereof.

According to an embodiment of the present disclosure, Rₓ is, at each occurrence identically or differently, selected from the group consisting of: hydrogen, deuterium, substituted or unsubstituted phenyl, substituted or unsubstituted naphthyl, substituted or unsubstituted biphenyl and combinations thereof.

According to an embodiment of the present disclosure, R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, R_{y} is, at each occurrence identically or differently, selected from the group consisting of: hydrogen, deuterium, substituted or unsubstituted aryl having 6 to 30 carbon atoms, substituted or unsubstituted heteroaryl having 3 to 30 carbon atoms and combinations thereof.

According to an embodiment of the present disclosure, R_{y} is, at each occurrence identically or differently, selected from the group consisting of: hydrogen, deuterium, substituted or unsubstituted phenyl, substituted or unsubstituted naphthyl, substituted or unsubstituted biphenyl and combinations thereof.

According to an embodiment of the present disclosure, R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof.

According to an embodiment of the present disclosure, R₁ is, at each occurrence identically or differently, selected from the group consisting of: hydrogen, deuterium, substituted or unsubstituted aryl having 6 to 30 carbon atoms, substituted or unsubstituted heteroaryl having 3 to 30 carbon atoms and combinations thereof.

According to an embodiment of the present disclosure, R₁ is, at each occurrence identically or differently, selected from the group consisting of: hydrogen, deuterium, substituted or unsubstituted phenyl, substituted or unsubstituted naphthyl, substituted or unsubstituted biphenyl and combinations thereof.

According to an embodiment of the present disclosure, R₂ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, cyano and combinations thereof.

According to an embodiment of the present disclosure, R₂ is, at each occurrence identically or differently, selected from the group consisting of: hydrogen, deuterium, substituted or unsubstituted aryl having 6 to 30 carbon atoms, substituted or unsubstituted heteroaryl having 3 to 30 carbon atoms, cyano and combinations thereof.

According to an embodiment of the present disclosure, R₂ is, at each occurrence identically or differently, selected from the group consisting of: hydrogen, deuterium, substituted or unsubstituted phenyl, substituted or unsubstituted naphthyl, substituted or unsubstituted biphenyl, cyano and combinations thereof.

According to an embodiment of the present disclosure, Ar is, at each occurrence identically or differently, selected from substituted or unsubstituted aryl having 6 to 20 carbon atoms, substituted or unsubstituted heteroaryl having 3 to 20 carbon atoms or a combination thereof.

According to an embodiment of the present disclosure, Ar is, at each occurrence identically or differently, selected from the group consisting of: substituted or unsubstituted phenyl, substituted or unsubstituted naphthyl, substituted or unsubstituted phenanthryl, substituted or unsubstituted biphenyl, substituted or unsubstituted terphenyl, substituted or unsubstituted triphenylenyl, substituted or unsubstituted fluorenyl, substituted or unsubstituted carbazolyl, substituted or unsubstituted dibenzofuryl, substituted or unsubstituted dibenzothienyl and combinations thereof.

According to an embodiment of the present disclosure, the compound is selected from the group consisting of Compound A-1 to Compound A-714, wherein the specific structures of Compound A-1 to Compound A-714 are referred to claim 8.

According to an embodiment of the present disclosure, hydrogens in Compound A-1 to Compound A-714 can be partially or fully substituted with deuterium.

According to an embodiment of the present disclosure, disclosed is an organic electroluminescent device comprising an anode, a cathode and an organic layer disposed between the anode and the cathode, wherein at least one layer of the organic layer comprises the compound in any one of the preceding embodiments.

According to an embodiment of the present disclosure, in the organic electroluminescent device, the organic layer is an emissive layer, the compound is a host compound, and the emissive layer comprises at least a first metal complex.

According to an embodiment of the present disclosure, the first metal complex has a general formula of M(Lₐ)ₘ(L_{b})ₙ(L_{c})_{q};

the metal M is selected from a metal with a relative atomic mass greater than 40;

Lₐ, L_{b} and L_{c} are a first ligand, a second ligand and a third ligand coordinated to the metal M, respectively; Lₐ, L_{b} and L_{c} may be the same or different;

Lₐ, L_{b} and L_{c} can be optionally joined to form a multidentate ligand; for example, any two of Lₐ, L_{b} and L_{c} may be joined to form a tetradentate ligand; in another example, Lₐ, L_{b} and L_{c} may be joined to each other to form a hexadentate ligand; in another example, none of Lₐ, L_{b} and L_{c} are joined so that no multidentate ligand is formed;

m is 1, 2 or 3, n is 0, 1 or 2, q is 0, 1 or 2, and m+n+q is equal to an oxidation state of the metal M; when m is greater than or equal to 2, a plurality of Lₐ may be the same or different; when n is 2, two L_{b} may be the same or different; when q is 2, two L_{c} may be the same or different;

the ligand Lₐ has a structure represented by Formula 2:

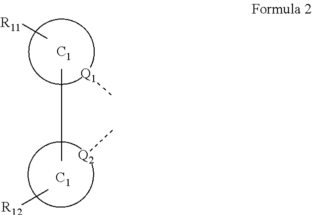

the ring C₁ and the ring C₂ are, at each occurrence identically or differently, selected from an aromatic ring having 5 to 30 ring atoms, a heteroaromatic ring having 5 to 30 ring atoms or a combination thereof;

Q₁ and Q₂ are, at each occurrence identically or differently, selected from C or N;

R₁₁ and R₁₂ represent, at each occurrence identically or differently, mono-substitution, multiple substitutions or non-substitution;

R₁₁ and R₁₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

adjacent substituents R₁₁, R₁₂ can be optionally joined to form a ring;

the ligands L_{b} and L_{c} are, at each occurrence identically or differently, selected from a monoanionic bidentate ligand.

According to an embodiment of the present disclosure, the ligands L_{b} and L_{c} are, at each occurrence identically or differently, selected from any one or two of the following structures:

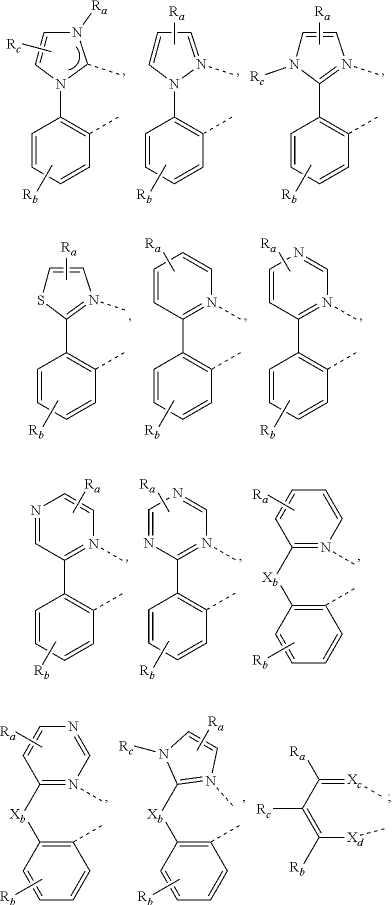

wherein

Rₐ, R_{b} and R_{c} represent, at each occurrence identically or differently, mono-substitution, multiple substitutions or non-substitution;

X_{b} is, at each occurrence identically or differently, selected from the group consisting of: 0, S, Se, NR_{N1} and CR_{C1}R_{C2};

X_{c} and X_{d} are, at each occurrence identically or differently, selected from the group consisting of: O, S, Se and NR_{N2};

Rₐ, R_{b}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and

adjacent substituents Rₐ, R_{b}, R_{N1}, R_{N2}, R_{C1} and R_{C2} can be optionally joined to form a ring.

In this embodiment, the expression that “adjacent substituents Rₐ, R_{b}, R_{c}, R_{N1}, R_{N2}, R_{C1} and R_{C2} can be optionally joined to form a ring” is intended to mean that any one or more of groups of adjacent substituents, such as two substituents Rₐ, two substituents R_{b}, two substituents substituents Rₐ and R_{b}, substituents Rₐ and R_{c}, substituents R_{b} and R_{c}, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{C2}, substituents R_{C1} and R_{C2}, substituents Rₐ and R_{N2}, and substituents R_{b} and R_{N2}, may be joined to form a ring. Obviously, it is possible that none of these substituents are joined to form a ring.

According to an embodiment of the present disclosure, the first metal complex is selected from the group consisting of, but not limited to, GD1 to GD76, wherein the specific structures of GD1 to GD76 are referred to claim 12.

According to an embodiment of the present disclosure, in the organic electroluminescent device, the organic layer is an electron transporting layer and the compound is an electron transporting compound.

According to an embodiment of the present disclosure, in the organic electroluminescent device, the emissive layer further comprises a second compound, wherein the second compound comprises at least one chemical group selected from the group consisting of: benzene, pyridine, pyrimidine, triazine, carbazole, azacarbazole, indolocarbazole, dibenzothiophene, aza-dibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene and combinations thereof.

According to an embodiment of the present disclosure, in the organic electroluminescent device, the emissive layer further comprises a second compound, wherein the second compound comprises at least one chemical group selected from the group consisting of: benzene, carbazole, indolocarbazole, fluorene, silafluorene and combinations thereof.

According to an embodiment of the present disclosure, in the organic electroluminescent device, the emissive layer further comprises a second compound, wherein the compound having a structure of Formula 1 and the second compound may be simultaneously deposited from two evaporation sources respectively to form the emissive layer, or the compound having a structure of Formula 1 and the second compound may be pre-mixed and stably co-deposited from a single evaporation source to form the emissive layer, the latter of which can further save an evaporation source.

According to an embodiment of the present disclosure, in the organic electroluminescent device, the second compound has a structure represented by Formula 3:

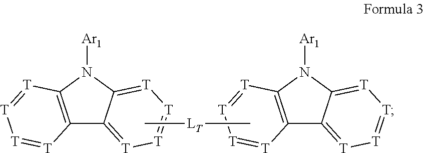

wherein

L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof;

T is, at each occurrence identically or differently, selected from C, CRₜ or N;

Rₜ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and

adjacent substituents Rₜ can be optionally joined to form a ring.

In the present disclosure, the expression that “adjacent substituents Rₜ can be optionally joined to form a ring” is intended to mean that any one or more of groups of adjacent substituents, such as any two substituents Rₜ, may be joined to form a ring. Obviously, it is possible that none of these substituents are joined to form a ring.

According to an embodiment of the present disclosure, in the organic electroluminescent device, the second compound has a structure represented by Formula 4:

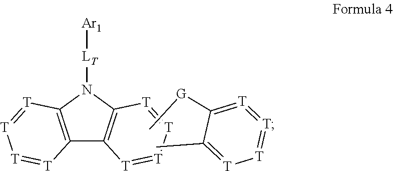

wherein

G is, at each occurrence identically or differently, selected from C(R_{g})₂, NR_{g}, O or S;

T is, at each occurrence identically or differently, selected from C, CRₜ or N;

L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof;

Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and

adjacent substituents Rₜ, R_{g} can be optionally joined to form a ring.

In the present disclosure, the expression that “adjacent substituents Rₜ, R_{g} can be optionally joined to form a ring” is intended to mean that any one or more of groups of adjacent substituents, such as adjacent substituents Rₜ and adjacent substituents Rₜ and R_{g}, may be joined to form a ring. Obviously, it is possible that none of these substituents are joined to form a ring.

According to an embodiment of the present disclosure, in the organic electroluminescent device, the second compound has a structure represented by one of Formulas 3-a to 3-j:

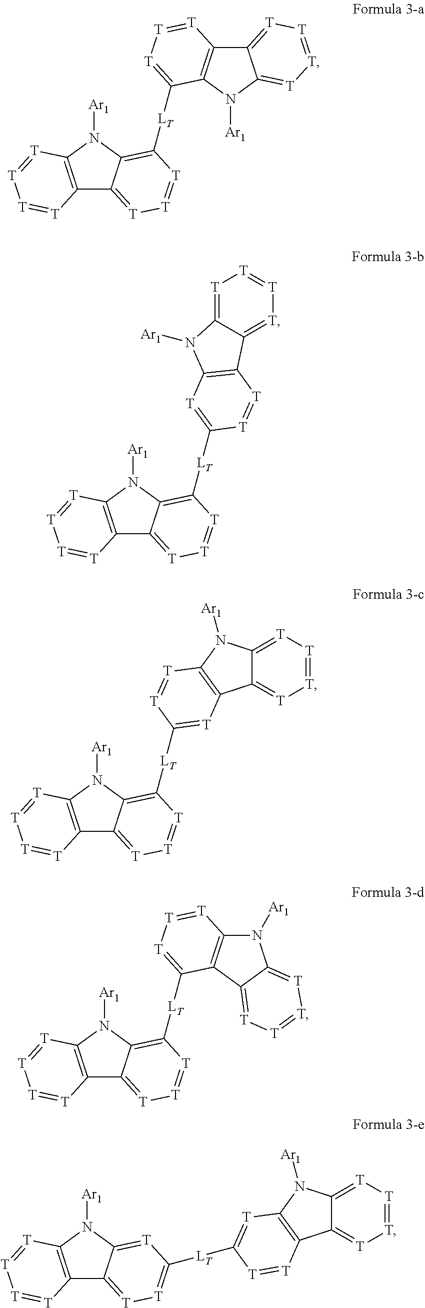

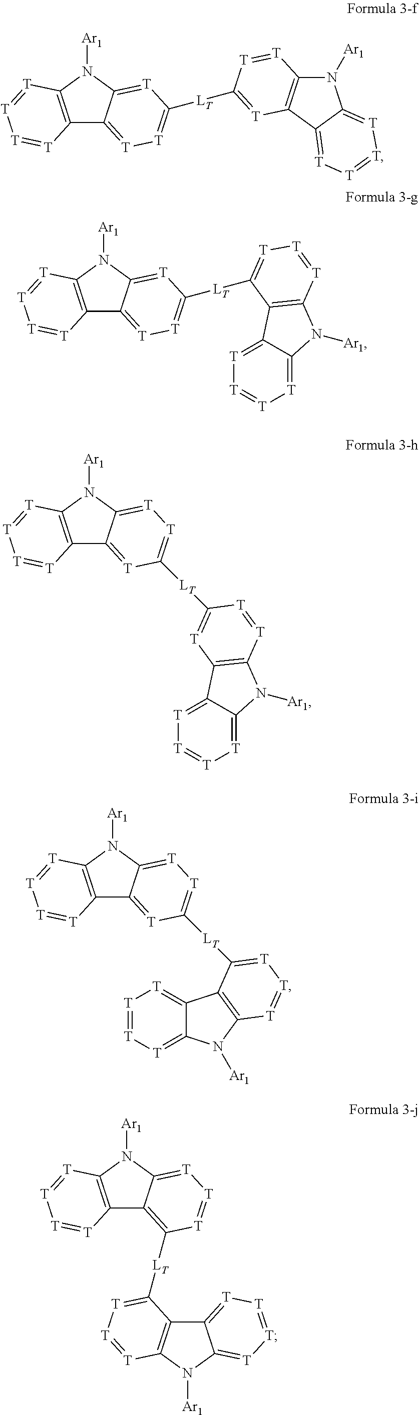

wherein

L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof;

T is, at each occurrence identically or differently, selected from CRₜ or N;

Rₜ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

Arₜ is, at each occurrence identically or differently, selected from substituted or unsubstituted aryl having 6 to 30 carbon atoms, substituted or unsubstituted heteroaryl having 3 to 30 carbon atoms or a combination thereof; and

adjacent substituents Rₜ can be optionally joined to form a ring.

According to an embodiment of the present disclosure, in the organic electroluminescent device, the second compound has a structure represented by one of Formulas 4-a to 4-f:

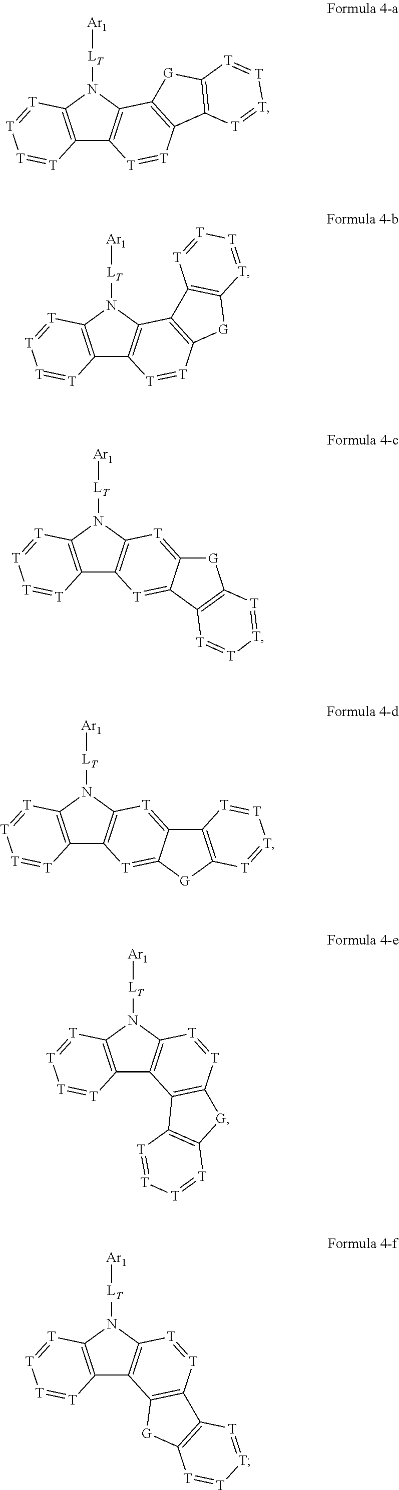

wherein

G is, at each occurrence identically or differently, selected from C(R_{g})₂, NR_{g}, O or S;

T is, at each occurrence identically or differently, selected from CRₜ or N;

L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof;

Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;

Ar₁ is, at each occurrence identically or differently, selected from substituted or unsubstituted aryl having 6 to 30 carbon atoms, substituted or unsubstituted heteroaryl having 3 to 30 carbon atoms or a combination thereof; and

adjacent substituents Rₜ, R_{g} can be optionally joined to form a ring.

According to an embodiment of the present disclosure, at least one of all T is selected from N. For example, one of all T is N, or two of all T are N.

According to an embodiment of the present disclosure, the organic electroluminescent device emits green light.

According to an embodiment of the present disclosure, the organic electroluminescent device emits white light.

According to an embodiment of the present disclosure, the first metal complex is doped in the compound and the second compound, wherein the first metal complex occupies 1% to 30% of the total weight of the emissive layer.

According to an embodiment of the present disclosure, the first metal complex is doped in the compound and the second compound, wherein the first metal complex occupies 3% to 13% of the total weight of the emissive layer.

According to an embodiment of the present disclosure, disclosed is a compound composition comprising the compound in any one of the preceding embodiments.

According to an embodiment of the present disclosure, disclosed is an electronic device comprising the organic electroluminescent device in any one of the preceding embodiments.

Combination with Other Materials

The materials described in the present disclosure for a particular layer in an organic light-emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light-emitting device may be used in combination with a variety of other materials present in the device. For example, dopants disclosed herein may be used in combination with a wide variety of hosts, transport layers, blocking layers, injection layers, electrodes and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FATAR, life testing system produced by SUZHOU FATAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this patent.

In the preparation of a device, when two or more than two host materials together with a luminescent material are to be co-deposited to form an emissive layer, this may be implemented through either of the following manners: (1) co-depositing the two or more than two host materials and the luminescent material from respective evaporation sources, to 46 form the emissive layer; or (2) pre-mixing the two or more than two host materials to obtain a pre-mixture, and co-depositing the pre-mixture from an evaporation source with the luminescent material from another evaporation source, to form the emissive layer. The latter pre-mixing method further save evaporation sources. In the present disclosure, it may be 5 implemented through either of the following manners: (1) co-depositing the first host material, the second host material and the luminescent material from respective evaporation sources, to form the emissive layer; or (2) pre-mixing the first host material and the second host material to obtain a pre-mixture, and co-depositing the pre-mixture from an evaporation source with the luminescent material from another evaporation source, to form the emissive layer.

Material Synthesis Example

The method for preparing a compound in the present disclosure is not limited herein. Typically, the following compounds are used as examples without limitation, and synthesis routes and preparation methods thereof are described below.

### Synthesis Example 1: Synthesis of Compound A-2

Step 1: Synthesis of Intermediate C

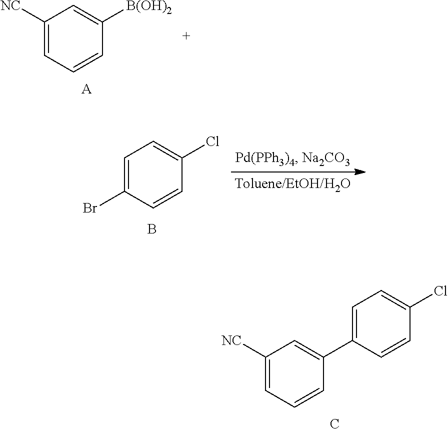

In a 500 mL three-necked round-bottom flask, A (25 g, 170 mmol), B (39 g, 204 mmol), Pd(PPh₃)₄ (3.93 g, 3.4 mmol) and Na₂CO₃ (36 g, 340 mmol) were added to toluene (80 mL), EtOH (20 mL) and H₂O (20 mL), purged three times with N₂, and heated to reflux overnight under N₂ protection. After the reaction was completed as confirmed by TLC plate, heating was stopped, and the solution was cooled to room temperature. Layers were separated, and the aqueous phase was extracted with DCM. Organic phases were combined, dried over anhydrous Na₂SO₄, filtered and concentrated under reduced pressure. The crude product was subjected to silica gel column chromatography (PE:DCM=5:1 to 3:1) to obtain Intermediate C (33.3 g, 155.8 mmol) as a white solid with a yield of 91.6%.

Step 2: Synthesis of Intermediate E

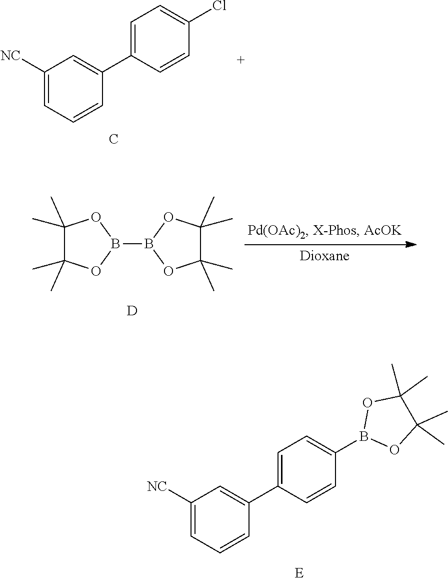

In a 1000 mL three-necked round-bottom flask, C (33.3 g, 155.8 mmol), D (59.3 g, 233.7 mmol), Pd(OAc)₂ (0.7 g, 3.1 mmol), 2-dicyclohexylphosphino-2,4,6-triisopropylbiphenyl (X-Phos) (3.0 g, 6.2 mmol) and AcOK (31 g, 311.6 mmol) were added to 1,4-dioxane (300 mL), purged three times with N₂, and heated to reflux overnight under N₂ protection. After the reaction was completed as confirmed by TLC plate, heating was stopped and the solution was cooled to room temperature. The reaction system was filtered through Celite, the filtrate was concentrated under reduced pressure, and the crude product was subjected to silica gel column chromatography (PE:DCM=5:1 to 2:1) to obtain Intermediate E (28.2 g, 92.5 mmol) as a white solid with a yield of 59.0%.

Step 3: Synthesis of Intermediate G

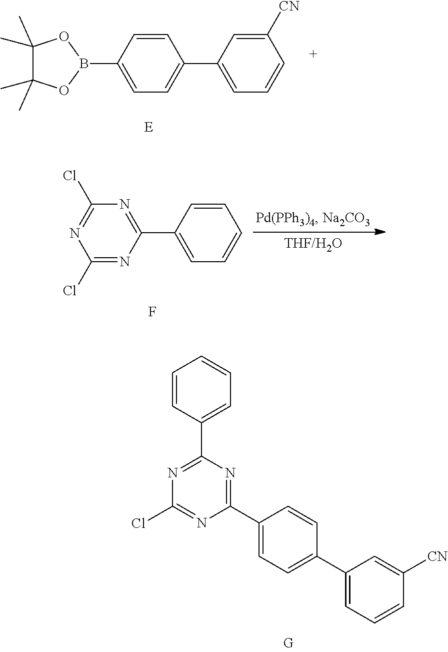

In a 1000 mL three-necked round-bottom flask, E (24.4 g, 80 mmol), F (27 g, 120 mmol), Pd(PPh₃)₄ (1.85 g, 1.6 mmol) and Na₂CO₃ (25 g, 240 mmol) were added to THF (400 mL) and H₂O (100 mL), purged three times with N₂, and heated to reflux overnight under N₂ protection. After the reaction was completed as confirmed by TLC plate, heating was stopped, and the solution was cooled to room temperature. Layers were separated, and the aqueous phase was extracted with DCM. Organic phases were combined, dried over anhydrous Na₂SO₄, filtered and concentrated under reduced pressure. The crude product was subjected to silica gel column chromatography (PE:DCM=5:1 to 1:1) to obtain Intermediate G (12.2 g, 33 mmol) as a white solid with a yield of 41.3%.

Step 4: Synthesis of Compound A-2

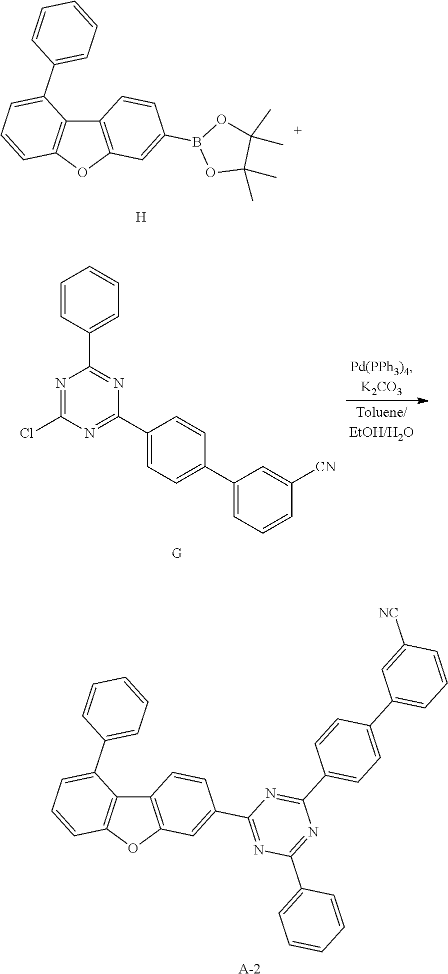

In a 250 mL three-necked round-bottom flask, H (3.52 g, 9.5 mmol), G (3.5 g, 9.5 mmol), Pd(PPh₃)₄ (0.22 g, 0.19 mmol) and K₂CO₃ (2.62 g, 19.0 mmol) were added to toluene (40 mL), EtOH (10 mL) and H₂O (10 mL), purged three times with N₂, and heated to reflux overnight under N₂ protection. After the reaction was completed as confirmed by TLC plate, heating was stopped, the system was cooled to room temperature and suction-filtered under reduced pressure, and the obtained solid was washed with water and ethanol in sequence. The solid was recrystallized from toluene/acetonitrile to obtain a white solid (5.0 g, 8.7 mmol) with a yield of 91.0%. The product was confirmed as the target product, Compound A-2, with a molecular weight of 576.2.

Synthesis Example 2: Synthesis of Compound A-5

Step 1: Synthesis of Intermediate J

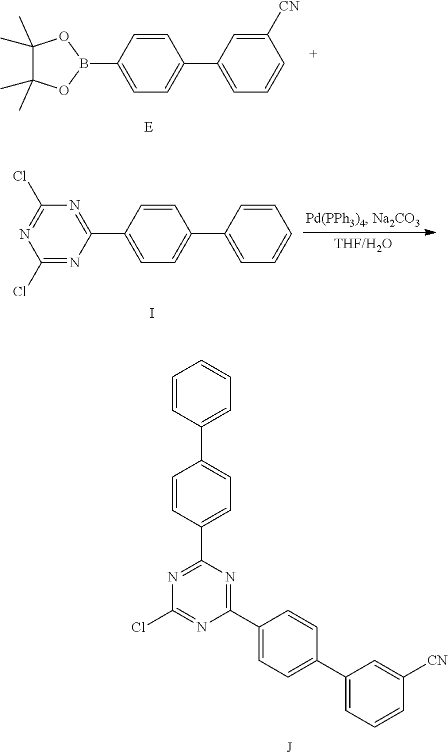

In a 1000 mL three-necked round-bottom flask, E (10.0 g, 32.8 mmol), I (11.9 g, 39.3 mmol), Pd(PPh₃)₄ (1.1 g, 0.98 mmol) and Na₂CO₃ (6.9 g, 65.6 mmol) were added to THF (320 mL) and H₂O (80 mL), purged three times with N₂, and heated to reflux overnight under N₂ protection. After the reaction was completed as confirmed by TLC plate, heating was stopped, and the solution was cooled to room temperature. Layers were separated, and the aqueous phase was extracted with DCM. Organic phases were combined, dried over anhydrous Na₂SO₄, filtered and concentrated under reduced pressure. The crude product was subjected to silica gel column chromatography (PE:DCM=5:1 to 1:1) to obtain Intermediate J (4.0 g, 9.0 mmol) as a white solid with a yield of 27.4%.

Step 2: Synthesis of Compound A-5

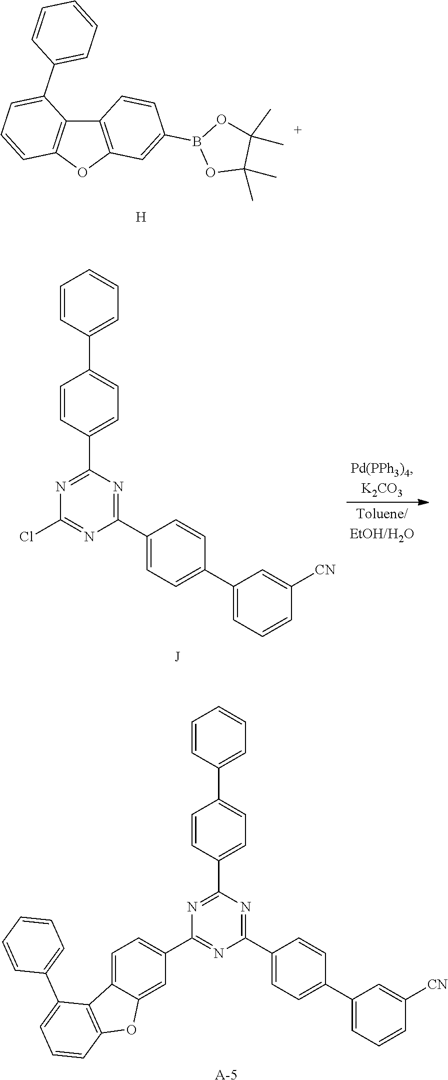

In a 250 mL three-necked round-bottom flask, H (3.3 g, 9.0 mmol), J (4.0 g, 9.0 mmol), Pd(PPh₃)₄ (0.21 g, 0.18 mmol) and K₂CO₃ (2.5 g, 18.0 mmol) were added to toluene (60 mL), EtOH (15 mL) and H₂O (15 mL), purged three times with N₂, and heated to reflux overnight under N₂ protection. After the reaction was completed as confirmed by TLC plate, heating was stopped, the system was cooled to room temperature and suction-filtered under reduced pressure, and the obtained solid was washed with water and methanol in sequence. The solid was recrystallized from toluene to obtain a white solid (4.0 g, 6.1 mmol) with a yield of 68.0%. The product was confirmed as the target product, Compound A-5, with a molecular weight of 652.2.

### Synthesis Example 3: Synthesis of Compound A-8

Step 1: Synthesis of Intermediate L

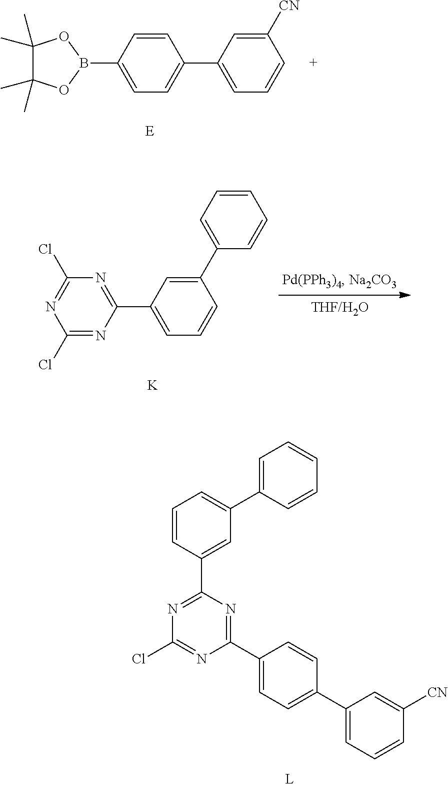

In a 1000 mL three-necked round-bottom flask, E (10.0 g, 32.8 mmol), K (11.9 g, 39.3 mmol), Pd(PPh₃)₄ (1.1 g, 0.98 mmol) and Na₂CO₃ (6.9 g, 65.6 mmol) were added to THF (320 mL) and H₂O (80 mL), purged three times with N₂, and heated to reflux overnight under N₂ protection. After the reaction was completed as confirmed by TLC plate, heating was stopped, and the solution was cooled to room temperature. Layers were separated, and the aqueous phase was extracted with DCM. Organic phases were combined, dried over anhydrous Na₂SO₄, filtered and concentrated under reduced pressure. The crude product was subjected to silica gel column chromatography (PE:DCM=5:1 to 1:1) to obtain Intermediate L (4.0 g, 9.0 mmol) as a white solid with a yield of 27.4%.

Step 2: Synthesis of Compound A-8

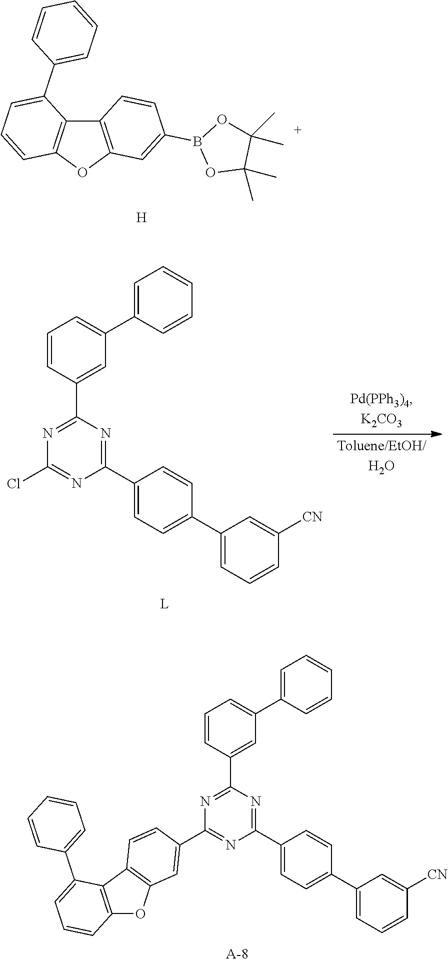

In a 250 mL three-necked round-bottom flask, H (3.3 g, 9.0 mmol), L (4.0 g, 9.0 mmol), Pd(PPh₃)₄ (0.21 g, 0.18 mmol) and K₂CO₃ (2.5 g, 18.0 mmol) were added to toluene (60 mL), EtOH (15 mL) and H₂O (15 mL), purged three times with N₂, and heated to reflux overnight under N₂ protection. After the reaction was completed as confirmed by TLC plate, heating was stopped, the system was cooled to room temperature and suction-filtered under reduced pressure, and the obtained solid was washed with water and methanol in sequence. The solid was recrystallized from toluene to obtain a white solid (3.9 g, 6.0 mmol) with a yield of 66.7%. The product was confirmed as the target product, Compound A-8, with a molecular weight of 652.2.

### Synthesis Example 4: Synthesis of Compound A-57

Step 1: Synthesis of Intermediate N

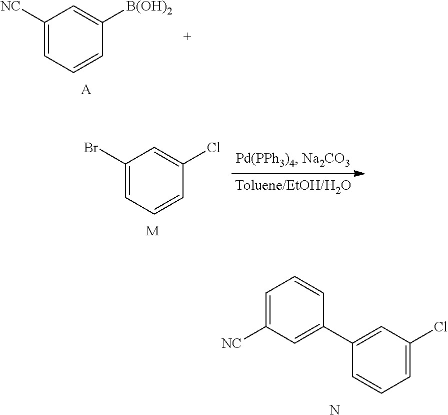

In a 1000 mL three-necked round-bottom flask, A (20.0 g, 136.1 mmol), M (31.3 g, 163.3 mmol), Pd(PPh₃)₄ (1.57 g, 1.36 mmol) and Na₂CO₃ (28.9 g, 272.2 mmol) were added to toluene (280 mL), EtOH (70 mL) and H₂O (70 mL), purged three times with N₂, and heated to reflux overnight under N₂ protection. After the reaction was completed as confirmed by TLC plate, heating was stopped, and the solution was cooled to room temperature. Layers were separated, and the aqueous phase was extracted with DCM. Organic phases were combined, dried over anhydrous Na₂SO₄, filtered and concentrated under reduced pressure. The crude product was subjected to silica gel column chromatography (PE:DCM=5:1 to 3:1) to obtain Intermediate N (26.0 g, 121.8 mmol) as a white solid with a yield of 91.6%.

Step 2: Synthesis of Intermediate O

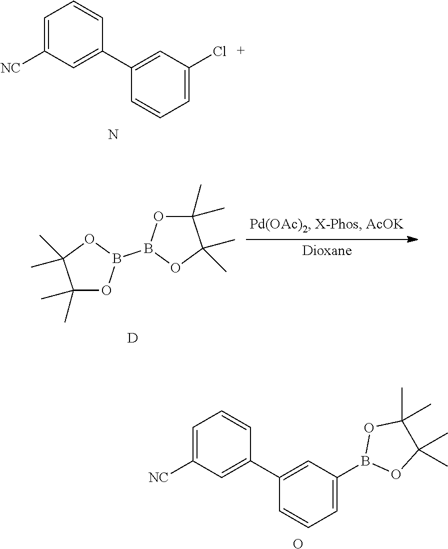

In a 500 mL three-necked round-bottom flask, N (26.0 g, 121.8 mmol), D (61.9 g, 243.6 mmol), Pd(OAc)₂ (1.4 g, 6.1 mmol), 2-dicyclohexylphosphino-2,4,6-triisopropylbiphenyl (X-Phos) (5.8 g, 12.2 mmol) and AcOK (23.9 g, 243.6 mmol) were added to 1,4-dioxane (200 mL), purged three times with N₂, and heated to reflux overnight under N₂ protection. After the reaction was completed as confirmed by TLC plate, heating was stopped and the solution was cooled to room temperature. The reaction system was filtered through Celite, the filtrate was concentrated under reduced pressure, and the crude product was subjected to silica gel column chromatography (PE:DCM=5:1 to 2:1) to obtain Intermediate 0 (28.0 g, 91.7 mmol) as a white solid with a yield of 75.3%.

Step 3: Synthesis of Intermediate P

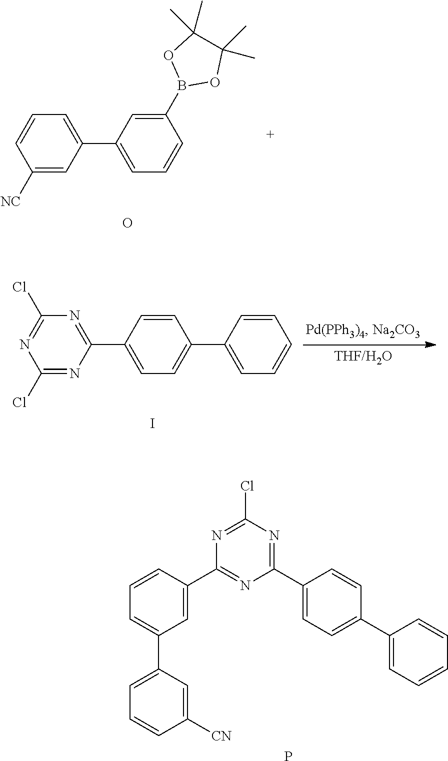

In a 500 mL three-necked round-bottom flask, 0 (6.1 g, 20.0 mmol), I (9.1 g, 30.0 mmol), Pd(PPh₃)₄ (1.1 g, 0.95 mmol) and Na₂CO₃ (6.4 g, 60.0 mmol) were added to THF (120 mL) and H₂O (30 mL), purged three times with N₂, and heated to reflux overnight under N₂ protection. After the reaction was completed as confirmed by TLC plate, heating was stopped, and the solution was cooled to room temperature. Layers were separated, and the aqueous phase was extracted with DCM. Organic phases were combined, dried over anhydrous Na₂SO₄, filtered and concentrated under reduced pressure. The crude product was subjected to silica gel column chromatography (PE:DCM=3:1 to 1:1) to obtain Intermediate P (6.0 g, 13.5 mmol) as a white solid with a yield of 67.5%.

Step 4: Synthesis of Compound A-57

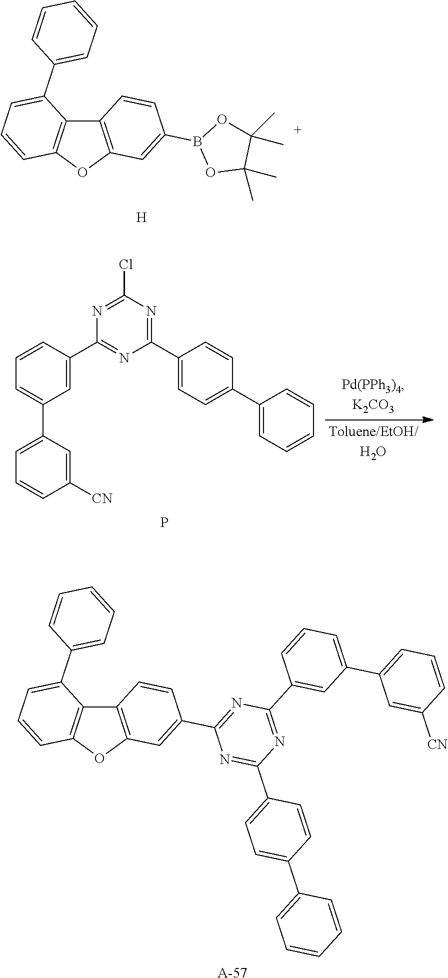

In a 250 mL three-necked round-bottom flask, H (3.7 g, 9.9 mmol), P (4.2 g, 9.4 mmol), Pd(PPh₃)₄ (0.54 g, 0.47 mmol) and K₂CO₃ (3.9 g, 28.2 mmol) were added to toluene (80 mL), EtOH (20 mL) and H₂O (20 mL), purged three times with N₂, and heated to reflux overnight under N₂ protection. After the reaction was completed as confirmed by TLC plate, heating was stopped, the system was cooled to room temperature and suction-filtered under reduced pressure, and the obtained solid was washed with water and methanol in sequence. The solid was recrystallized from toluene to obtain a white solid (4.7 g, 7.2 mmol) with a yield of 76.5%. The product was confirmed as the target product, Compound A-57, with a molecular weight of 652.2.

### Synthesis Example 5: Synthesis of Compound A-60

Step 1: Synthesis of Intermediate Q

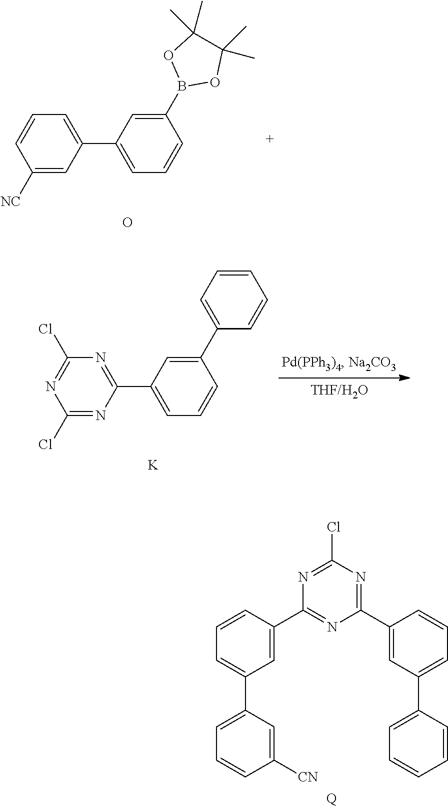

In a 250 mL three-necked round-bottom flask, 0 (6.1 g, 20.0 mmol), K (9.1 g, 30.0 mmol), Pd(PPh₃)₄ (1.1 g, 0.98 mmol) and Na₂CO₃ (6.4 g, 60.0 mmol) were added to THF (120 mL) and H₂O (30 mL), purged three times with N₂, and heated to reflux overnight under N₂ protection. After the reaction was completed as confirmed by TLC plate, heating was stopped, and the solution was cooled to room temperature. Layers were separated, and the aqueous phase was extracted with DCM. Organic phases were combined, dried over anhydrous Na₂SO₄, filtered and concentrated under reduced pressure. The crude product was subjected to silica gel column chromatography (PE:DCM=5:1 to 1:1) to obtain Intermediate Q (6.0 g, 13.5 mmol) as a white solid with a yield of 67.5%.

Step 2: Synthesis of Compound A-60

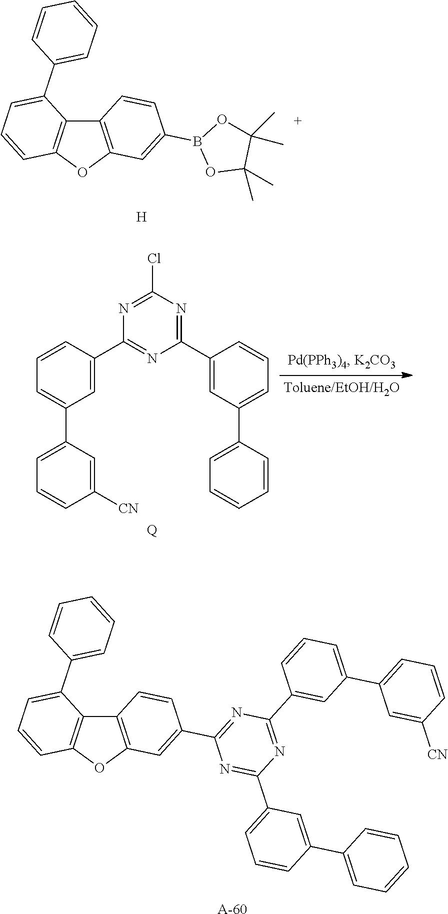

In a 250 mL three-necked round-bottom flask, H (3.9 g, 10.5 mmol), Q (4.45 g, 10.0 mmol), Pd(PPh₃)₄ (0.54 g, 0.47 mmol) and K₂CO₃ (3.9 g, 28.2 mmol) were added to toluene (80 mL), EtOH (20 mL) and H₂O (20 mL), purged three times with N₂, and heated to reflux overnight under N₂ protection. After the reaction was completed as confirmed by TLC plate, heating was stopped, the system was cooled to room temperature and suction-filtered under reduced pressure, and the obtained solid was washed with water and methanol in sequence. The solid was recrystallized from toluene to obtain a white solid (5.7 g, 8.7 mmol) with a yield of 87.0%. The product was confirmed as the target product, Compound A-60, with a molecular weight of 652.2.

### Synthesis Example 6: Synthesis of Compound A-177

Step 1: Synthesis of Intermediate S

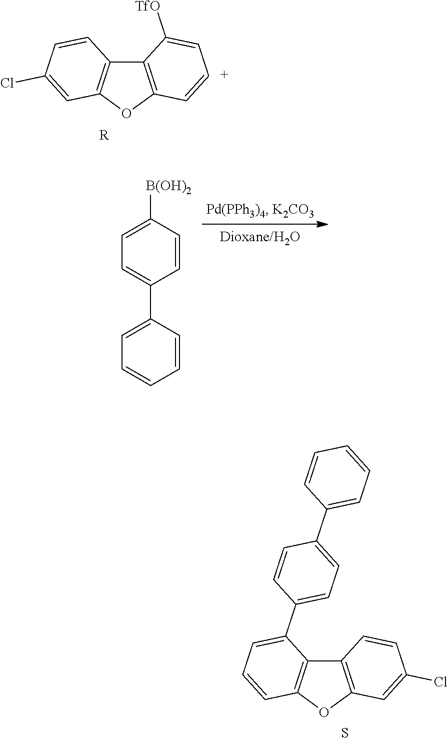

In a 250 mL three-necked round-bottom flask, R (8.0 g, 22.9 mmol), 4-biphenylboronic acid (5.9 g, 29.7 mmol), Pd(PPh₃)₄ (1.3 g, 1.1 mmol) and K₂CO₃ (9.5 g, 68.7 mmol) were added to 1,4-dioxane (100 mL) and H₂O (25 mL) and heated to reflux overnight under nitrogen protection. Heating was stopped and the system was cooled to room temperature. The organic phase was taken, and the aqueous phase was added with DCM and extracted multiple times. Organic phases were combined, dried over anhydrous Na₂SO₄, filtered and concentrated under reduced pressure. The crude product was purified through column chromatography (PE:DCM=40:1 to 15:1) to obtain Intermediate S (7.0 g, 19.7 mmol) as a white solid with a yield of 86.1%.

Step 2: Synthesis of Intermediate T

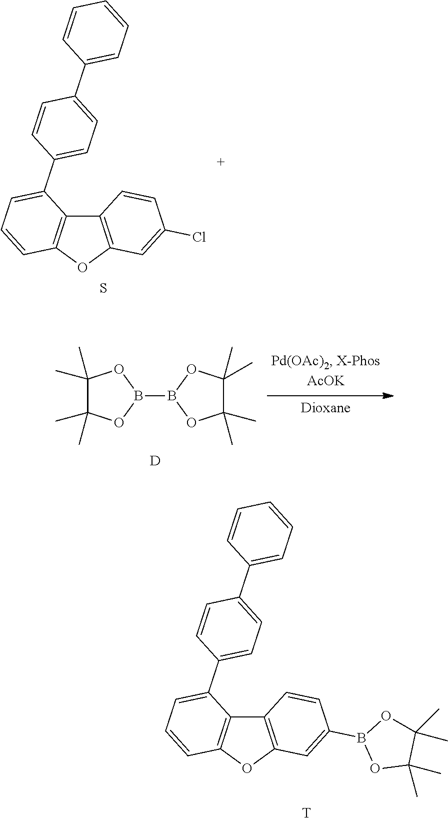

In a 250 mL three-necked round-bottom flask, S (7.0 g, 19.7 mmol), D (10.0 g, 39.4 mmol), Pd(OAc)₂ (0.2 g, 1.0 mmol), 2-dicyclohexylphosphino-2,4,6-triisopropylbiphenyl (X-Phos) (0.9 g, 2.0 mmol) and AcOK (5.8 g, 59.1 mmol) were added to 1,4-dioxane (100 mL) and heated to reflux overnight under nitrogen protection. Heating was stopped and the system was cooled to room temperature. The reaction system was filtered through Celite. The filtrate was concentrated under reduced pressure. The crude product was purified through column chromatography (PE:DCM=4:1 to 2:1) to obtain Intermediate T (6.0 g, 13.4 mmol) as a white solid with a yield of 68.2%.

Step 3: Synthesis of Compound A-177

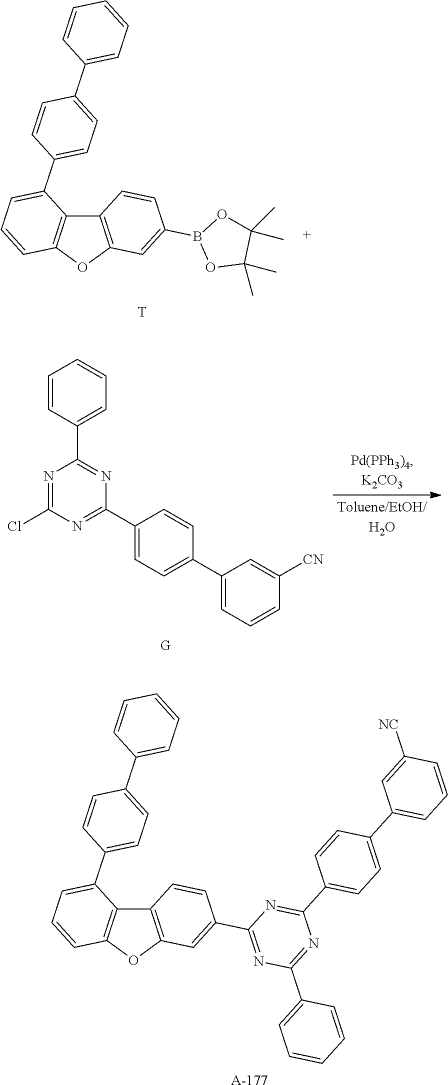

In a 250 mL three-necked round-bottom flask, T (4.5 g, 10.0 mmol), G (3.5 g, 9.5 mmol), Pd(PPh₃)₄ (0.5 g, 0.43 mmol) and K₂CO₃ (3.9 g, 28.5 mmol) were added to toluene (80 mL), EtOH (20 mL) and H₂O (20 mL), purged three times with N₂, and heated to reflux overnight under N₂ protection. After the reaction was completed as confirmed by TLC plate, heating was stopped, the system was cooled to room temperature and suction-filtered under reduced pressure, and the obtained solid was washed with water and ethanol in sequence. The solid was recrystallized from toluene/acetonitrile to obtain a white solid (4.5 g, 6.9 mmol) with a yield of 72.6%. The product was confirmed as the target product, Compound A-177, with a molecular weight of 652.2.

### Synthesis Example 7: Synthesis of Compound A-352

Step 1: Synthesis of Intermediate U

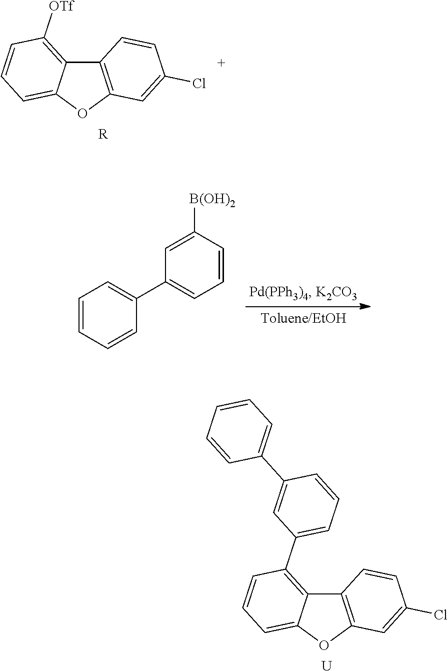

In a 250 mL three-necked round-bottom flask, R (6.0 g, 17.1 mmol), 3-biphenylboronic acid (3.70 g, 18.81 mmol), Pd(PPh₃)₄ (0.59 g, 0.51 mmol) and K₂CO₃ (4.72 g, 34.2 mmol) were added to toluene (58 mL), EtOH (14 mL) and H₂O (14 mL) and heated to reflux overnight under nitrogen protection. Heating was stopped and the system was cooled to room temperature. The organic phase was taken, and the aqueous phase was added with DCM and extracted multiple times. Organic phases were combined, dried over anhydrous Na₂SO₄, filtered and concentrated under reduced pressure. The crude product was purified through column chromatography (PE:DCM=50:1) to obtain Intermediate U (5.6 g, 15.8 mmol) as a colorless oil with a yield of 92.3%.

Step 2: Synthesis of Intermediate V

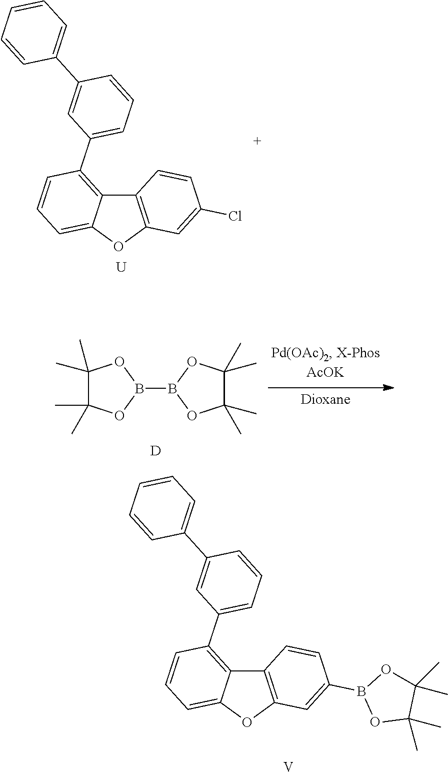

In a 250 mL three-necked round-bottom flask, U (6.0 g, 17.47 mmol), D (6.65 g, 26.2 mmol), Pd(OAc)₂ (0.08 g, 0.35 mmol), 2-dicyclohexylphosphino-2,4,6-triisopropylbiphenyl (X-Phos) (0.33 g, 0.67 mmol) and AcOK (3.43 g, 34.94 mmol) were added to 1,4-dioxane (87 mL) and heated to reflux overnight under nitrogen protection. Heating was stopped and the system was cooled to room temperature. The reaction system was filtered through Celite. The filtrate was concentrated under reduced pressure. The crude product was purified through column chromatography (PE:DCM=4:1 to 2:1) to obtain Intermediate V (4.71 g, 10.55 mmol) as a white solid with a yield of 60.4%.

Step 3: Synthesis of Compound A-352

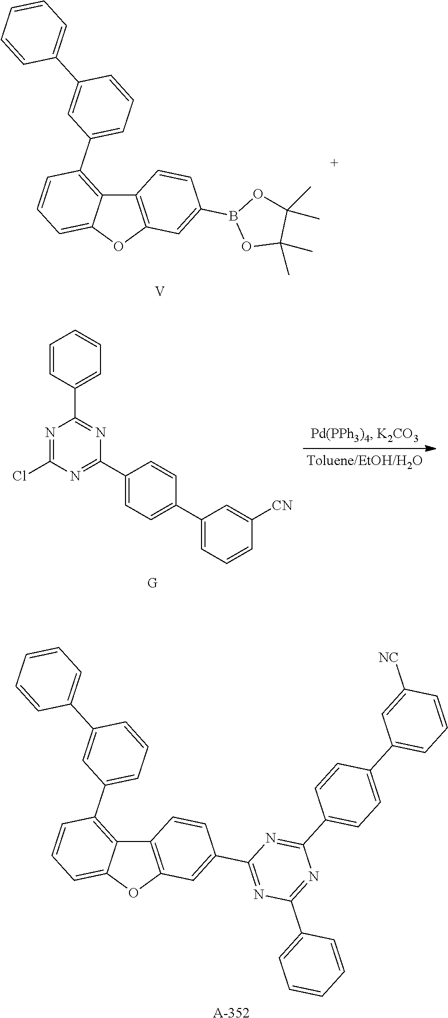

In a 250 mL three-necked round-bottom flask, V (4.46 g, 10.0 mmol), G (3.68 g, 10.0 mmol), Pd(PPh₃)₄ (0.23 g, 0.20 mmol) and K₂CO₃ (2.76 g, 20.0 mmol) were added to toluene (48 mL), EtOH (12 mL) and H₂O (12 mL), purged three times with N₂, and heated to reflux overnight under N₂ protection. After the reaction was completed as confirmed by TLC plate, heating was stopped, the system was cooled to room temperature and suction-filtered under reduced pressure, and the obtained solid was washed with water and ethanol in sequence. The solid was recrystallized from toluene/acetonitrile to obtain a white solid (5.9 g, 9.0 mmol) with a yield of 90.4%. The product was confirmed as the target product, Compound A-352, with a molecular weight of 652.2.

### Synthesis Example 8: Synthesis of Compound A-1

Step 1: Synthesis of Intermediate X

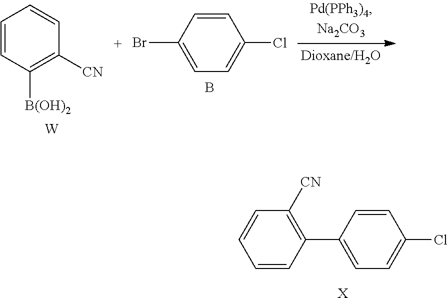

In a 500 mL three-necked round-bottom flask, W (12.5 g, 85.1 mmol), B (15.5 g, 81.0 mmol), Pd(PPh₃)₄ (1.8 g, 1.6 mmol) and Na₂CO₃ (27.9 g, 202.5 mmol) were added to 1,4-dioxane (120 mL) and H₂O (30 mL) and heated to reflux overnight under N₂ protection. After the reaction was completed as confirmed by TLC plate, heating was stopped, the solution was cooled to room temperature, the system was extracted with ethyl acetate, and organic phases were dried over anhydrous Na₂SO₄, filtered and concentrated under reduced pressure. The crude product was subjected to silica gel column chromatography (PE:DCM=5:1 to 3:1) to obtain Intermediate X (6.7 g, 31.4 mmol) as a white solid with a yield of 38.8%.

Step 2: Synthesis of Intermediate Y

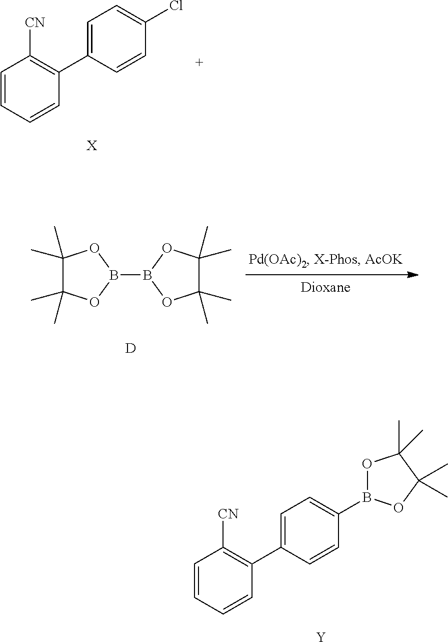

In a 250 mL three-necked round-bottom flask, X (6.7 g, 31.4 mmol), D (12.0 g, 47.1 mmol), Pd(OAc)₂ (0.35 g, 1.6 mmol), 2-dicyclohexylphosphino-2,4,6-triisopropylbiphenyl (X-Phos) (1.5 g, 3.1 mmol) and AcOK (6.2 g, 62.8 mmol) were added to 1,4-dioxane (60 mL) and heated to reflux overnight under N₂ protection. After the reaction was completed as confirmed by TLC plate, heating was stopped and the solution was cooled to room temperature. The reaction system was filtered through Celite, the filtrate was concentrated under reduced pressure, and the crude product was subjected to silica gel column chromatography (PE:DCM=5:1 to 2:1) to obtain Intermediate Y (6.9 g, 22.6 mmol) as a white solid with a yield of 72.0%.

Step 3: Synthesis of Intermediate Z

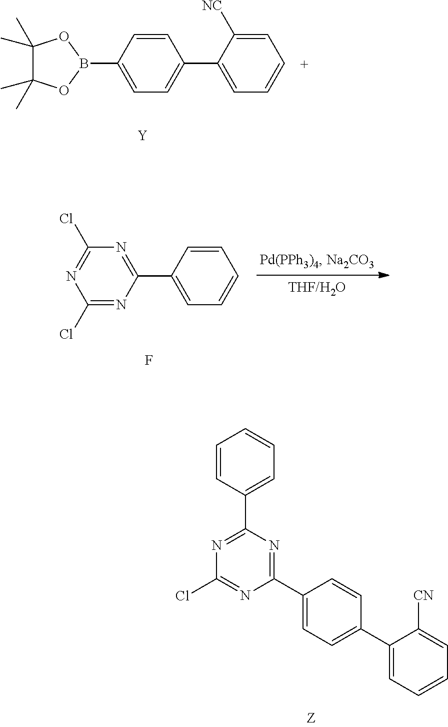

In a 250 mL three-necked round-bottom flask, Y (6.9 g, 22.6 mmol), F (10.2 g, 45.2 mmol), Pd(PPh₃)₄ (1.3 g, 1.1 mmol) and Na₂CO₃ (4.8 g, 45.2 mmol) were added to THF (80 mL) and H₂O (20 mL) and heated to reflux under N₂ protection. After 12 h, the reaction was completed as confirmed by TLC plate, heating was stopped, and the solution was cooled to room temperature. Layers were separated, and the aqueous phase was extracted with DCM. Organic phases were combined, dried over anhydrous Na₂SO₄, filtered and concentrated under reduced pressure. The crude product was subjected to silica gel column chromatography (PE:DCM=2:1 to 1:1) to obtain Intermediate Z (2.8 g, 7.6 mmol) as a white solid with a yield of 33.6%.

Step 4: Synthesis of Compound A-1

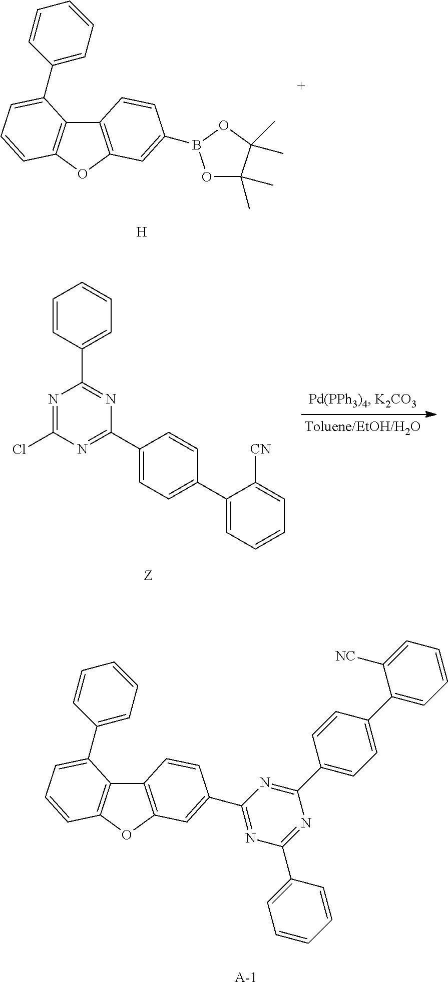

In a 250 mL three-necked round-bottom flask, H (2.75 g, 7.4 mmol), Z (2.74 g, 7.4 mmol), Pd(PPh₃)₄ (0.43 g, 0.37 mmol) and K₂CO₃ (2.0 g, 14.8 mmol) were added to toluene (40 mL), EtOH (10 mL) and H₂O (10 mL), purged three times with N₂, and heated to reflux overnight under N₂ protection. After the reaction was completed as confirmed by TLC plate, heating was stopped, the system was cooled to room temperature and suction-filtered under reduced pressure, and the obtained solid was washed with water and ethanol in sequence. The solid was recrystallized from toluene to obtain a white solid (2.8 g, 4.9 mmol) with a yield of 65.6%. The product was confirmed as the target product, Compound A-1, with a molecular weight of 576.2.

### Synthesis Example 9: Synthesis of Compound A-3

Step 1: Synthesis of Intermediate AB

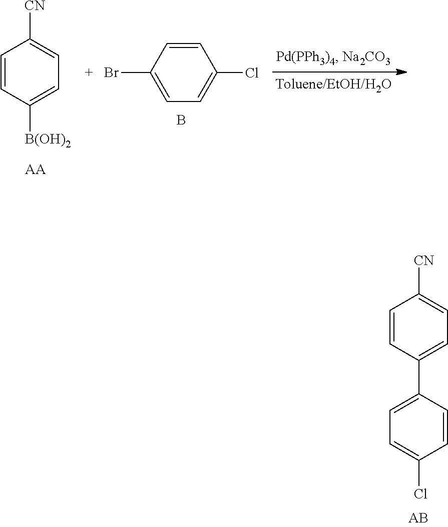

In a 500 mL three-necked round-bottom flask, AA (18.0 g, 122.5 mmol), B (28.0 g, 147.0 mmol), Pd(PPh₃)₄ (2.83 g, 2.45 mmol) and Na₂CO₃ (26.0 g, 245.0 mmol) were added to toluene (120 mL), EtOH (30 mL) and H₂O (30 mL) and heated to reflux overnight under N₂ protection. After the reaction was completed as confirmed by TLC plate, heating was stopped and the solution was cooled to room temperature. The organic phase was taken, and the aqueous phase was added with DCM and extracted multiple times. Organic phases were combined, dried over anhydrous Na₂SO₄, filtered and concentrated under reduced pressure. The crude product was subjected to silica gel column chromatography (PE:DCM=10:1 to 4:1) to obtain Intermediate AB (22.0 g, 103.0 mmol) as a white solid with a yield of 84.1%.

Step 2: Synthesis of Intermediate AC

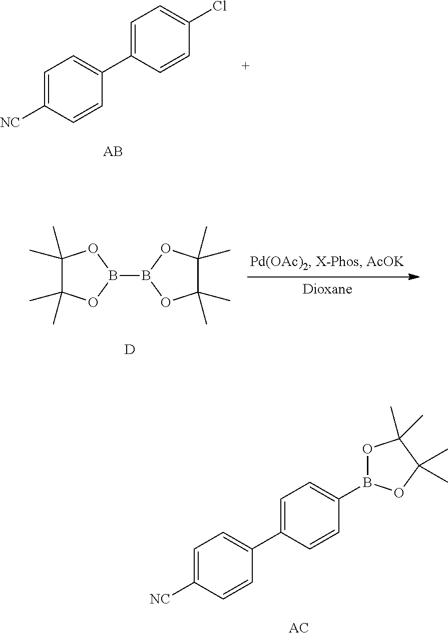

In a 250 mL three-necked round-bottom flask, AB (22.0 g, 103.0 mmol), D (39.2 g, 154.5 mmol), Pd(OAc)₂ (0.46 g, 2.1 mmol), 2-dicyclohexylphosphino-2,4,6-triisopropylbiphenyl (X-Phos) (1.96 g, 4.12 mmol) and AcOK (20.2 g, 206.0 mmol) were added to 1,4-dioxane (200 mL) and heated to reflux overnight under N₂ protection. After the reaction was completed as confirmed by TLC plate, heating was stopped and the solution was cooled to room temperature. The reaction system was filtered through Celite, the filtrate was concentrated under reduced pressure, and the crude product was subjected to silica gel column chromatography (PE:DCM=5:1 to 2:1) to obtain Intermediate AC (28.5 g, 93.4 mmol) as a white solid with a yield of 90.7%.

Step 3: Synthesis of Intermediate AD

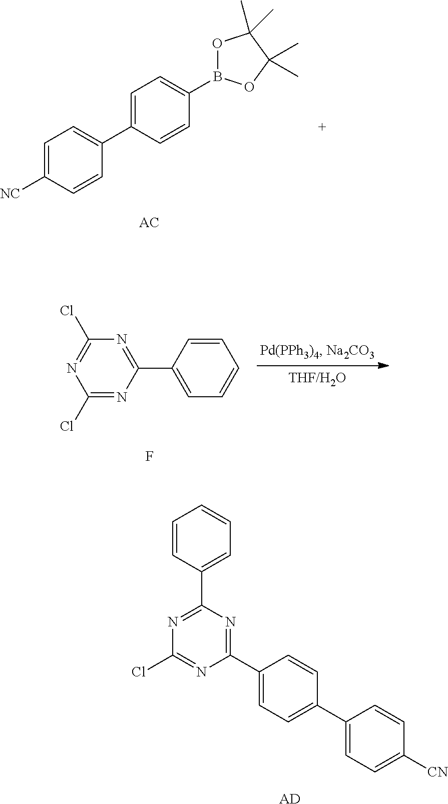

In a 500 mL three-necked round-bottom flask, AC (5.0 g, 16.4 mmol), F (9.3 g, 41.0 mmol), Pd(PPh₃)₄ (0.57 g, 0.49 mmol) and Na₂CO₃ (3.48 g, 32.8 mmol) were added to THF (128 mL) and H₂O (32 mL) and heated to reflux under N₂ protection. After 4 h, the reaction was completed as confirmed by TLC plate, heating was stopped, and the solution was cooled to room temperature. Layers were separated, and the aqueous phase was extracted with DCM. Organic phases were combined, dried over anhydrous Na₂SO₄, filtered and concentrated under reduced pressure. The crude product was subjected to silica gel column chromatography (PE:DCM=3:1 to 1:1) to obtain Intermediate AD (3.7 g, 10.0 mmol) as a white solid with a yield of 61.2%.

Step 4: Synthesis of Compound A-3

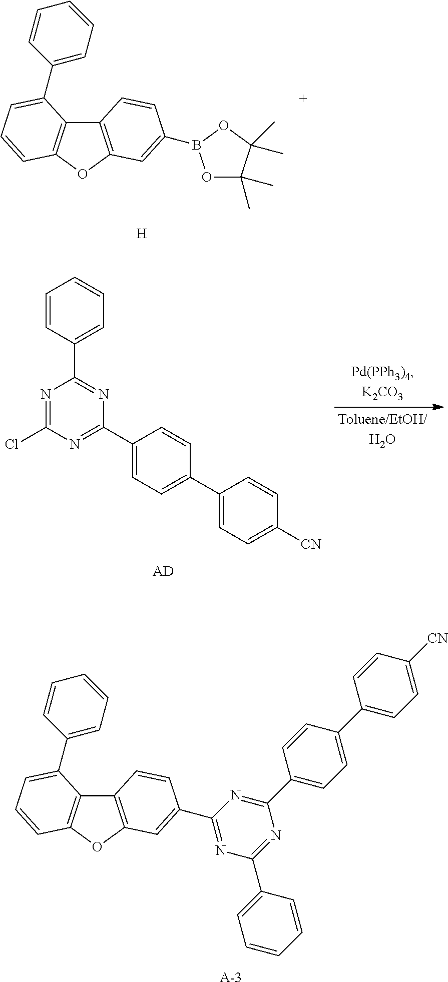

In a 250 mL three-necked round-bottom flask, H (3.70 g, 10.0 mmol), AD (3.69 g, 10.0 mmol), Pd(PPh₃)₄ (0.23 g, 0.20 mmol) and K₂CO₃ (2.76 g, 20.0 mmol) were added to toluene (48 mL), EtOH (12 mL) and H₂O (12 mL), purged three times with N₂, and heated to reflux overnight under N₂ protection. After the reaction was completed as confirmed by TLC plate, heating was stopped, the system was cooled to room temperature and suction-filtered under reduced pressure, and the obtained solid was washed with water and ethanol in sequence.

The solid was recrystallized from toluene to obtain a white solid (5.2 g, 9.0 mmol) with a yield of 90.1%. The product was confirmed as the target product, Compound A-3, with a molecular weight of 576.2.

### Synthesis Example 10: Synthesis of Compound A-54

Step 1: Synthesis of Intermediate AE

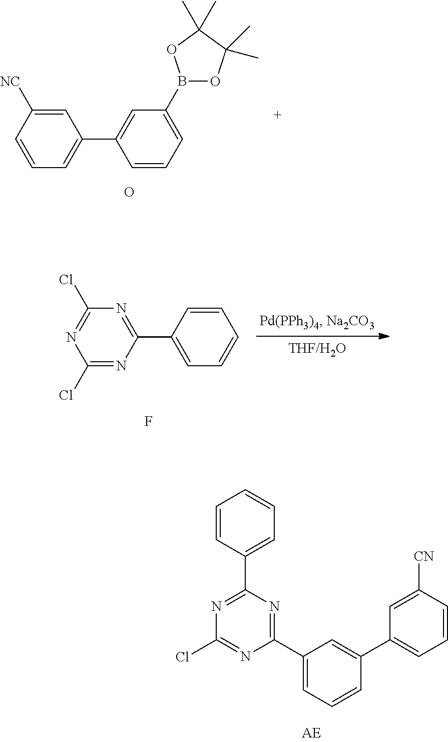

In a 500 mL three-necked round-bottom flask, 0 (14.0 g, 45.87 mmol), F (16.6 g, 73.4 mmol), Pd(PPh₃)₄ (1.59 g, 1.38 mmol) and Na₂CO₃ (9.72 g, 91.74 mmol) were added to THF (240 mL) and H₂O (60 mL) and heated to reflux under N₂ protection. After 8 h, the reaction was completed as confirmed by TLC plate, heating was stopped, and the solution was cooled to room temperature. Layers were separated, and the aqueous phase was extracted with DCM. Organic phases were combined, dried over anhydrous Na₂SO₄, filtered and concentrated under reduced pressure. The crude product was subjected to silica gel column chromatography (PE:DCM=2:1 to 1:1) to obtain Intermediate AE (10.6 g, 28.74 mmol) as a white solid with a yield of 62.7%.

Step 2: Synthesis of Compound A-54

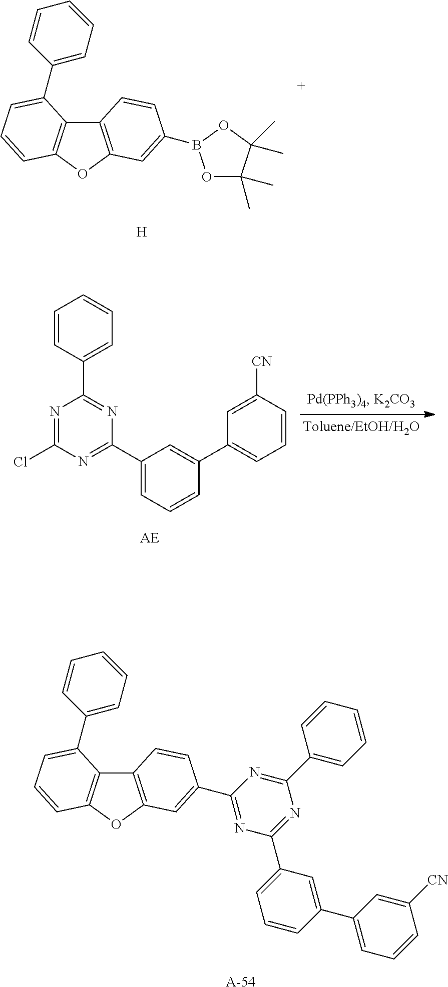

In a 250 mL three-necked round-bottom flask, H (3.70 g, 10.0 mmol), AE (3.69 g, 10.0 mmol), Pd(PPh₃)₄ (0.35 g, 0.30 mmol) and K₂CO₃ (2.76 g, 20.0 mmol) were added to toluene (40 mL), Et0H (10 mL) and H₂O (10 mL) and heated to reflux overnight under N₂ protection.

After the reaction was completed as confirmed by TLC plate, heating was stopped, the system was cooled to room temperature and suction-filtered under reduced pressure, and the obtained solid was washed with water and methanol in sequence. The solid was recrystallized from toluene to obtain a white solid (4.7 g, 8.2 mmol) with a yield of 82.0%. The product was confirmed as the target product, Compound A-54, with a molecular weight of 576.2.

### Synthesis Example 11: Synthesis of Compound A-55

Step 1: Synthesis of Intermediate AF

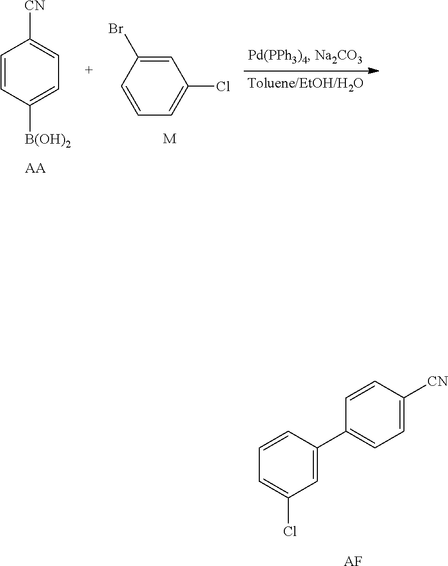

In a 500 mL three-necked round-bottom flask, AA (21.0 g, 143.0 mmol), M (32.8 g, 171.6 mmol), Pd(PPh₃)₄ (3.3 g, 2.86 mmol) and Na₂CO₃ (30.3 g, 286.0 mmol) were added to toluene (200 mL), EtOH (50 mL) and H₂O (50 mL) and heated to reflux overnight under N₂ protection. After the reaction was completed as confirmed by TLC plate, heating was stopped and the solution was cooled to room temperature. The organic phase was taken, and the aqueous phase was added with DCM and extracted multiple times. Organic phases were combined, dried over anhydrous Na₂SO₄, filtered and concentrated under reduced pressure. The crude product was subjected to silica gel column chromatography (PE:DCM=10:1 to 3:1) to obtain Intermediate AF (27.6 g, 129.2 mmol) as a white solid with a yield of 90.3%.

Step 2: Synthesis of Intermediate AG

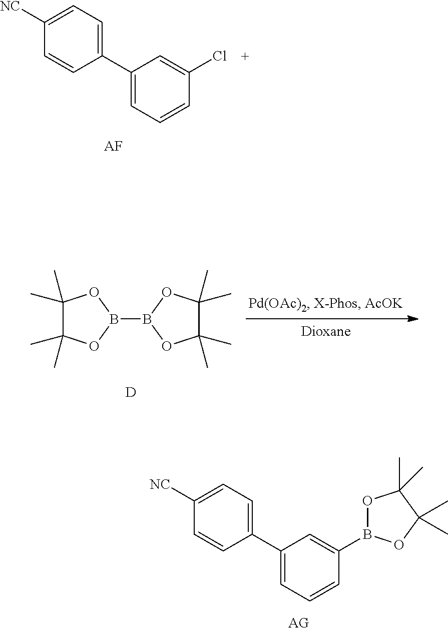

In a 500 mL three-necked round-bottom flask, AF (27.6 g, 129.2 mmol), D (49.2 g, 193.8 mmol), Pd(OAc)₂ (0.58 g, 2.58 mmol), 2-dicyclohexylphosphino-2,4,6-triisopropylbiphenyl (X-Phos) (2.46 g, 5.16 mmol) and AcOK (25.2 g, 256.4 mmol) were added to 1,4-dioxane (260 mL) and heated to reflux overnight under

N₂ protection. After the reaction was completed as confirmed by TLC plate, heating was stopped and the solution was cooled to room temperature. The reaction system was filtered through Celite, the filtrate was concentrated under reduced pressure, and the crude product was subjected to silica gel column chromatography (PE:DCM=5:1 to 2:1) to obtain Intermediate AG (34.2 g, 112.0 mmol) as a white solid with a yield of 86.7%.

Step 3: Synthesis of Intermediate AH

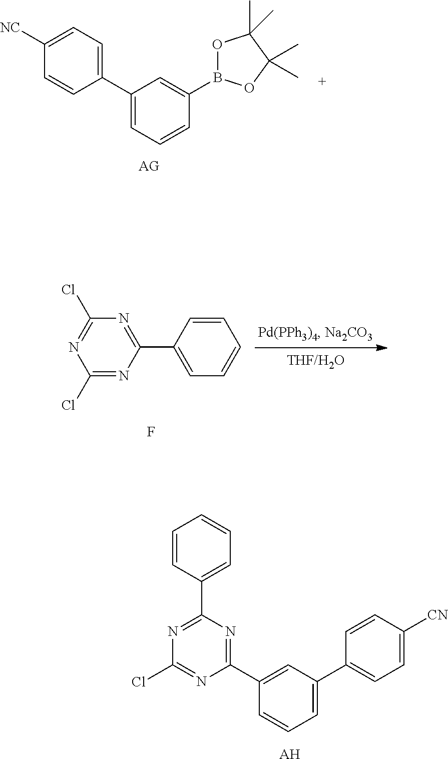

In a 500 mL three-necked round-bottom flask, AG (12.2 g, 40.0 mmol), F (14.5 g, 64.0 mmol), Pd(PPh₃)₄ (1.39 g, 1.2 mmol) and Na₂CO₃ (8.5 g, 80.2 mmol) were added to THF (200 mL) and H₂O (50 mL) and heated to reflux under N₂ protection. After 7 h, the reaction was completed as confirmed by TLC plate, heating was stopped, the system was cooled to room temperature and suction-filtered under reduced pressure, and the obtained solid was washed with water and ethanol in sequence. The solid was recrystallized from ethanol to obtain a white solid (8.4 g, 22.8 mmol) with a yield of 57.0%.

Step 4: Synthesis of Compound A-55

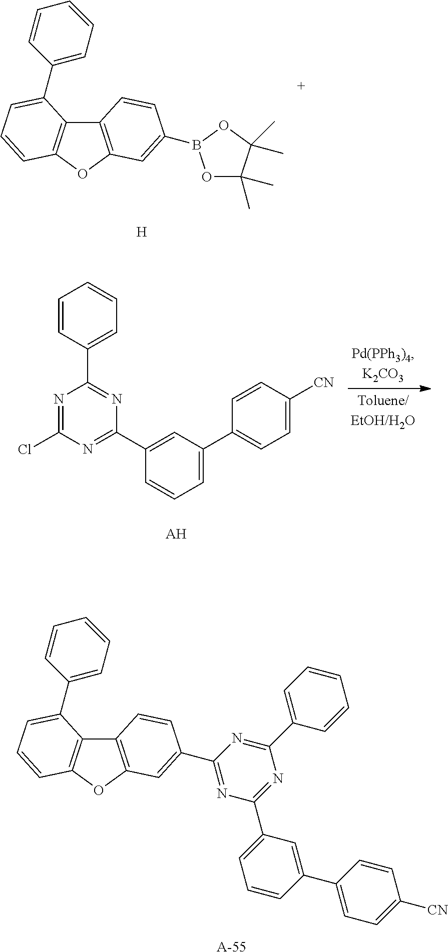

In a 250 mL three-necked round-bottom flask, H (4.44 g, 12.0 mmol), AH (4.4 g, 12.0 mmol), Pd(PPh₃)₄ (0.28 g, 0.24 mmol) and K₂CO₃ (3.3 g, 24.0 mmol) were added to toluene (40 mL), EtOH (10 mL) and H₂O (10 mL) and heated to reflux overnight under N₂ protection. After the reaction was completed as confirmed by TLC plate, heating was stopped, the system was cooled to room temperature and suction-filtered under reduced pressure, and the obtained solid was washed with water and ethanol in sequence. The solid was recrystallized from toluene to obtain a white solid (5.9 g, 10.2 mmol) with a yield of 85.3%. The product was confirmed as the target product, Compound A-55, with a molecular weight of 576.2.

### Synthesis Example 12: Synthesis of Compound A-229

Step 1: Synthesis of Compound A-229

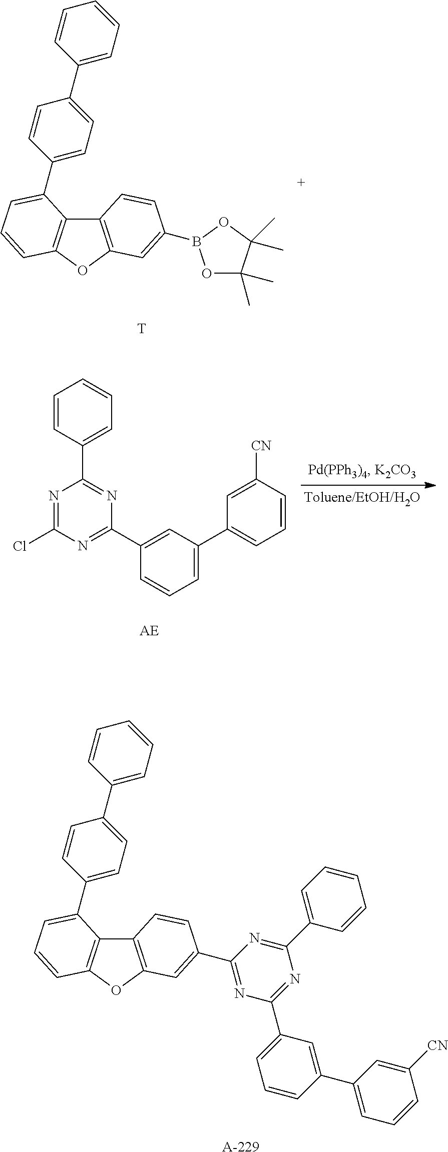

In a 250 mL three-necked round-bottom flask, T (4.1 g, 9.1 mmol), AE (3.2 g, 8.7 mmol), Pd(PPh₃)₄ (0.50 g, 0.44 mmol) and K₂CO₃ (3.6 g, 26.1 mmol) were added to toluene (80 mL), EtOH (20 mL) and H₂O (20 mL) and heated to reflux overnight under N₂ protection. After the reaction was completed as confirmed by TLC plate, heating was stopped, the system was cooled to room temperature and suction-filtered under reduced pressure, and the obtained solid was washed with water and ethanol in sequence. The solid was recrystallized from toluene to obtain a white solid (4.5 g, 6.9 mmol) with a yield of 79.2%. The product was confirmed as the target product, Compound A-229, with a molecular weight of 652.2.

### Synthesis Example 13: Synthesis of Compound A-404

Step 1: Synthesis of Compound A-404

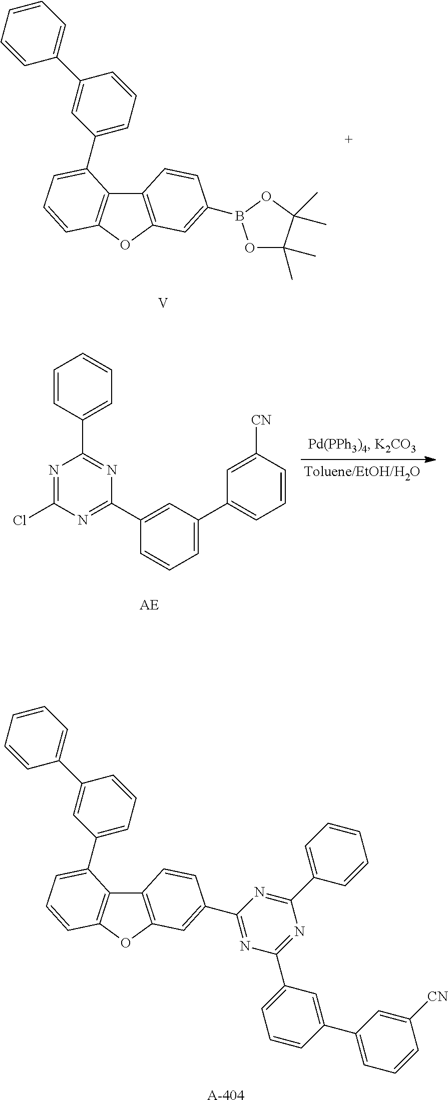

In a 250 mL three-necked round-bottom flask, V (3.63 g, 8.13 mmol), AE (3.0 g, 8.13 mmol), Pd(PPh₃)₄ (0.28 g, 0.24 mmol) and K₂CO₃ (2.26 g, 16.26 mmol) were added to toluene (40 mL), EtOH (10 mL) and H₂O (10 mL) and heated to reflux overnight under N₂ protection. After the reaction was completed as confirmed by TLC plate, heating was stopped and the solution was cooled to room temperature. The organic phase was taken, and the aqueous phase was added with DCM and extracted multiple times. Organic phases were combined, dried over anhydrous Na₂SO₄, filtered and concentrated under reduced pressure. The crude product was subjected to silica gel column chromatography (PE:DCM=4:1 to 1:1) to obtain a white solid (4.0 g, 6.13 mmol) with a yield of 75.4%. The product was confirmed as the target product, Compound A-404, with a molecular weight of 652.2.

Those skilled in the art will appreciate that the above preparation methods are merely exemplary. Those skilled in the art can obtain other compound structures of the present disclosure through the modifications of the preparation methods.

### DEVICE EXAMPLE

### Device Example 1

Firstly, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second and a vacuum degree of about 10⁻⁸ torr. Compound HI was used as a hole injection layer (HIL). Compound HT was used as a hole transporting layer (HTL). Compound H1 was used as an electron blocking layer (EBL). Compound GD1 was doped in Compound H1 and Compound A-2 of the present disclosure, all of which were co-deposited for use as an emissive layer (EML). Compound H2 was used as a hole blocking layer (HBL). On the HBL, Compound ET and 8-hydroxyquinolinolato-lithium (Liq) were co-deposited for use as an electron transporting layer (ETL). Finally, 8-hydroxyquinolinolato-lithium (Liq) was deposited as an electron injection layer with a thickness of 1 nm and Al was deposited as a cathode with a thickness of 120 nm. The device was transferred back to the glovebox and encapsulated with a glass lid to complete the device.

### Device Example 2

Device Example 2 was prepared by the same method as Device Example 1 except that in the EML, Compound A-2 was replaced with Compound A-5.

### Device Example 3

Device Example 3 was prepared by the same method as Device Example 1 except that in the EML, Compound A-2 was replaced with Compound A-8.

### Device Example 4

Device Example 4 was prepared by the same method as Device Example 1 except that in the EML, Compound A-2 was replaced with Compound A-57.

### Device Example 5

Device Example 5 was prepared by the same method as Device Example 1 except that in the EML, Compound A-2 was replaced with Compound A-60.

### Device Example 6

Device Example 6 was prepared by the same method as Device Example 1 except that in the EML, Compound A-2 was replaced with Compound A-177.

### Device Example 7

Device Example 7 was prepared by the same method as Device Example 1 except that in the EML, Compound A-2 was replaced with Compound A-352.

### Device Comparative Example 1

Device Comparative Example 1 was prepared by the same method as Device Example 1 except that in the EML, Compound A-2 was replaced with Compound C-1.

### Device Comparative Example 2

Device Comparative Example 2 was prepared by the same method as Device Example 1 except that in the EML, Compound A-2 was replaced with Compound C-2.

### Device Comparative Example 3

Device Comparative Example 3 was prepared by the same method as Device Example 1 except that in the EML, Compound A-2 was replaced with Compound C-3.

Detailed structures and thicknesses of layers of the devices are shown in the following table. A layer using more than one material is obtained by doping different compounds at their weight ratio as recorded.

TABLE 1
Device structures of Device Examples 1 to 7 and Comparative Examples 1 to 3

| Device ID | HIL | HTL | EBL | EML | HBL | ETL |
|---|---|---|---|---|---|---|
| Example 1 | Compound | Compound | Compound | Compound | Compound | Compound |
| | HI | HT | H1 | H1:Compound | H2 | ET:Liq |
| | (100 Å) | (350 Å) | (50 Å) | A-2:Compound | (50 Å) | (40:60) |
| | | | | GD1 (71:23:6) | | (350 Å) |
| | | | | (400 Å) | | |
| Example 2 | Compound | Compound | Compound | Compound | Compound | Compound |
| | HI | HT | H1 | H1:Compound | H2 | ET:Liq |
| | (100 Å) | (350 Å) | (50 Å) | A-5:Compound | (50 Å) | (40:60) |
| | | | | GD1 (71:23:6) | | (350 Å) |
| | | | | (400 Å) | | |
| Example 3 | Compound | Compound | Compound | Compound | Compound | Compound |
| | HI | HT | H1 | H1:Compound | H2 | ET:Liq |
| | (100 Å) | (350 Å) | (50 Å) | A-8:Compound | (50 Å) | (40:60) |
| | | | | GD1 (71:23:6) | | (350 Å) |
| | | | | (400 Å) | | |
| Example 4 | Compound | Compound | Compound | Compound | Compound | Compound |
| | HI | HT | H1 | H1:Compound | H2 | ET:Liq |
| | (100 Å) | (350 Å) | (50 Å) | A-57:Compound | (50 Å) | (40:60) |
| | | | | GD1 (71:23:6) | | (350 Å) |
| | | | | (400 Å) | | |
| Example 5 | Compound | Compound | Compound | Compound | Compound | Compound |
| | HI | HT | H1 | H1:Compound | H2 | ET:Liq |
| | (100 Å) | (350 Å) | (50 Å) | A-60:Compound | (50 Å) | (40:60) |
| | | | | GD1 (71:23:6) | | (350 Å) |
| | | | | (400 Å) | | |
| Example 6 | Compound | Compound | Compound | Compound | Compound | Compound |
| | HI | HT | H1 | H1:Compound | H2 | ET:Liq |
| | (100 Å) | (350 Å) | (50 Å) | A-177:Compound | (50 Å) | (40:60) |
| | | | | GD1 (71:23:6) | | (350 Å) |
| | | | | (400 Å) | | |
| Example 7 | Compound | Compound | Compound | Compound | Compound | Compound |
| | HI | HT | H1 | H1:Compound | H2 | ET:Liq (40:60) |
| | (100 Å) | (350 Å) | (50 Å) | A-352:Compound | (50 Å) | (350 Å) |
| | | | | GD1 (71:23:6) | | |
| | | | | (400 Å) | | |
| Comparative | Compound | Compound | Compound | Compound | Compound | Compound |
| Example 1 | HI | HT | H1 | H1:Compound | H2 | ET:Liq |
| | (100 Å) | (350 Å) | (50 Å) | C-1:Compound | (50 Å) | (40:60) |
| | | | | GD1 (71:23:6) | | (350 Å) |
| | | | | (400 Å) | | |
| Comparative | Compound | Compound | Compound | Compound | Compound | Compound |
| Example 2 | HI | HT | H1 | H1:Compound | H2 | ET:Liq |
| | (100 Å) | (350 Å) | (50 Å) | C-2:Compound | (50 Å) | (40:60) |
| | | | | GD1 (71:23:6) | | (350 Å) |
| | | | | (400 Å) | | |
| Comparative | Compound | Compound | Compound | Compound | Compound | Compound |
| Example 3 | HI | HT | H1 | H1:Compound | H2 | ET:Liq |
| | (100 Å) | (350 Å) | (50 Å) | C-3:Compound | (50 Å) | (40:60) |
| | | | | GD1 (71:23:6) | | (350 Å) |
| | | | | (400 Å) | | |

The materials used in the devices have the following structures:

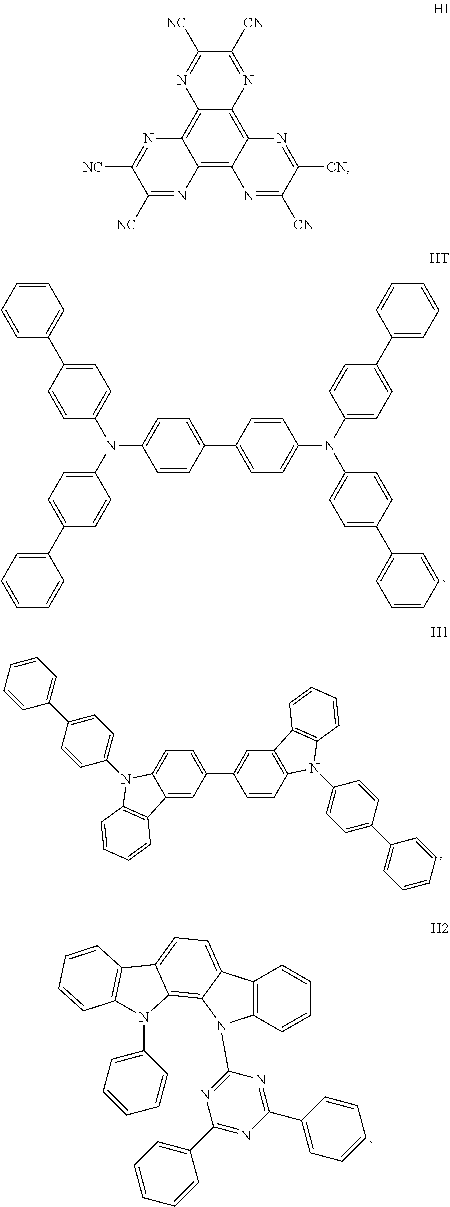 [SMILES: N#Cc1nc2c3nc(C#N)c(C#N)nc3c3nc(C#N)c(C#N)nc3c2nc1C#N ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2nc(-c3ccccc3)nc(-n3c4ccccc4c4ccc5c6ccccc6n(-c6ccccc6)c5c43)n2)cc1]

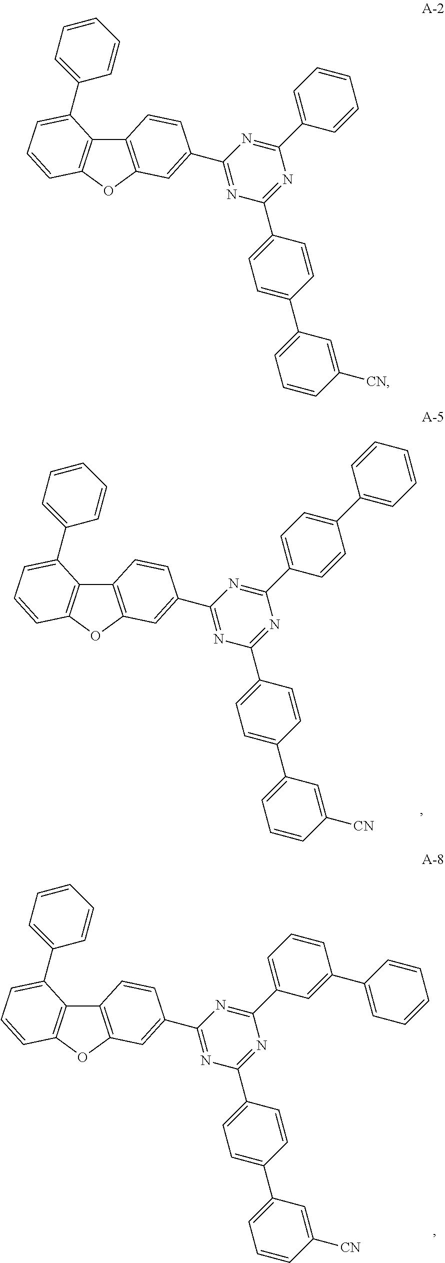 [SMILES: N#Cc1cccc(-c2ccc(-c3nc(-c4ccccc4)nc(-c4ccc5c(c4)oc4cccc(-c6ccccc6)c45)n3)cc2)c1 ; N#Cc1cccc(-c2ccc(-c3nc(-c4ccc(-c5ccccc5)cc4)nc(-c4ccc5c(c4)oc4cccc(-c6ccccc6)c45)n3)cc2)c1 ; N#Cc1cccc(-c2ccc(-c3nc(-c4cccc(-c5ccccc5)c4)nc(-c4ccc5c(c4)oc4cccc(-c6ccccc6)c45)n3)cc2)c1]

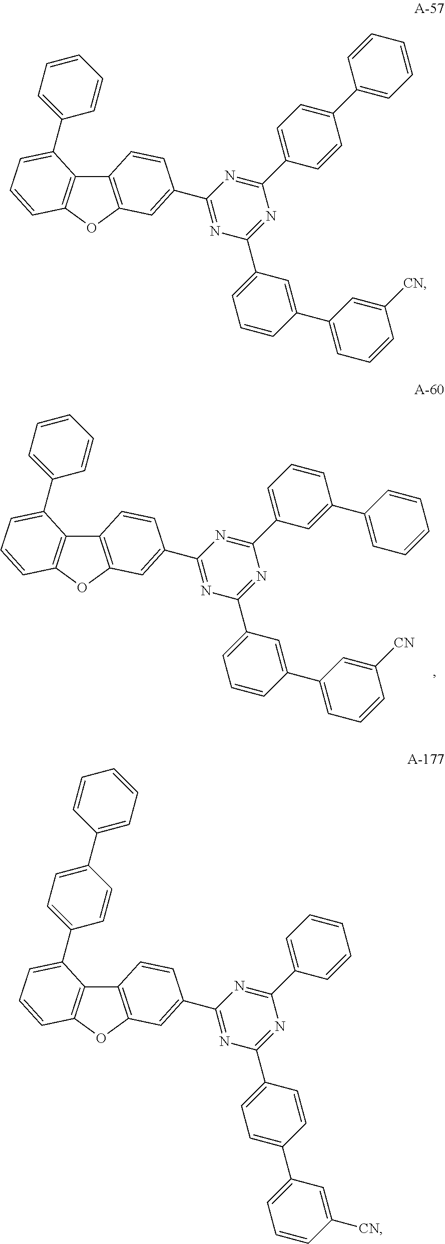 [SMILES: N#Cc1cccc(-c2cccc(-c3nc(-c4ccc(-c5ccccc5)cc4)nc(-c4ccc5c(c4)oc4cccc(-c6ccccc6)c45)n3)c2)c1 ; N#Cc1cccc(-c2cccc(-c3nc(-c4cccc(-c5ccccc5)c4)nc(-c4ccc5c(c4)oc4cccc(-c6ccccc6)c45)n3)c2)c1 ; N#Cc1cccc(-c2ccc(-c3nc(-c4ccccc4)nc(-c4ccc5c(c4)oc4cccc(-c6ccc(-c7ccccc7)cc6)c45)n3)cc2)c1]

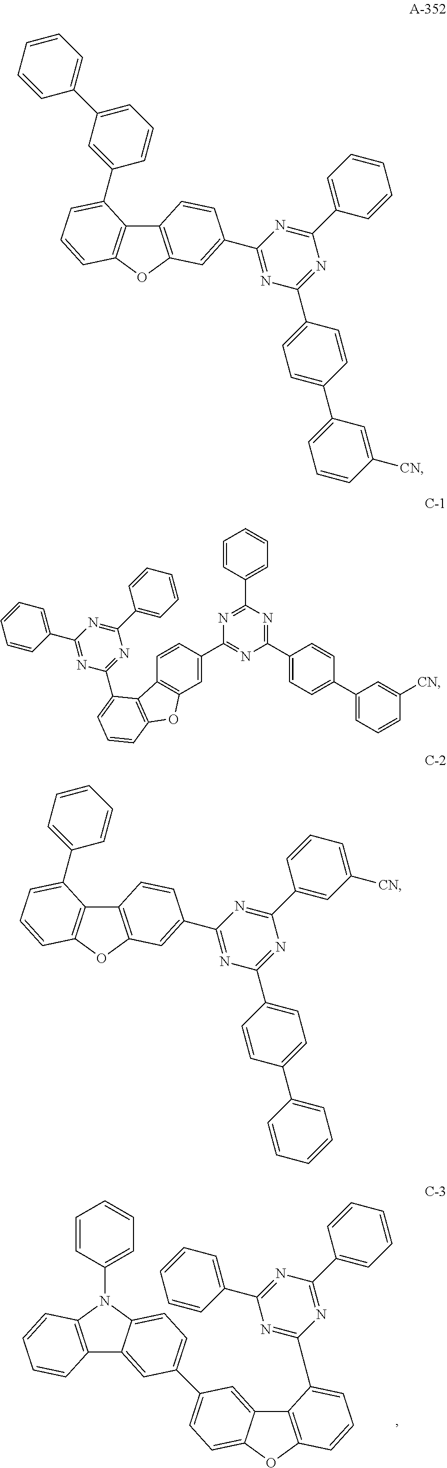 [SMILES: N#Cc1cccc(-c2ccc(-c3nc(-c4ccccc4)nc(-c4ccc5c(c4)oc4cccc(-c6cccc(-c7ccccc7)c6)c45)n3)cc2)c1 ; N#Cc1cccc(-c2ccc(-c3nc(-c4ccccc4)nc(-c4ccc5c(c4)oc4cccc(-c6nc(-c7ccccc7)nc(-c7ccccc7)n6)c45)n3)cc2)c1 ; N#Cc1cccc(-c2nc(-c3ccc(-c4ccccc4)cc3)nc(-c3ccc4c(c3)oc3cccc(-c5ccccc5)c34)n2)c1 ; c1ccc(-c2nc(-c3ccccc3)nc(-c3cccc4oc5ccc(-c6ccc7c(c6)c6ccccc6n7-c6ccccc6)cc5c34)n2)cc1]

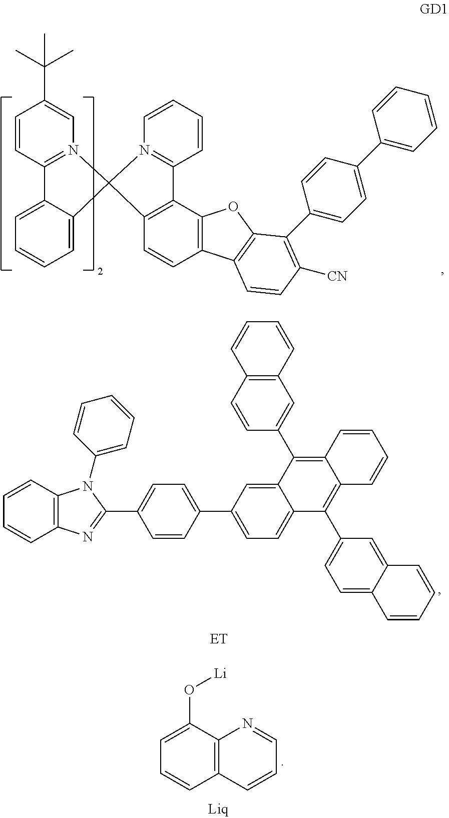

Table 2 shows the CIE data, driving voltage, external quantum efficiency (EQE), current efficiency (CE) and power efficiency (PE) measured at a constant current of 15 mA/cm².

TABLE 2
Device data of Examples 1 to 7 and Comparative Examples 1 to 3

| | | | Voltage | EQE | CE | PE |
|---|---|---|---|---|---|---|
| Device ID | EML | CIE (x, y) | (V) | (%) | (cd/A) | (lm/W) |
| Example 1 | H1:A-2:GD1 | (0.340, 0.636) | 3.96 | 24.43 | 97 | 77 |
| | (71:23:6) | | | | | |
| Example 2 | H1:A-5:GD1 | (0.344, 0.633) | 3.78 | 24.57 | 97 | 81 |
| | (71:23:6) | | | | | |
| Example 3 | H1:A-8:GD1 | (0.344, 0.634) | 3.94 | 24.90 | 99 | 79 |
| | (71:23:6) | | | | | |
| Example 4 | H1:A-57:GD1 | (0.344, 0.633) | 3.76 | 25.07 | 99 | 83 |
| | (71:23:6) | | | | | |
| Example 5 | H1:A-60:GD1 | (0.339, 0.637) | 3.86 | 24.73 | 98 | 80 |
| | (71:23:6) | | | | | |
| Example 6 | H1:A-177:GD1 | (0.345, 0.633) | 3.81 | 24.89 | 98 | 81 |
| | (71:23:6) | | | | | |
| Example 7 | H1:A-352:GD1 | (0.342, 0.635) | 3.81 | 25.10 | 99 | 82 |
| | (71:23:6) | | | | | |
| Comparative | H1:C-1:GD1 | (0.345, 0.632) | 4.05 | 18.62 | 73 | 57 |
| Example 1 | (71:23:6) | | | | | |
| Comparative | H1:C-2:GD1 | (0.344, 0.634) | 4.15 | 21.56 | 85 | 64 |
| Example 2 | (71:23:6) | | | | | |
| Comparative | H1:C-3:GD1 | (0.339, 0.637) | 3.76 | 21.91 | 87 | 72 |
| Example 3 | (71:23:6) | | | | | |

Discussion:

In Examples 1 to 7 and Comparative Example 1, the first metal complex GD1 was separately doped in a series of compounds of the present disclosure and Compound C-1 which is not claimed by the present disclosure. Compared with Comparative Example 1, Examples 1 to 7 have the EQE increased by 31.2% to 34.8%, the CE increased by about 32.9%, the PE increased by 35.1% to 45.6% and reduced driving voltages. The above indicates that compared with a compound having a heteroaryl substituent at position 1 of dibenzofuran, the compound of the present disclosure which has an aryl substituent at position 1 of dibenzofuran can improve device performance, particularly device efficiency (EQE, PE and CE), when applied to an electroluminescent device.

In Examples 1 to 7 and Comparative Example 2, the first metal complex GD1 was separately doped in the series of compounds of the present disclosure and Compound C-2 which is not claimed by the present disclosure. Compared with Comparative Example 2, Examples 1 to 7 have the EQE increased by 13.3% to 16.4%, the CE increased by about 14.1%, the PE increased by 20.3% to 29.6% and reduced driving voltages. This indicates that compared with Compound C-2 having a cyano substituent on the ring A in the comparative example, the compound of the present disclosure which has a cyano substituent on the ring B can improve device performance, particularly device efficiency, when applied to an electroluminescent device.

In Examples 1 to 7 and Comparative Example 3, the first metal complex GD1 was separately doped in the series of compounds of the present disclosure and Compound C-3 which is not claimed by the present disclosure. It is to be particularly noted that C-3 is a commercially available host material at present. Compared with Comparative Example 3, Examples 1 to 7 have the EQE increased by 11.5% to 14.6%, the CE increased by about 11.5% and the increased PE despite slightly increased driving voltages. It can be seen that the compound of the present disclosure can satisfy the requirement for performance in commercial use, has superior device efficiency, and is a type of commercially promising compound.

### Device Example 8

Device Example 8 was prepared by the same method as Device Example 2 except that in the EML, Compound GD1 was replaced with Compound GD2 and H1:A-5:GD2=72:24:4.

### Device Example 9

Device Example 9 was prepared by the same method as Device Example 8 except that in the EML, Compound A-5 was replaced with Compound A-57.

### Device Example 10

Device Example 10 was prepared by the same method as Device Example 8 except that in the EML, Compound A-5 was replaced with Compound A-177.

### Device Example 11

Device Example 11 was prepared by the same method as Device Example 8 except that in the EML, Compound A-5 was replaced with Compound A-352.

### Device Comparative Example 4

Device Comparative Example 4 was prepared by the same method as Device Example 8 except that in the EML, Compound A-5 was replaced with Compound C-2.

### Device Comparative Example 5

Device Comparative Example 5 was prepared by the same method as Device Example 8 except that in the EML, Compound A-5 was replaced with Compound C-3.

Detailed structures and thicknesses of layers of the devices are shown in the following table. A layer using more than one material is obtained by doping different compounds at their weight ratio as recorded.

TABLE 3
Device structures of Device Examples 8 to 11 and Comparative Examples 4 to 5

| Device ID | HIL | HTL | EBL | EML | HBL | ETL |
|---|---|---|---|---|---|---|
| Example 8 | Compound | Compound | Compound | Compound | Compound | Compound |
| | HI | HT | H1 | H1:Compound | H2 | ET:Liq |
| | (100 Å) | (350 Å) | (50 Å) | A-5:Compound | (50 Å) | (40:60) |
| | | | | GD2 (72:24:4) | | (350 Å) |
| | | | | (400 Å) | | |
| Example 9 | Compound | Compound | Compound | Compound | Compound | Compound |
| | HI | HT | H1 | H1:Compound | H2 | ET:Liq |
| | (100 Å) | (350 Å) | (50 Å) | A-57:Compound | (50 Å) | (40:60) |
| | | | | GD2 (72:24:4) | | (350 Å) |
| | | | | (400 Å) | | |
| Example 10 | Compound | Compound | Compound | Compound | Compound | Compound |
| | HI | HT | H1 | H1:Compound | H2 | ET:Liq |
| | (100 Å) | (350 Å) | (50 Å) | A-177:Compound | (50 Å) | (40:60) |
| | | | | GD2 (72:24:4) | | (350 Å) |
| | | | | (400 Å) | | |
| Example 11 | Compound | Compound | Compound | Compound | Compound | Compound |
| | HI | HT | H1 | H1:Compound | H2 | ET:Liq |
| | (100 Å) | (350 Å) | (50 Å) | A-352:Compound | (50 Å) | (40:60) |
| | | | | GD2 (72:24:4) | | (350 Å) |
| | | | | (400 Å) | | |
| Comparative | Compound | Compound | Compound | Compound | Compound | Compound |
| Example 4 | HI | HT | H1 | H1:Compound | H2 | ET:Liq |
| | (100 Å) | (350 Å) | (50 Å) | C-2:Compound | (50 Å) | (40:60) |
| | | | | GD2 (72:24:4) | | (350 Å) |
| | | | | (400 Å) | | |
| Comparative | Compound | Compound | Compound | Compound | Compound | Compound |
| Example 5 | HI | HT | H1 | H1:Compound | H2 | ET:Liq |
| | (100 Å) | (350 Å) | (50 Å) | C-3:Compound | (50 Å) | (40:60) |
| | | | | GD2 (72:24:4) | | (350 Å) |
| | | | | (400 Å) | | |

The new material used in the devices has the following structure:

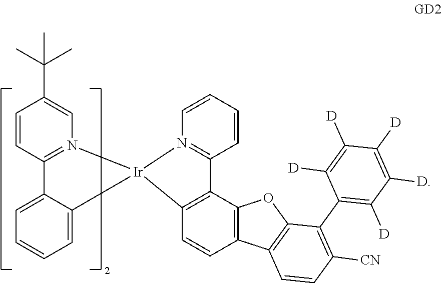

Table 4 shows the CIE data, driving voltage, external quantum efficiency (EQE), current efficiency (CE) and power efficiency (PE) measured at a constant current of 15 mA/cm².

TABLE 4
Device data of Examples 8 to 11 and Comparative Examples 4 to 5

| | | | Voltage | EQE | CE | PE |
|---|---|---|---|---|---|---|
| Device ID | EML | CIE (x, y) | (V) | (%) | (cd/Å) | (lm/W) |
| Example 8 | H1:A-5:GD2 | (0.343, 0.633) | 3.62 | 23.68 | 93 | 81 |
| | (72:24:4) | | | | | |
| Example 9 | H1:A-57:GD2 | (0.338, 0.637) | 3.68 | 24.13 | 95 | 81 |
| | (72:24:4) | | | | | |
| Example 10 | H1:A-177:GD2 | (0.340, 0.635) | 3.72 | 23.98 | 95 | 80 |
| | (72:24:4) | | | | | |
| Example 11 | H1:A-352:GD2 | (0.336, 0.638) | 3.72 | 23.95 | 95 | 80 |
| | (72:24:4) | | | | | |
| Comparative | H1:C-2:GD2 | (0.341, 0.635) | 4.05 | 21.85 | 86 | 67 |
| Example 4 | (72:24:4) | | | | | |
| Comparative | H1:C-3:GD2 | (0.333, 0.640) | 3.68 | 21.40 | 85 | 72 |
| Example 5 | (72:24:4) | | | | | |

Discussion:

In Examples 8 to 11 and Comparative Example 4, the first metal complex GD2 was separately doped in Compounds A-5, A-57, A-177 and A-352 of the present disclosure and Compound C-2 which is not claimed by the present disclosure. Compared with Comparative Example 4, Examples 8 to 11 have the EQE increased by 8.4%, 10.4%, 9.7% and 9.6% respectively, the increased CE and PE and reduced driving voltages. The above indicates that compared with Compound C-2 having a cyano substituent on the ring A in the comparative example, the compound of the present disclosure which has a cyano substituent on the ring B can improve device performance, particularly EQE, when applied to an electroluminescent device.

In Examples 8 to 11 and Comparative Example 5, the first metal complex GD2 was separately doped in Compounds A-5, A-57, A-177 and A-352 of the present disclosure and Compound C-3 which is not claimed by the present disclosure. Compared with Comparative Example 5, Examples 8 to 11 have comparable driving voltages, the EQE increased by 10.7%, 12.8%, 12.1% and 11.9% respectively, and the increased CE and PE. It can be seen that the compound of the present disclosure can satisfy the requirement for performance in commercial use, has superior device efficiency, and is a type of commercially promising compound.

### Device Example 12

Device Example 12 was prepared by the same method as Device Example 1 except that in the EML, Compound GD1 was replaced with Compound GD3.

### Device Example 13

Device Example 13 was prepared by the same method as Device Example 12 except that in the EML, Compound A-2 was replaced with Compound A-5.

### Device Example 14

Device Example 14 was prepared by the same method as Device Example 12 except that in the EML, Compound A-2 was replaced with Compound A-8.

### Device Example 15

Device Example 15 was prepared by the same method as Device Example 12 except that in the EML, Compound A-2 was replaced with Compound A-57.

### Device Example 16

Device Example 16 was prepared by the same method as Device Example 12 except that in the EML, Compound A-2 was replaced with Compound A-60.

### Device Comparative Example 6

Device Comparative Example 6 was prepared by the same method as Device Example 12 except that in the EML, Compound A-2 was replaced with Compound C-1.

### Device Comparative Example 7

Device Comparative Example 7 was prepared by the same method as Device Example 12 except that in the EML, Compound A-2 was replaced with Compound C-2.

### Device Comparative Example 8

Device Comparative Example 8 was prepared by the same method as Device Example 12 except that in the EML, Compound A-2 was replaced with Compound C-3.

Detailed structures and thicknesses of layers of the devices are shown in the following table. A layer using more than one material is obtained by doping different compounds at their weight ratio as recorded.

TABLE 5
Device structures of Device Examples 12 to 16 and Comparative Examples 6 to 8

| Device ID | HIL | HTL | EBL | EML | HBL | ETL |
|---|---|---|---|---|---|---|
| Example 12 | Compound | Compound | Compound | Compound | Compound | Compound |
| | HI | HT | H1 | H1:Compound | H2 | ET:Liq |
| | (100 Å) | (350 Å) | (50 Å) | A-2:Compound | (50 Å) | (40:60) |
| | | | | GD3 (71:23:6) | | (350 Å) |
| | | | | (400 Å) | | |
| Example 13 | Compound | Compound | Compound | Compound | Compound | Compound |
| | HI | HT | H1 | H1:Compound | H2 | ET:Liq |
| | (100 Å) | (350 Å) | (50 Å) | A-5:Compound | (50 Å) | (40:60) |
| | | | | GD3 (71:23:6) | | (350 Å) |
| | | | | (400 Å) | | |
| Example 14 | Compound | Compound | Compound | Compound | Compound | Compound |
| | HI | HT | H1 | H1:Compound | H2 | ET:Liq |
| | (100 Å) | (350 Å) | (50 Å) | A-8:Compound | (50 Å) | (40:60) |
| | | | | GD3 (71:23:6) | | (350 Å) |
| | | | | (400 Å) | | |
| Example 15 | Compound | Compound | Compound | Compound | Compound | Compound |
| | HI | HT | H1 | H1:Compound | H2 | ET:Liq |
| | (100 Å) | (350 Å) | (50 Å) | A-57:Compound | (50 Å) | (40:60) |
| | | | | GD3 (71:23:6) | | (350 Å) |
| | | | | (400 Å) | | |
| Example 16 | Compound | Compound | Compound | Compound | Compound | Compound |
| | HI | HT | H1 | H1:Compound | H2 | ET:Liq |
| | (100 Å) | (350 Å) | (50 Å) | A-60:Compound | (50 Å) | (40:60) |
| | | | | GD3 (71:23:6) | | (350 Å) |
| | | | | (400 Å) | | |
| Comparative | Compound | Compound | Compound | Compound | Compound | Compound |
| Example 6 | HI | HT | H1 | H1:Compound | H2 | ET:Liq |
| | (100 Å) | (350 Å) | (50 Å) | C-1:Compound | (50 Å) | (40:60) |
| | | | | GD3 (71:23:6) | | (350 Å) |
| | | | | (400 Å) | | |
| Comparative | Compound | Compound | Compound | Compound | Compound | Compound |
| Example 7 | HI | HT | H1 | H1:Compound | H2 | ET:Liq |
| | (100 Å) | (350 Å) | (50 Å) | C-2:Compound | (50 Å) | (40:60) |
| | | | | GD3 (71:23:6) | | (350 Å) |
| | | | | (400 Å) | | |
| Comparative | Compound | Compound | Compound | Compound | Compound | Compound |
| Example 8 | HI | HT | H1 | H1:Compound | H2 | ET:Liq |
| | (100 Å) | (350 Å) | (50 Å) | C-3:Compound | (50 Å) | (40:60) |
| | | | | GD3 (71:23:6) | | (350 Å) |
| | | | | (400 Å) | | |

The new material used in the devices has the following structure:

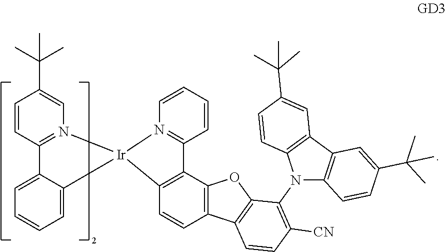

Table 6 shows the CIE data, driving voltage, external quantum efficiency (EQE), current efficiency (CE) and power efficiency (PE) measured at a constant current of 15 mA/cm².

TABLE 6
Device data of Examples 12 to 16 and Comparative Examples 6 to 8

| | | | Voltage | EQE | CE | PE |
|---|---|---|---|---|---|---|
| Device ID | EML | CIE (x, y) | (V) | (%) | (cd/A) | (lm/W) |
| Example 12 | H1:A-2:GD3 | (0.339, 0.636) | 3.73 | 24.87 | 98 | 83 |
| | (71:23:6) | | | | | |
| Example 13 | H1:A-5:GD3 | (0.343, 0.633) | 3.67 | 24.92 | 98 | 84 |
| | (71:23:6) | | | | | |
| Example 14 | H1:A-8:GD3 | (0.345, 0.632) | 3.62 | 25.16 | 99 | 86 |
| | (71:23:6) | | | | | |
| Example 15 | H1:A-57:GD3 | (0.344, 0.633) | 3.59 | 25.37 | 100 | 87 |
| | (71:23:6) | | | | | |
| Example 16 | H1:A-60:GD3 | (0.338, 0.637) | 3.79 | 24.17 | 95 | 79 |
| | (71:23:6) | | | | | |
| Comparative | H1:C-1:GD3 | (0.342, 0.634) | 3.99 | 20.43 | 80 | 63 |
| Example 6 | (71:23:6) | | | | | |
| Comparative | H1:C-2:GD3 | (0.342, 0.634) | 3.95 | 22.36 | 88 | 70 |
| Example 7 | (71:23:6) | | | | | |
| Comparative | H1:C-3:GD3 | (0.335, 0.639) | 3.56 | 21.45 | 84 | 74 |
| Example 8 | (71:23:6) | | | | | |

Discussion:

In Examples 12 to 16 and Comparative Example 6, the first metal complex GD3 was separately doped in a series of compounds of the present disclosure and Compound C-1 which is not claimed by the present disclosure. Compared with Comparative Example 6, Examples 12 to 16 have the EQE increased by 18.3% to 24.2%, the CE increased by 18.7% to 25%, the PE increased by 25.4% to 35.1% and reduced driving voltages. This indicates that compared with a compound having a heteroaryl substituent at position 1 of dibenzofuran, the compound of the present disclosure which has an aryl substituent at position 1 of dibenzofuran can improve device performance, particularly EQE, when applied to an electroluminescent device.

In Examples 12 to 16 and Comparative Example 7, the first metal complex GD3 was separately doped in the series of compounds of the present disclosure and Compound C-2 which is not claimed by the present disclosure. Compared with Comparative Example 7, Examples 12 to 16 have the EQE increased by 8.1% to 13.5%, the significantly increased CE and PE and reduced driving voltages. The above indicates that compared with Compound C-2 having a cyano substituent on the ring A in the comparative example, the compound of the present disclosure which has a cyano substituent on the ring B can improve device performance, particularly EQE, when applied to an electroluminescent device.

In Examples 12 to 16 and Comparative Example 8, the first metal complex GD3 was separately doped in the series of compounds of the present disclosure and Compound C-3 which is not claimed by the present disclosure. Compared with Comparative Example 8, Examples 12 to 16 have the EQE increased by 12.7% to 18.3% and the increased CE and PE despite slightly increased driving voltages. It can be seen that the compound of the present disclosure can satisfy the requirement for performance in commercial use, has superior device efficiency, and is a type of commercially promising compound.

### Device Example 17

Device Example 17 was prepared by the same method as Device Example 1 except that in the EML, Compound GD1 was replaced with Compound GD4.

### Device Example 18

Device Example 18 was prepared by the same method as Device Example 17 except that in the EML, Compound A-2 was replaced with Compound A-5.

### Device Example 19

Device Example 19 was prepared by the same method as Device Example 17 except that in the EML, Compound A-2 was replaced with Compound A-60.

### Device Comparative Example 9

Device Comparative Example 9 was prepared by the same method as Device Example 17 except that in the EML, Compound A-2 was replaced with Compound C-1.

### Device Comparative Example 10

Device Comparative Example 10 was prepared by the same method as Device Example 17 except that in the EML, Compound A-2 was replaced with Compound C-2.

### Device Comparative Example 11

Device Comparative Example 11 was prepared by the same method as Device Example 17 except that in the EML, Compound A-2 was replaced with Compound C-3.

Detailed structures and thicknesses of layers of the devices are shown in the following table. A layer using more than one material is obtained by doping different compounds at their weight ratio as recorded.

TABLE 7
Device structures of Device Examples 17 to 19 and Comparative Examples 9 to 11

| Device ID | HIL | HTL | EBL | EML | HBL | ETL |
|---|---|---|---|---|---|---|
| Example 17 | Compound | Compound | Compound | Compound | Compound | Compound |
| | HI | HT | H1 | H1:Compound | H2 | ET:Liq |
| | (100 Å) | (350 Å) | (50 Å) | A-2:Compound | (50 Å) | (40:60) |
| | | | | GD4 (71:23:6) | | (350 Å) |
| | | | | (400 Å) | | |
| Example 18 | Compound | Compound | Compound | Compound | Compound | Compound |
| | HI | HT | H1 | H1:Compound | H2 | ET:Liq |
| | (100 Å) | (350 Å) | (50 Å) | A-5:Compound | (50 Å) | (40:60) |
| | | | | GD4 (71:23:6) | | (350 Å) |
| | | | | (400 Å) | | |
| Example 19 | Compound | Compound | Compound | Compound | Compound | Compound |
| | HI | HT | H1 | H1:Compound | H2 | ET:Liq |
| | (100 Å) | (350 Å) | (50 Å) | A-60:Compound | (50 Å) | (40:60) |
| | | | | GD4 (71:23:6) | | (350 Å) |
| | | | | (400 Å) | | |
| Comparative | Compound | Compound | Compound | Compound | Compound | Compound |
| Example 9 | HI | HT | H1 | H1:Compound | H2 | ET:Liq |
| | (100 Å) | (350 Å) | (50 Å) | C-1:Compound | (50 Å) | (40:60) |
| | | | | GD4 (71:23:6) | | (350 Å) |
| | | | | (400 Å) | | |
| Comparative | Compound | Compound | Compound | Compound | Compound | Compound |
| Example 10 | HI | HT | H1 | H1:Compound | H2 | ET:Liq |
| | (100 Å) | (350 Å) | (50 Å) | C-2:Compound | (50 Å) | (40:60) |
| | | | | GD4 (71:23:6) | | (350 Å) |
| | | | | (400 Å) | | |
| Comparative | Compound | Compound | Compound | Compound | Compound | Compound |
| Example 11 | HI | HT | H1 | H1:Compound | H2 | ET:Liq |
| | (100 Å) | (350 Å) | (50 Å) | C-3:Compound | (50 Å) | (40:60) |
| | | | | GD4 (71:23:6) | | (350 Å) |
| | | | | (400 Å) | | |

The new material used in the devices has the following structure:

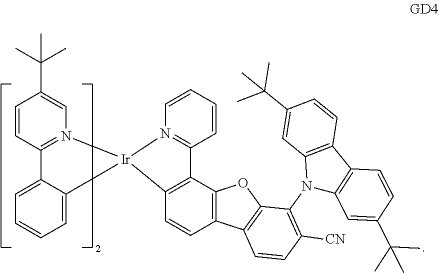

Table 8 shows the CIE data, driving voltage, external quantum efficiency (EQE), current efficiency (CE) and power efficiency (PE) measured at a constant current of 15 mA/cm².

TABLE 8
Device data of Examples 17 to 19 and Comparative Examples 9 to 11

| | | | Voltage | | CE | PE |
|---|---|---|---|---|---|---|
| Device ID | EML | CIE (x, y) | (V) | EQE (%) | (cd/A) | (lm/W) |
| Example 17 | H1:A-2:GD4 | (0.331, 0.643) | 3.85 | 24.66 | 98 | 80 |
| | (71:23:6) | | | | | |
| Example 18 | H1:A-5:GD4 | (0.339, 0.637) | 3.63 | 24.81 | 98 | 85 |
| | (71:23:6) | | | | | |
| Example 19 | H1:A-60:GD4 | (0.330, 0.643) | 3.91 | 24.42 | 97 | 78 |
| | (71:23:6) | | | | | |
| Comparative | H1:C-1:GD4 | (0.335, 0.639) | 4.11 | 20.48 | 81 | 62 |
| Example 9 | (71:23:6) | | | | | |
| Comparative | H1:C-2:GD4 | (0.335, 0.639) | 3.91 | 22.54 | 89 | 72 |
| Example 10 | (71:23:6) | | | | | |
| Comparative | H1:C-3:GD4 | (0.329, 0.644) | 3.52 | 21.55 | 85 | 76 |
| Example 11 | (71:23:6) | | | | | |

Discussion:

In Examples 17 to 19 and Comparative Example 9, the first metal complex GD4 was separately doped in Compounds A-2, A-5 and A-60 of the present disclosure and Compound C-1 which is not claimed by the present disclosure. Compared with Comparative Example 9, Examples 17 to 19 have the EQE increased by 20.4%, 21.1% and 19.2% respectively, the significantly increased CE and PE and reduced driving voltages. This indicates that compared with a compound having a heteroaryl substituent at position 1 of dibenzofuran, the compound of the present disclosure which has an aryl substituent at position 1 of dibenzofuran can improve device performance, particularly EQE, when applied to an electroluminescent device.

In Examples 17 to 19 and Comparative Example 10, the first metal complex GD4 was separately doped in Compounds A-2, A-5 and A-60 of the present disclosure and Compound C-2 which is not claimed by the present disclosure. Compared with Comparative Example 10, Examples 17 to 19 have comparable or reduced driving voltages, the EQE increased by 9.4%, 10.1% and 8.3% respectively, and the increased CE and PE. The above indicates that compared with Compound C-2 having a cyano substituent on the ring A in the comparative example, the compound of the present disclosure which has a cyano substituent on the ring B can improve device performance, particularly EQE, when applied to an electroluminescent device.

In Examples 17 to 19 and Comparative Example 11, the first metal complex GD4 was separately doped in Compounds A-2, A-5 and A-60 of the present disclosure and Compound

C-3 which is not claimed by the present disclosure. Compared with Comparative Example 11, Examples 17 to 19 have the EQE increased by 14.4%, 15.1% and 13.3% respectively and the increased CE and PE despite slightly increased driving voltages. It can be seen that the compound of the present disclosure can satisfy the requirement for performance in commercial use, has superior device efficiency, and is a type of commercially promising compound.

### Device Example 20

Device Example 20 was prepared by the same method as Device Example 8 except that in the EML, Compound A-5 was replaced with Compound A-1.

### Device Example 21

Device Example 21 was prepared by the same method as Device Example 8 except that in the EML, Compound A-5 was replaced with Compound A-55.

Detailed structures and thicknesses of layers of the devices are shown in the following table. A layer using more than one material is obtained by doping different compounds at their weight ratio as recorded.

TABLE 9
Device structures of Device Examples 20 and 21

| Device ID | HIL | HTL | EBL | EML | HBL | ETL |
|---|---|---|---|---|---|---|
| Example 20 | Compound | Compound | Compound | Compound | Compound | Compound |
| | HI | HT | H1 | H1:Compound | H2 | ET:Liq |
| | (100 Å) | (350 Å) | (50 Å) | A-1:Compound | (50 Å) | (40:60) |
| | | | | GD2 (72:24:4) | | (350 Å) |
| | | | | (400 Å) | | |
| Example 21 | Compound | Compound | Compound | Compound | Compound | Compound |
| | HI | HT | H1 | H1:Compound | H2 | ET:Liq |
| | (100 Å) | (350 Å) | (50 Å) | A-55:Compound | (50 Å) | (40:60) |
| | | | | GD2 (72:24:4) | | (350 Å) |
| | | | | (400 Å) | | |

The new materials used in the devices have the following structures:

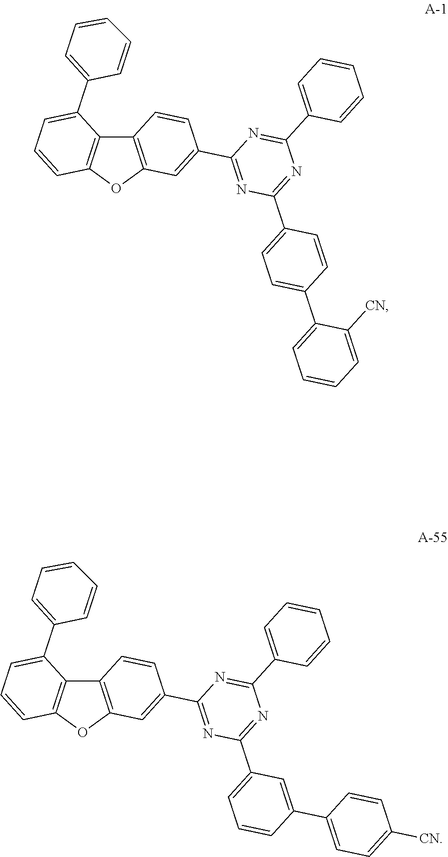 [SMILES: N#Cc1ccccc1-c1ccc(-c2nc(-c3ccccc3)nc(-c3ccc4c(c3)oc3cccc(-c5ccccc5)c34)n2)cc1 ; N#Cc1ccc(-c2cccc(-c3nc(-c4ccccc4)nc(-c4ccc5c(c4)oc4cccc(-c6ccccc6)c45)n3)c2)cc1]

Table 10 shows the CIE data, driving voltage, external quantum efficiency (EQE), current efficiency (CE) and power efficiency (PE) measured at a constant current of 15 mA/cm².

TABLE 10
Device data of Examples 20 and 21 and Comparative Examples 4 and 5

| | | | Voltage | EQE | CE | PE |
|---|---|---|---|---|---|---|
| Device ID | EML | CIE (x, y) | (V) | (%) | (cd/Å) | (lm/W) |
| Example 20 | H1:A-1:GD2 | (0.345, 0.632) | 3.73 | 23.72 | 93.6 | 78.81 |
| | (72:24:4) | | | | | |
| Example 21 | H1:A-55:GD2 | (0.345, 0.632) | 3.62 | 23.90 | 94.24 | 81.82 |
| | (72:24:4) | | | | | |
| Comparative | H1:C-2:GD2 | (0.341, 0.635) | 4.05 | 21.85 | 86 | 67 |
| Example 4 | (72:24:4) | | | | | |
| Comparative | H1:C-3:GD2 | (0.333, 0.640) | 3.68 | 21.40 | 85 | 72 |
| Example 5 | (72:24:4) | | | | | |

In Examples 20 and 21, the first metal complex GD2 was separately doped in Compounds A-1 and A-55 of the present disclosure. Both the devices have relatively high luminescence efficiency, particularly relatively high EQE. In Examples 20 to 21 and Comparative Example 4, the first metal complex GD2 was separately doped in Compounds A-1 and A-55 of the present disclosure and Compound C-2 which is not claimed by the present disclosure. Compared with Comparative Example 4, Examples 20 to 21 have the EQE increased by 8.5% and 9.3% respectively, the CE increased by 8.8% and 9.6% respectively, the PE increased by 17.6% and 22% respectively, and reduced driving voltages. The above indicates that compared with Compound C-2 having a cyano substituent on the ring A in the comparative example, the compound of the present disclosure which has a cyano substituent on the ring B can improve device performance, particularly device efficiency, in all aspects when applied to an electroluminescent device.

In Examples 20 to 21 and Comparative Example 5, the first metal complex GD2 was separately doped in Compounds A-1 and A-55 of the present disclosure and Compound C-3 which is not claimed by the present disclosure. Compared with Comparative Example 5, Examples 20 to 21 have comparable driving voltages, the EQE increased by 10.8% and 11.7% respectively, the CE increased by 10.1% and 10.8% respectively, and the PE increased by 9.4% and 13.6% respectively. It can be seen that the compound of the present disclosure can satisfy the requirement for performance in commercial use, has superior device efficiency, and is a type of commercially promising compound.

To sum up, when applied to an organic electroluminescent device, the compound of the present disclosure having the structure of Formula 1 improves the electron and hole transporting balance capability of a material and significantly improves device performance compared with the compound which is not claimed by the present disclosure (which has a cyano substitution at a non-specific position or does not have the skeleton of Formula 1), where the device has significantly improved EQE and significantly improved CE and PE. The compound of the present disclosure is of great help to the industry.

### Device Example 22

Firstly, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second and a vacuum degree of about 10⁻⁸ torr. Compound HI was used as a hole injection layer (HIL). Compound HT was used as a hole transporting layer (HTL). Compound H1 was used as an electron blocking layer (EBL). Compound GD23 was doped in Compound H1 and Compound NH-1, all of which were co-deposited for use as an emissive layer (EML). Compound H2 was used as a hole blocking layer (HBL). On the HBL, Compound A-2 of the present disclosure and 8-hydroxyquinolinolato-lithium (Liq) were co-deposited for use as an electron transporting layer (ETL). Finally, 8-hydroxyquinolinolato-lithium (Liq) was deposited as an electron injection layer with a thickness of 1 nm and Al was deposited as a cathode with a thickness of 120 nm. The device was transferred back to the glovebox and encapsulated with a glass lid to complete the device.

### Device Comparative Example 12

Device Comparative Example 12 was prepared by the same method as Device Example 22 except that in the ETL, Compound A-2 was replaced with Compound ET.

Detailed structures and thicknesses of layers of the devices are shown in the following table. A layer using more than one material is obtained by doping different compounds at their weight ratio as recorded.

TABLE 11
Part of device structures of Device Example 22 and Comparative Example 12

| Device ID | HIL | HTL | EBL | EML | HBL | ETL |
|---|---|---|---|---|---|---|
| Example 22 | Compound | Compound | Compound | Compound | Compound | Compound |
| | HI | HT | H1 | H1:Compound | H2 | A-2:Liq |
| | (100 Å) | (350 Å) | (50 Å) | NH-1:Compound | (50 Å) | (40:60) |
| | | | | GD23 (46:46:8) | | (350 Å) |
| | | | | (400 Å) | | |
| Comparative | Compound | Compound | Compound | Compound | Compound | Compound |
| Example 12 | HI | HT | H1 | H1:Compound | H2 | ET:Liq |
| | (100 Å) | (350 Å) | (50 Å) | NH-1:Compound | (50 Å) | (40:60) |
| | | | | GD23 (46:46:8) | | (350 Å) |
| | | | | (400 Å) | | |

The new materials used in the devices have the following structures:

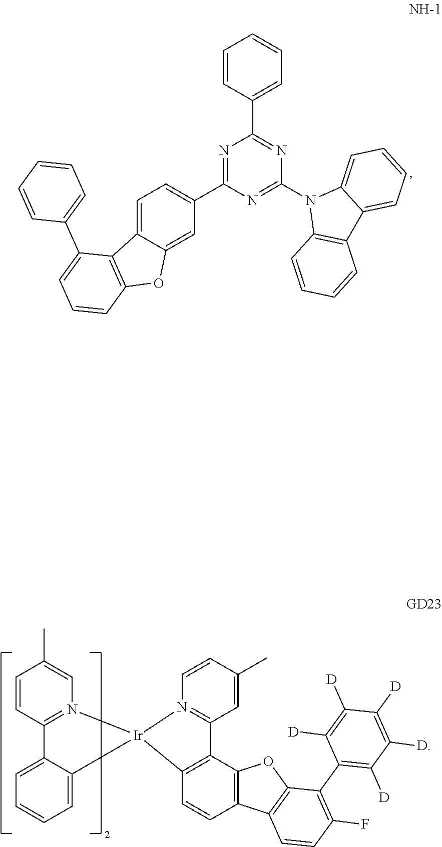

Table 12 shows the CIE data, driving voltage (V) and external quantum efficiency (EQE) measured at a constant current of 15 mA/cm² and the device lifetime (LT97) measured at a constant current of 80 mA/cm².

TABLE 12
Device data of Example 22 and Comparative Example 12

| | | CIE | Driving | EQE | Lifetime |
|---|---|---|---|---|---|
| Device ID | ETL | (x, y) | Voltage (V) | (%) | LT97 (h) |
| Example 22 | A-2:Liq | (0.356, 0.619) | 4.1 | 21.0 | 46.7 |
| | (40:60) | | | | |
| Comparative | ET:Liq | (0.359, 0.617) | 3.7 | 20.9 | 42.8 |
| Example 12 | (40:60) | | | | |

Discussion:

Compound A-2 of the present disclosure and Compound ET which is not claimed by the present disclosure are used as an electron transporting material in Example 22 and

Comparative Example 12, respectively. Compared with Comparative Example 12, Example 22 has comparable EQE and the device lifetime improved by 9% despite a slightly increased driving voltage. It is to be noted that Compound ET is a commercially available electron transporting material at present. Compared with Compound ET, the compound of the present disclosure can further improve the device lifetime when applied to an electroluminescent device.

It can be seen that the compound of the present disclosure is also an excellent type of electron transporting material.

It is to be understood that various embodiments described herein are merely examples and not intended to limit the scope of the present disclosure. Therefore, it is apparent to the persons skilled in the art that the present disclosure as claimed may include variations of specific embodiments and preferred embodiments described herein. Many of materials and structures described herein may be substituted with other materials and structures without departing from the spirit of the present disclosure. It is to be understood that various theories as to why the present disclosure works are not intended to be limitative.

What is claimed is:

1. A compound having a structure of Formula 1: 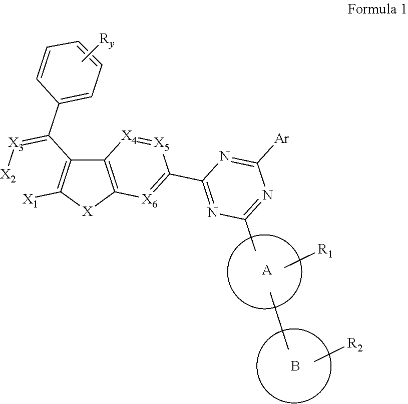
wherein
X is selected from O, S or Se;
X₁ to X₆ are, at each occurrence identically or differently, selected from CRₓ or N;
Ar is, at each occurrence identically or differently, selected from substituted or unsubstituted aryl having 6 to 30 carbon atoms, substituted or unsubstituted heteroaryl having 3 to 30 carbon atoms or a combination thereof;
the ring A and the ring B are, at each occurrence identically or differently, selected from an aromatic ring having 6 to 30 carbon atoms, a heteroaromatic ring having 3 to 30 carbon atoms or a combination thereof;
R_{y} and R₁ represent, at each occurrence identically or differently, mono-substitution, multiple substitutions or non-substitution;
R₂ represents, at each occurrence identically or differently, mono-substitution or multiple substitutions;
at least one of R₂ is selected from cyano;
Rₓ and R₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a sulfanyl group, a sulfinyl group, a sulfonyl group, a phosphino group and combinations thereof;
R₁ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, an isocyano group, a hydroxyl group, a sulfanyl group, a sulfinyl group, a sulfonyl group, a phosphino group and combinations thereof;
adjacent substituents Rₓ, R_{y} can be optionally joined to form a ring; and
adjacent substituents R₁, R₂ can be optionally joined to form a ring.

2. The compound according to claim 1, wherein X is selected from O or S; preferably, X is O.

3. The compound according to claim 1, wherein X₁ to X₆ are, at each occurrence identically or differently, selected from CRₓ.

4. The compound according to claim 1, wherein the ring A and/or the ring B are, at each occurrence identically or differently, selected from an aromatic ring having 6 to 12 carbon atoms or a heteroaromatic ring having 3 to 12 carbon atoms; preferably, the ring A and/or the ring B are, at each occurrence identically or differently, selected from a benzene ring or a 6-membered heteroaromatic ring.

5. The compound according to claim 1, wherein R₁, Rₓ and/or R_{y} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms and combinations thereof;
preferably, R₁, Rₓ and/or R_{y} are, at each occurrence identically or differently, selected from the group consisting of: hydrogen, deuterium, substituted or unsubstituted aryl having 6 to 30 carbon atoms, substituted or unsubstituted heteroaryl having 3 to 30 carbon atoms and combinations thereof; and
more preferably, R₁, Rₓ and/or R_{y} are, at each occurrence identically or differently, selected from the group consisting of: hydrogen, deuterium, substituted or unsubstituted phenyl, substituted or unsubstituted naphthyl, substituted or unsubstituted biphenyl and combinations thereof.

6. The compound according to claim 1, wherein R₂ is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms, substituted or unsubstituted heteroaryl having 3 to 30 carbon atoms, cyano and combinations thereof;
preferably, R₂ is, at each occurrence identically or differently, selected from the group consisting of: hydrogen, deuterium, substituted or unsubstituted aryl having 6 to 30 carbon atoms, substituted or unsubstituted heteroaryl having 3 to 30 carbon atoms, cyano and combinations thereof; and
more preferably, R₂ is, at each occurrence identically or differently, selected from the group consisting of: hydrogen, deuterium, substituted or unsubstituted phenyl, substituted or unsubstituted naphthyl, substituted or unsubstituted biphenyl, cyano and combinations thereof.

7. The compound according to claim 1, wherein Ar is, at each occurrence identically or differently, selected from substituted or unsubstituted aryl having 6 to 20 carbon atoms, substituted or unsubstituted heteroaryl having 3 to 20 carbon atoms or a combination thereof; and
preferably, Ar is, at each occurrence identically or differently, selected from the group consisting of: substituted or unsubstituted phenyl, substituted or unsubstituted naphthyl, substituted or unsubstituted phenanthryl, substituted or unsubstituted biphenyl, substituted or unsubstituted terphenyl, substituted or unsubstituted triphenylenyl, substituted or unsubstituted fluorenyl, substituted or unsubstituted carbazolyl, substituted or unsubstituted dibenzofuryl, substituted or unsubstituted dibenzothienyl and combinations thereof.

8. The compound according to claim 1, wherein the compound is selected from the group consisting of the following compounds: 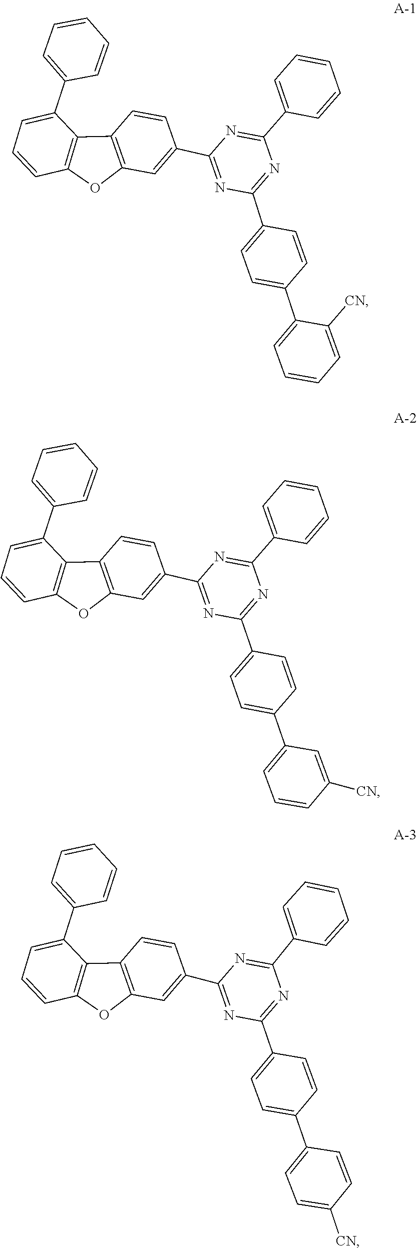 [SMILES: N#Cc1ccccc1-c1ccc(-c2nc(-c3ccccc3)nc(-c3ccc4c(c3)oc3cccc(-c5ccccc5)c34)n2)cc1 ; N#Cc1cccc(-c2ccc(-c3nc(-c4ccccc4)nc(-c4ccc5c(c4)oc4cccc(-c6ccccc6)c45)n3)cc2)c1 ; N#Cc1ccc(-c2ccc(-c3nc(-c4ccccc4)nc(-c4ccc5c(c4)oc4cccc(-c6ccccc6)c45)n3)cc2)cc1] 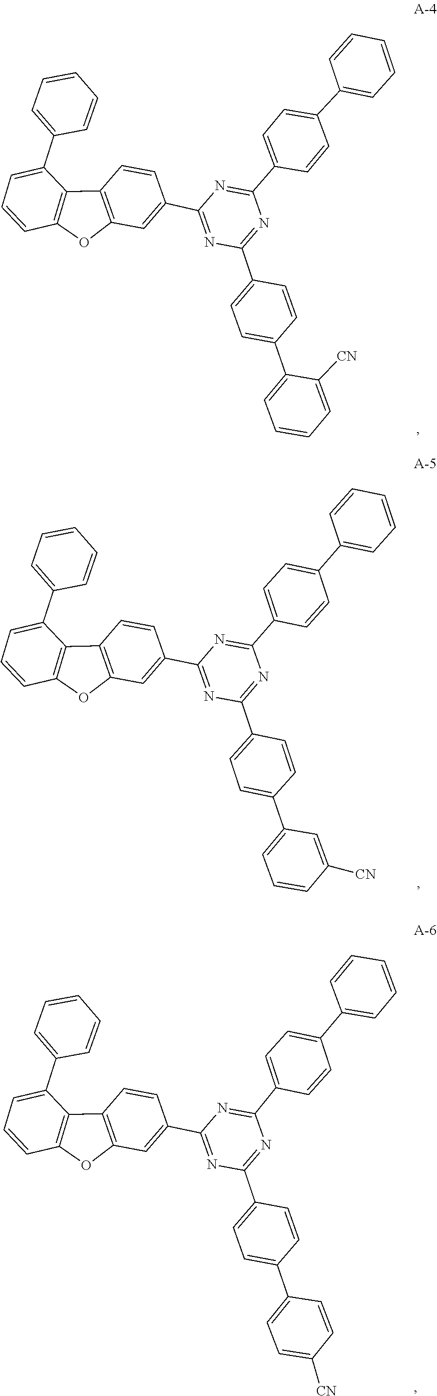 [SMILES: N#Cc1ccccc1-c1ccc(-c2nc(-c3ccc(-c4ccccc4)cc3)nc(-c3ccc4c(c3)oc3cccc(-c5ccccc5)c34)n2)cc1 ; N#Cc1cccc(-c2ccc(-c3nc(-c4ccc(-c5ccccc5)cc4)nc(-c4ccc5c(c4)oc4cccc(-c6ccccc6)c45)n3)cc2)c1 ; N#Cc1ccc(-c2ccc(-c3nc(-c4ccc(-c5ccccc5)cc4)nc(-c4ccc5c(c4)oc4cccc(-c6ccccc6)c45)n3)cc2)cc1] 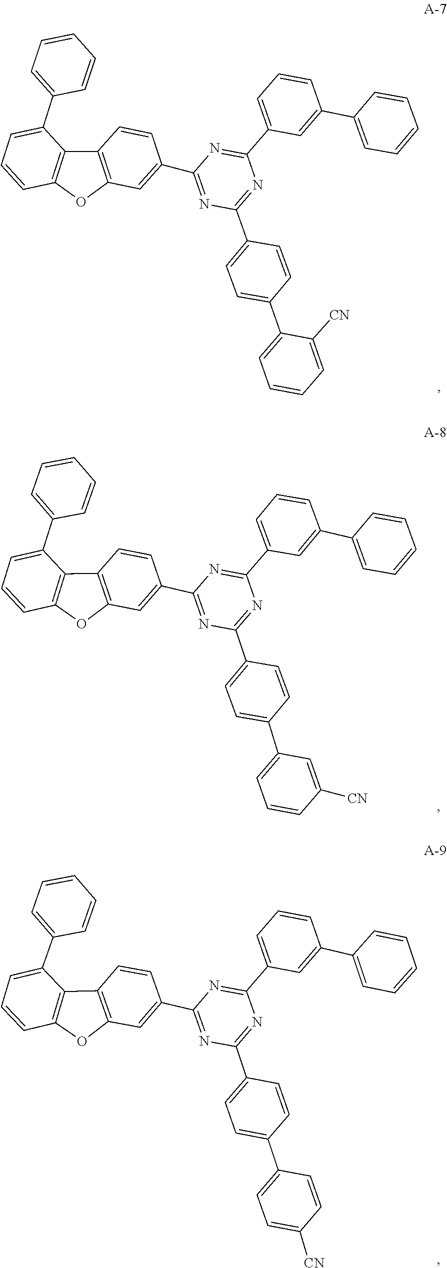 [SMILES: N#Cc1ccccc1-c1ccc(-c2nc(-c3cccc(-c4ccccc4)c3)nc(-c3ccc4c(c3)oc3cccc(-c5ccccc5)c34)n2)cc1 ; N#Cc1cccc(-c2ccc(-c3nc(-c4cccc(-c5ccccc5)c4)nc(-c4ccc5c(c4)oc4cccc(-c6ccccc6)c45)n3)cc2)c1 ; N#Cc1ccc(-c2ccc(-c3nc(-c4cccc(-c5ccccc5)c4)nc(-c4ccc5c(c4)oc4cccc(-c6ccccc6)c45)n3)cc2)cc1] 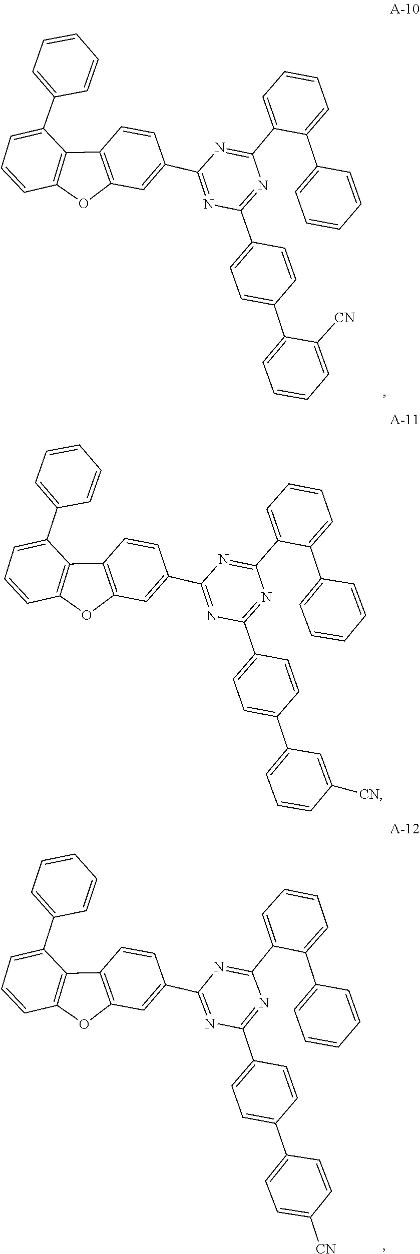 [SMILES: N#Cc1ccccc1-c1ccc(-c2nc(-c3ccc4c(c3)oc3cccc(-c5ccccc5)c34)nc(-c3ccccc3-c3ccccc3)n2)cc1 ; N#Cc1cccc(-c2ccc(-c3nc(-c4ccc5c(c4)oc4cccc(-c6ccccc6)c45)nc(-c4ccccc4-c4ccccc4)n3)cc2)c1 ; N#Cc1ccc(-c2ccc(-c3nc(-c4ccc5c(c4)oc4cccc(-c6ccccc6)c45)nc(-c4ccccc4-c4ccccc4)n3)cc2)cc1] 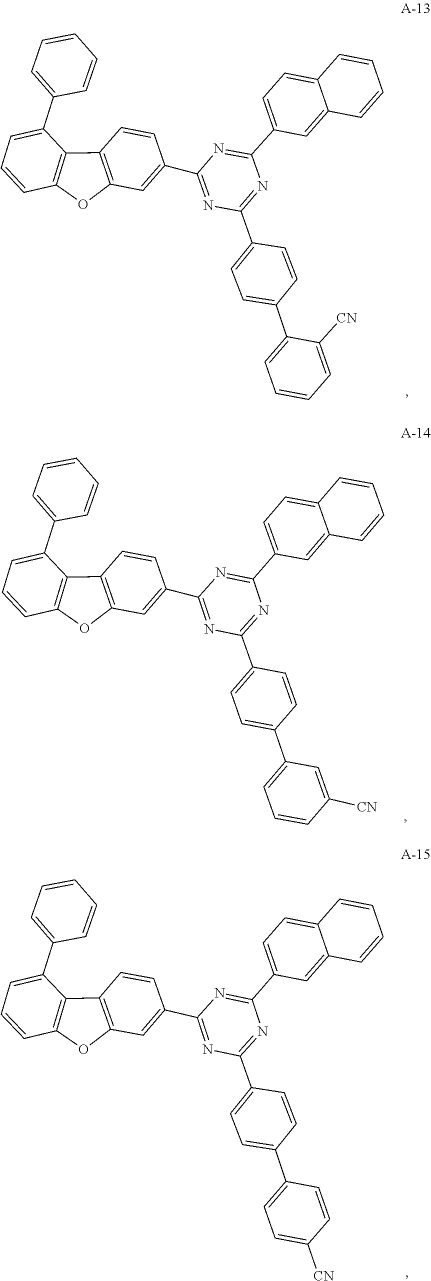 [SMILES: N#Cc1ccccc1-c1ccc(-c2nc(-c3ccc4ccccc4c3)nc(-c3ccc4c(c3)oc3cccc(-c5ccccc5)c34)n2)cc1 ; N#Cc1cccc(-c2ccc(-c3nc(-c4ccc5ccccc5c4)nc(-c4ccc5c(c4)oc4cccc(-c6ccccc6)c45)n3)cc2)c1 ; N#Cc1ccc(-c2ccc(-c3nc(-c4ccc5ccccc5c4)nc(-c4ccc5c(c4)oc4cccc(-c6ccccc6)c45)n3)cc2)cc1] 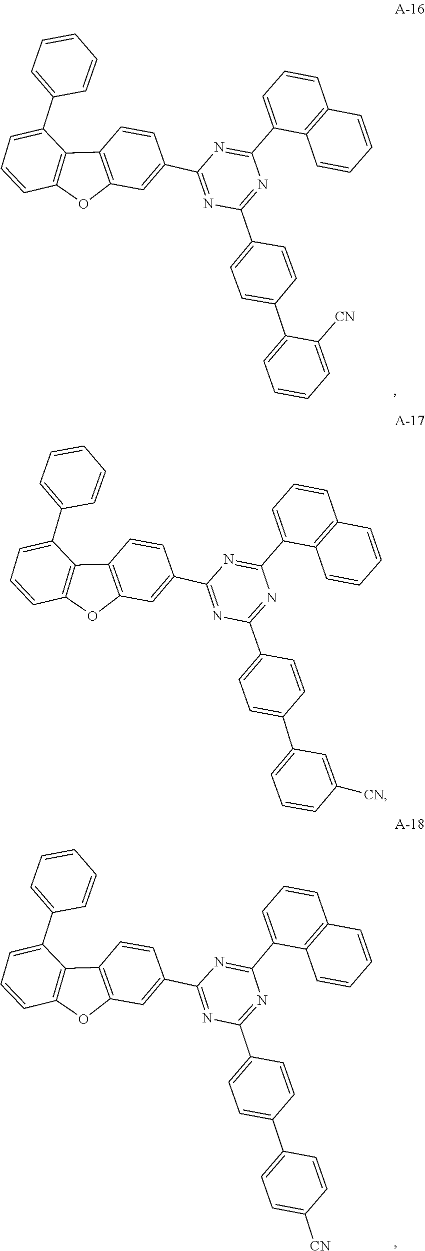 [SMILES: N#Cc1ccccc1-c1ccc(-c2nc(-c3ccc4c(c3)oc3cccc(-c5ccccc5)c34)nc(-c3cccc4ccccc34)n2)cc1 ; N#Cc1cccc(-c2ccc(-c3nc(-c4ccc5c(c4)oc4cccc(-c6ccccc6)c45)nc(-c4cccc5ccccc45)n3)cc2)c1 ; N#Cc1ccc(-c2ccc(-c3nc(-c4ccc5c(c4)oc4cccc(-c6ccccc6)c45)nc(-c4cccc5ccccc45)n3)cc2)cc1] 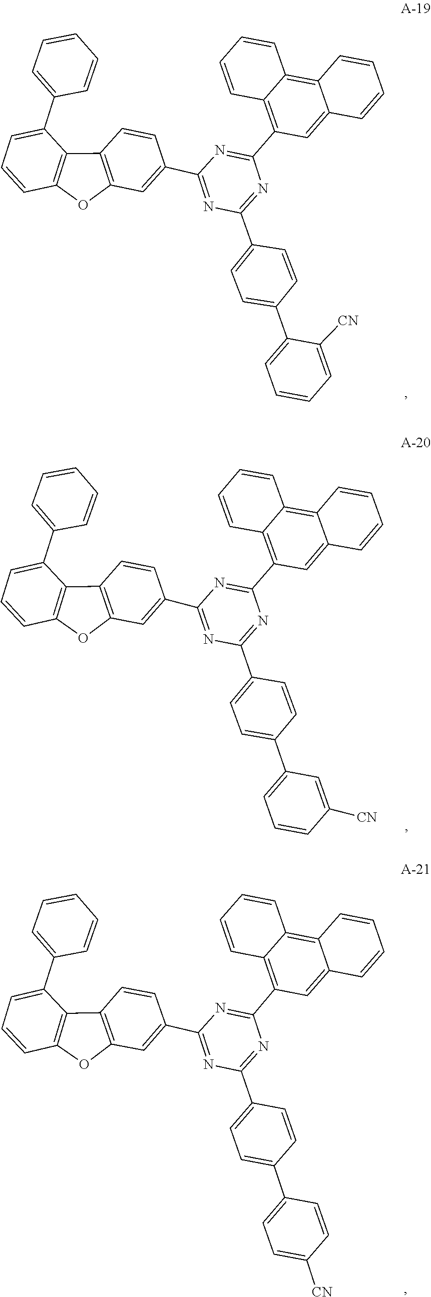 [SMILES: N#Cc1ccccc1-c1ccc(-c2nc(-c3ccc4c(c3)oc3cccc(-c5ccccc5)c34)nc(-c3cc4ccccc4c4ccccc34)n2)cc1 ; N#Cc1cccc(-c2ccc(-c3nc(-c4ccc5c(c4)oc4cccc(-c6ccccc6)c45)nc(-c4cc5ccccc5c5ccccc45)n3)cc2)c1 ; N#Cc1ccc(-c2ccc(-c3nc(-c4ccc5c(c4)oc4cccc(-c6ccccc6)c45)nc(-c4cc5ccccc5c5ccccc45)n3)cc2)cc1] 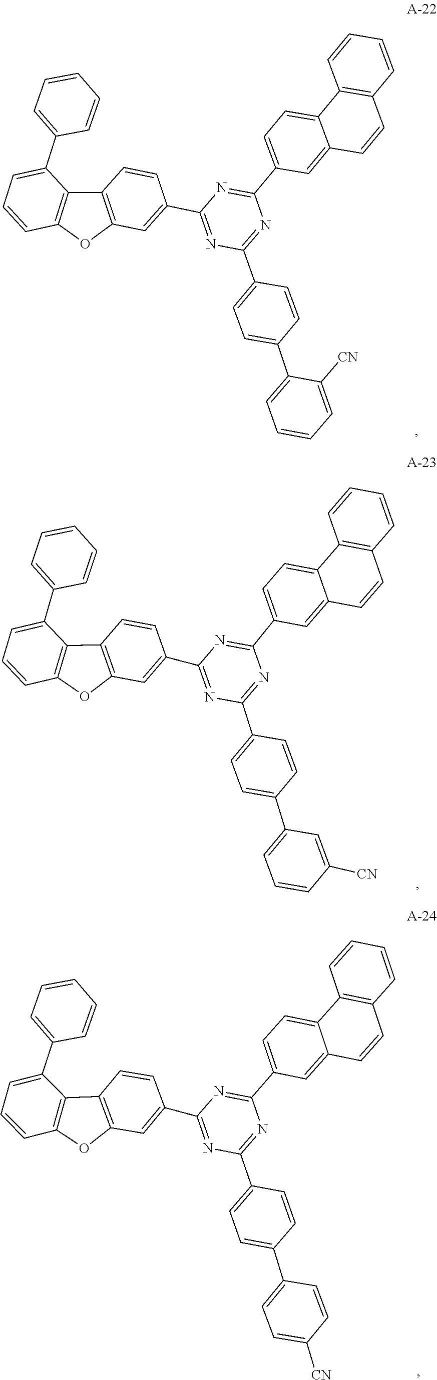 [SMILES: N#Cc1ccccc1-c1ccc(-c2nc(-c3ccc4c(ccc5ccccc54)c3)nc(-c3ccc4c(c3)oc3cccc(-c5ccccc5)c34)n2)cc1 ; N#Cc1cccc(-c2ccc(-c3nc(-c4ccc5c(ccc6ccccc65)c4)nc(-c4ccc5c(c4)oc4cccc(-c6ccccc6)c45)n3)cc2)c1 ; N#Cc1ccc(-c2ccc(-c3nc(-c4ccc5c(ccc6ccccc65)c4)nc(-c4ccc5c(c4)oc4cccc(-c6ccccc6)c45)n3)cc2)cc1] 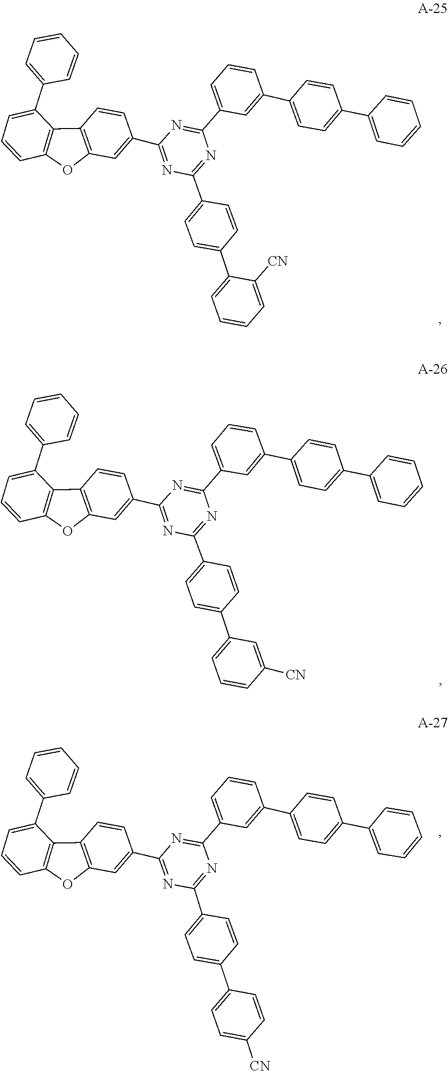 [SMILES: N#Cc1ccccc1-c1ccc(-c2nc(-c3cccc(-c4ccc(-c5ccccc5)cc4)c3)nc(-c3ccc4c(c3)oc3cccc(-c5ccccc5)c34)n2)cc1 ; N#Cc1cccc(-c2ccc(-c3nc(-c4cccc(-c5ccc(-c6ccccc6)cc5)c4)nc(-c4ccc5c(c4)oc4cccc(-c6ccccc6)c45)n3)cc2)c1 ; N#Cc1ccc(-c2ccc(-c3nc(-c4cccc(-c5ccc(-c6ccccc6)cc5)c4)nc(-c4ccc5c(c4)oc4cccc(-c6ccccc6)c45)n3)cc2)cc1] 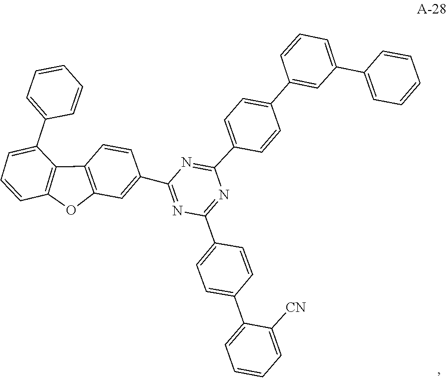 [SMILES: N#Cc1ccccc1-c1ccc(-c2nc(-c3ccc(-c4cccc(-c5ccccc5)c4)cc3)nc(-c3ccc4c(c3)oc3cccc(-c5ccccc5)c34)n2)cc1] 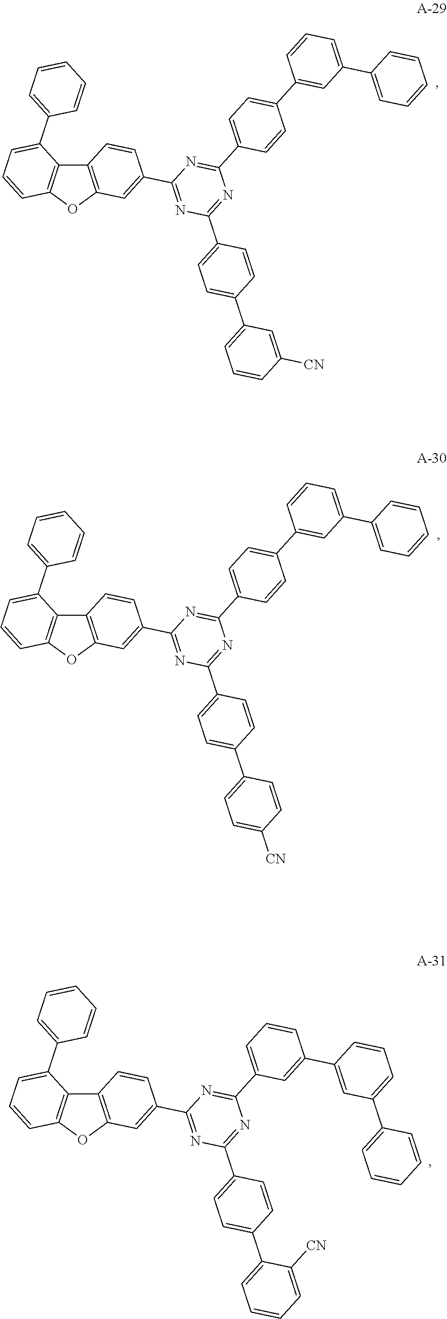 [SMILES: N#Cc1cccc(-c2ccc(-c3nc(-c4ccc(-c5cccc(-c6ccccc6)c5)cc4)nc(-c4ccc5c(c4)oc4cccc(-c6ccccc6)c45)n3)cc2)c1 ; N#Cc1ccc(-c2ccc(-c3nc(-c4ccc(-c5cccc(-c6ccccc6)c5)cc4)nc(-c4ccc5c(c4)oc4cccc(-c6ccccc6)c45)n3)cc2)cc1 ; N#Cc1ccccc1-c1ccc(-c2nc(-c3cccc(-c4cccc(-c5ccccc5)c4)c3)nc(-c3ccc4c(c3)oc3cccc(-c5ccccc5)c34)n2)cc1] 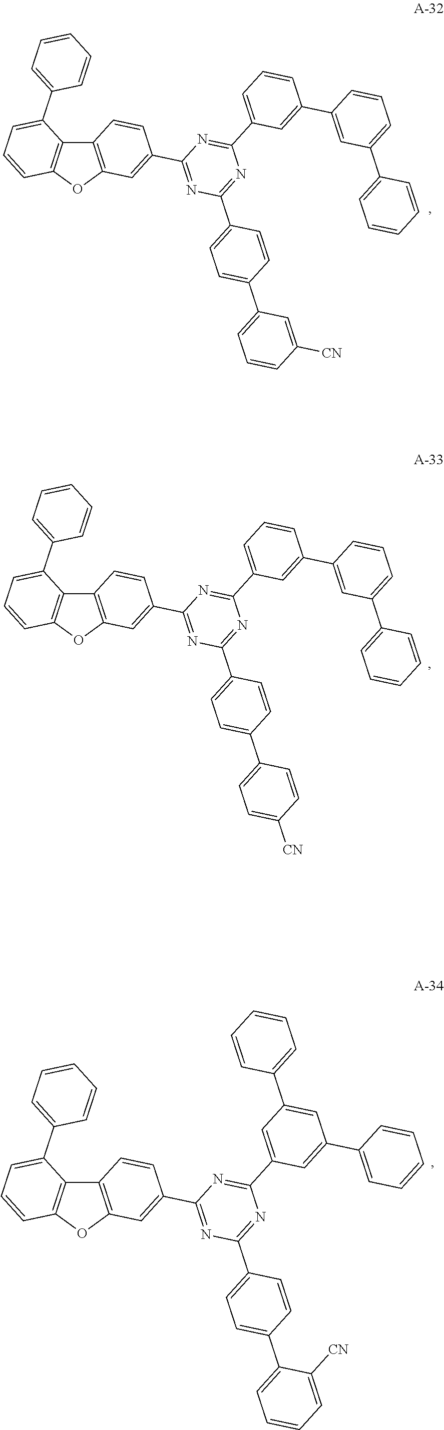 [SMILES: N#Cc1cccc(-c2ccc(-c3nc(-c4cccc(-c5cccc(-c6ccccc6)c5)c4)nc(-c4ccc5c(c4)oc4cccc(-c6ccccc6)c45)n3)cc2)c1 ; N#Cc1ccc(-c2ccc(-c3nc(-c4cccc(-c5cccc(-c6ccccc6)c5)c4)nc(-c4ccc5c(c4)oc4cccc(-c6ccccc6)c45)n3)cc2)cc1 ; N#Cc1ccccc1-c1ccc(-c2nc(-c3cc(-c4ccccc4)cc(-c4ccccc4)c3)nc(-c3ccc4c(c3)oc3cccc(-c5ccccc5)c34)n2)cc1] 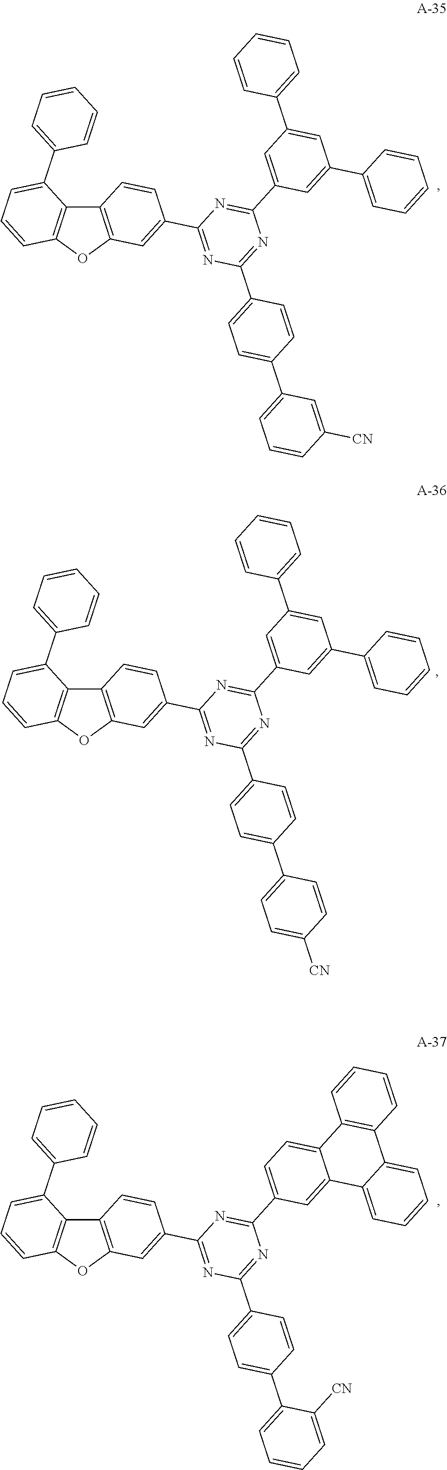 [SMILES: N#Cc1cccc(-c2ccc(-c3nc(-c4cc(-c5ccccc5)cc(-c5ccccc5)c4)nc(-c4ccc5c(c4)oc4cccc(-c6ccccc6)c45)n3)cc2)c1 ; N#Cc1ccc(-c2ccc(-c3nc(-c4cc(-c5ccccc5)cc(-c5ccccc5)c4)nc(-c4ccc5c(c4)oc4cccc(-c6ccccc6)c45)n3)cc2)cc1 ; N#Cc1ccccc1-c1ccc(-c2nc(-c3ccc4c(c3)oc3cccc(-c5ccccc5)c34)nc(-c3ccc4c5ccccc5c5ccccc5c4c3)n2)cc1] 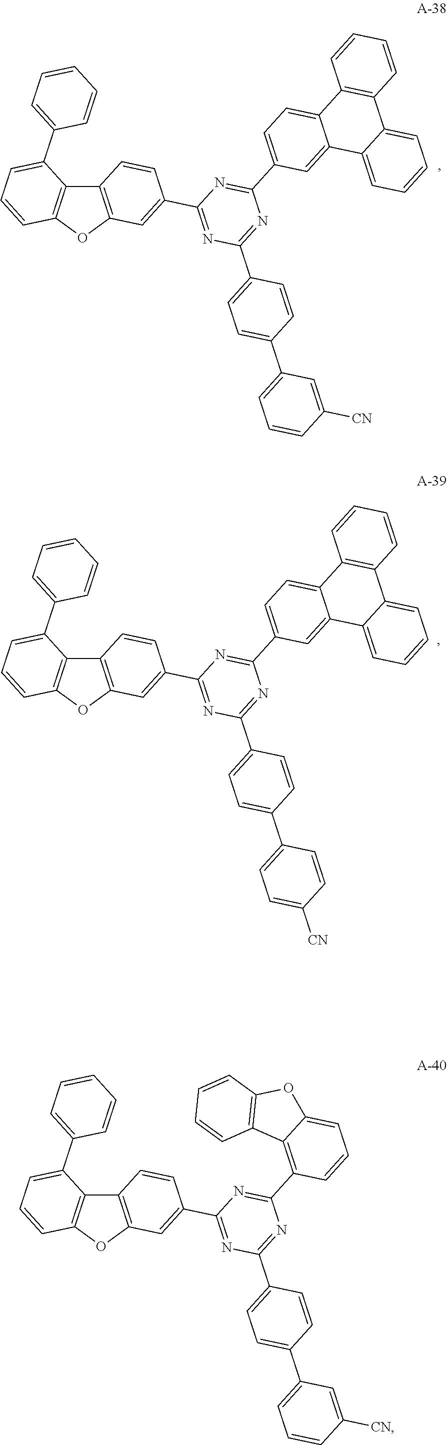 [SMILES: N#Cc1cccc(-c2ccc(-c3nc(-c4ccc5c(c4)oc4cccc(-c6ccccc6)c45)nc(-c4ccc5c6ccccc6c6ccccc6c5c4)n3)cc2)c1 ; N#Cc1ccc(-c2ccc(-c3nc(-c4ccc5c(c4)oc4cccc(-c6ccccc6)c45)nc(-c4ccc5c6ccccc6c6ccccc6c5c4)n3)cc2)cc1 ; N#Cc1cccc(-c2ccc(-c3nc(-c4ccc5c(c4)oc4cccc(-c6ccccc6)c45)nc(-c4cccc5oc6ccccc6c45)n3)cc2)c1] 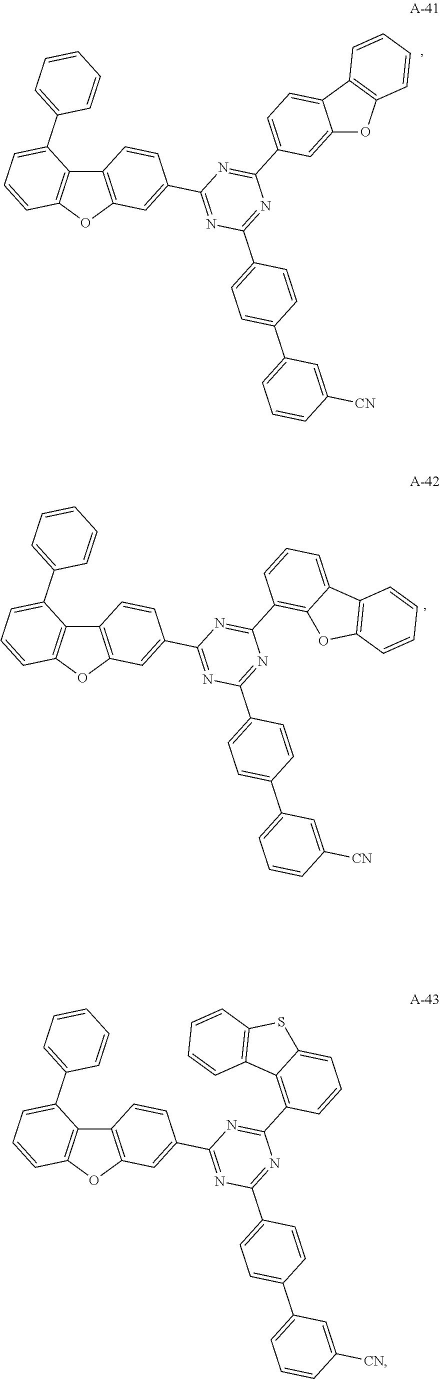 [SMILES: N#Cc1cccc(-c2ccc(-c3nc(-c4ccc5c(c4)oc4ccccc45)nc(-c4ccc5c(c4)oc4cccc(-c6ccccc6)c45)n3)cc2)c1 ; N#Cc1cccc(-c2ccc(-c3nc(-c4ccc5c(c4)oc4cccc(-c6ccccc6)c45)nc(-c4cccc5c4oc4ccccc45)n3)cc2)c1 ; N#Cc1cccc(-c2ccc(-c3nc(-c4ccc5c(c4)oc4cccc(-c6ccccc6)c45)nc(-c4cccc5sc6ccccc6c45)n3)cc2)c1] 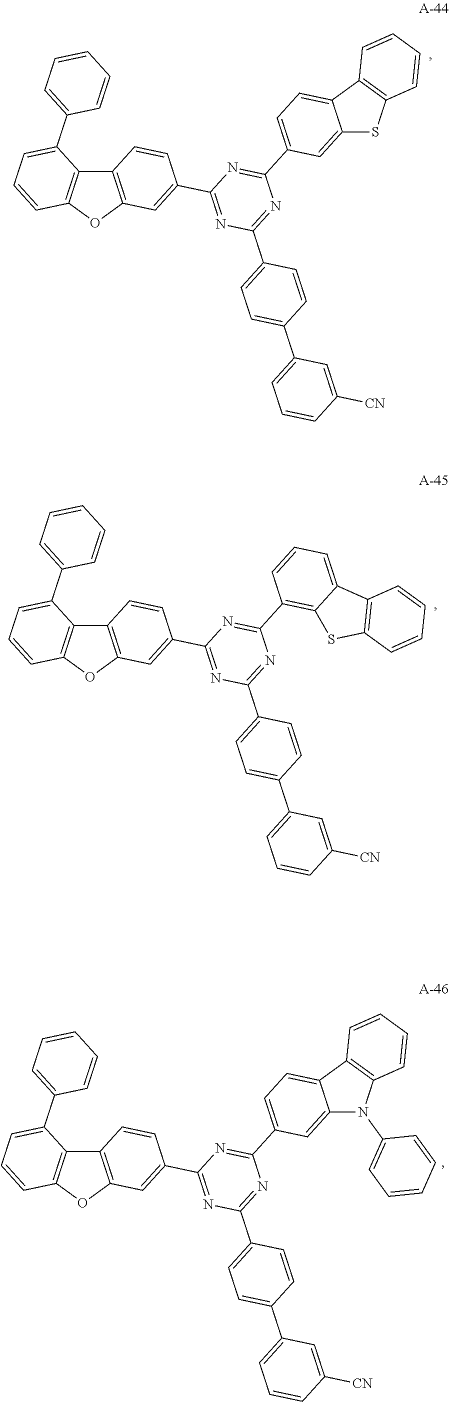 [SMILES: N#Cc1cccc(-c2ccc(-c3nc(-c4ccc5c(c4)oc4cccc(-c6ccccc6)c45)nc(-c4ccc5c(c4)sc4ccccc45)n3)cc2)c1 ; N#Cc1cccc(-c2ccc(-c3nc(-c4ccc5c(c4)oc4cccc(-c6ccccc6)c45)nc(-c4cccc5c4sc4ccccc45)n3)cc2)c1 ; N#Cc1cccc(-c2ccc(-c3nc(-c4ccc5c(c4)oc4cccc(-c6ccccc6)c45)nc(-c4ccc5c6ccccc6n(-c6ccccc6)c5c4)n3)cc2)c1] 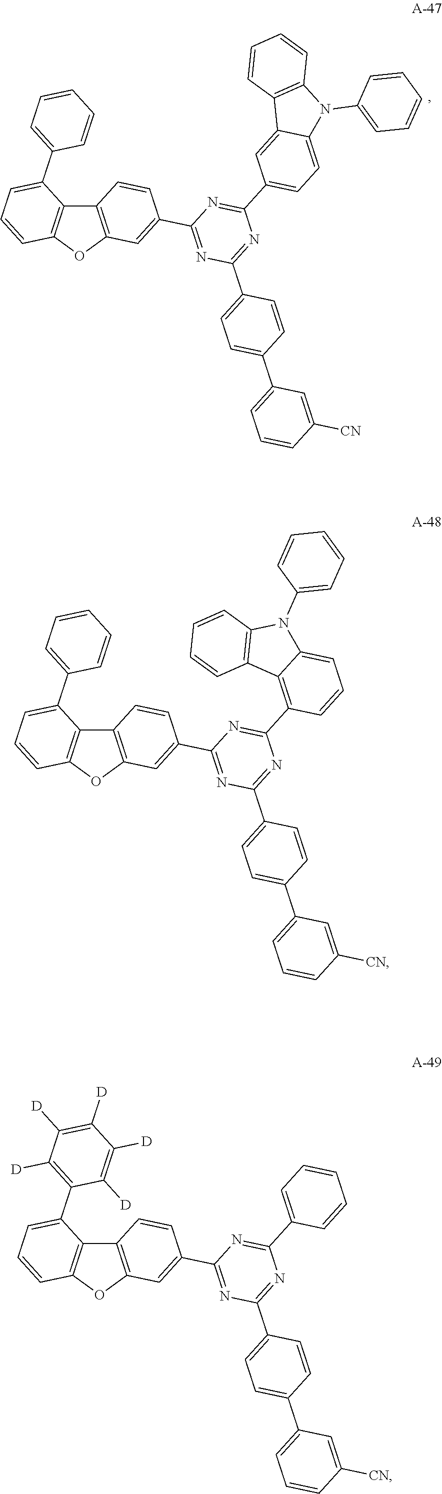 [SMILES: N#Cc1cccc(-c2ccc(-c3nc(-c4ccc5c(c4)oc4cccc(-c6ccccc6)c45)nc(-c4ccc5c(c4)c4ccccc4n5-c4ccccc4)n3)cc2)c1 ; N#Cc1cccc(-c2ccc(-c3nc(-c4ccc5c(c4)oc4cccc(-c6ccccc6)c45)nc(-c4cccc5c4c4ccccc4n5-c4ccccc4)n3)cc2)c1 ; [2H]c1c([2H])c([2H])c(-c2cccc3oc4cc(-c5nc(-c6ccccc6)nc(-c6ccc(-c7cccc(C#N)c7)cc6)n5)ccc4c23)c([2H])c1[2H]] 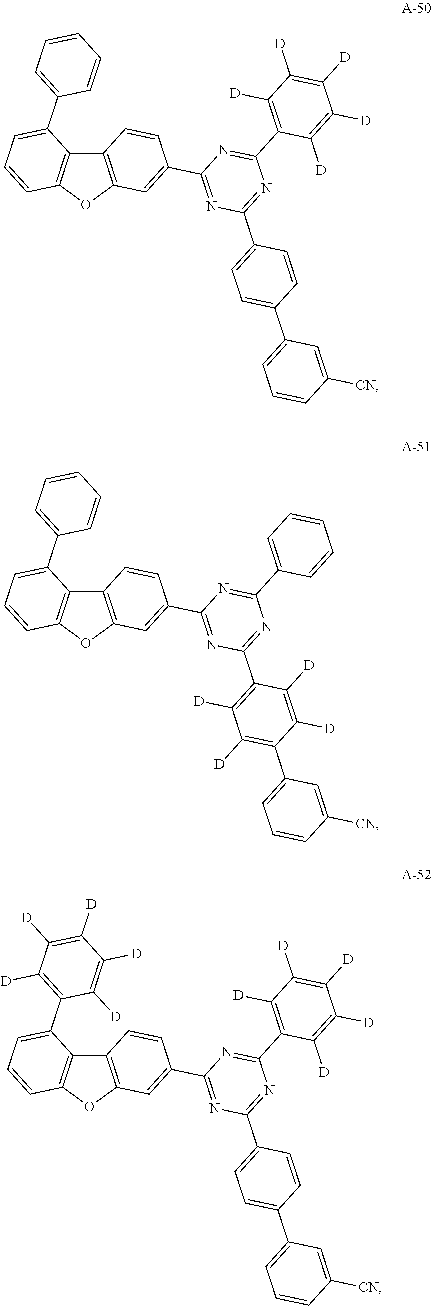 [SMILES: [2H]c1c([2H])c([2H])c(-c2nc(-c3ccc(-c4cccc(C#N)c4)cc3)nc(-c3ccc4c(c3)oc3cccc(-c5ccccc5)c34)n2)c([2H])c1[2H] ; [2H]c1c([2H])c(-c2nc(-c3ccccc3)nc(-c3ccc4c(c3)oc3cccc(-c5ccccc5)c34)n2)c([2H])c([2H])c1-c1cccc(C#N)c1 ; [2H]c1c([2H])c([2H])c(-c2nc(-c3ccc(-c4cccc(C#N)c4)cc3)nc(-c3ccc4c(c3)oc3cccc(-c5c([2H])c([2H])c([2H])c([2H])c5[2H])c34)n2)c([2H])c1[2H]] 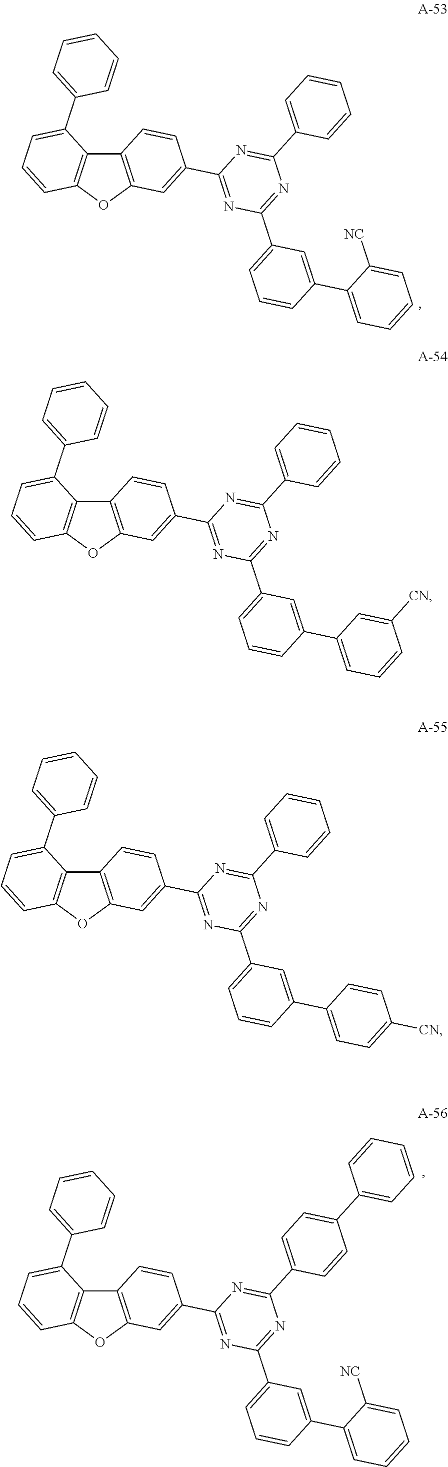 [SMILES: N#Cc1ccccc1-c1cccc(-c2nc(-c3ccccc3)nc(-c3ccc4c(c3)oc3cccc(-c5ccccc5)c34)n2)c1 ; N#Cc1cccc(-c2cccc(-c3nc(-c4ccccc4)nc(-c4ccc5c(c4)oc4cccc(-c6ccccc6)c45)n3)c2)c1 ; N#Cc1ccc(-c2cccc(-c3nc(-c4ccccc4)nc(-c4ccc5c(c4)oc4cccc(-c6ccccc6)c45)n3)c2)cc1 ; N#Cc1ccccc1-c1cccc(-c2nc(-c3ccc(-c4ccccc4)cc3)nc(-c3ccc4c(c3)oc3cccc(-c5ccccc5)c34)n2)c1] 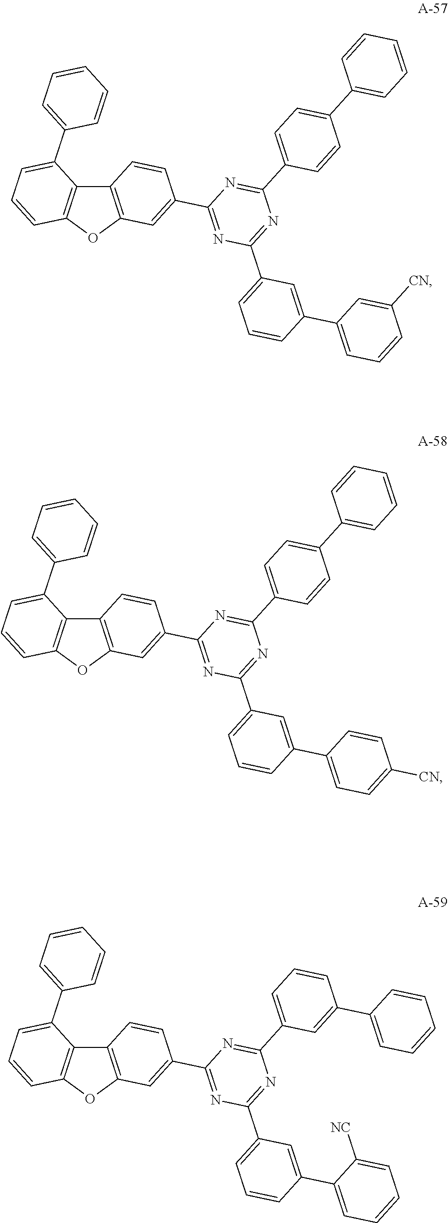 [SMILES: N#Cc1cccc(-c2cccc(-c3nc(-c4ccc(-c5ccccc5)cc4)nc(-c4ccc5c(c4)oc4cccc(-c6ccccc6)c45)n3)c2)c1 ; N#Cc1ccc(-c2cccc(-c3nc(-c4ccc(-c5ccccc5)cc4)nc(-c4ccc5c(c4)oc4cccc(-c6ccccc6)c45)n3)c2)cc1 ; N#Cc1ccccc1-c1cccc(-c2nc(-c3cccc(-c4ccccc4)c3)nc(-c3ccc4c(c3)oc3cccc(-c5ccccc5)c34)n2)c1] 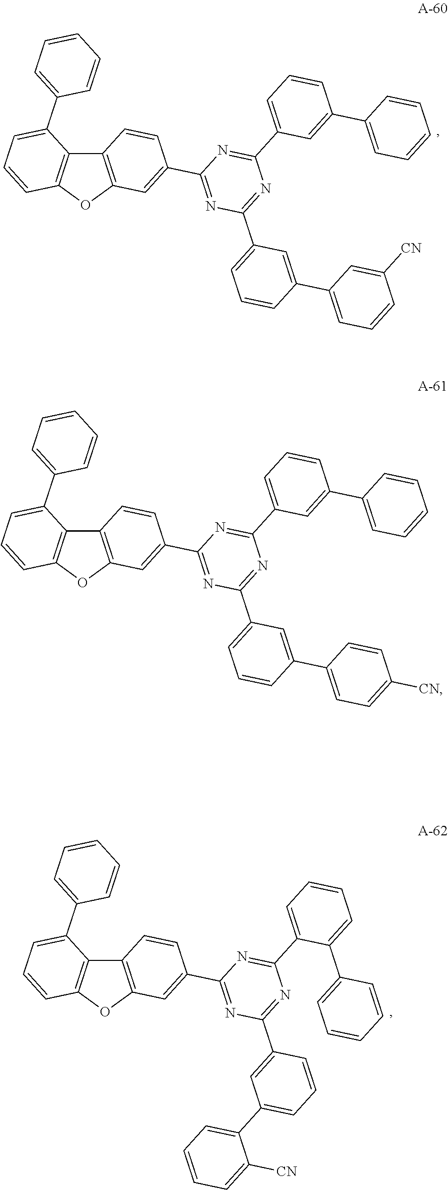 [SMILES: N#Cc1cccc(-c2cccc(-c3nc(-c4cccc(-c5ccccc5)c4)nc(-c4ccc5c(c4)oc4cccc(-c6ccccc6)c45)n3)c2)c1 ; N#Cc1ccc(-c2cccc(-c3nc(-c4cccc(-c5ccccc5)c4)nc(-c4ccc5c(c4)oc4cccc(-c6ccccc6)c45)n3)c2)cc1 ; N#Cc1ccccc1-c1cccc(-c2nc(-c3ccc4c(c3)oc3cccc(-c5ccccc5)c34)nc(-c3ccccc3-c3ccccc3)n2)c1] 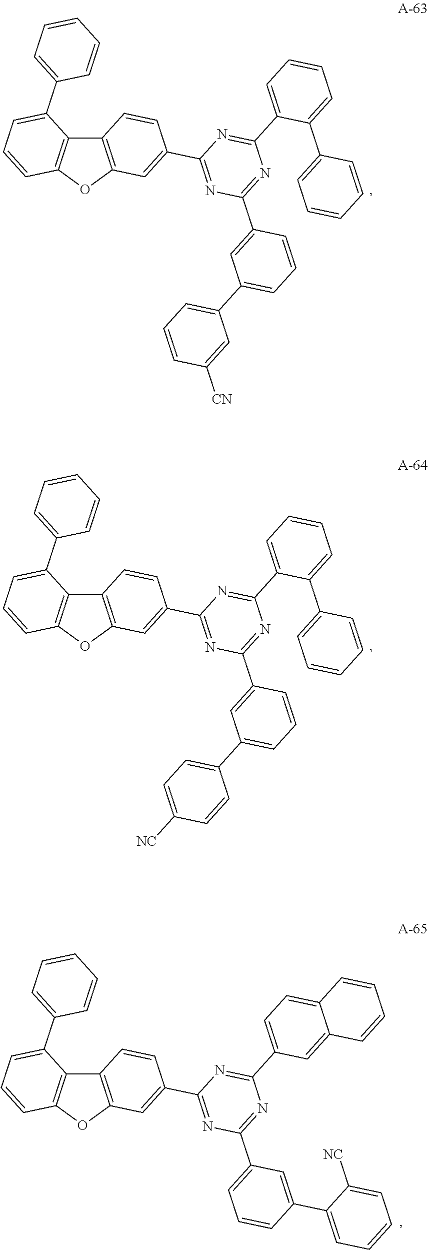 [SMILES: N#Cc1cccc(-c2cccc(-c3nc(-c4ccc5c(c4)oc4cccc(-c6ccccc6)c45)nc(-c4ccccc4-c4ccccc4)n3)c2)c1 ; N#Cc1ccc(-c2cccc(-c3nc(-c4ccc5c(c4)oc4cccc(-c6ccccc6)c45)nc(-c4ccccc4-c4ccccc4)n3)c2)cc1 ; N#Cc1ccccc1-c1cccc(-c2nc(-c3ccc4ccccc4c3)nc(-c3ccc4c(c3)oc3cccc(-c5ccccc5)c34)n2)c1] 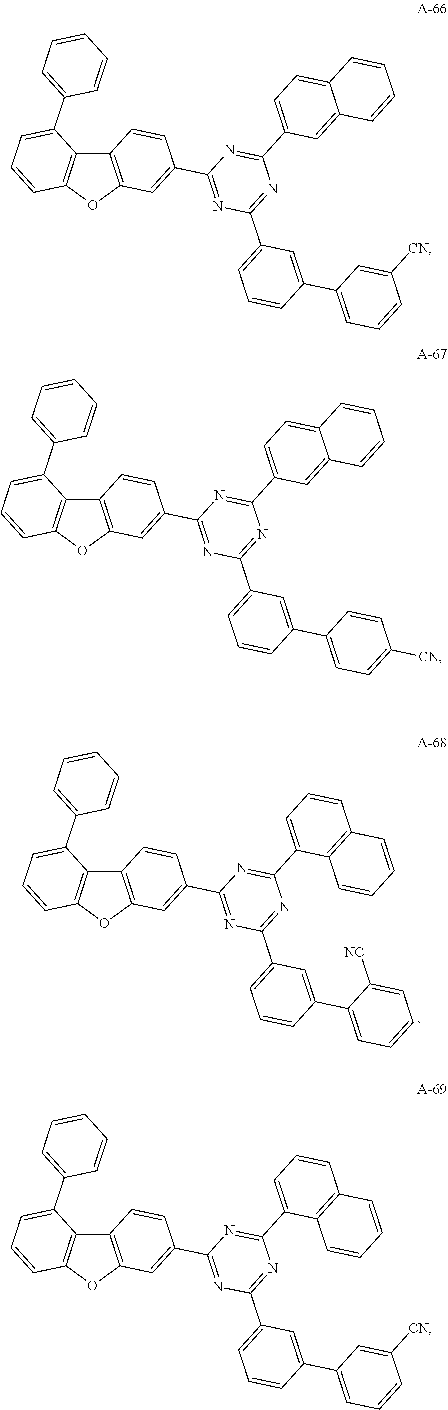 [SMILES: N#Cc1cccc(-c2cccc(-c3nc(-c4ccc5ccccc5c4)nc(-c4ccc5c(c4)oc4cccc(-c6ccccc6)c45)n3)c2)c1 ; N#Cc1ccc(-c2cccc(-c3nc(-c4ccc5ccccc5c4)nc(-c4ccc5c(c4)oc4cccc(-c6ccccc6)c45)n3)c2)cc1 ; N#Cc1ccccc1-c1cccc(-c2nc(-c3ccc4c(c3)oc3cccc(-c5ccccc5)c34)nc(-c3cccc4ccccc34)n2)c1 ; N#Cc1cccc(-c2cccc(-c3nc(-c4ccc5c(c4)oc4cccc(-c6ccccc6)c45)nc(-c4cccc5ccccc45)n3)c2)c1] 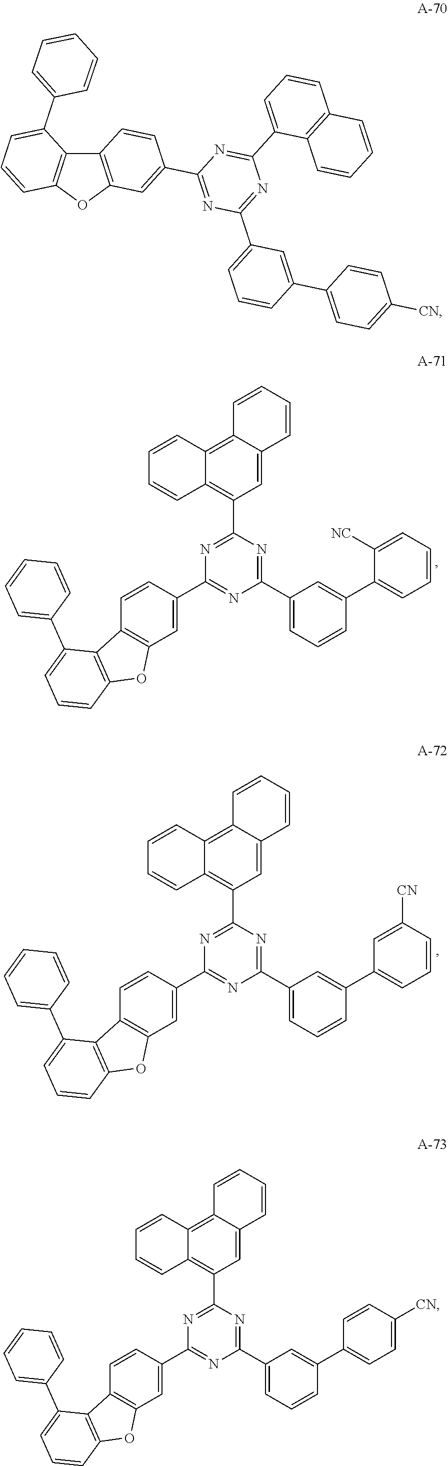 [SMILES: N#Cc1ccc(-c2cccc(-c3nc(-c4ccc5c(c4)oc4cccc(-c6ccccc6)c45)nc(-c4cccc5ccccc45)n3)c2)cc1 ; N#Cc1ccccc1-c1cccc(-c2nc(-c3ccc4c(c3)oc3cccc(-c5ccccc5)c34)nc(-c3cc4ccccc4c4ccccc34)n2)c1 ; N#Cc1cccc(-c2cccc(-c3nc(-c4ccc5c(c4)oc4cccc(-c6ccccc6)c45)nc(-c4cc5ccccc5c5ccccc45)n3)c2)c1 ; N#Cc1ccc(-c2cccc(-c3nc(-c4ccc5c(c4)oc4cccc(-c6ccccc6)c45)nc(-c4cc5ccccc5c5ccccc45)n3)c2)cc1] 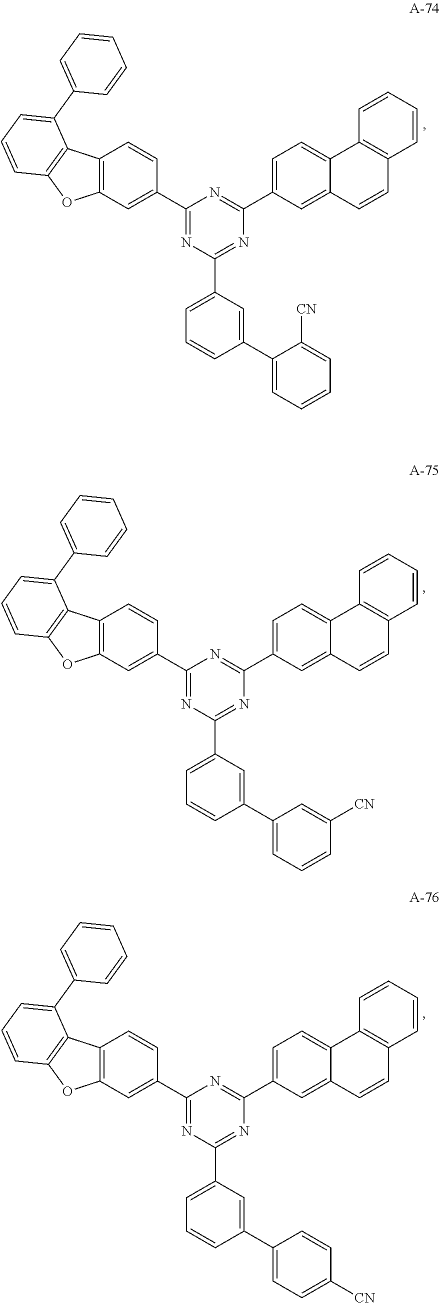 [SMILES: N#Cc1ccccc1-c1cccc(-c2nc(-c3ccc4c(ccc5ccccc54)c3)nc(-c3ccc4c(c3)oc3cccc(-c5ccccc5)c34)n2)c1 ; N#Cc1cccc(-c2cccc(-c3nc(-c4ccc5c(ccc6ccccc65)c4)nc(-c4ccc5c(c4)oc4cccc(-c6ccccc6)c45)n3)c2)c1 ; N#Cc1ccc(-c2cccc(-c3nc(-c4ccc5c(ccc6ccccc65)c4)nc(-c4ccc5c(c4)oc4cccc(-c6ccccc6)c45)n3)c2)cc1] 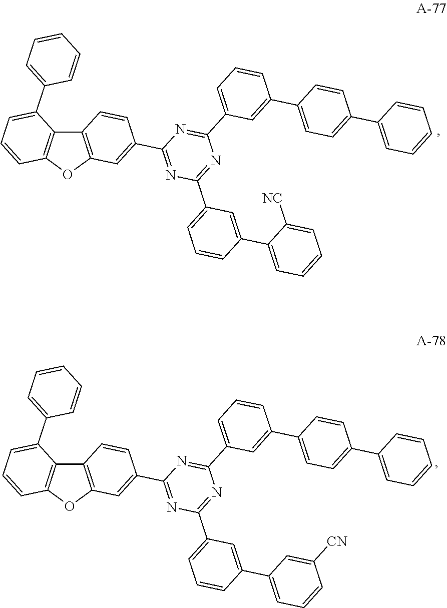 [SMILES: N#Cc1ccccc1-c1cccc(-c2nc(-c3cccc(-c4ccc(-c5ccccc5)cc4)c3)nc(-c3ccc4c(c3)oc3cccc(-c5ccccc5)c34)n2)c1 ; N#Cc1cccc(-c2cccc(-c3nc(-c4cccc(-c5ccc(-c6ccccc6)cc5)c4)nc(-c4ccc5c(c4)oc4cccc(-c6ccccc6)c45)n3)c2)c1] 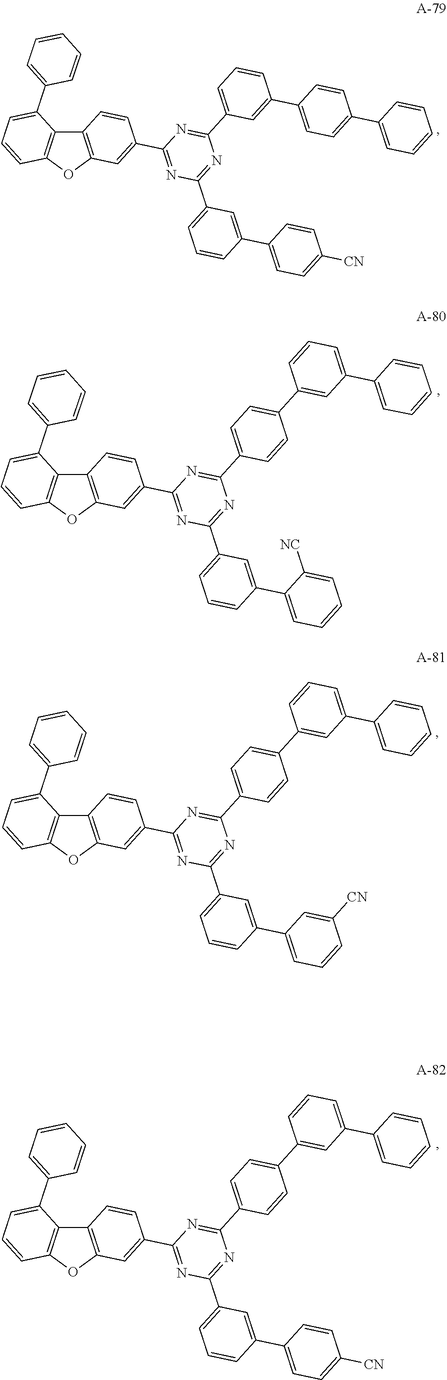 [SMILES: N#Cc1ccc(-c2cccc(-c3nc(-c4cccc(-c5ccc(-c6ccccc6)cc5)c4)nc(-c4ccc5c(c4)oc4cccc(-c6ccccc6)c45)n3)c2)cc1 ; N#Cc1ccccc1-c1cccc(-c2nc(-c3ccc(-c4cccc(-c5ccccc5)c4)cc3)nc(-c3ccc4c(c3)oc3cccc(-c5ccccc5)c34)n2)c1 ; N#Cc1cccc(-c2cccc(-c3nc(-c4ccc(-c5cccc(-c6ccccc6)c5)cc4)nc(-c4ccc5c(c4)oc4cccc(-c6ccccc6)c45)n3)c2)c1 ; N#Cc1ccc(-c2cccc(-c3nc(-c4ccc(-c5cccc(-c6ccccc6)c5)cc4)nc(-c4ccc5c(c4)oc4cccc(-c6ccccc6)c45)n3)c2)cc1] 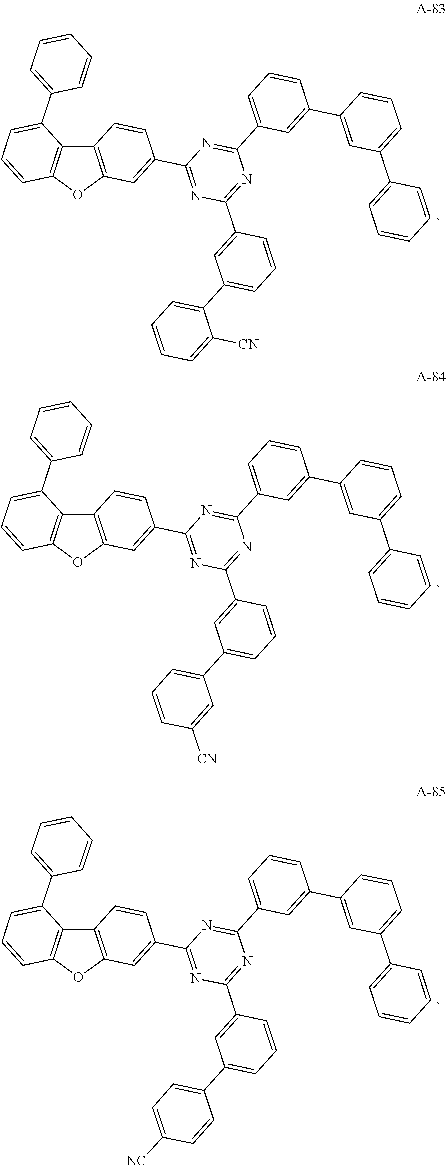 [SMILES: N#Cc1ccccc1-c1cccc(-c2nc(-c3cccc(-c4cccc(-c5ccccc5)c4)c3)nc(-c3ccc4c(c3)oc3cccc(-c5ccccc5)c34)n2)c1 ; N#Cc1cccc(-c2cccc(-c3nc(-c4cccc(-c5cccc(-c6ccccc6)c5)c4)nc(-c4ccc5c(c4)oc4cccc(-c6ccccc6)c45)n3)c2)c1 ; N#Cc1ccc(-c2cccc(-c3nc(-c4cccc(-c5cccc(-c6ccccc6)c5)c4)nc(-c4ccc5c(c4)oc4cccc(-c6ccccc6)c45)n3)c2)cc1] 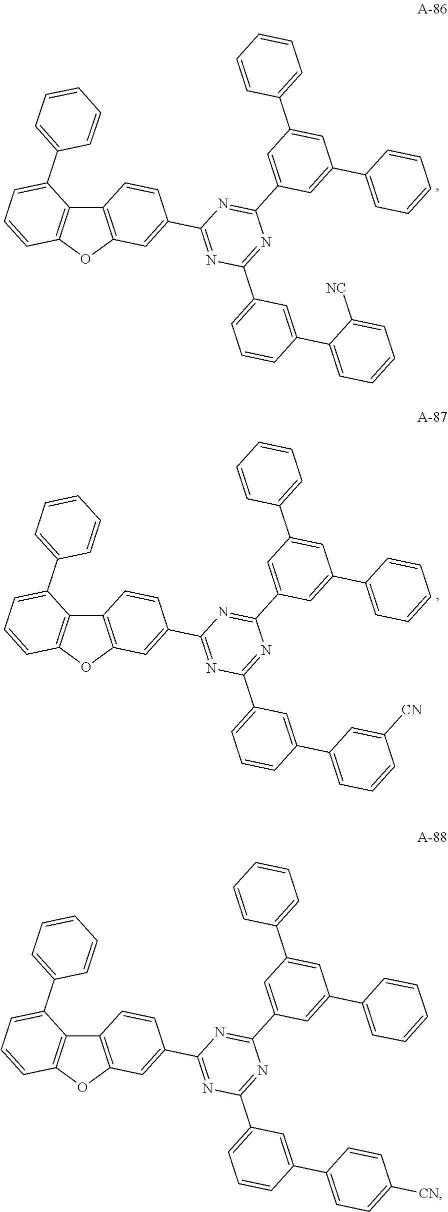 [SMILES: N#Cc1ccccc1-c1cccc(-c2nc(-c3cc(-c4ccccc4)cc(-c4ccccc4)c3)nc(-c3ccc4c(c3)oc3cccc(-c5ccccc5)c34)n2)c1 ; N#Cc1cccc(-c2cccc(-c3nc(-c4cc(-c5ccccc5)cc(-c5ccccc5)c4)nc(-c4ccc5c(c4)oc4cccc(-c6ccccc6)c45)n3)c2)c1 ; N#Cc1ccc(-c2cccc(-c3nc(-c4cc(-c5ccccc5)cc(-c5ccccc5)c4)nc(-c4ccc5c(c4)oc4cccc(-c6ccccc6)c45)n3)c2)cc1] 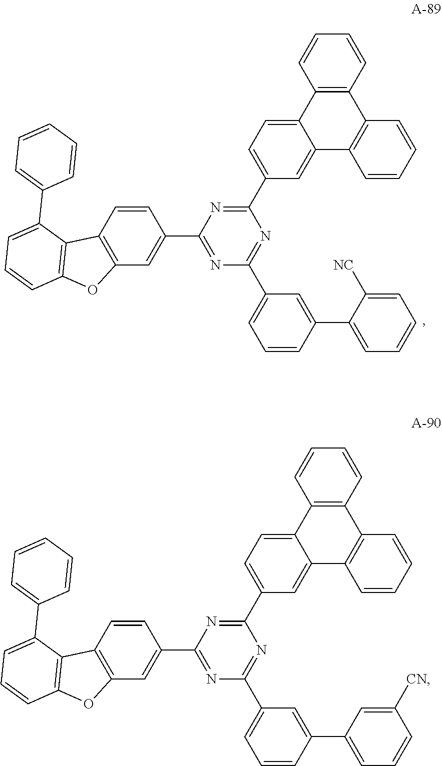 [SMILES: N#Cc1ccccc1-c1cccc(-c2nc(-c3ccc4c(c3)oc3cccc(-c5ccccc5)c34)nc(-c3ccc4c5ccccc5c5ccccc5c4c3)n2)c1 ; N#Cc1cccc(-c2cccc(-c3nc(-c4ccc5c(c4)oc4cccc(-c6ccccc6)c45)nc(-c4ccc5c6ccccc6c6ccccc6c5c4)n3)c2)c1] 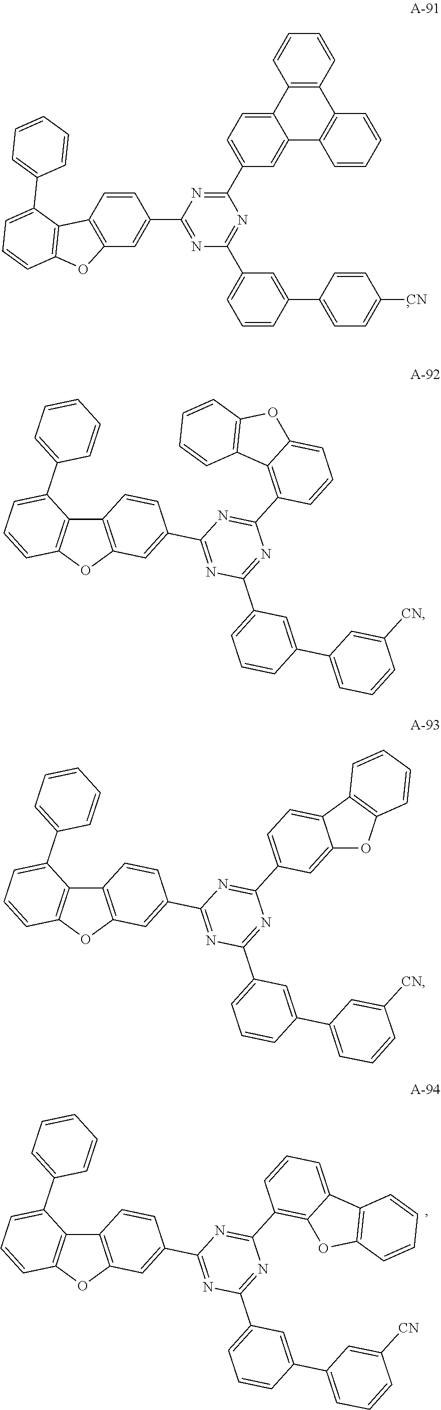 [SMILES: N#Cc1ccc(-c2cccc(-c3nc(-c4ccc5c(c4)oc4cccc(-c6ccccc6)c45)nc(-c4ccc5c6ccccc6c6ccccc6c5c4)n3)c2)cc1 ; N#Cc1cccc(-c2cccc(-c3nc(-c4ccc5c(c4)oc4cccc(-c6ccccc6)c45)nc(-c4cccc5oc6ccccc6c45)n3)c2)c1 ; N#Cc1cccc(-c2cccc(-c3nc(-c4ccc5c(c4)oc4ccccc45)nc(-c4ccc5c(c4)oc4cccc(-c6ccccc6)c45)n3)c2)c1 ; N#Cc1cccc(-c2cccc(-c3nc(-c4ccc5c(c4)oc4cccc(-c6ccccc6)c45)nc(-c4cccc5c4oc4ccccc45)n3)c2)c1] 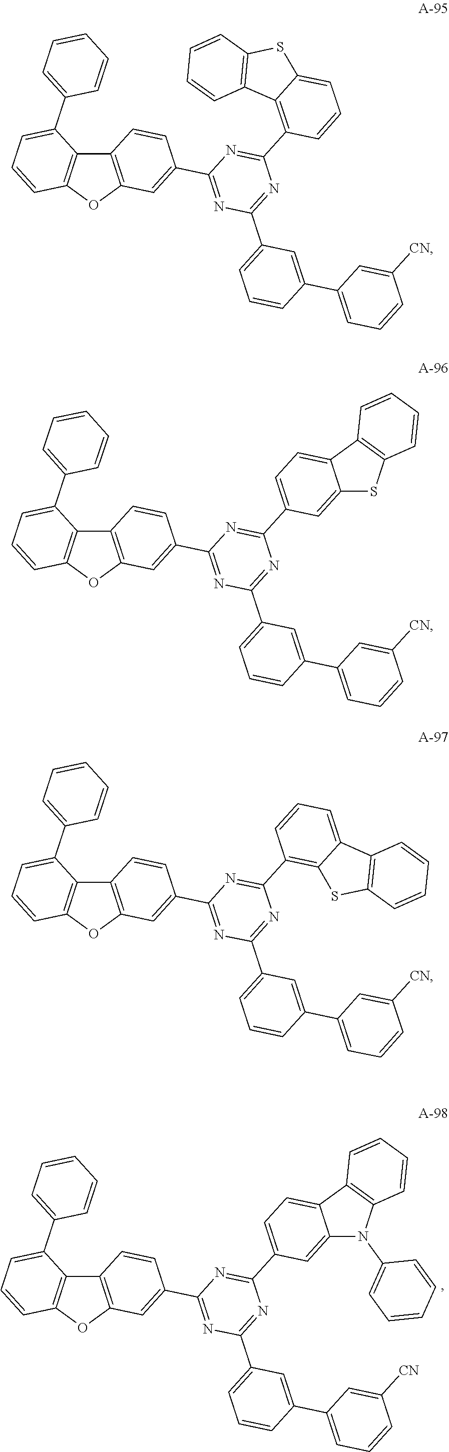 [SMILES: N#Cc1cccc(-c2cccc(-c3nc(-c4ccc5c(c4)oc4cccc(-c6ccccc6)c45)nc(-c4cccc5sc6ccccc6c45)n3)c2)c1 ; N#Cc1cccc(-c2cccc(-c3nc(-c4ccc5c(c4)oc4cccc(-c6ccccc6)c45)nc(-c4ccc5c(c4)sc4ccccc45)n3)c2)c1 ; N#Cc1cccc(-c2cccc(-c3nc(-c4ccc5c(c4)oc4cccc(-c6ccccc6)c45)nc(-c4cccc5c4sc4ccccc45)n3)c2)c1 ; N#Cc1cccc(-c2cccc(-c3nc(-c4ccc5c(c4)oc4cccc(-c6ccccc6)c45)nc(-c4ccc5c6ccccc6n(-c6ccccc6)c5c4)n3)c2)c1] 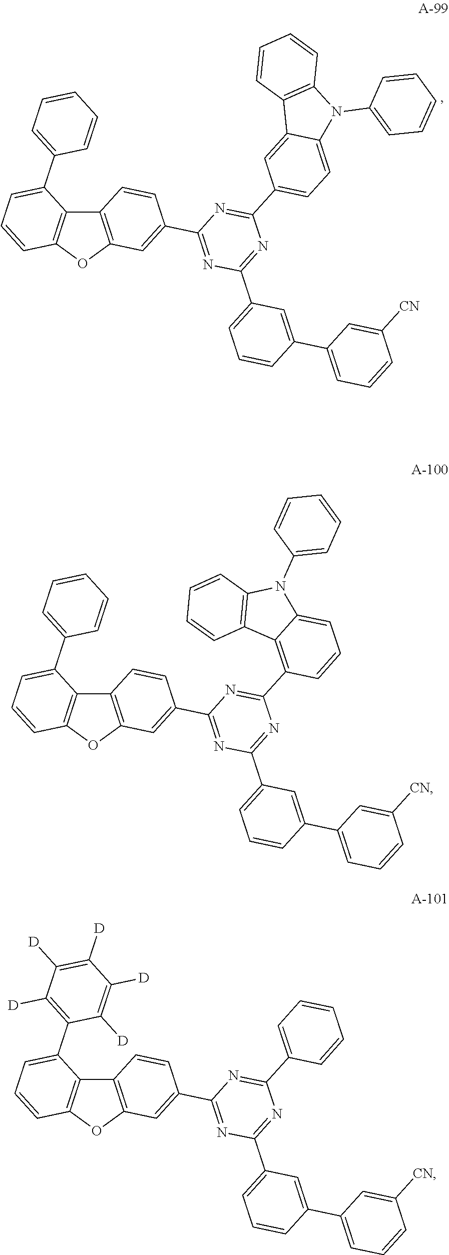 [SMILES: N#Cc1cccc(-c2cccc(-c3nc(-c4ccc5c(c4)oc4cccc(-c6ccccc6)c45)nc(-c4ccc5c(c4)c4ccccc4n5-c4ccccc4)n3)c2)c1 ; N#Cc1cccc(-c2cccc(-c3nc(-c4ccc5c(c4)oc4cccc(-c6ccccc6)c45)nc(-c4cccc5c4c4ccccc4n5-c4ccccc4)n3)c2)c1 ; [2H]c1c([2H])c([2H])c(-c2cccc3oc4cc(-c5nc(-c6ccccc6)nc(-c6cccc(-c7cccc(C#N)c7)c6)n5)ccc4c23)c([2H])c1[2H]] 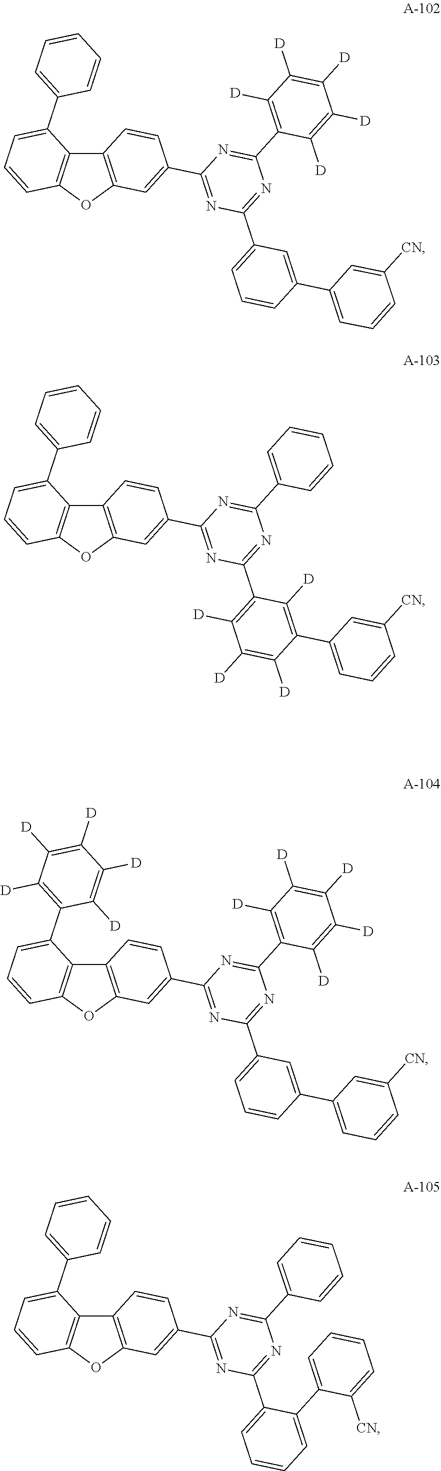 [SMILES: [2H]c1c([2H])c([2H])c(-c2nc(-c3cccc(-c4cccc(C#N)c4)c3)nc(-c3ccc4c(c3)oc3cccc(-c5ccccc5)c34)n2)c([2H])c1[2H] ; [2H]c1c([2H])c(-c2cccc(C#N)c2)c([2H])c(-c2nc(-c3ccccc3)nc(-c3ccc4c(c3)oc3cccc(-c5ccccc5)c34)n2)c1[2H] ; [2H]c1c([2H])c([2H])c(-c2nc(-c3cccc(-c4cccc(C#N)c4)c3)nc(-c3ccc4c(c3)oc3cccc(-c5c([2H])c([2H])c([2H])c([2H])c5[2H])c34)n2)c([2H])c1[2H] ; N#Cc1ccccc1-c1ccccc1-c1nc(-c2ccccc2)nc(-c2ccc3c(c2)oc2cccc(-c4ccccc4)c23)n1] 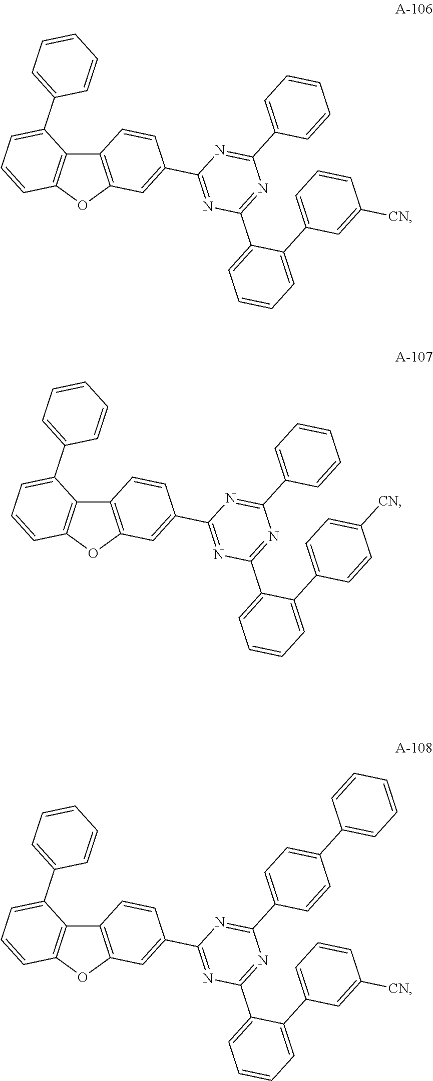 [SMILES: N#Cc1cccc(-c2ccccc2-c2nc(-c3ccccc3)nc(-c3ccc4c(c3)oc3cccc(-c5ccccc5)c34)n2)c1 ; N#Cc1ccc(-c2ccccc2-c2nc(-c3ccccc3)nc(-c3ccc4c(c3)oc3cccc(-c5ccccc5)c34)n2)cc1 ; N#Cc1cccc(-c2ccccc2-c2nc(-c3ccc(-c4ccccc4)cc3)nc(-c3ccc4c(c3)oc3cccc(-c5ccccc5)c34)n2)c1] 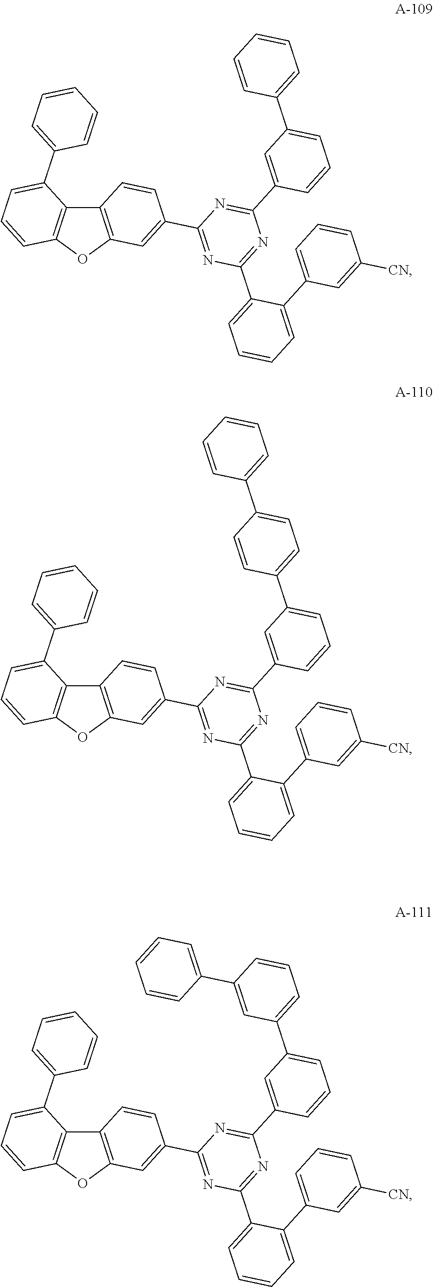 [SMILES: N#Cc1cccc(-c2ccccc2-c2nc(-c3cccc(-c4ccccc4)c3)nc(-c3ccc4c(c3)oc3cccc(-c5ccccc5)c34)n2)c1 ; N#Cc1cccc(-c2ccccc2-c2nc(-c3cccc(-c4ccc(-c5ccccc5)cc4)c3)nc(-c3ccc4c(c3)oc3cccc(-c5ccccc5)c34)n2)c1 ; N#Cc1cccc(-c2ccccc2-c2nc(-c3cccc(-c4cccc(-c5ccccc5)c4)c3)nc(-c3ccc4c(c3)oc3cccc(-c5ccccc5)c34)n2)c1] 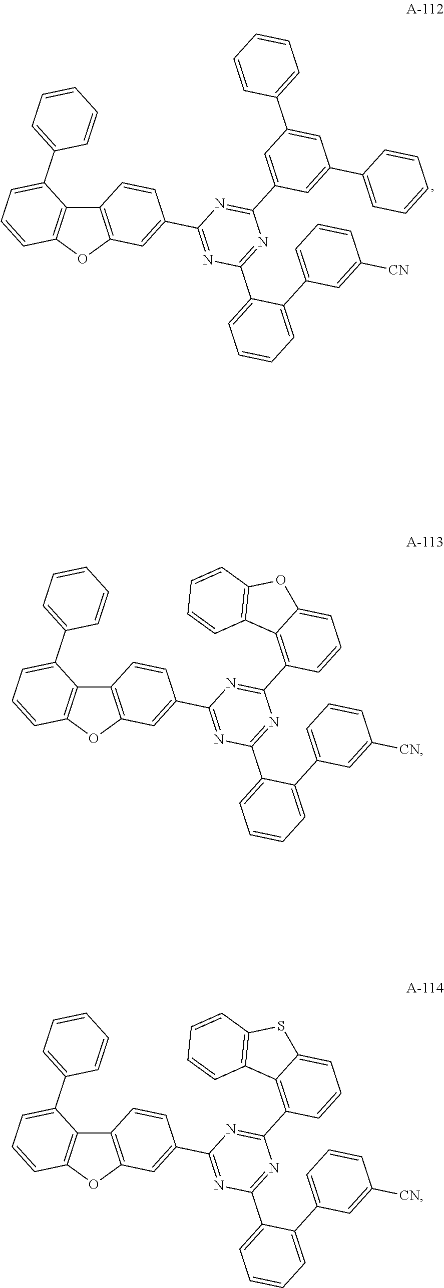 [SMILES: N#Cc1cccc(-c2ccccc2-c2nc(-c3cc(-c4ccccc4)cc(-c4ccccc4)c3)nc(-c3ccc4c(c3)oc3cccc(-c5ccccc5)c34)n2)c1 ; N#Cc1cccc(-c2ccccc2-c2nc(-c3ccc4c(c3)oc3cccc(-c5ccccc5)c34)nc(-c3cccc4oc5ccccc5c34)n2)c1 ; N#Cc1cccc(-c2ccccc2-c2nc(-c3ccc4c(c3)oc3cccc(-c5ccccc5)c34)nc(-c3cccc4sc5ccccc5c34)n2)c1] 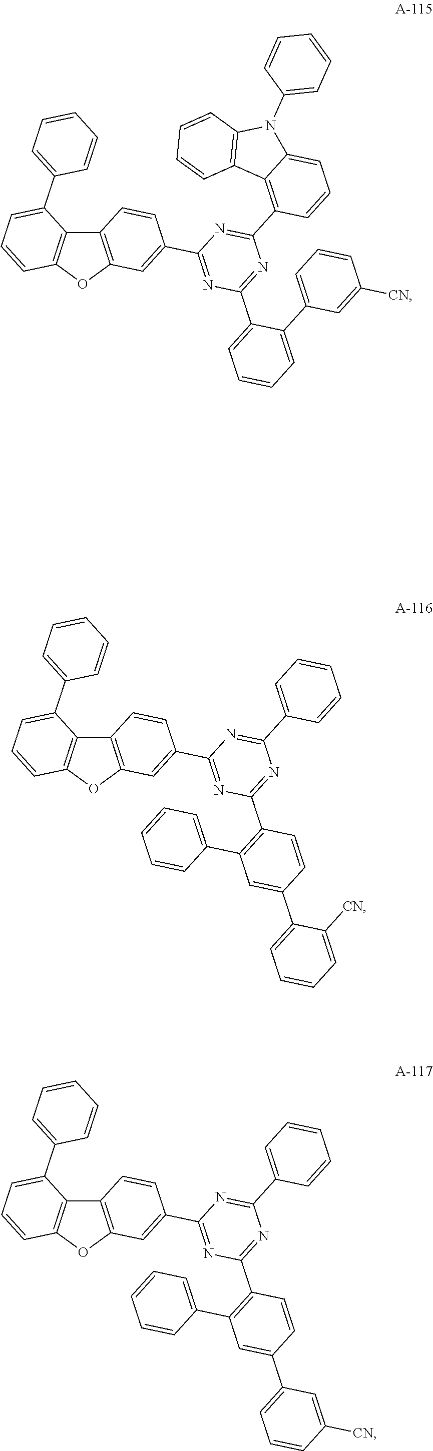 [SMILES: N#Cc1cccc(-c2ccccc2-c2nc(-c3ccc4c(c3)oc3cccc(-c5ccccc5)c34)nc(-c3cccc4c3c3ccccc3n4-c3ccccc3)n2)c1 ; N#Cc1ccccc1-c1ccc(-c2nc(-c3ccccc3)nc(-c3ccc4c(c3)oc3cccc(-c5ccccc5)c34)n2)c(-c2ccccc2)c1 ; N#Cc1cccc(-c2ccc(-c3nc(-c4ccccc4)nc(-c4ccc5c(c4)oc4cccc(-c6ccccc6)c45)n3)c(-c3ccccc3)c2)c1] 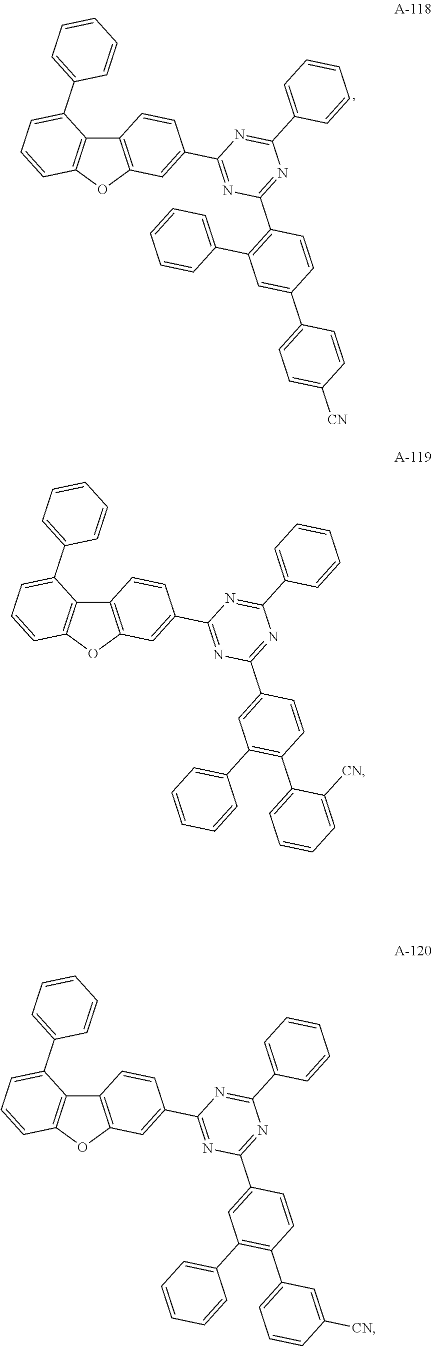 [SMILES: N#Cc1ccc(-c2ccc(-c3nc(-c4ccccc4)nc(-c4ccc5c(c4)oc4cccc(-c6ccccc6)c45)n3)c(-c3ccccc3)c2)cc1 ; N#Cc1ccccc1-c1ccc(-c2nc(-c3ccccc3)nc(-c3ccc4c(c3)oc3cccc(-c5ccccc5)c34)n2)cc1-c1ccccc1 ; N#Cc1cccc(-c2ccc(-c3nc(-c4ccccc4)nc(-c4ccc5c(c4)oc4cccc(-c6ccccc6)c45)n3)cc2-c2ccccc2)c1] 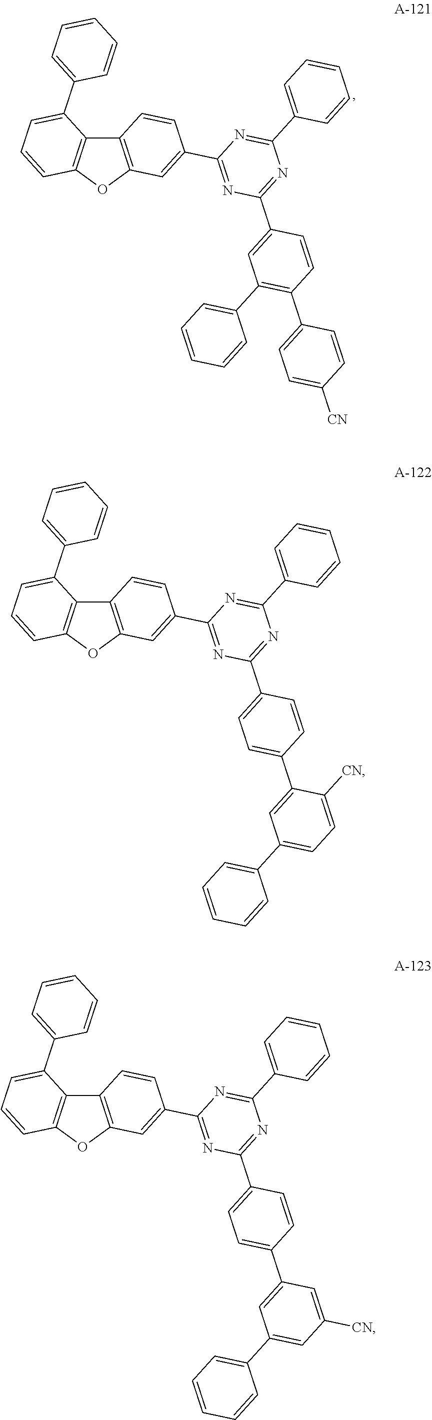 [SMILES: N#Cc1ccc(-c2ccc(-c3nc(-c4ccccc4)nc(-c4ccc5c(c4)oc4cccc(-c6ccccc6)c45)n3)cc2-c2ccccc2)cc1 ; N#Cc1ccc(-c2ccccc2)cc1-c1ccc(-c2nc(-c3ccccc3)nc(-c3ccc4c(c3)oc3cccc(-c5ccccc5)c34)n2)cc1 ; N#Cc1cc(-c2ccccc2)cc(-c2ccc(-c3nc(-c4ccccc4)nc(-c4ccc5c(c4)oc4cccc(-c6ccccc6)c45)n3)cc2)c1] 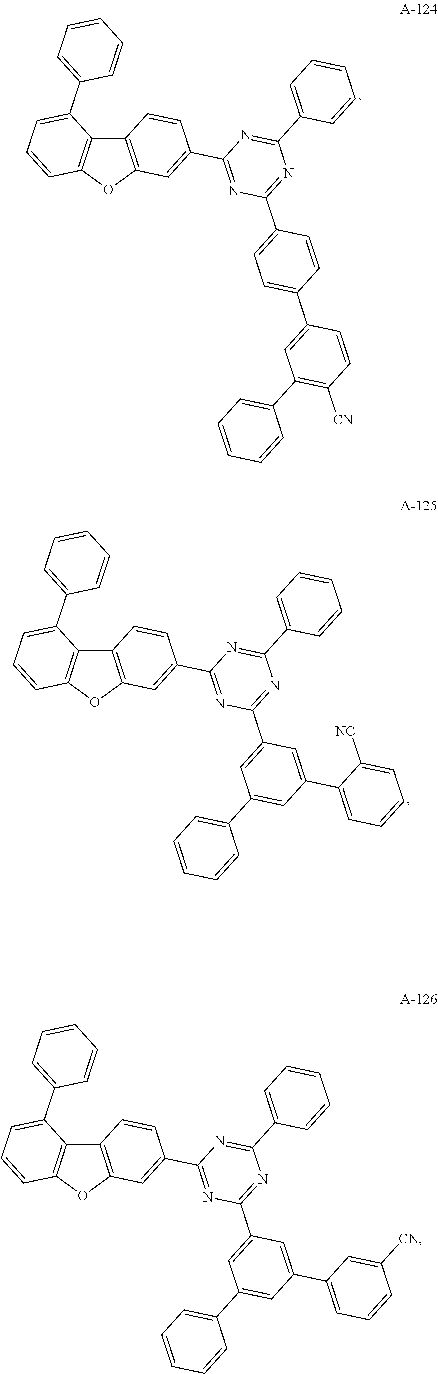 [SMILES: N#Cc1ccc(-c2ccc(-c3nc(-c4ccccc4)nc(-c4ccc5c(c4)oc4cccc(-c6ccccc6)c45)n3)cc2)cc1-c1ccccc1 ; N#Cc1ccccc1-c1cc(-c2ccccc2)cc(-c2nc(-c3ccccc3)nc(-c3ccc4c(c3)oc3cccc(-c5ccccc5)c34)n2)c1 ; N#Cc1cccc(-c2cc(-c3ccccc3)cc(-c3nc(-c4ccccc4)nc(-c4ccc5c(c4)oc4cccc(-c6ccccc6)c45)n3)c2)c1] 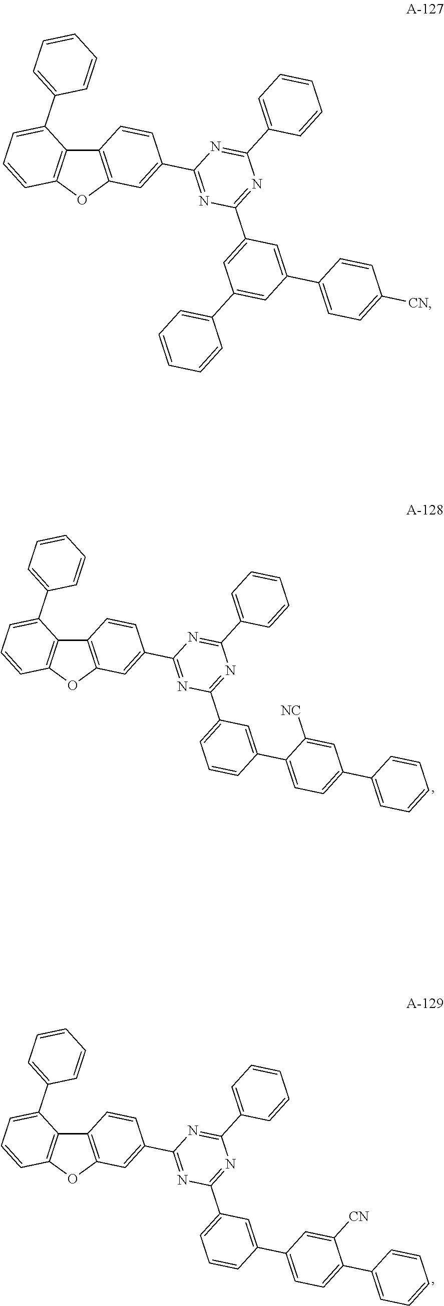 [SMILES: N#Cc1ccc(-c2cc(-c3ccccc3)cc(-c3nc(-c4ccccc4)nc(-c4ccc5c(c4)oc4cccc(-c6ccccc6)c45)n3)c2)cc1 ; N#Cc1cc(-c2ccccc2)ccc1-c1cccc(-c2nc(-c3ccccc3)nc(-c3ccc4c(c3)oc3cccc(-c5ccccc5)c34)n2)c1 ; N#Cc1cc(-c2cccc(-c3nc(-c4ccccc4)nc(-c4ccc5c(c4)oc4cccc(-c6ccccc6)c45)n3)c2)ccc1-c1ccccc1] 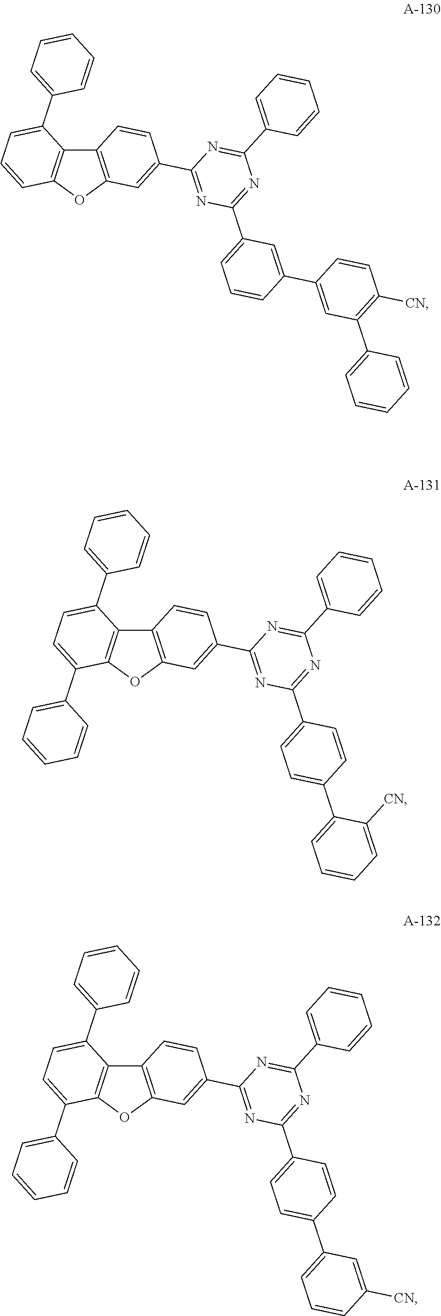 [SMILES: N#Cc1ccc(-c2cccc(-c3nc(-c4ccccc4)nc(-c4ccc5c(c4)oc4cccc(-c6ccccc6)c45)n3)c2)cc1-c1ccccc1 ; N#Cc1ccccc1-c1ccc(-c2nc(-c3ccccc3)nc(-c3ccc4c(c3)oc3c(-c5ccccc5)ccc(-c5ccccc5)c34)n2)cc1 ; N#Cc1cccc(-c2ccc(-c3nc(-c4ccccc4)nc(-c4ccc5c(c4)oc4c(-c6ccccc6)ccc(-c6ccccc6)c45)n3)cc2)c1] 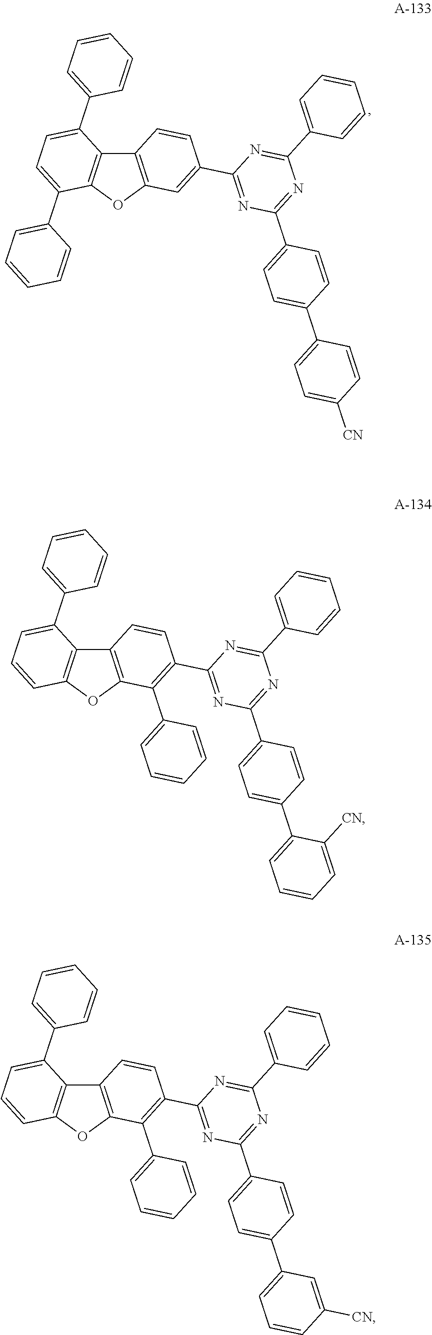 [SMILES: N#Cc1ccc(-c2ccc(-c3nc(-c4ccccc4)nc(-c4ccc5c(c4)oc4c(-c6ccccc6)ccc(-c6ccccc6)c45)n3)cc2)cc1 ; N#Cc1ccccc1-c1ccc(-c2nc(-c3ccccc3)nc(-c3ccc4c(oc5cccc(-c6ccccc6)c54)c3-c3ccccc3)n2)cc1 ; N#Cc1cccc(-c2ccc(-c3nc(-c4ccccc4)nc(-c4ccc5c(oc6cccc(-c7ccccc7)c65)c4-c4ccccc4)n3)cc2)c1] 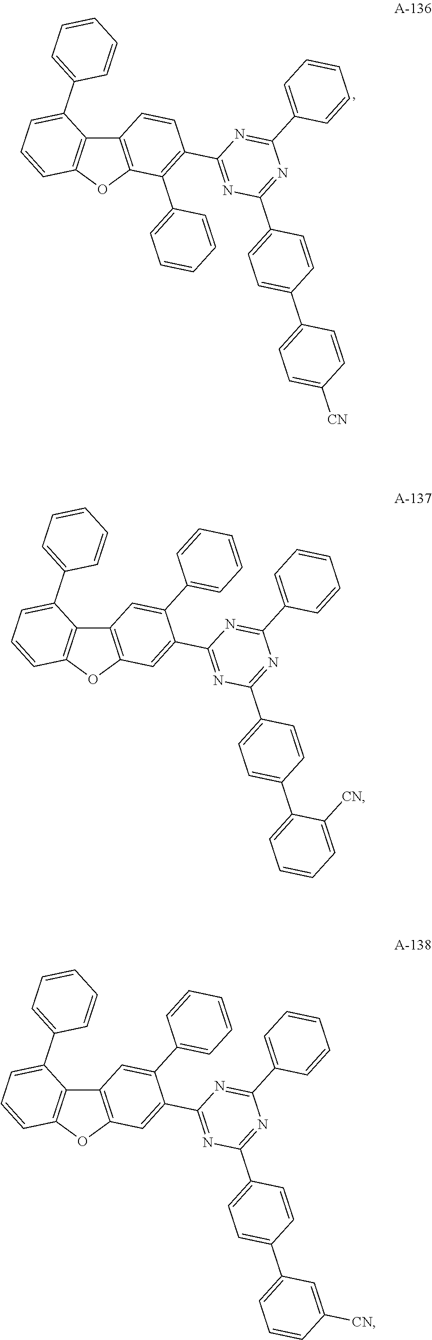 [SMILES: N#Cc1ccc(-c2ccc(-c3nc(-c4ccccc4)nc(-c4ccc5c(oc6cccc(-c7ccccc7)c65)c4-c4ccccc4)n3)cc2)cc1 ; N#Cc1ccccc1-c1ccc(-c2nc(-c3ccccc3)nc(-c3cc4oc5cccc(-c6ccccc6)c5c4cc3-c3ccccc3)n2)cc1 ; N#Cc1cccc(-c2ccc(-c3nc(-c4ccccc4)nc(-c4cc5oc6cccc(-c7ccccc7)c6c5cc4-c4ccccc4)n3)cc2)c1] 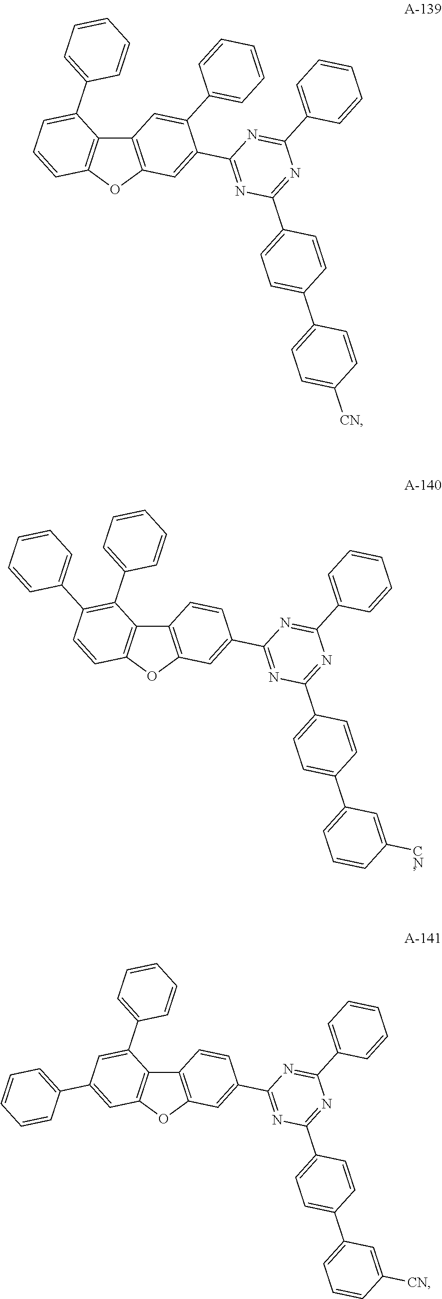 [SMILES: N#Cc1ccc(-c2ccc(-c3nc(-c4ccccc4)nc(-c4cc5oc6cccc(-c7ccccc7)c6c5cc4-c4ccccc4)n3)cc2)cc1 ; N#Cc1cccc(-c2ccc(-c3nc(-c4ccccc4)nc(-c4ccc5c(c4)oc4ccc(-c6ccccc6)c(-c6ccccc6)c45)n3)cc2)c1 ; N#Cc1cccc(-c2ccc(-c3nc(-c4ccccc4)nc(-c4ccc5c(c4)oc4cc(-c6ccccc6)cc(-c6ccccc6)c45)n3)cc2)c1] 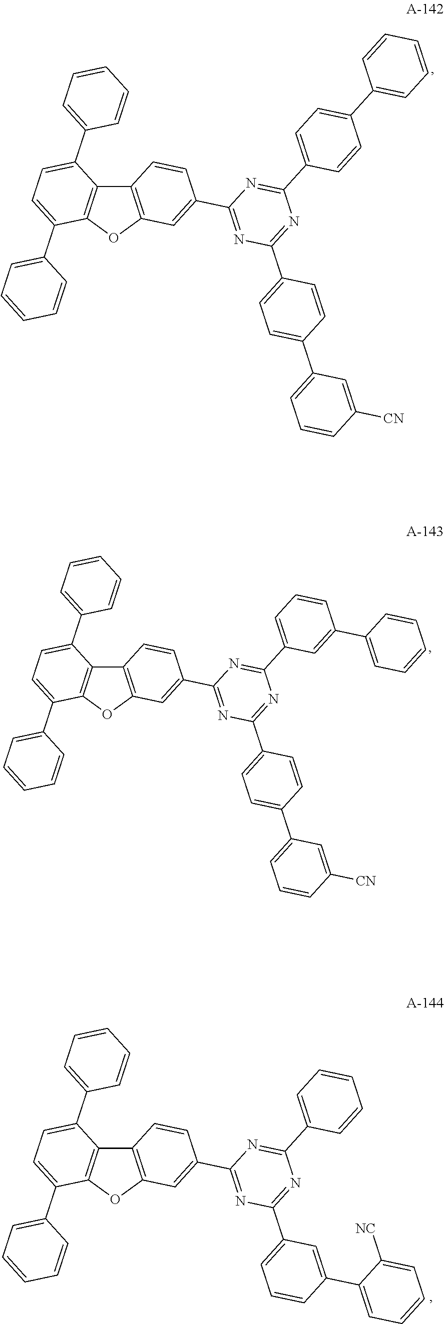 [SMILES: N#Cc1cccc(-c2ccc(-c3nc(-c4ccc(-c5ccccc5)cc4)nc(-c4ccc5c(c4)oc4c(-c6ccccc6)ccc(-c6ccccc6)c45)n3)cc2)c1 ; N#Cc1cccc(-c2ccc(-c3nc(-c4cccc(-c5ccccc5)c4)nc(-c4ccc5c(c4)oc4c(-c6ccccc6)ccc(-c6ccccc6)c45)n3)cc2)c1 ; N#Cc1ccccc1-c1cccc(-c2nc(-c3ccccc3)nc(-c3ccc4c(c3)oc3c(-c5ccccc5)ccc(-c5ccccc5)c34)n2)c1] 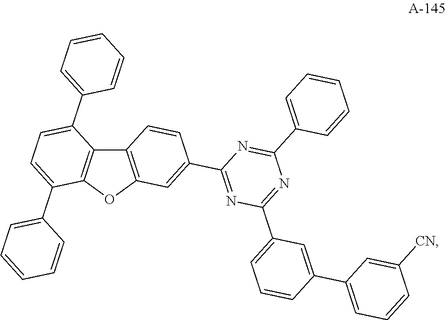 [SMILES: N#Cc1cccc(-c2cccc(-c3nc(-c4ccccc4)nc(-c4ccc5c(c4)oc4c(-c6ccccc6)ccc(-c6ccccc6)c45)n3)c2)c1] 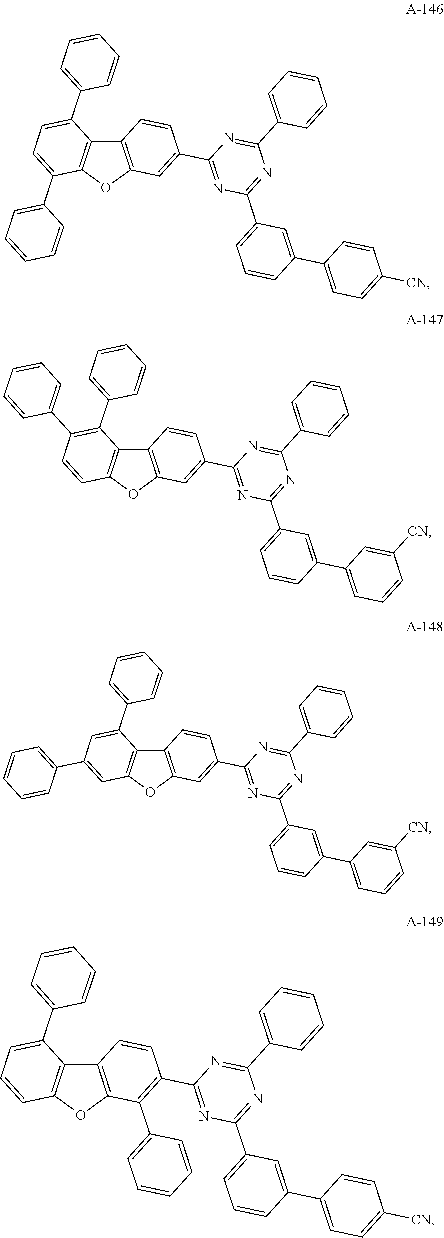 [SMILES: N#Cc1ccc(-c2cccc(-c3nc(-c4ccccc4)nc(-c4ccc5c(c4)oc4c(-c6ccccc6)ccc(-c6ccccc6)c45)n3)c2)cc1 ; N#Cc1cccc(-c2cccc(-c3nc(-c4ccccc4)nc(-c4ccc5c(c4)oc4ccc(-c6ccccc6)c(-c6ccccc6)c45)n3)c2)c1 ; N#Cc1cccc(-c2cccc(-c3nc(-c4ccccc4)nc(-c4ccc5c(c4)oc4cc(-c6ccccc6)cc(-c6ccccc6)c45)n3)c2)c1 ; N#Cc1ccc(-c2cccc(-c3nc(-c4ccccc4)nc(-c4ccc5c(oc6cccc(-c7ccccc7)c65)c4-c4ccccc4)n3)c2)cc1] 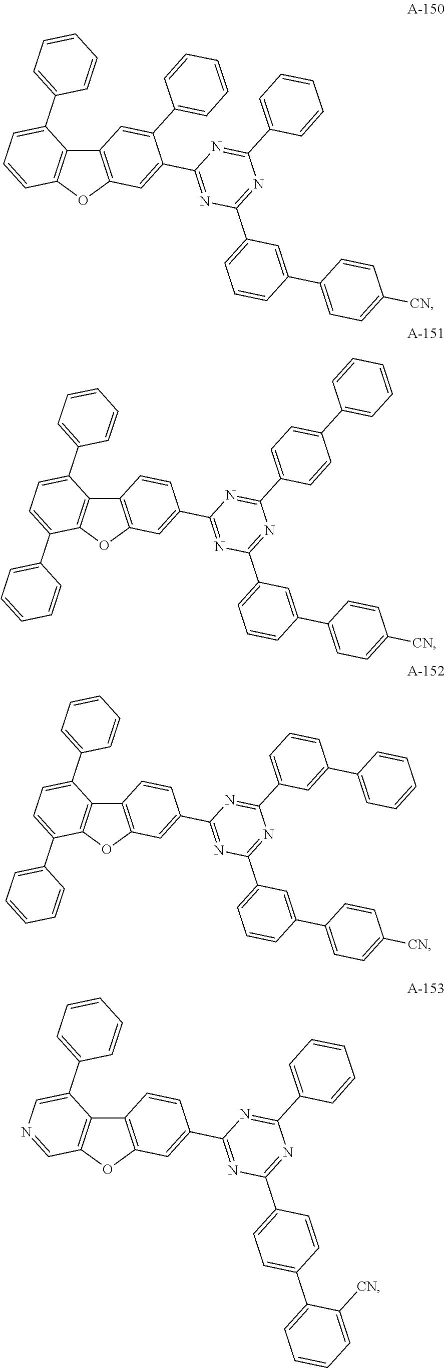 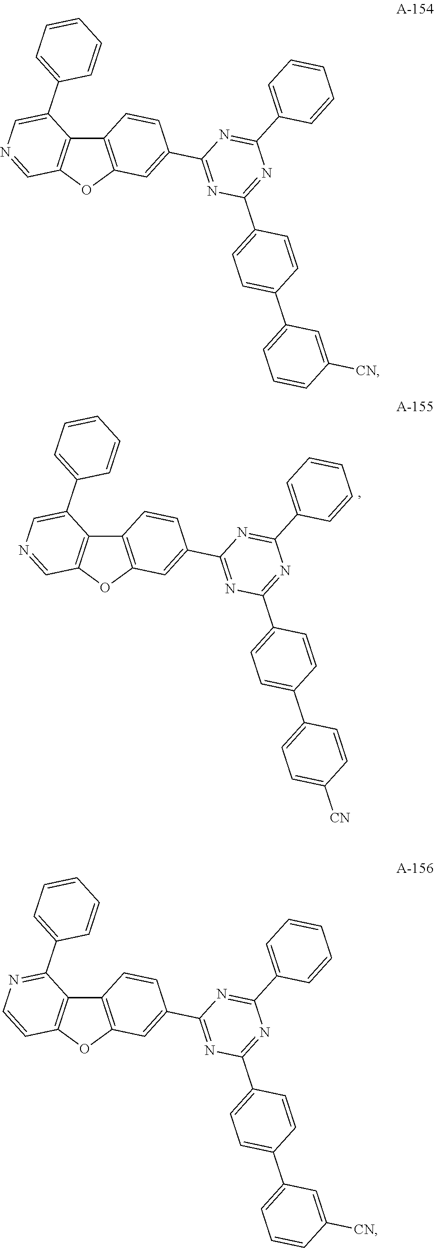 [SMILES: N#Cc1cccc(-c2ccc(-c3nc(-c4ccccc4)nc(-c4ccc5c(c4)oc4cncc(-c6ccccc6)c45)n3)cc2)c1 ; N#Cc1ccc(-c2ccc(-c3nc(-c4ccccc4)nc(-c4ccc5c(c4)oc4cncc(-c6ccccc6)c45)n3)cc2)cc1 ; N#Cc1cccc(-c2ccc(-c3nc(-c4ccccc4)nc(-c4ccc5c(c4)oc4ccnc(-c6ccccc6)c45)n3)cc2)c1] 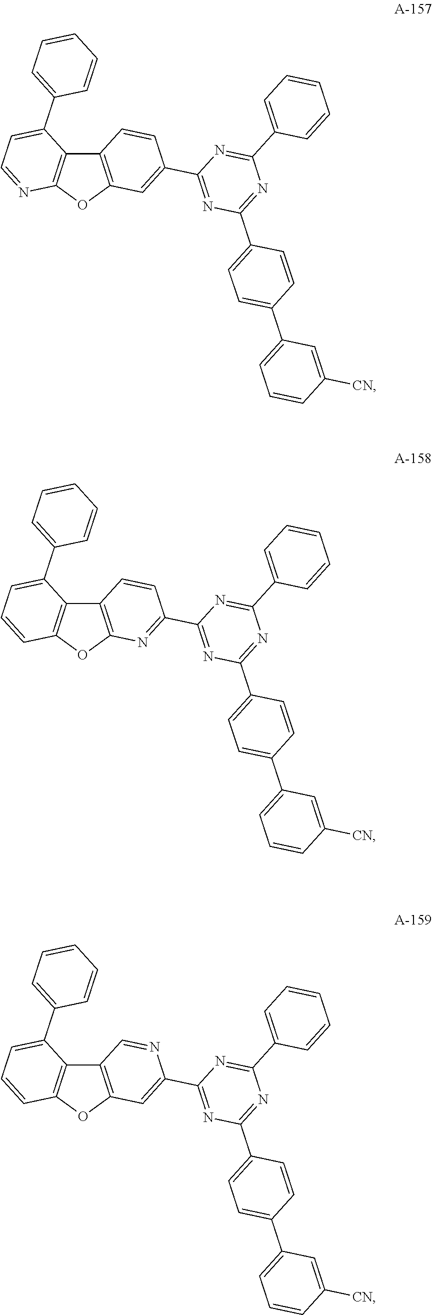 [SMILES: N#Cc1cccc(-c2ccc(-c3nc(-c4ccccc4)nc(-c4ccc5c(c4)oc4nccc(-c6ccccc6)c45)n3)cc2)c1 ; N#Cc1cccc(-c2ccc(-c3nc(-c4ccccc4)nc(-c4ccc5c(n4)oc4cccc(-c6ccccc6)c45)n3)cc2)c1 ; N#Cc1cccc(-c2ccc(-c3nc(-c4ccccc4)nc(-c4cc5oc6cccc(-c7ccccc7)c6c5cn4)n3)cc2)c1] 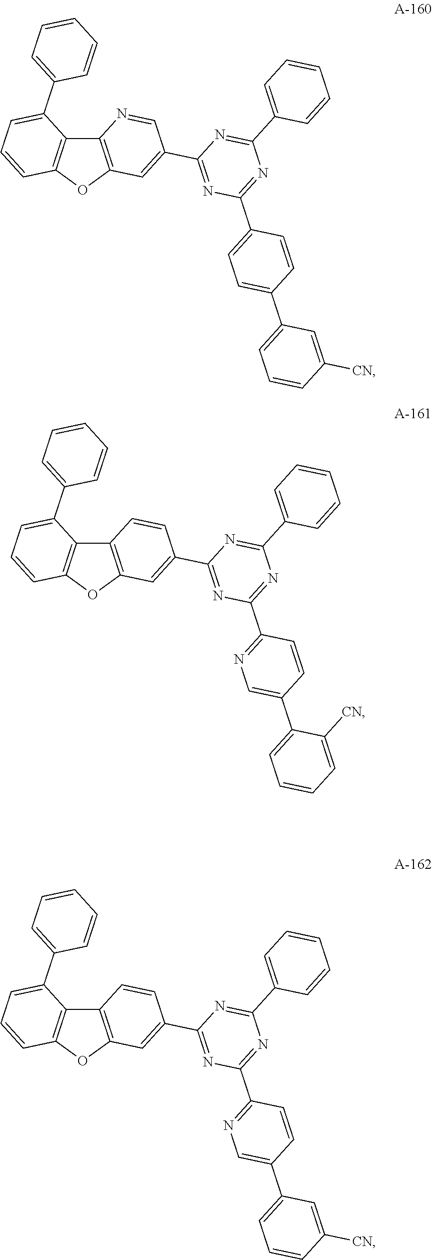 [SMILES: N#Cc1cccc(-c2ccc(-c3nc(-c4ccccc4)nc(-c4cnc5c(c4)oc4cccc(-c6ccccc6)c45)n3)cc2)c1 ; N#Cc1ccccc1-c1ccc(-c2nc(-c3ccccc3)nc(-c3ccc4c(c3)oc3cccc(-c5ccccc5)c34)n2)nc1 ; N#Cc1cccc(-c2ccc(-c3nc(-c4ccccc4)nc(-c4ccc5c(c4)oc4cccc(-c6ccccc6)c45)n3)nc2)c1] 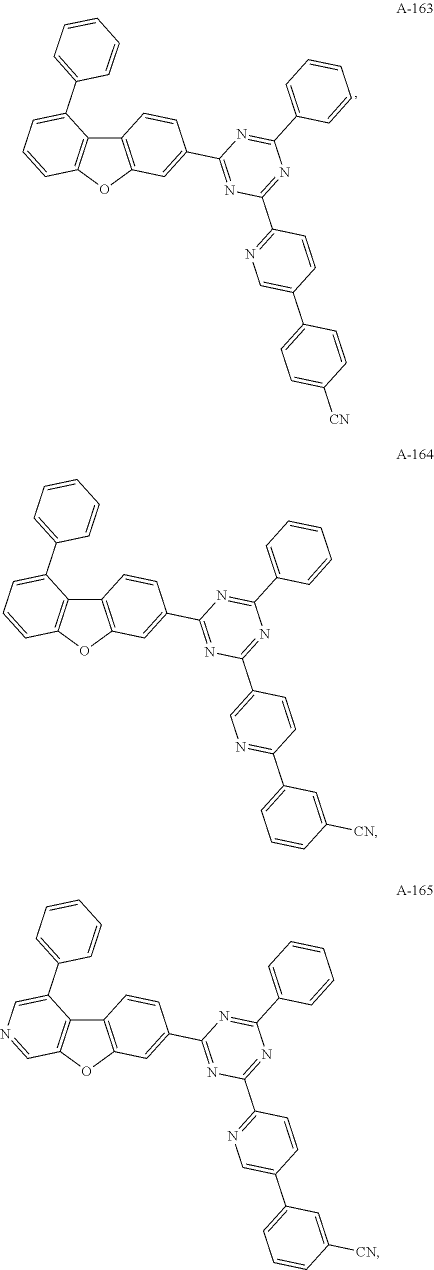 [SMILES: N#Cc1ccc(-c2ccc(-c3nc(-c4ccccc4)nc(-c4ccc5c(c4)oc4cccc(-c6ccccc6)c45)n3)nc2)cc1 ; N#Cc1cccc(-c2ccc(-c3nc(-c4ccccc4)nc(-c4ccc5c(c4)oc4cccc(-c6ccccc6)c45)n3)cn2)c1 ; N#Cc1cccc(-c2ccc(-c3nc(-c4ccccc4)nc(-c4ccc5c(c4)oc4cncc(-c6ccccc6)c45)n3)nc2)c1] 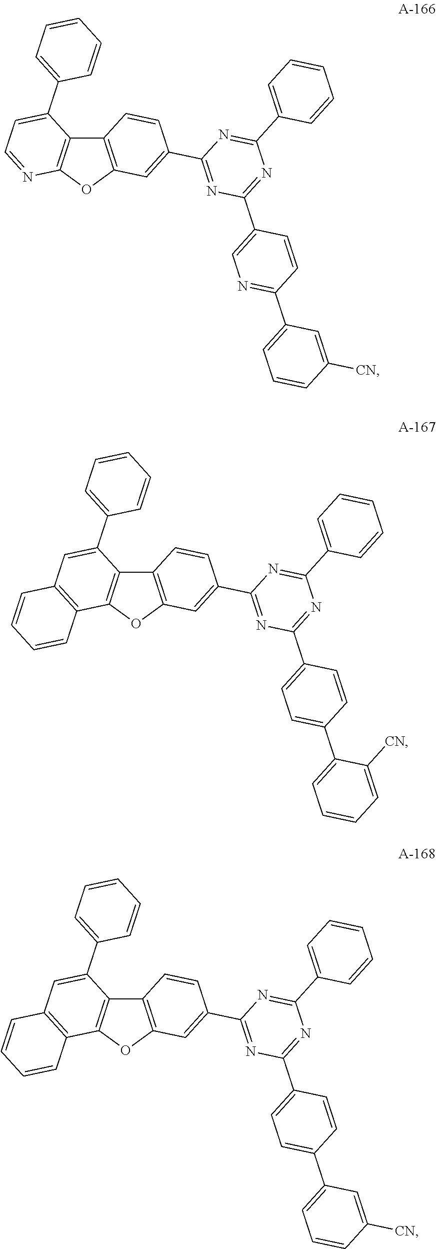 [SMILES: N#Cc1cccc(-c2ccc(-c3nc(-c4ccccc4)nc(-c4ccc5c(c4)oc4nccc(-c6ccccc6)c45)n3)cn2)c1 ; N#Cc1ccccc1-c1ccc(-c2nc(-c3ccccc3)nc(-c3ccc4c(c3)oc3c5ccccc5cc(-c5ccccc5)c43)n2)cc1 ; N#Cc1cccc(-c2ccc(-c3nc(-c4ccccc4)nc(-c4ccc5c(c4)oc4c6ccccc6cc(-c6ccccc6)c54)n3)cc2)c1] 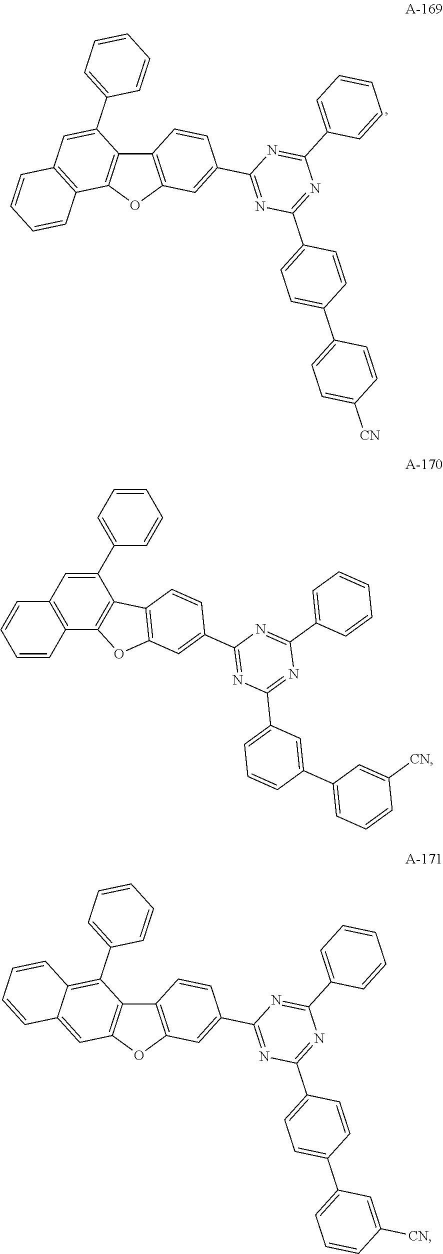 [SMILES: N#Cc1ccc(-c2ccc(-c3nc(-c4ccccc4)nc(-c4ccc5c(c4)oc4c6ccccc6cc(-c6ccccc6)c54)n3)cc2)cc1 ; N#Cc1cccc(-c2cccc(-c3nc(-c4ccccc4)nc(-c4ccc5c(c4)oc4c6ccccc6cc(-c6ccccc6)c54)n3)c2)c1 ; N#Cc1cccc(-c2ccc(-c3nc(-c4ccccc4)nc(-c4ccc5c(c4)oc4cc6ccccc6c(-c6ccccc6)c45)n3)cc2)c1] 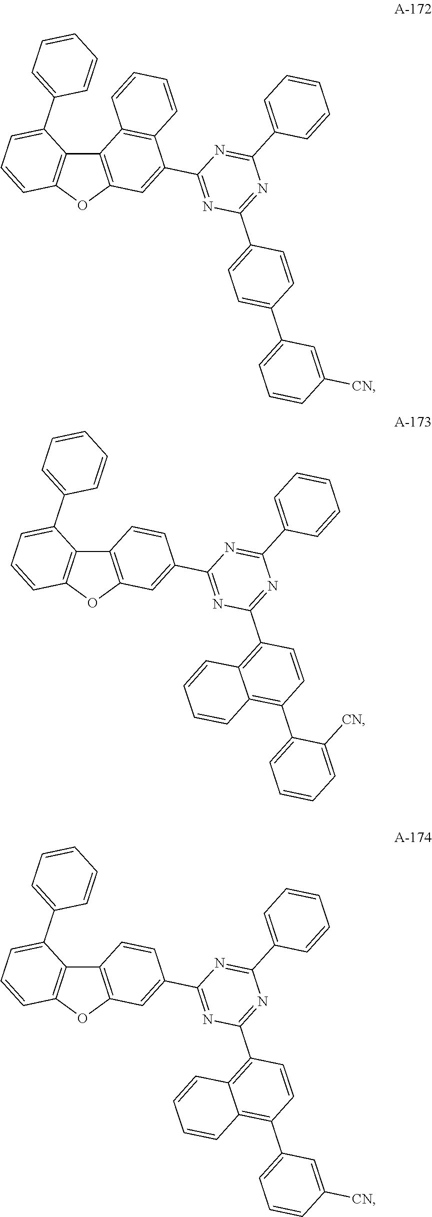 [SMILES: N#Cc1cccc(-c2ccc(-c3nc(-c4ccccc4)nc(-c4cc5oc6cccc(-c7ccccc7)c6c5c5ccccc45)n3)cc2)c1 ; N#Cc1ccccc1-c1ccc(-c2nc(-c3ccccc3)nc(-c3ccc4c(c3)oc3cccc(-c5ccccc5)c34)n2)c2ccccc12 ; N#Cc1cccc(-c2ccc(-c3nc(-c4ccccc4)nc(-c4ccc5c(c4)oc4cccc(-c6ccccc6)c45)n3)c3ccccc23)c1] 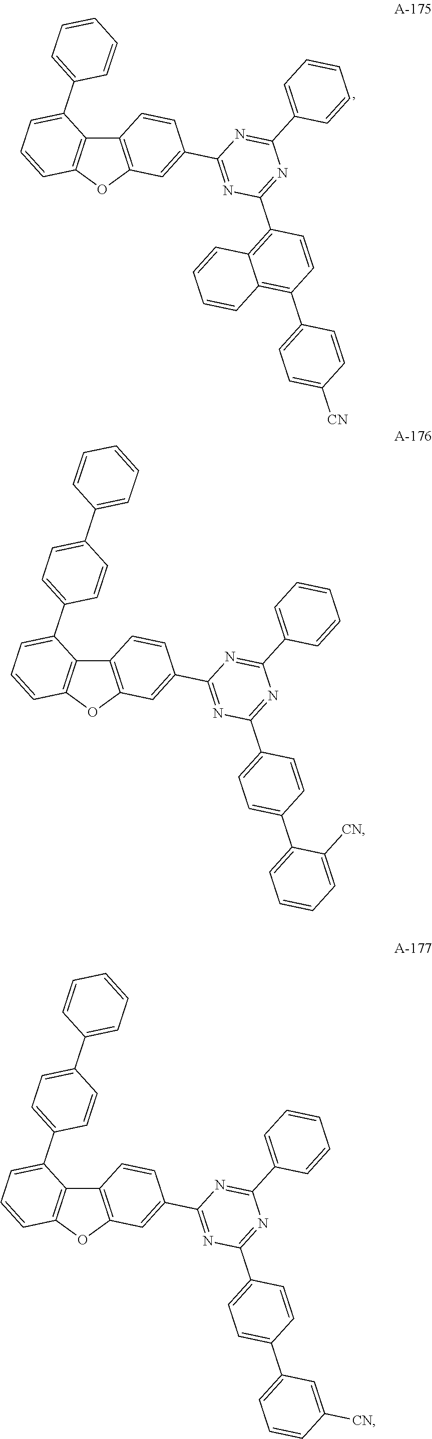 [SMILES: N#Cc1ccc(-c2ccc(-c3nc(-c4ccccc4)nc(-c4ccc5c(c4)oc4cccc(-c6ccccc6)c45)n3)c3ccccc23)cc1 ; N#Cc1ccccc1-c1ccc(-c2nc(-c3ccccc3)nc(-c3ccc4c(c3)oc3cccc(-c5ccc(-c6ccccc6)cc5)c34)n2)cc1 ; N#Cc1cccc(-c2ccc(-c3nc(-c4ccccc4)nc(-c4ccc5c(c4)oc4cccc(-c6ccc(-c7ccccc7)cc6)c45)n3)cc2)c1] 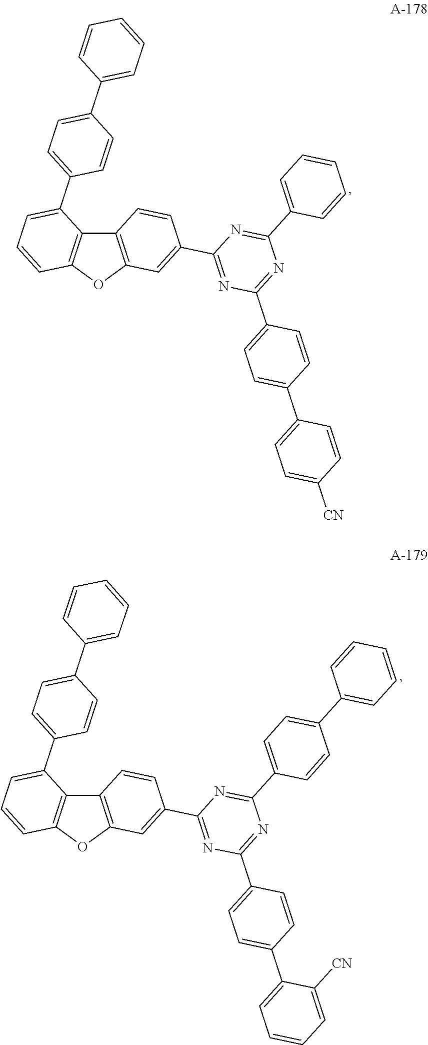 [SMILES: N#Cc1ccc(-c2ccc(-c3nc(-c4ccccc4)nc(-c4ccc5c(c4)oc4cccc(-c6ccc(-c7ccccc7)cc6)c45)n3)cc2)cc1 ; N#Cc1ccccc1-c1ccc(-c2nc(-c3ccc(-c4ccccc4)cc3)nc(-c3ccc4c(c3)oc3cccc(-c5ccc(-c6ccccc6)cc5)c34)n2)cc1] 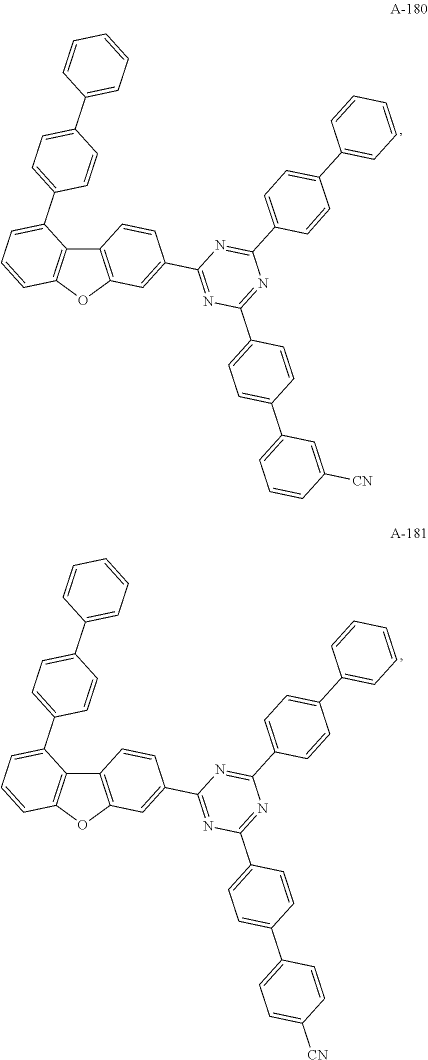 [SMILES: N#Cc1cccc(-c2ccc(-c3nc(-c4ccc(-c5ccccc5)cc4)nc(-c4ccc5c(c4)oc4cccc(-c6ccc(-c7ccccc7)cc6)c45)n3)cc2)c1 ; N#Cc1ccc(-c2ccc(-c3nc(-c4ccc(-c5ccccc5)cc4)nc(-c4ccc5c(c4)oc4cccc(-c6ccc(-c7ccccc7)cc6)c45)n3)cc2)cc1] 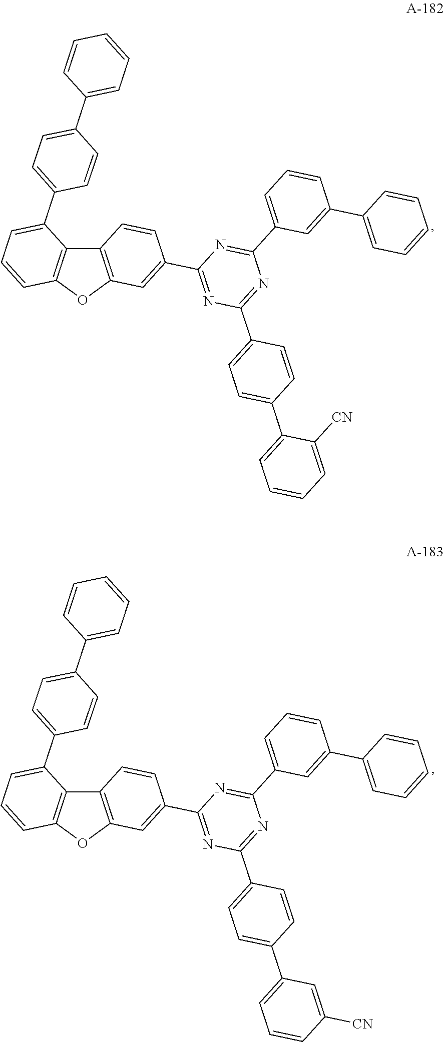 [SMILES: N#Cc1ccccc1-c1ccc(-c2nc(-c3cccc(-c4ccccc4)c3)nc(-c3ccc4c(c3)oc3cccc(-c5ccc(-c6ccccc6)cc5)c34)n2)cc1 ; N#Cc1cccc(-c2ccc(-c3nc(-c4cccc(-c5ccccc5)c4)nc(-c4ccc5c(c4)oc4cccc(-c6ccc(-c7ccccc7)cc6)c45)n3)cc2)c1] 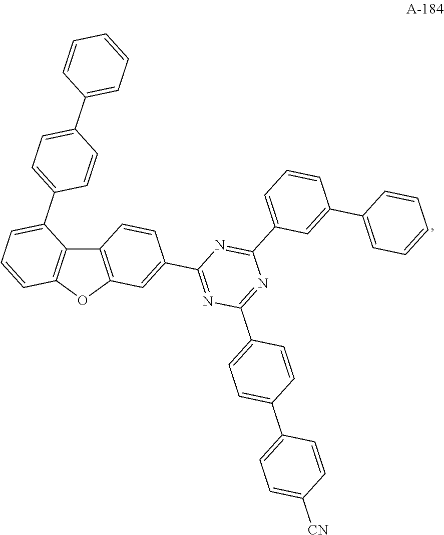 [SMILES: N#Cc1ccc(-c2ccc(-c3nc(-c4cccc(-c5ccccc5)c4)nc(-c4ccc5c(c4)oc4cccc(-c6ccc(-c7ccccc7)cc6)c45)n3)cc2)cc1] 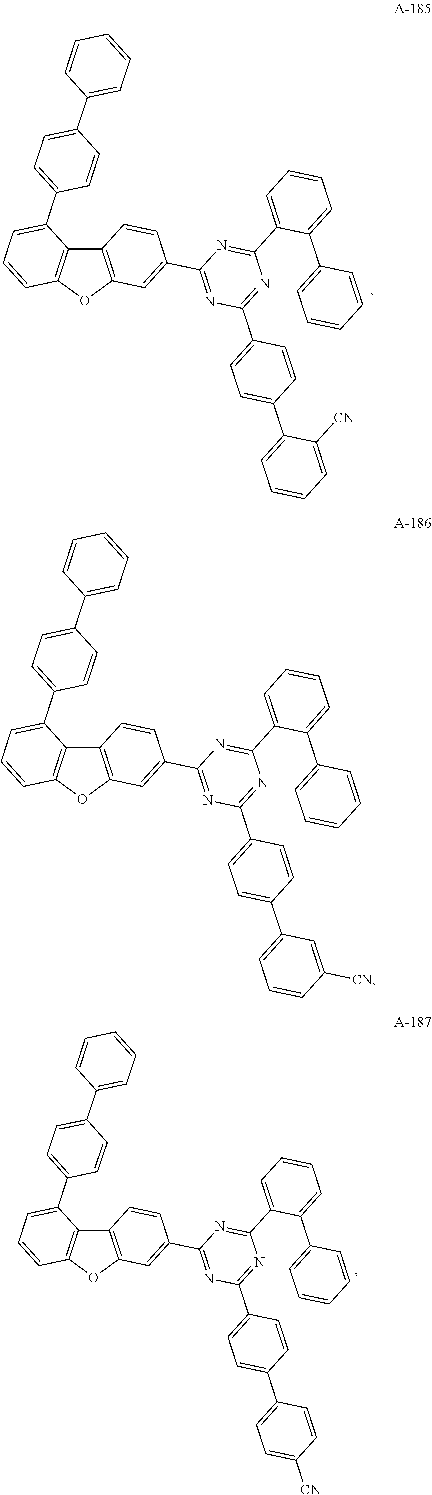 [SMILES: N#Cc1ccccc1-c1ccc(-c2nc(-c3ccc4c(c3)oc3cccc(-c5ccc(-c6ccccc6)cc5)c34)nc(-c3ccccc3-c3ccccc3)n2)cc1 ; N#Cc1cccc(-c2ccc(-c3nc(-c4ccc5c(c4)oc4cccc(-c6ccc(-c7ccccc7)cc6)c45)nc(-c4ccccc4-c4ccccc4)n3)cc2)c1 ; N#Cc1ccc(-c2ccc(-c3nc(-c4ccc5c(c4)oc4cccc(-c6ccc(-c7ccccc7)cc6)c45)nc(-c4ccccc4-c4ccccc4)n3)cc2)cc1] 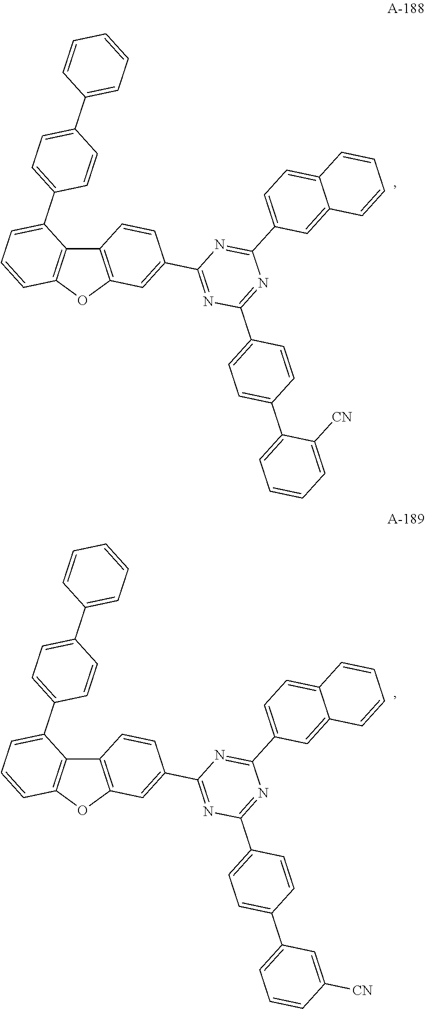 [SMILES: N#Cc1ccccc1-c1ccc(-c2nc(-c3ccc4ccccc4c3)nc(-c3ccc4c(c3)oc3cccc(-c5ccc(-c6ccccc6)cc5)c34)n2)cc1 ; N#Cc1cccc(-c2ccc(-c3nc(-c4ccc5ccccc5c4)nc(-c4ccc5c(c4)oc4cccc(-c6ccc(-c7ccccc7)cc6)c45)n3)cc2)c1] 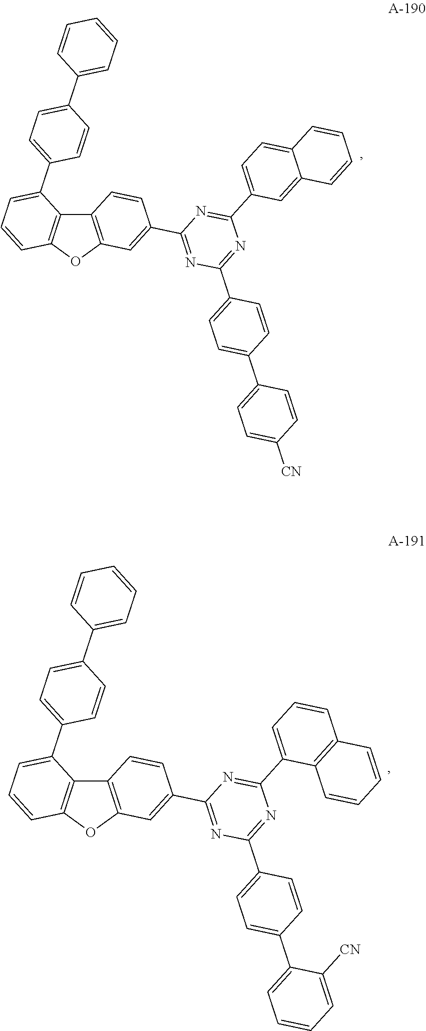 [SMILES: N#Cc1ccc(-c2ccc(-c3nc(-c4ccc5ccccc5c4)nc(-c4ccc5c(c4)oc4cccc(-c6ccc(-c7ccccc7)cc6)c45)n3)cc2)cc1 ; N#Cc1ccccc1-c1ccc(-c2nc(-c3ccc4c(c3)oc3cccc(-c5ccc(-c6ccccc6)cc5)c34)nc(-c3cccc4ccccc34)n2)cc1] 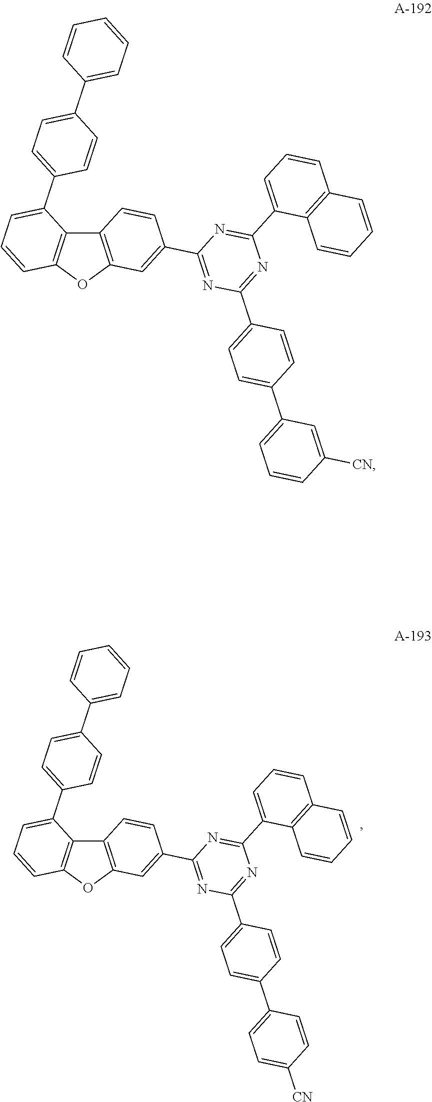 [SMILES: N#Cc1cccc(-c2ccc(-c3nc(-c4ccc5c(c4)oc4cccc(-c6ccc(-c7ccccc7)cc6)c45)nc(-c4cccc5ccccc45)n3)cc2)c1 ; N#Cc1ccc(-c2ccc(-c3nc(-c4ccc5c(c4)oc4cccc(-c6ccc(-c7ccccc7)cc6)c45)nc(-c4cccc5ccccc45)n3)cc2)cc1] 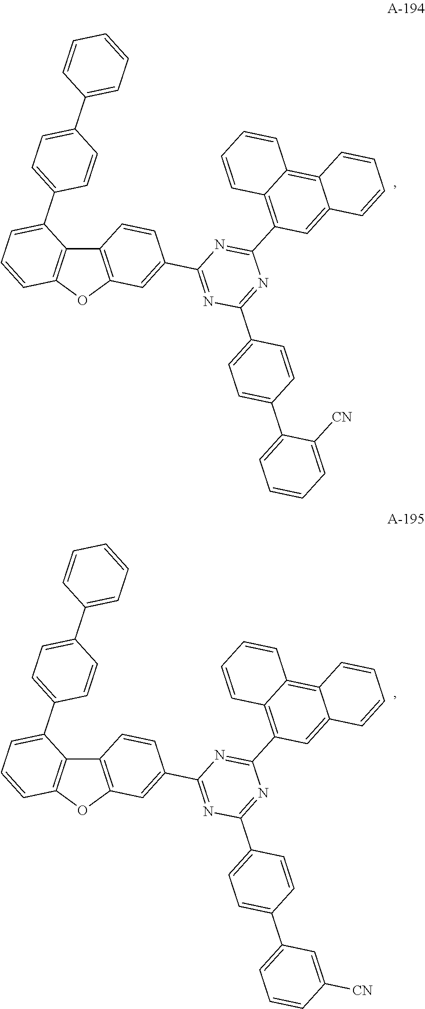 [SMILES: N#Cc1ccccc1-c1ccc(-c2nc(-c3ccc4c(c3)oc3cccc(-c5ccc(-c6ccccc6)cc5)c34)nc(-c3cc4ccccc4c4ccccc34)n2)cc1 ; N#Cc1cccc(-c2ccc(-c3nc(-c4ccc5c(c4)oc4cccc(-c6ccc(-c7ccccc7)cc6)c45)nc(-c4cc5ccccc5c5ccccc45)n3)cc2)c1] 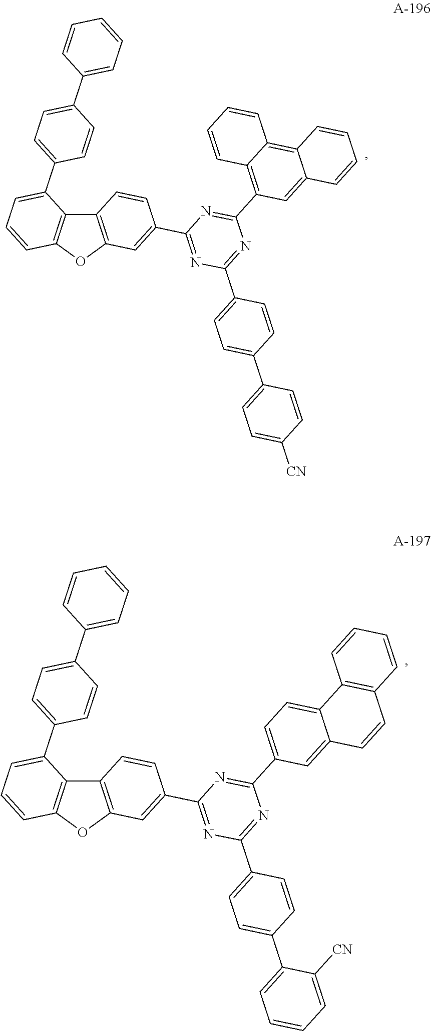 [SMILES: N#Cc1ccc(-c2ccc(-c3nc(-c4ccc5c(c4)oc4cccc(-c6ccc(-c7ccccc7)cc6)c45)nc(-c4cc5ccccc5c5ccccc45)n3)cc2)cc1 ; N#Cc1ccccc1-c1ccc(-c2nc(-c3ccc4c(ccc5ccccc54)c3)nc(-c3ccc4c(c3)oc3cccc(-c5ccc(-c6ccccc6)cc5)c34)n2)cc1] 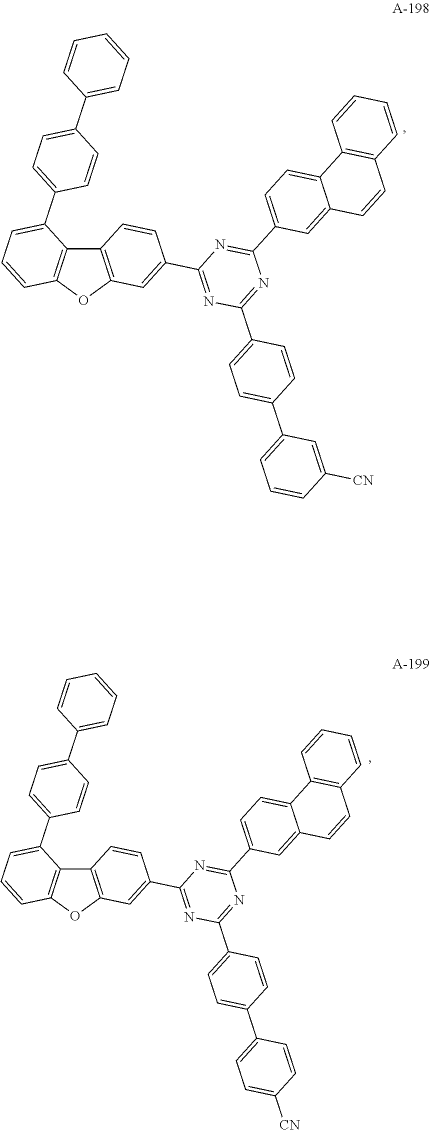 [SMILES: N#Cc1cccc(-c2ccc(-c3nc(-c4ccc5c(ccc6ccccc65)c4)nc(-c4ccc5c(c4)oc4cccc(-c6ccc(-c7ccccc7)cc6)c45)n3)cc2)c1 ; N#Cc1ccc(-c2ccc(-c3nc(-c4ccc5c(ccc6ccccc65)c4)nc(-c4ccc5c(c4)oc4cccc(-c6ccc(-c7ccccc7)cc6)c45)n3)cc2)cc1] 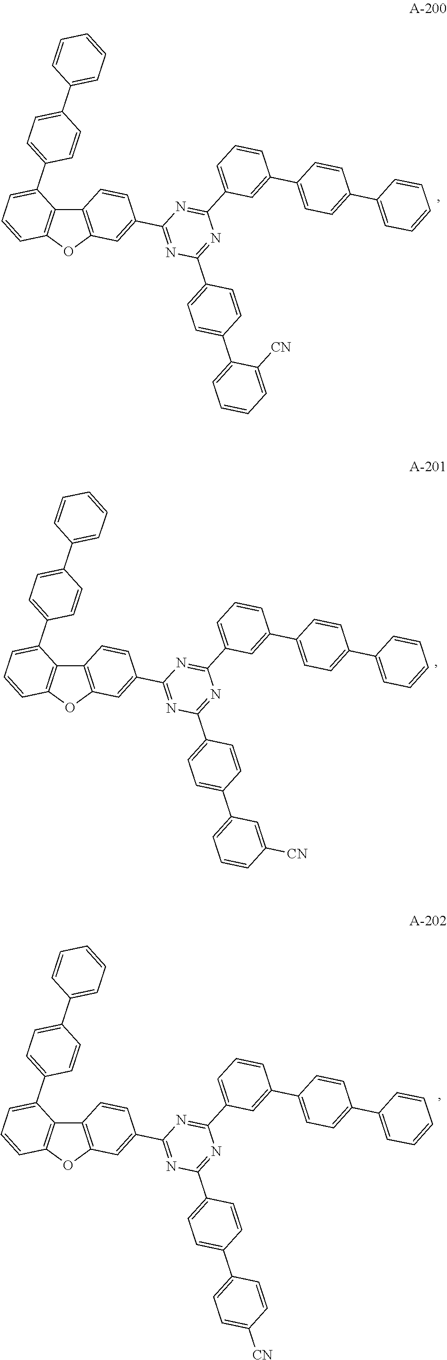 [SMILES: N#Cc1ccccc1-c1ccc(-c2nc(-c3cccc(-c4ccc(-c5ccccc5)cc4)c3)nc(-c3ccc4c(c3)oc3cccc(-c5ccc(-c6ccccc6)cc5)c34)n2)cc1 ; N#Cc1cccc(-c2ccc(-c3nc(-c4cccc(-c5ccc(-c6ccccc6)cc5)c4)nc(-c4ccc5c(c4)oc4cccc(-c6ccc(-c7ccccc7)cc6)c45)n3)cc2)c1 ; N#Cc1ccc(-c2ccc(-c3nc(-c4cccc(-c5ccc(-c6ccccc6)cc5)c4)nc(-c4ccc5c(c4)oc4cccc(-c6ccc(-c7ccccc7)cc6)c45)n3)cc2)cc1] 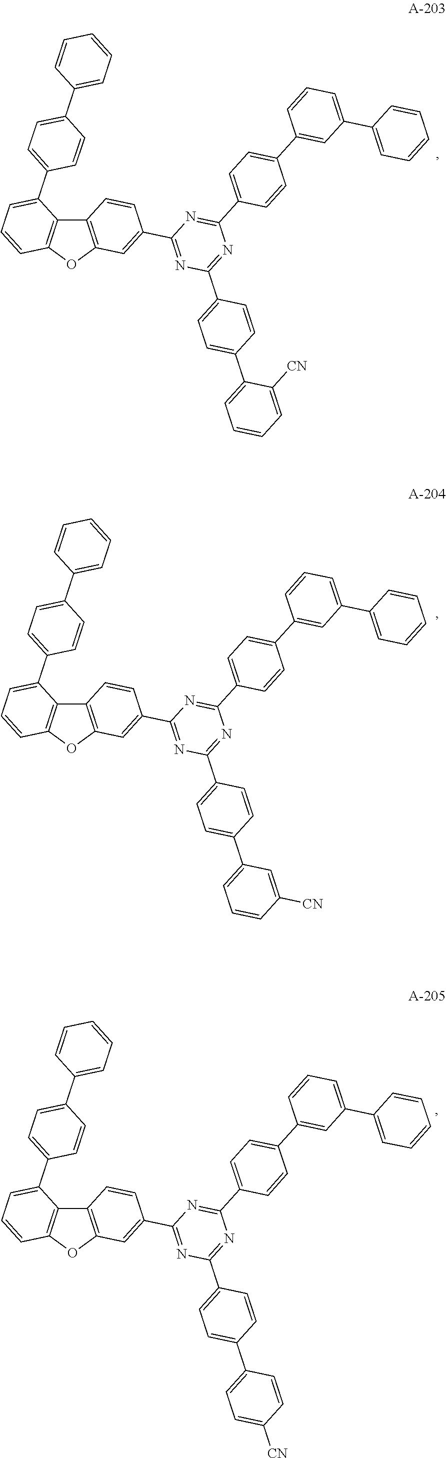 [SMILES: N#Cc1ccccc1-c1ccc(-c2nc(-c3ccc(-c4cccc(-c5ccccc5)c4)cc3)nc(-c3ccc4c(c3)oc3cccc(-c5ccc(-c6ccccc6)cc5)c34)n2)cc1 ; N#Cc1cccc(-c2ccc(-c3nc(-c4ccc(-c5cccc(-c6ccccc6)c5)cc4)nc(-c4ccc5c(c4)oc4cccc(-c6ccc(-c7ccccc7)cc6)c45)n3)cc2)c1 ; N#Cc1ccc(-c2ccc(-c3nc(-c4ccc(-c5cccc(-c6ccccc6)c5)cc4)nc(-c4ccc5c(c4)oc4cccc(-c6ccc(-c7ccccc7)cc6)c45)n3)cc2)cc1] 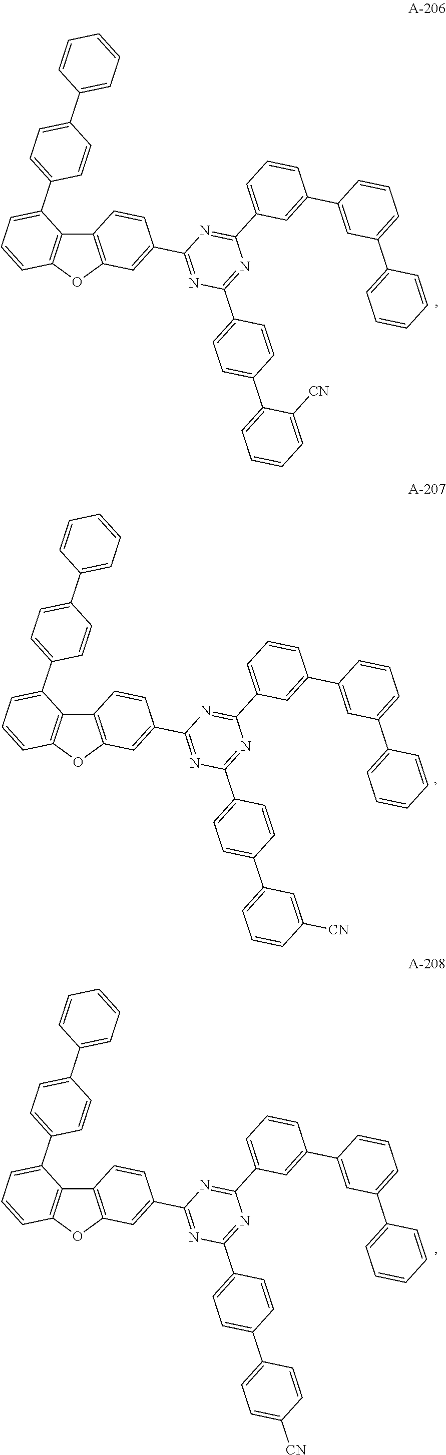 [SMILES: N#Cc1ccccc1-c1ccc(-c2nc(-c3cccc(-c4cccc(-c5ccccc5)c4)c3)nc(-c3ccc4c(c3)oc3cccc(-c5ccc(-c6ccccc6)cc5)c34)n2)cc1 ; N#Cc1cccc(-c2ccc(-c3nc(-c4cccc(-c5cccc(-c6ccccc6)c5)c4)nc(-c4ccc5c(c4)oc4cccc(-c6ccc(-c7ccccc7)cc6)c45)n3)cc2)c1 ; N#Cc1ccc(-c2ccc(-c3nc(-c4cccc(-c5cccc(-c6ccccc6)c5)c4)nc(-c4ccc5c(c4)oc4cccc(-c6ccc(-c7ccccc7)cc6)c45)n3)cc2)cc1] 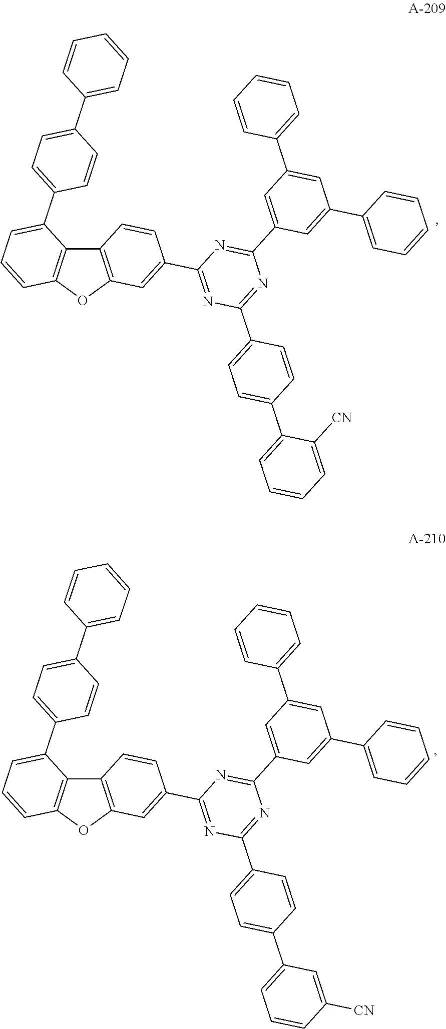 [SMILES: N#Cc1ccccc1-c1ccc(-c2nc(-c3cc(-c4ccccc4)cc(-c4ccccc4)c3)nc(-c3ccc4c(c3)oc3cccc(-c5ccc(-c6ccccc6)cc5)c34)n2)cc1 ; N#Cc1cccc(-c2ccc(-c3nc(-c4cc(-c5ccccc5)cc(-c5ccccc5)c4)nc(-c4ccc5c(c4)oc4cccc(-c6ccc(-c7ccccc7)cc6)c45)n3)cc2)c1] 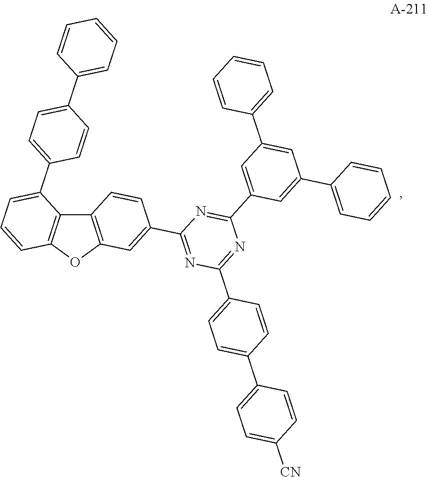 [SMILES: N#Cc1ccc(-c2ccc(-c3nc(-c4cc(-c5ccccc5)cc(-c5ccccc5)c4)nc(-c4ccc5c(c4)oc4cccc(-c6ccc(-c7ccccc7)cc6)c45)n3)cc2)cc1] 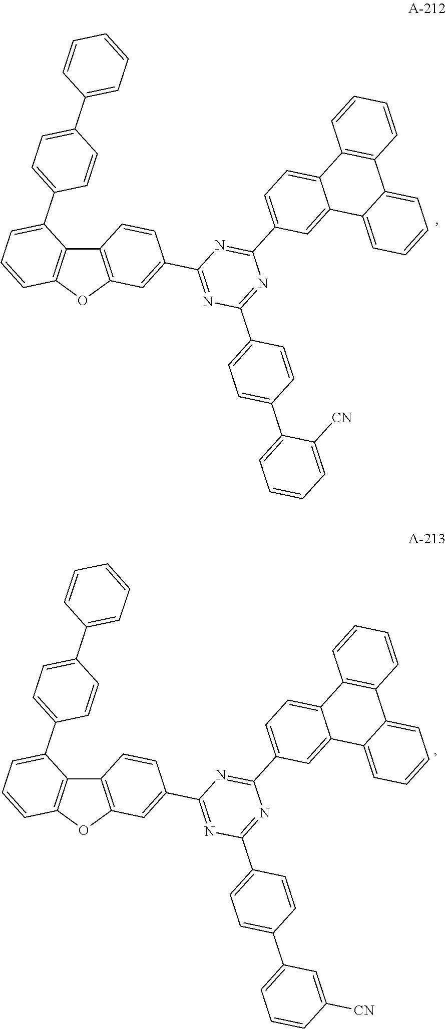 [SMILES: N#Cc1ccccc1-c1ccc(-c2nc(-c3ccc4c(c3)oc3cccc(-c5ccc(-c6ccccc6)cc5)c34)nc(-c3ccc4c5ccccc5c5ccccc5c4c3)n2)cc1 ; N#Cc1cccc(-c2ccc(-c3nc(-c4ccc5c(c4)oc4cccc(-c6ccc(-c7ccccc7)cc6)c45)nc(-c4ccc5c6ccccc6c6ccccc6c5c4)n3)cc2)c1] 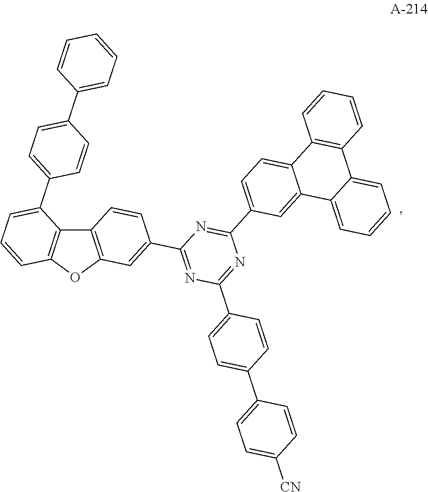 [SMILES: N#Cc1ccc(-c2ccc(-c3nc(-c4ccc5c(c4)oc4cccc(-c6ccc(-c7ccccc7)cc6)c45)nc(-c4ccc5c6ccccc6c6ccccc6c5c4)n3)cc2)cc1] 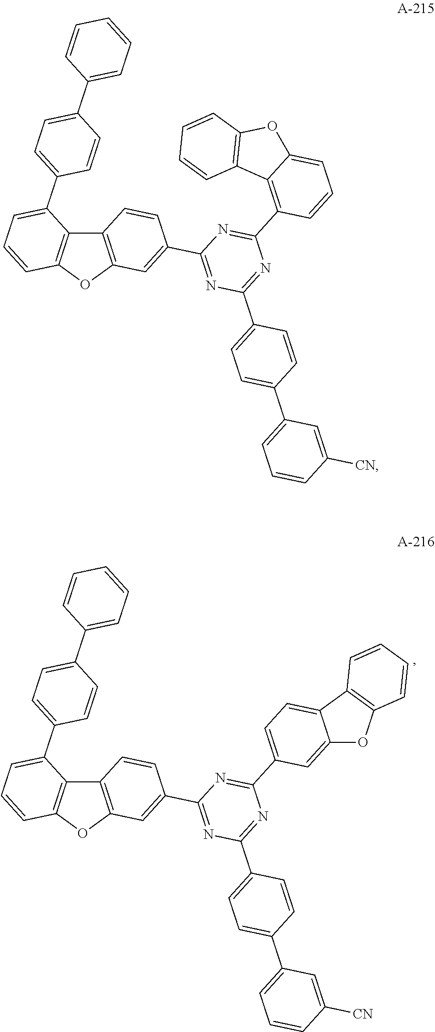 [SMILES: N#Cc1cccc(-c2ccc(-c3nc(-c4ccc5c(c4)oc4cccc(-c6ccc(-c7ccccc7)cc6)c45)nc(-c4cccc5oc6ccccc6c45)n3)cc2)c1 ; N#Cc1cccc(-c2ccc(-c3nc(-c4ccc5c(c4)oc4ccccc45)nc(-c4ccc5c(c4)oc4cccc(-c6ccc(-c7ccccc7)cc6)c45)n3)cc2)c1] 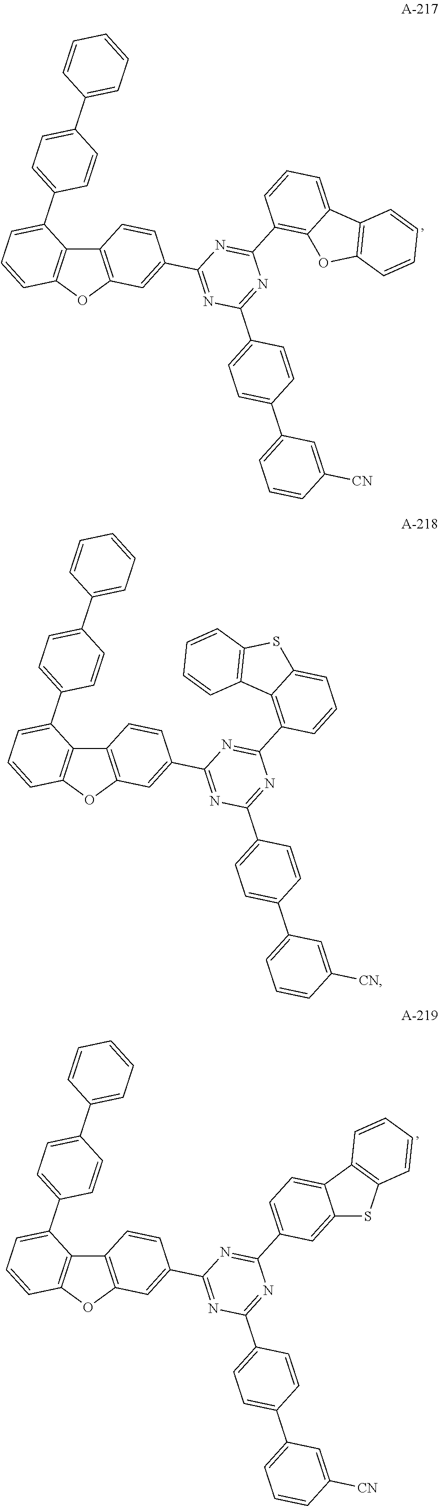 [SMILES: N#Cc1cccc(-c2ccc(-c3nc(-c4ccc5c(c4)oc4cccc(-c6ccc(-c7ccccc7)cc6)c45)nc(-c4cccc5c4oc4ccccc45)n3)cc2)c1 ; N#Cc1cccc(-c2ccc(-c3nc(-c4ccc5c(c4)oc4cccc(-c6ccc(-c7ccccc7)cc6)c45)nc(-c4cccc5sc6ccccc6c45)n3)cc2)c1 ; N#Cc1cccc(-c2ccc(-c3nc(-c4ccc5c(c4)oc4cccc(-c6ccc(-c7ccccc7)cc6)c45)nc(-c4ccc5c(c4)sc4ccccc45)n3)cc2)c1] 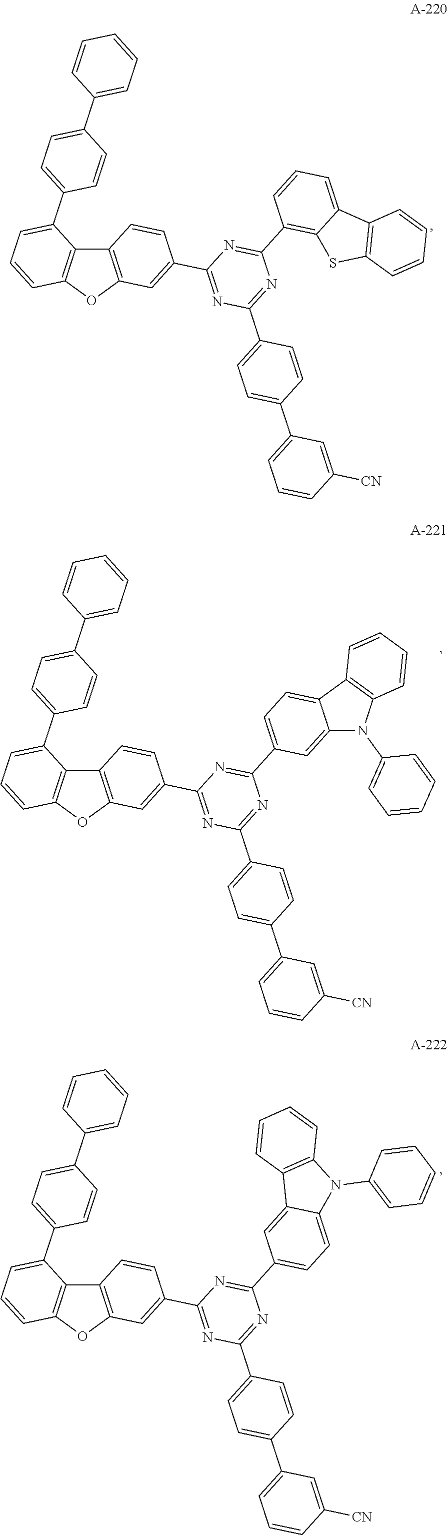 [SMILES: N#Cc1cccc(-c2ccc(-c3nc(-c4ccc5c(c4)oc4cccc(-c6ccc(-c7ccccc7)cc6)c45)nc(-c4cccc5c4sc4ccccc45)n3)cc2)c1 ; N#Cc1cccc(-c2ccc(-c3nc(-c4ccc5c(c4)oc4cccc(-c6ccc(-c7ccccc7)cc6)c45)nc(-c4ccc5c6ccccc6n(-c6ccccc6)c5c4)n3)cc2)c1 ; N#Cc1cccc(-c2ccc(-c3nc(-c4ccc5c(c4)oc4cccc(-c6ccc(-c7ccccc7)cc6)c45)nc(-c4ccc5c(c4)c4ccccc4n5-c4ccccc4)n3)cc2)c1] 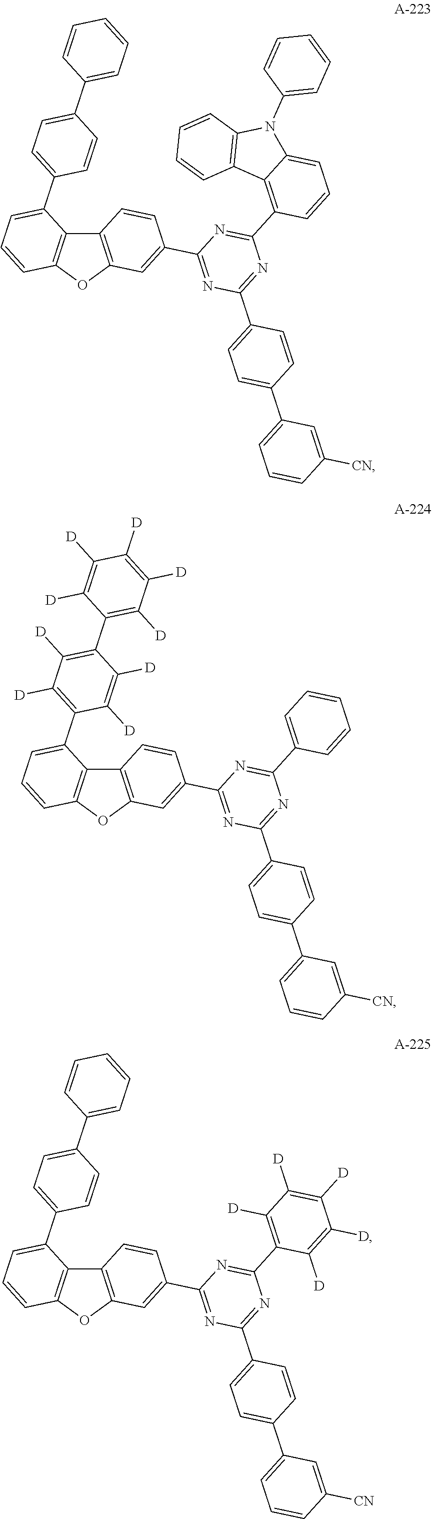 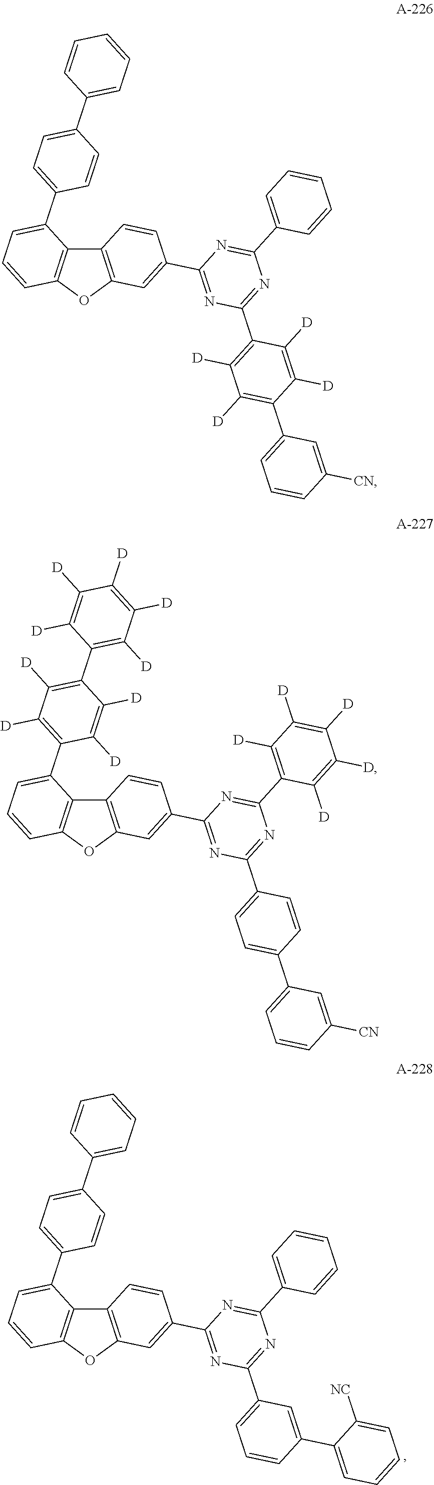 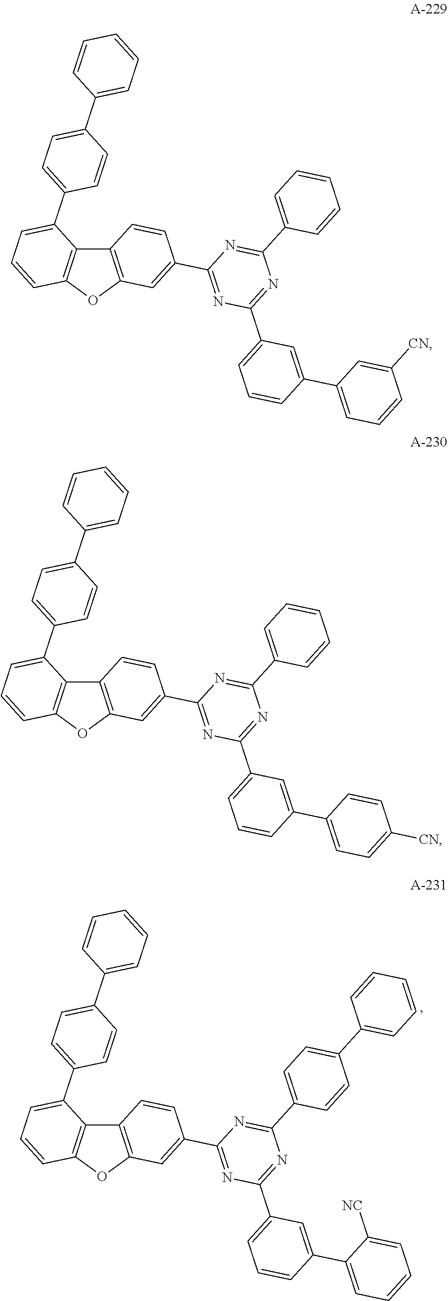 [SMILES: N#Cc1cccc(-c2cccc(-c3nc(-c4ccccc4)nc(-c4ccc5c(c4)oc4cccc(-c6ccc(-c7ccccc7)cc6)c45)n3)c2)c1 ; N#Cc1ccc(-c2cccc(-c3nc(-c4ccccc4)nc(-c4ccc5c(c4)oc4cccc(-c6ccc(-c7ccccc7)cc6)c45)n3)c2)cc1 ; N#Cc1ccccc1-c1cccc(-c2nc(-c3ccc(-c4ccccc4)cc3)nc(-c3ccc4c(c3)oc3cccc(-c5ccc(-c6ccccc6)cc5)c34)n2)c1] 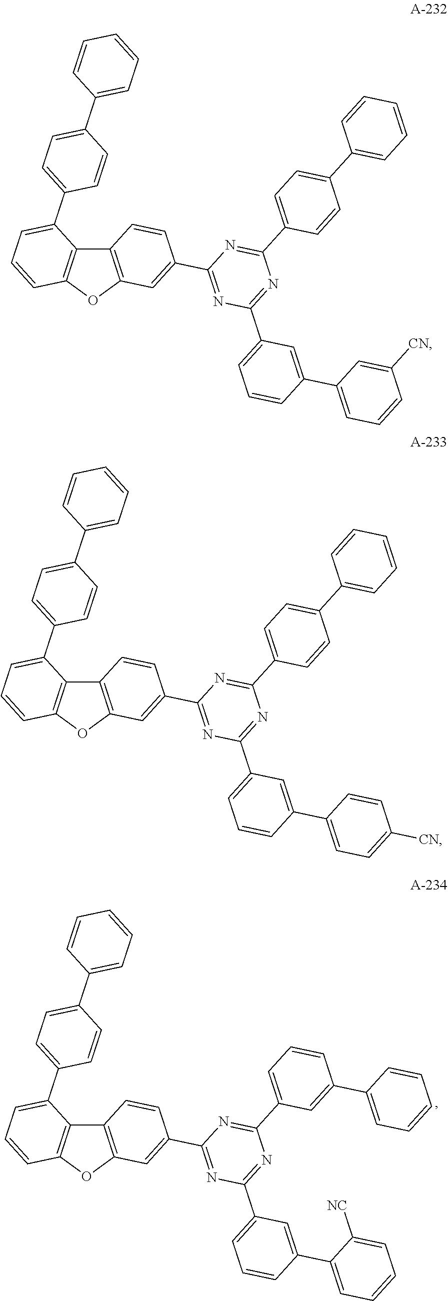 [SMILES: N#Cc1cccc(-c2cccc(-c3nc(-c4ccc(-c5ccccc5)cc4)nc(-c4ccc5c(c4)oc4cccc(-c6ccc(-c7ccccc7)cc6)c45)n3)c2)c1 ; N#Cc1ccc(-c2cccc(-c3nc(-c4ccc(-c5ccccc5)cc4)nc(-c4ccc5c(c4)oc4cccc(-c6ccc(-c7ccccc7)cc6)c45)n3)c2)cc1 ; N#Cc1ccccc1-c1cccc(-c2nc(-c3cccc(-c4ccccc4)c3)nc(-c3ccc4c(c3)oc3cccc(-c5ccc(-c6ccccc6)cc5)c34)n2)c1] 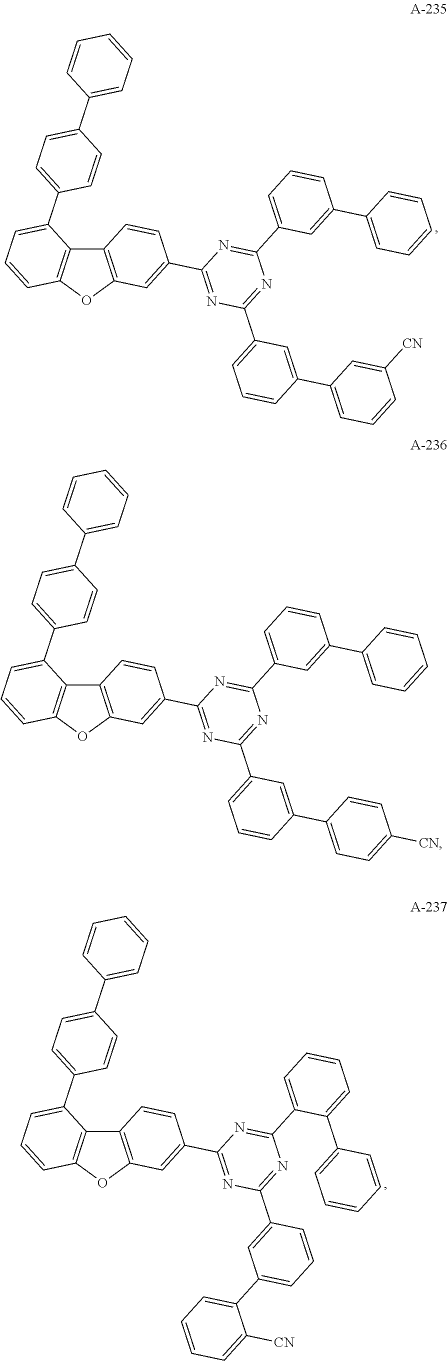 [SMILES: N#Cc1cccc(-c2cccc(-c3nc(-c4cccc(-c5ccccc5)c4)nc(-c4ccc5c(c4)oc4cccc(-c6ccc(-c7ccccc7)cc6)c45)n3)c2)c1 ; N#Cc1ccc(-c2cccc(-c3nc(-c4cccc(-c5ccccc5)c4)nc(-c4ccc5c(c4)oc4cccc(-c6ccc(-c7ccccc7)cc6)c45)n3)c2)cc1 ; N#Cc1ccccc1-c1cccc(-c2nc(-c3ccc4c(c3)oc3cccc(-c5ccc(-c6ccccc6)cc5)c34)nc(-c3ccccc3-c3ccccc3)n2)c1] 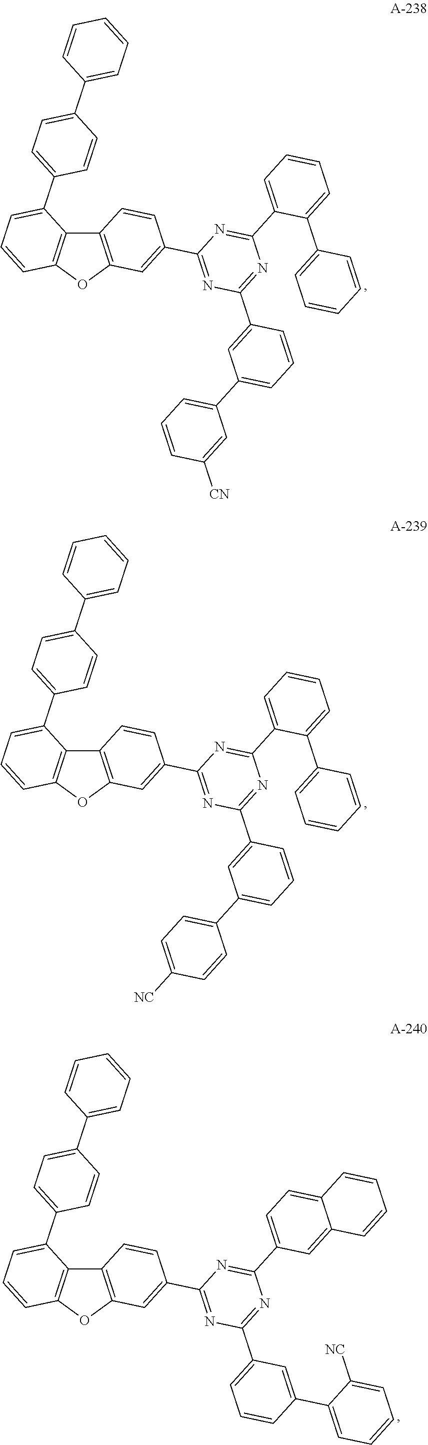 [SMILES: N#Cc1cccc(-c2cccc(-c3nc(-c4ccc5c(c4)oc4cccc(-c6ccc(-c7ccccc7)cc6)c45)nc(-c4ccccc4-c4ccccc4)n3)c2)c1 ; N#Cc1ccc(-c2cccc(-c3nc(-c4ccc5c(c4)oc4cccc(-c6ccc(-c7ccccc7)cc6)c45)nc(-c4ccccc4-c4ccccc4)n3)c2)cc1 ; N#Cc1ccccc1-c1cccc(-c2nc(-c3ccc4ccccc4c3)nc(-c3ccc4c(c3)oc3cccc(-c5ccc(-c6ccccc6)cc5)c34)n2)c1] 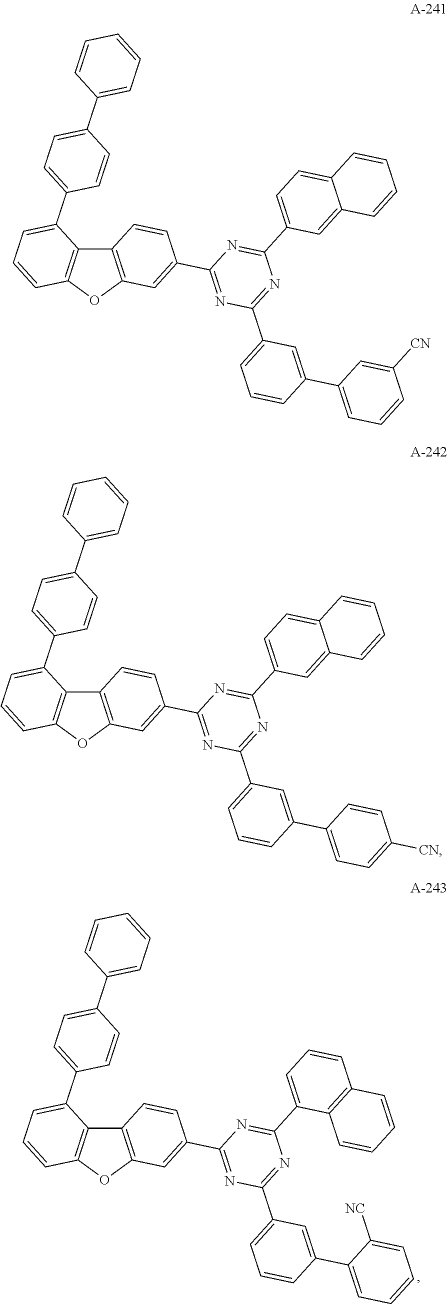 [SMILES: N#Cc1cccc(-c2cccc(-c3nc(-c4ccc5ccccc5c4)nc(-c4ccc5c(c4)oc4cccc(-c6ccc(-c7ccccc7)cc6)c45)n3)c2)c1 ; N#Cc1ccc(-c2cccc(-c3nc(-c4ccc5ccccc5c4)nc(-c4ccc5c(c4)oc4cccc(-c6ccc(-c7ccccc7)cc6)c45)n3)c2)cc1 ; N#Cc1ccccc1-c1cccc(-c2nc(-c3ccc4c(c3)oc3cccc(-c5ccc(-c6ccccc6)cc5)c34)nc(-c3cccc4ccccc34)n2)c1] 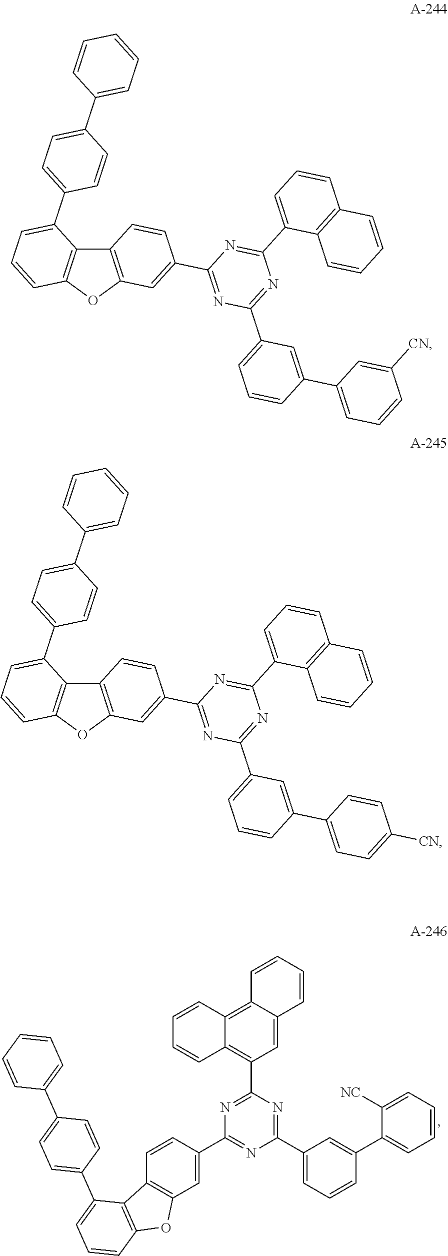 [SMILES: N#Cc1cccc(-c2cccc(-c3nc(-c4ccc5c(c4)oc4cccc(-c6ccc(-c7ccccc7)cc6)c45)nc(-c4cccc5ccccc45)n3)c2)c1 ; N#Cc1ccc(-c2cccc(-c3nc(-c4ccc5c(c4)oc4cccc(-c6ccc(-c7ccccc7)cc6)c45)nc(-c4cccc5ccccc45)n3)c2)cc1 ; N#Cc1ccccc1-c1cccc(-c2nc(-c3ccc4c(c3)oc3cccc(-c5ccc(-c6ccccc6)cc5)c34)nc(-c3cc4ccccc4c4ccccc34)n2)c1] 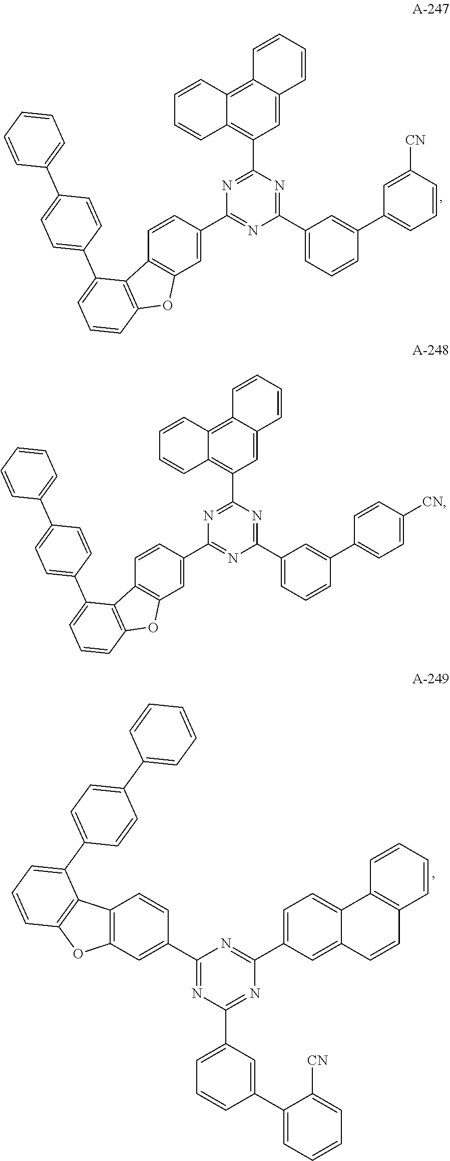 [SMILES: N#Cc1cccc(-c2cccc(-c3nc(-c4ccc5c(c4)oc4cccc(-c6ccc(-c7ccccc7)cc6)c45)nc(-c4cc5ccccc5c5ccccc45)n3)c2)c1 ; N#Cc1ccc(-c2cccc(-c3nc(-c4ccc5c(c4)oc4cccc(-c6ccc(-c7ccccc7)cc6)c45)nc(-c4cc5ccccc5c5ccccc45)n3)c2)cc1 ; N#Cc1ccccc1-c1cccc(-c2nc(-c3ccc4c(ccc5ccccc54)c3)nc(-c3ccc4c(c3)oc3cccc(-c5ccc(-c6ccccc6)cc5)c34)n2)c1] 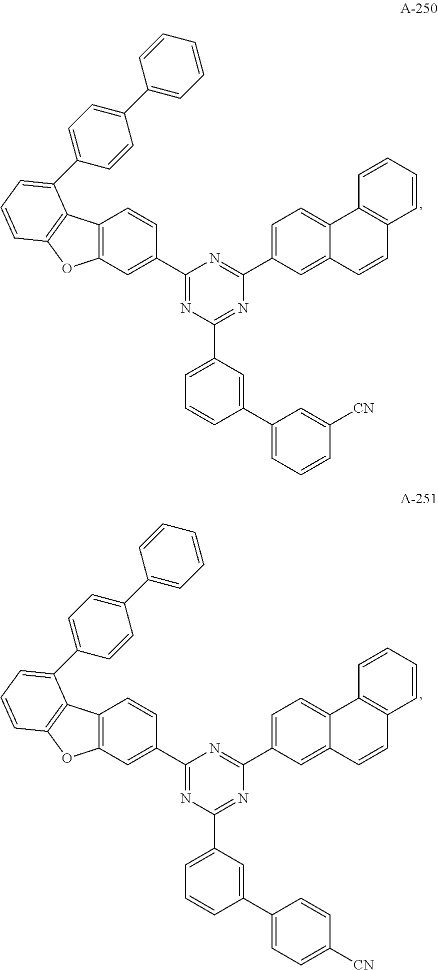 [SMILES: N#Cc1cccc(-c2cccc(-c3nc(-c4ccc5c(ccc6ccccc65)c4)nc(-c4ccc5c(c4)oc4cccc(-c6ccc(-c7ccccc7)cc6)c45)n3)c2)c1 ; N#Cc1ccc(-c2cccc(-c3nc(-c4ccc5c(ccc6ccccc65)c4)nc(-c4ccc5c(c4)oc4cccc(-c6ccc(-c7ccccc7)cc6)c45)n3)c2)cc1] 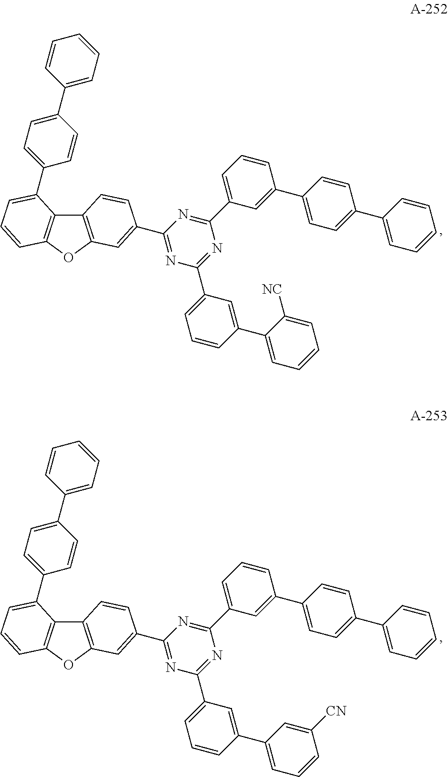 [SMILES: N#Cc1ccccc1-c1cccc(-c2nc(-c3cccc(-c4ccc(-c5ccccc5)cc4)c3)nc(-c3ccc4c(c3)oc3cccc(-c5ccc(-c6ccccc6)cc5)c34)n2)c1 ; N#Cc1cccc(-c2cccc(-c3nc(-c4cccc(-c5ccc(-c6ccccc6)cc5)c4)nc(-c4ccc5c(c4)oc4cccc(-c6ccc(-c7ccccc7)cc6)c45)n3)c2)c1] 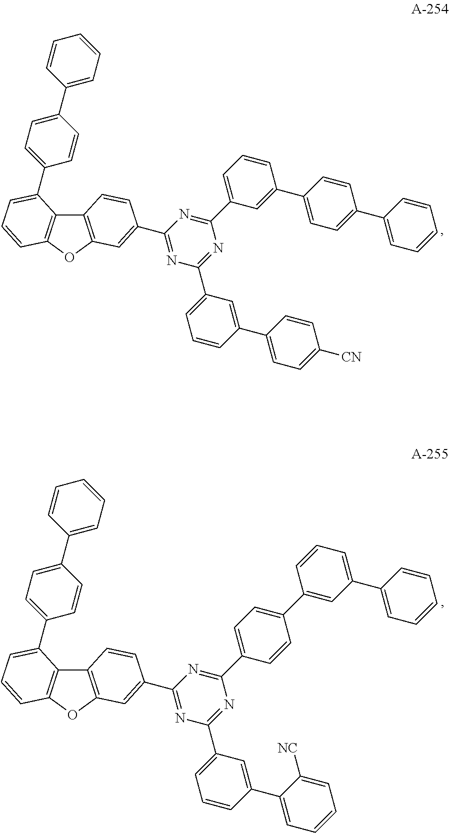 [SMILES: N#Cc1ccc(-c2cccc(-c3nc(-c4cccc(-c5ccc(-c6ccccc6)cc5)c4)nc(-c4ccc5c(c4)oc4cccc(-c6ccc(-c7ccccc7)cc6)c45)n3)c2)cc1 ; N#Cc1ccccc1-c1cccc(-c2nc(-c3ccc(-c4cccc(-c5ccccc5)c4)cc3)nc(-c3ccc4c(c3)oc3cccc(-c5ccc(-c6ccccc6)cc5)c34)n2)c1] 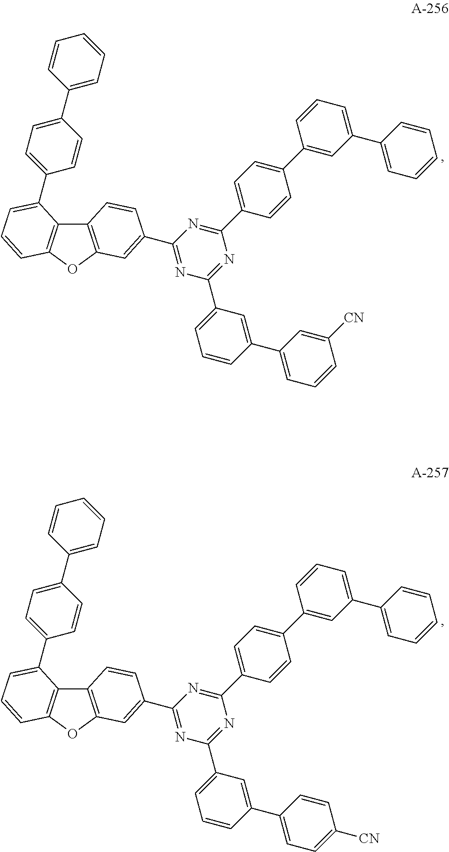 [SMILES: N#Cc1cccc(-c2cccc(-c3nc(-c4ccc(-c5cccc(-c6ccccc6)c5)cc4)nc(-c4ccc5c(c4)oc4cccc(-c6ccc(-c7ccccc7)cc6)c45)n3)c2)c1 ; N#Cc1ccc(-c2cccc(-c3nc(-c4ccc(-c5cccc(-c6ccccc6)c5)cc4)nc(-c4ccc5c(c4)oc4cccc(-c6ccc(-c7ccccc7)cc6)c45)n3)c2)cc1] 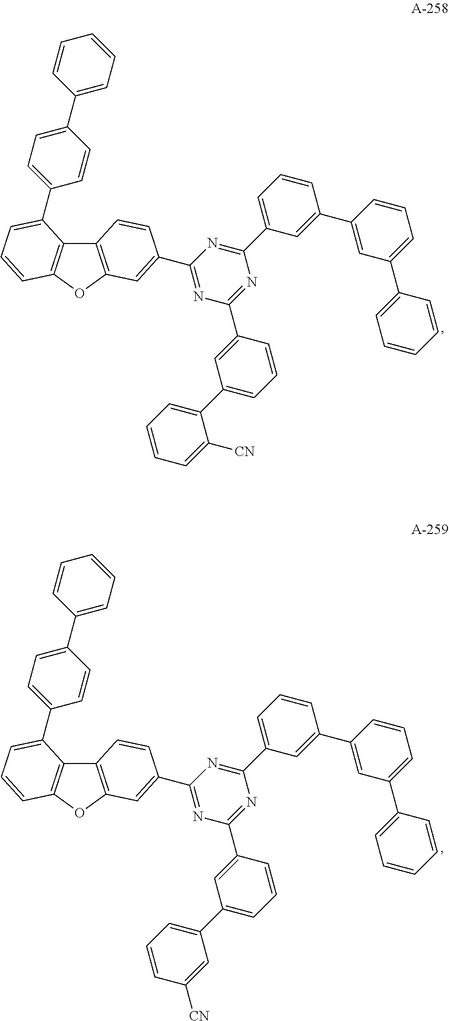 [SMILES: N#Cc1ccccc1-c1cccc(-c2nc(-c3cccc(-c4cccc(-c5ccccc5)c4)c3)nc(-c3ccc4c(c3)oc3cccc(-c5ccc(-c6ccccc6)cc5)c34)n2)c1 ; N#Cc1cccc(-c2cccc(-c3nc(-c4cccc(-c5cccc(-c6ccccc6)c5)c4)nc(-c4ccc5c(c4)oc4cccc(-c6ccc(-c7ccccc7)cc6)c45)n3)c2)c1] 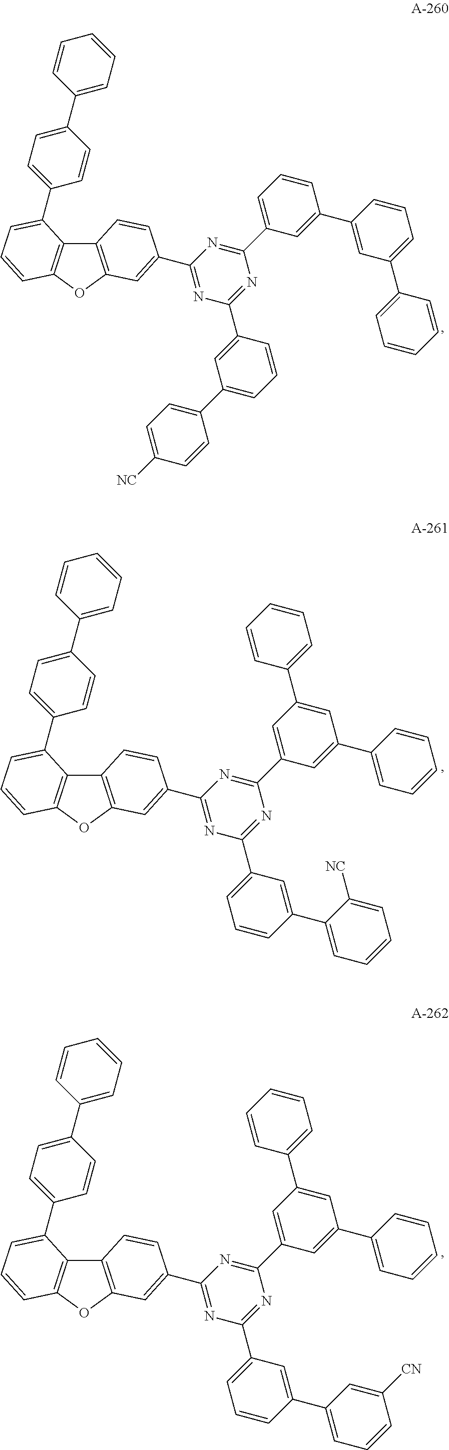 [SMILES: N#Cc1ccc(-c2cccc(-c3nc(-c4cccc(-c5cccc(-c6ccccc6)c5)c4)nc(-c4ccc5c(c4)oc4cccc(-c6ccc(-c7ccccc7)cc6)c45)n3)c2)cc1 ; N#Cc1ccccc1-c1cccc(-c2nc(-c3cc(-c4ccccc4)cc(-c4ccccc4)c3)nc(-c3ccc4c(c3)oc3cccc(-c5ccc(-c6ccccc6)cc5)c34)n2)c1 ; N#Cc1cccc(-c2cccc(-c3nc(-c4cc(-c5ccccc5)cc(-c5ccccc5)c4)nc(-c4ccc5c(c4)oc4cccc(-c6ccc(-c7ccccc7)cc6)c45)n3)c2)c1] 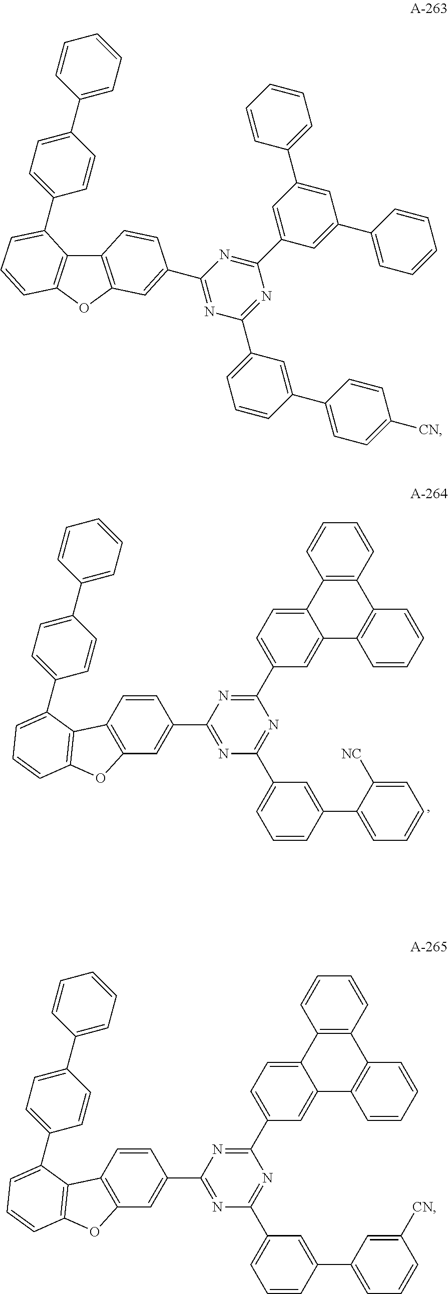 [SMILES: N#Cc1ccc(-c2cccc(-c3nc(-c4cc(-c5ccccc5)cc(-c5ccccc5)c4)nc(-c4ccc5c(c4)oc4cccc(-c6ccc(-c7ccccc7)cc6)c45)n3)c2)cc1 ; N#Cc1ccccc1-c1cccc(-c2nc(-c3ccc4c(c3)oc3cccc(-c5ccc(-c6ccccc6)cc5)c34)nc(-c3ccc4c5ccccc5c5ccccc5c4c3)n2)c1 ; N#Cc1cccc(-c2cccc(-c3nc(-c4ccc5c(c4)oc4cccc(-c6ccc(-c7ccccc7)cc6)c45)nc(-c4ccc5c6ccccc6c6ccccc6c5c4)n3)c2)c1] 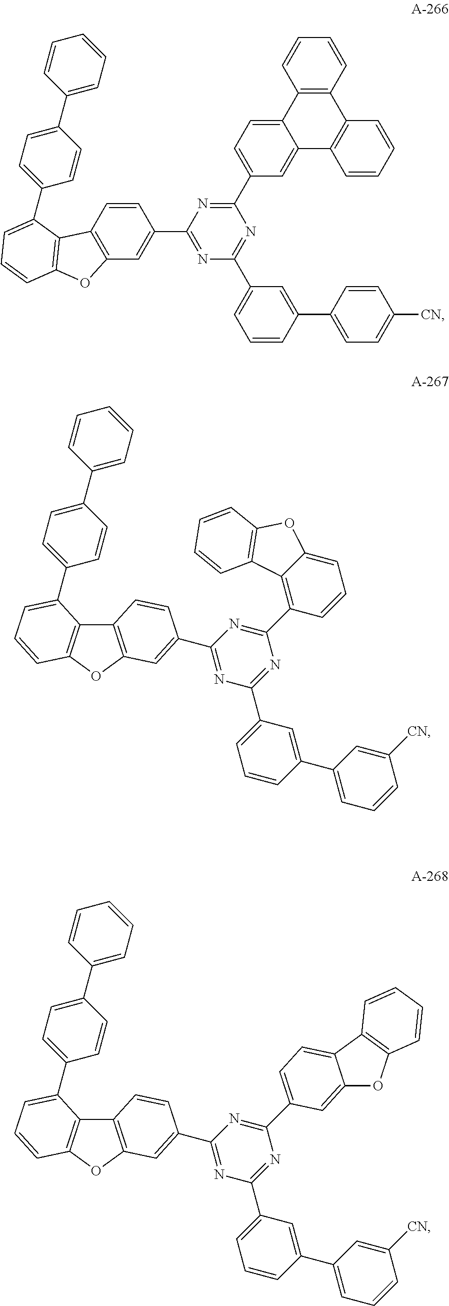 [SMILES: N#Cc1ccc(-c2cccc(-c3nc(-c4ccc5c(c4)oc4cccc(-c6ccc(-c7ccccc7)cc6)c45)nc(-c4ccc5c6ccccc6c6ccccc6c5c4)n3)c2)cc1 ; N#Cc1cccc(-c2cccc(-c3nc(-c4ccc5c(c4)oc4cccc(-c6ccc(-c7ccccc7)cc6)c45)nc(-c4cccc5oc6ccccc6c45)n3)c2)c1 ; N#Cc1cccc(-c2cccc(-c3nc(-c4ccc5c(c4)oc4ccccc45)nc(-c4ccc5c(c4)oc4cccc(-c6ccc(-c7ccccc7)cc6)c45)n3)c2)c1] 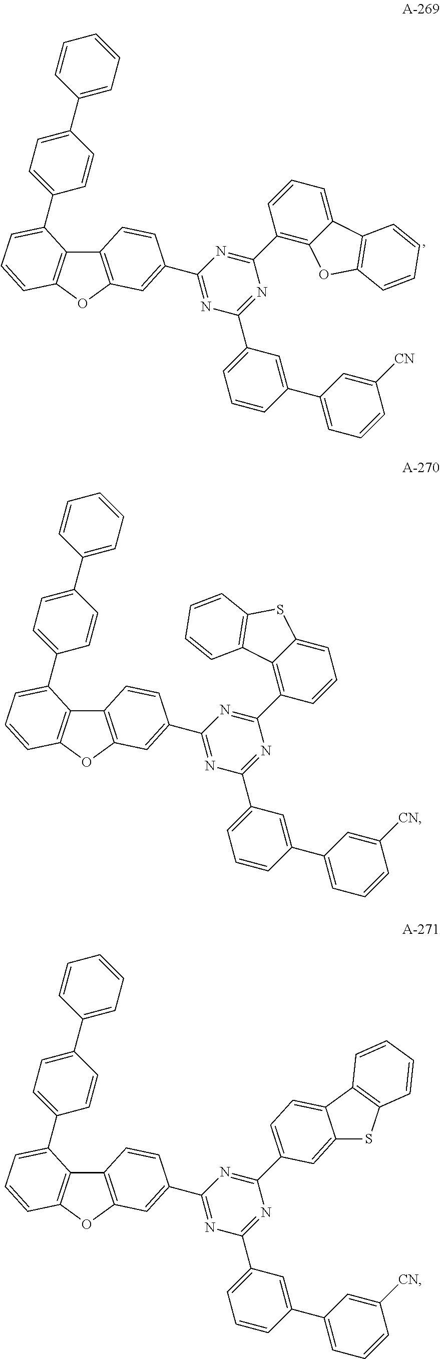 [SMILES: N#Cc1cccc(-c2cccc(-c3nc(-c4ccc5c(c4)oc4cccc(-c6ccc(-c7ccccc7)cc6)c45)nc(-c4cccc5c4oc4ccccc45)n3)c2)c1 ; N#Cc1cccc(-c2cccc(-c3nc(-c4ccc5c(c4)oc4cccc(-c6ccc(-c7ccccc7)cc6)c45)nc(-c4cccc5sc6ccccc6c45)n3)c2)c1 ; N#Cc1cccc(-c2cccc(-c3nc(-c4ccc5c(c4)oc4cccc(-c6ccc(-c7ccccc7)cc6)c45)nc(-c4ccc5c(c4)sc4ccccc45)n3)c2)c1] 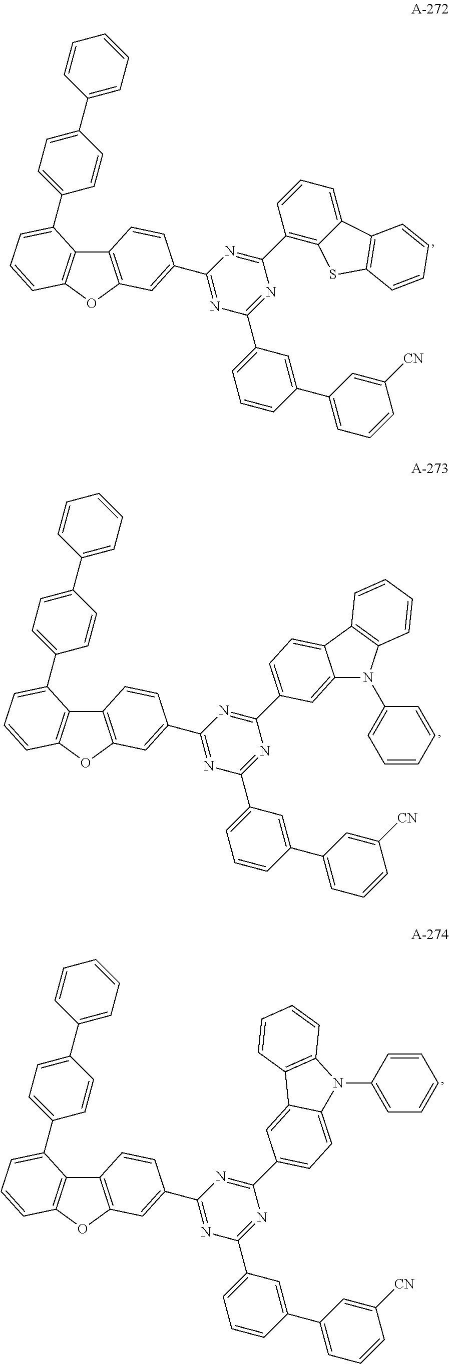 [SMILES: N#Cc1cccc(-c2cccc(-c3nc(-c4ccc5c(c4)oc4cccc(-c6ccc(-c7ccccc7)cc6)c45)nc(-c4cccc5c4sc4ccccc45)n3)c2)c1 ; N#Cc1cccc(-c2cccc(-c3nc(-c4ccc5c(c4)oc4cccc(-c6ccc(-c7ccccc7)cc6)c45)nc(-c4ccc5c6ccccc6n(-c6ccccc6)c5c4)n3)c2)c1 ; N#Cc1cccc(-c2cccc(-c3nc(-c4ccc5c(c4)oc4cccc(-c6ccc(-c7ccccc7)cc6)c45)nc(-c4ccc5c(c4)c4ccccc4n5-c4ccccc4)n3)c2)c1] 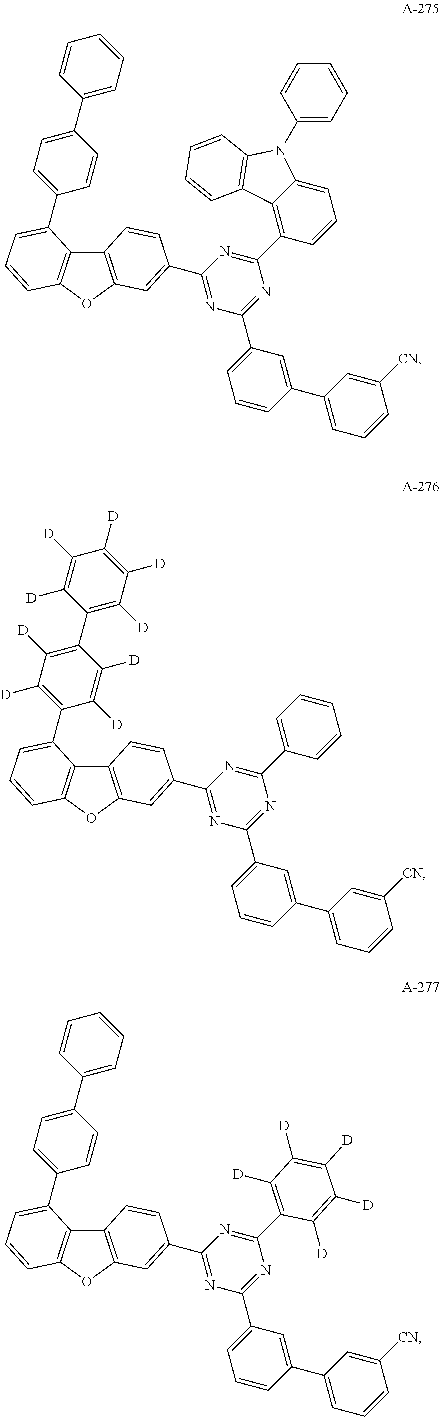 [SMILES: N#Cc1cccc(-c2cccc(-c3nc(-c4ccc5c(c4)oc4cccc(-c6ccc(-c7ccccc7)cc6)c45)nc(-c4cccc5c4c4ccccc4n5-c4ccccc4)n3)c2)c1 ; [2H]c1c([2H])c([2H])c(-c2c([2H])c([2H])c(-c3cccc4oc5cc(-c6nc(-c7ccccc7)nc(-c7cccc(-c8cccc(C#N)c8)c7)n6)ccc5c34)c([2H])c2[2H])c([2H])c1[2H] ; [2H]c1c([2H])c([2H])c(-c2nc(-c3cccc(-c4cccc(C#N)c4)c3)nc(-c3ccc4c(c3)oc3cccc(-c5ccc(-c6ccccc6)cc5)c34)n2)c([2H])c1[2H]] 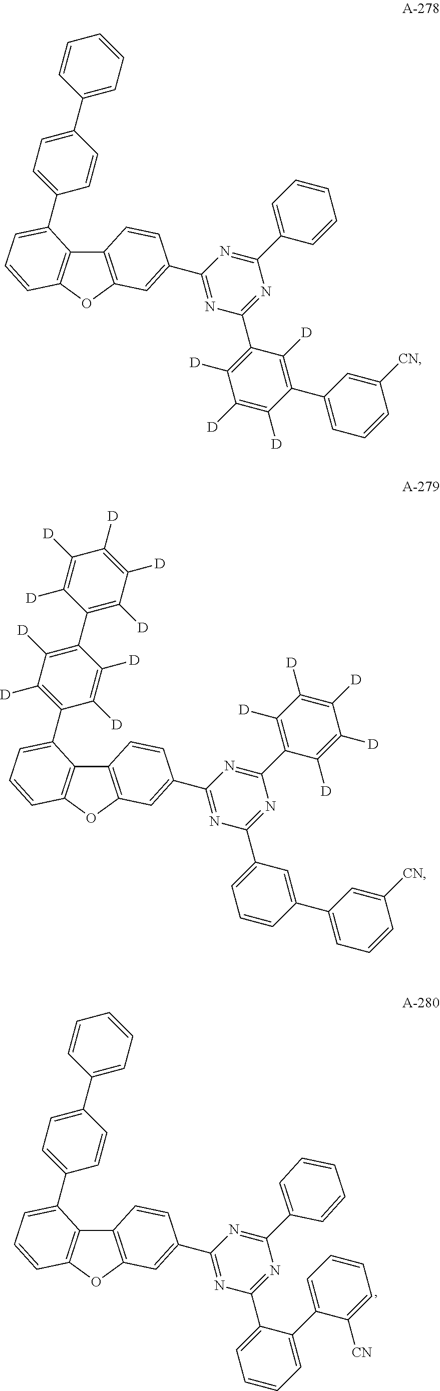 [SMILES: [2H]c1c([2H])c(-c2cccc(C#N)c2)c([2H])c(-c2nc(-c3ccccc3)nc(-c3ccc4c(c3)oc3cccc(-c5ccc(-c6ccccc6)cc5)c34)n2)c1[2H] ; [2H]c1c([2H])c([2H])c(-c2nc(-c3cccc(-c4cccc(C#N)c4)c3)nc(-c3ccc4c(c3)oc3cccc(-c5c([2H])c([2H])c(-c6c([2H])c([2H])c([2H])c([2H])c6[2H])c([2H])c5[2H])c34)n2)c([2H])c1[2H] ; N#Cc1ccccc1-c1ccccc1-c1nc(-c2ccccc2)nc(-c2ccc3c(c2)oc2cccc(-c4ccc(-c5ccccc5)cc4)c23)n1] 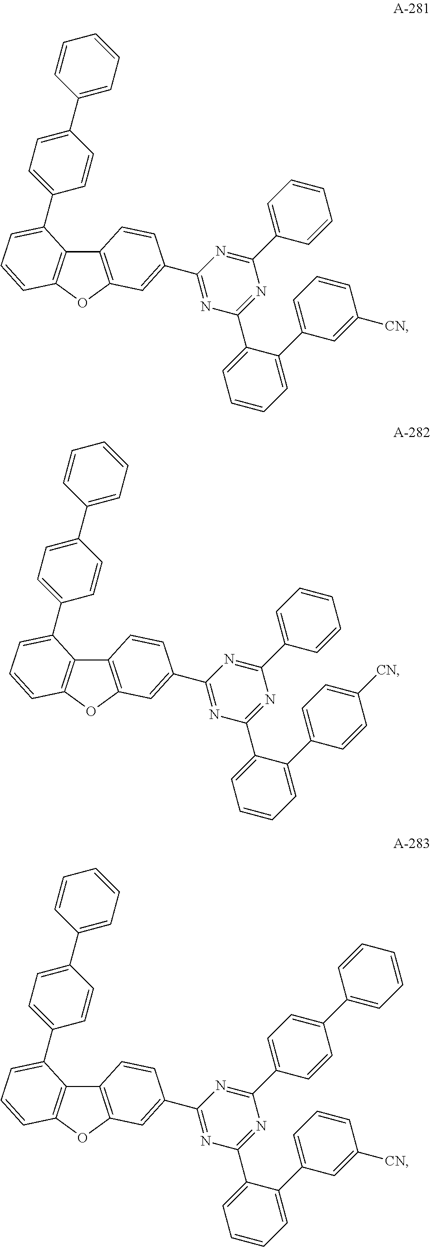 [SMILES: N#Cc1cccc(-c2ccccc2-c2nc(-c3ccccc3)nc(-c3ccc4c(c3)oc3cccc(-c5ccc(-c6ccccc6)cc5)c34)n2)c1 ; N#Cc1ccc(-c2ccccc2-c2nc(-c3ccccc3)nc(-c3ccc4c(c3)oc3cccc(-c5ccc(-c6ccccc6)cc5)c34)n2)cc1 ; N#Cc1cccc(-c2ccccc2-c2nc(-c3ccc(-c4ccccc4)cc3)nc(-c3ccc4c(c3)oc3cccc(-c5ccc(-c6ccccc6)cc5)c34)n2)c1] 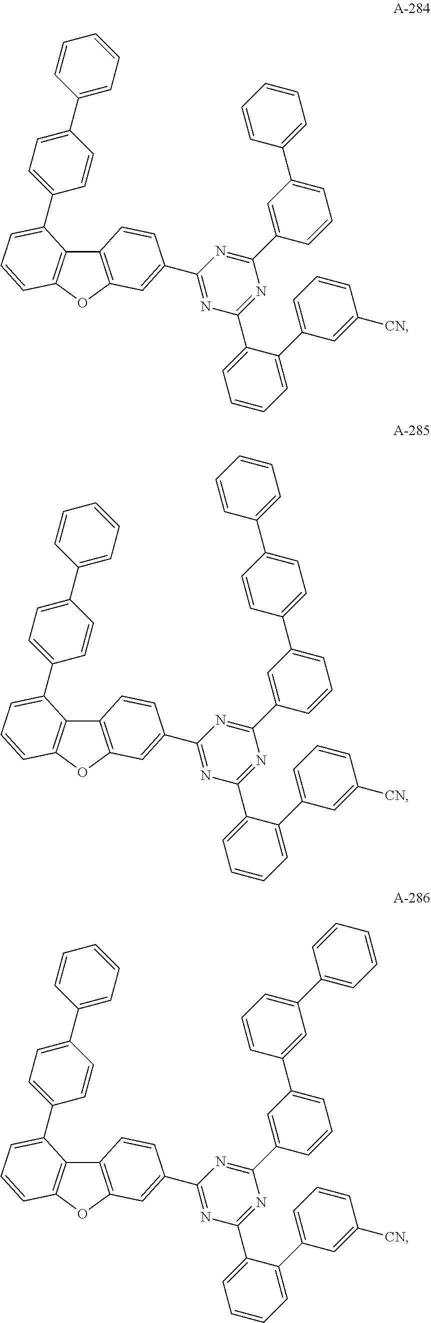 [SMILES: N#Cc1cccc(-c2ccccc2-c2nc(-c3cccc(-c4ccccc4)c3)nc(-c3ccc4c(c3)oc3cccc(-c5ccc(-c6ccccc6)cc5)c34)n2)c1 ; N#Cc1cccc(-c2ccccc2-c2nc(-c3cccc(-c4ccc(-c5ccccc5)cc4)c3)nc(-c3ccc4c(c3)oc3cccc(-c5ccc(-c6ccccc6)cc5)c34)n2)c1 ; N#Cc1cccc(-c2ccccc2-c2nc(-c3cccc(-c4cccc(-c5ccccc5)c4)c3)nc(-c3ccc4c(c3)oc3cccc(-c5ccc(-c6ccccc6)cc5)c34)n2)c1] 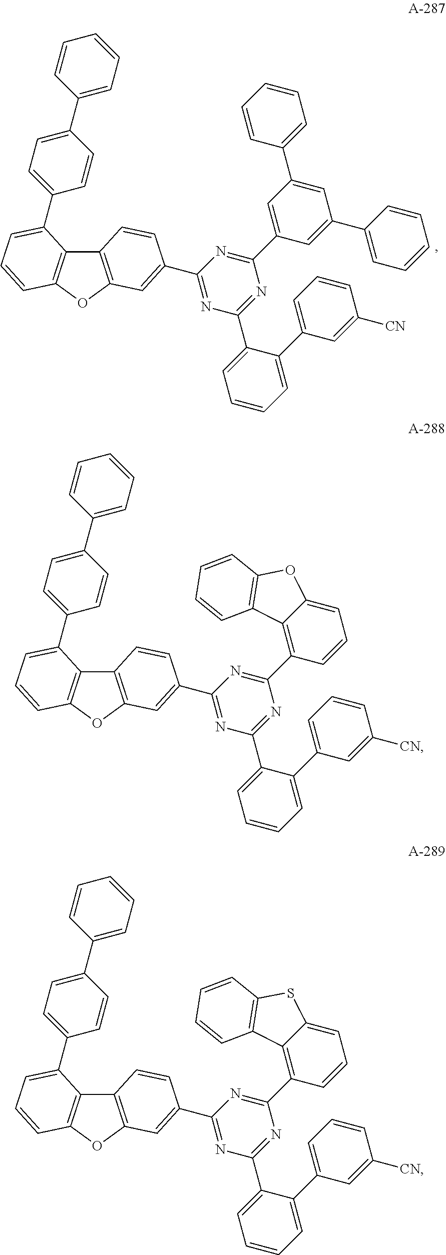 [SMILES: N#Cc1cccc(-c2ccccc2-c2nc(-c3cc(-c4ccccc4)cc(-c4ccccc4)c3)nc(-c3ccc4c(c3)oc3cccc(-c5ccc(-c6ccccc6)cc5)c34)n2)c1 ; N#Cc1cccc(-c2ccccc2-c2nc(-c3ccc4c(c3)oc3cccc(-c5ccc(-c6ccccc6)cc5)c34)nc(-c3cccc4oc5ccccc5c34)n2)c1 ; N#Cc1cccc(-c2ccccc2-c2nc(-c3ccc4c(c3)oc3cccc(-c5ccc(-c6ccccc6)cc5)c34)nc(-c3cccc4sc5ccccc5c34)n2)c1] 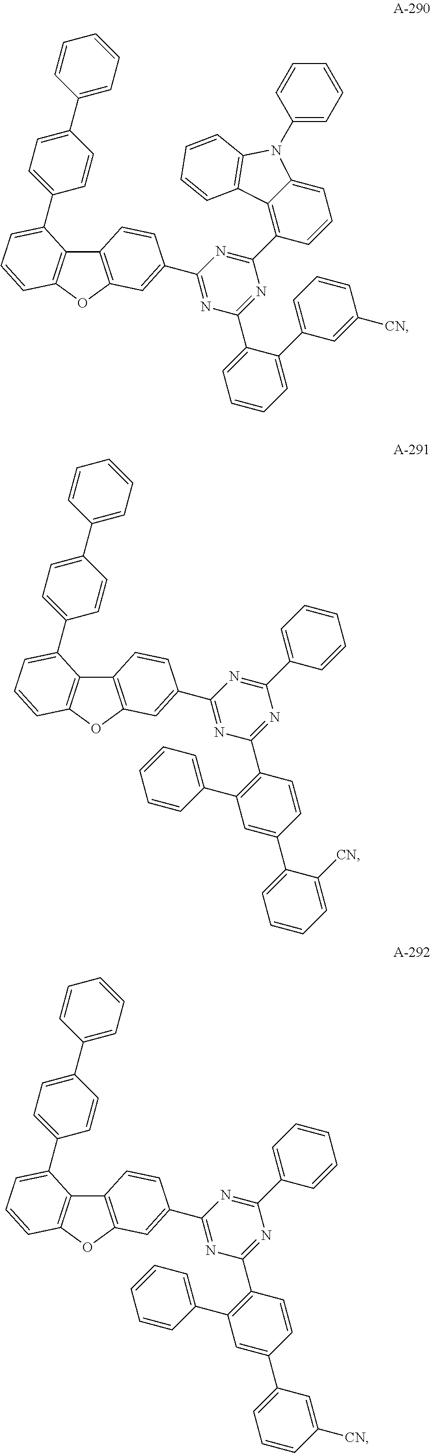 [SMILES: N#Cc1cccc(-c2ccccc2-c2nc(-c3ccc4c(c3)oc3cccc(-c5ccc(-c6ccccc6)cc5)c34)nc(-c3cccc4c3c3ccccc3n4-c3ccccc3)n2)c1 ; N#Cc1ccccc1-c1ccc(-c2nc(-c3ccccc3)nc(-c3ccc4c(c3)oc3cccc(-c5ccc(-c6ccccc6)cc5)c34)n2)c(-c2ccccc2)c1 ; N#Cc1cccc(-c2ccc(-c3nc(-c4ccccc4)nc(-c4ccc5c(c4)oc4cccc(-c6ccc(-c7ccccc7)cc6)c45)n3)c(-c3ccccc3)c2)c1] 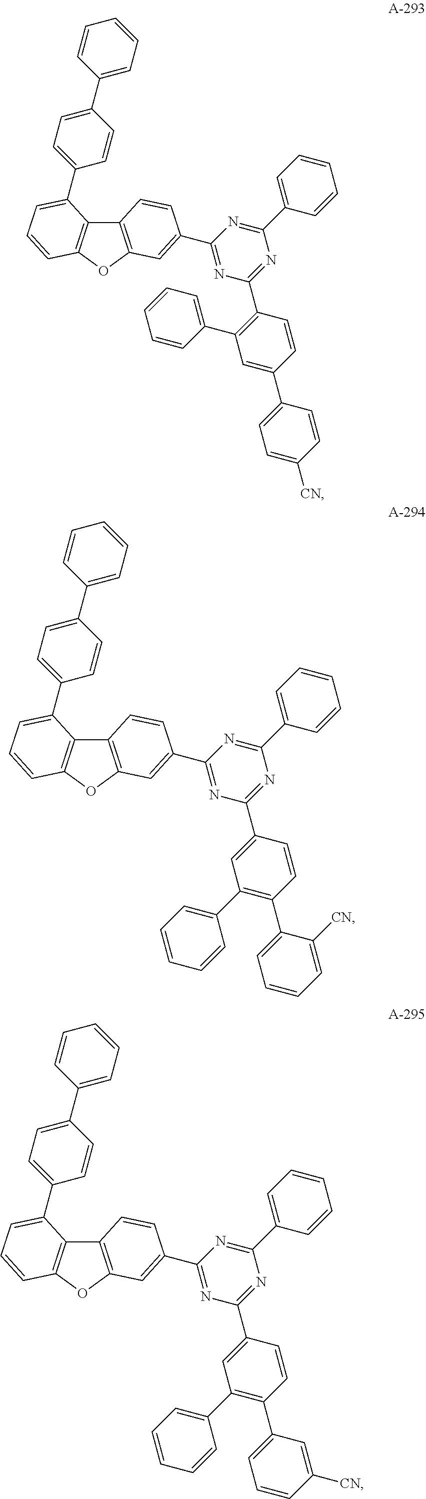 [SMILES: N#Cc1ccc(-c2ccc(-c3nc(-c4ccccc4)nc(-c4ccc5c(c4)oc4cccc(-c6ccc(-c7ccccc7)cc6)c45)n3)c(-c3ccccc3)c2)cc1 ; N#Cc1ccccc1-c1ccc(-c2nc(-c3ccccc3)nc(-c3ccc4c(c3)oc3cccc(-c5ccc(-c6ccccc6)cc5)c34)n2)cc1-c1ccccc1 ; N#Cc1cccc(-c2ccc(-c3nc(-c4ccccc4)nc(-c4ccc5c(c4)oc4cccc(-c6ccc(-c7ccccc7)cc6)c45)n3)cc2-c2ccccc2)c1] 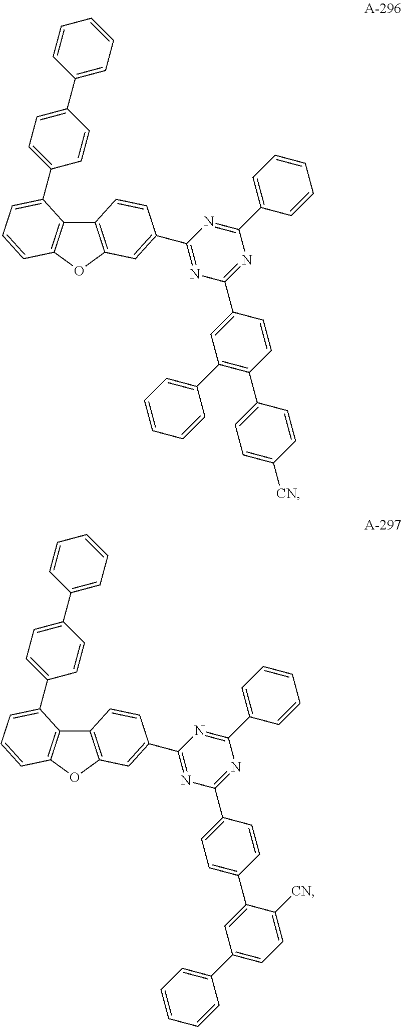 [SMILES: N#Cc1ccc(-c2ccc(-c3nc(-c4ccccc4)nc(-c4ccc5c(c4)oc4cccc(-c6ccc(-c7ccccc7)cc6)c45)n3)cc2-c2ccccc2)cc1 ; N#Cc1ccc(-c2ccccc2)cc1-c1ccc(-c2nc(-c3ccccc3)nc(-c3ccc4c(c3)oc3cccc(-c5ccc(-c6ccccc6)cc5)c34)n2)cc1] 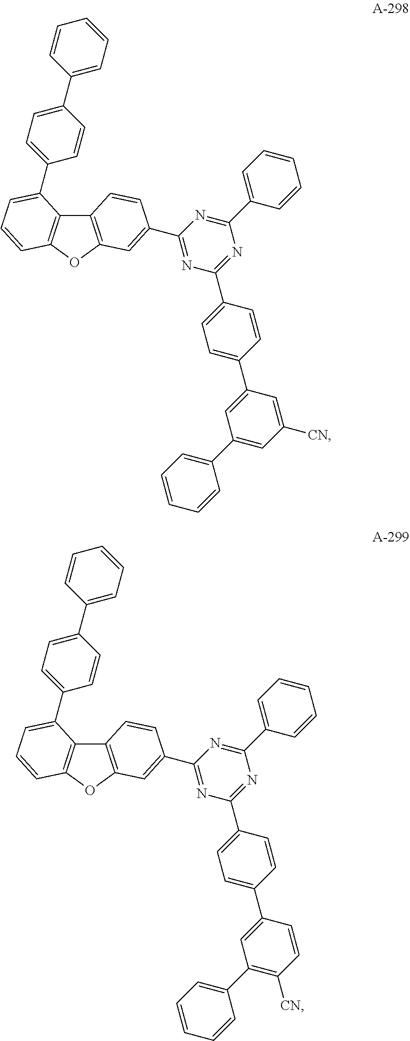 [SMILES: N#Cc1cc(-c2ccccc2)cc(-c2ccc(-c3nc(-c4ccccc4)nc(-c4ccc5c(c4)oc4cccc(-c6ccc(-c7ccccc7)cc6)c45)n3)cc2)c1 ; N#Cc1ccc(-c2ccc(-c3nc(-c4ccccc4)nc(-c4ccc5c(c4)oc4cccc(-c6ccc(-c7ccccc7)cc6)c45)n3)cc2)cc1-c1ccccc1] 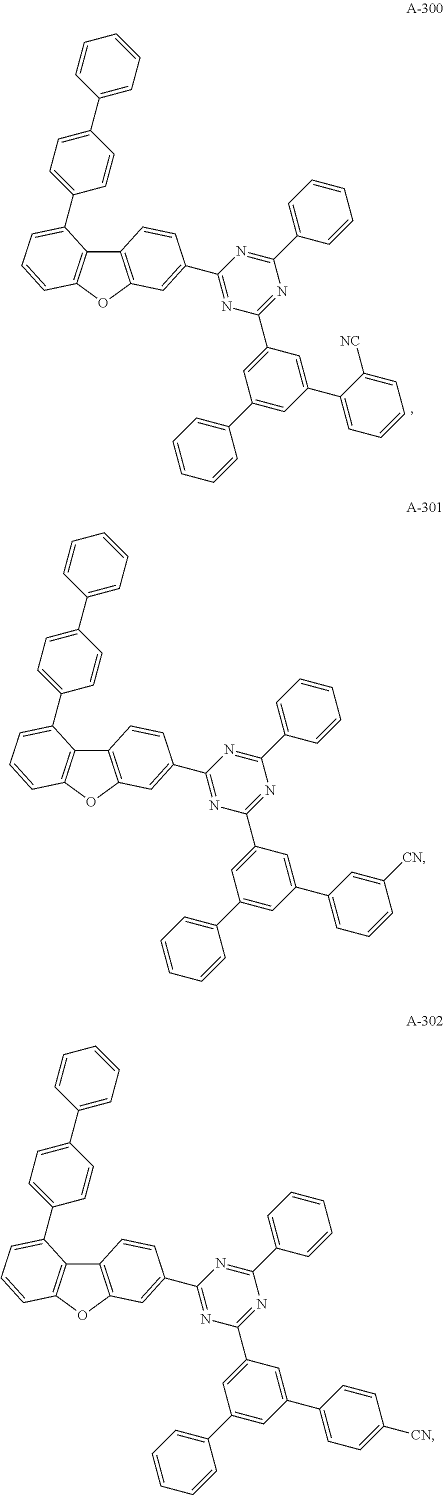 [SMILES: N#Cc1ccccc1-c1cc(-c2ccccc2)cc(-c2nc(-c3ccccc3)nc(-c3ccc4c(c3)oc3cccc(-c5ccc(-c6ccccc6)cc5)c34)n2)c1 ; N#Cc1cccc(-c2cc(-c3ccccc3)cc(-c3nc(-c4ccccc4)nc(-c4ccc5c(c4)oc4cccc(-c6ccc(-c7ccccc7)cc6)c45)n3)c2)c1 ; N#Cc1ccc(-c2cc(-c3ccccc3)cc(-c3nc(-c4ccccc4)nc(-c4ccc5c(c4)oc4cccc(-c6ccc(-c7ccccc7)cc6)c45)n3)c2)cc1] 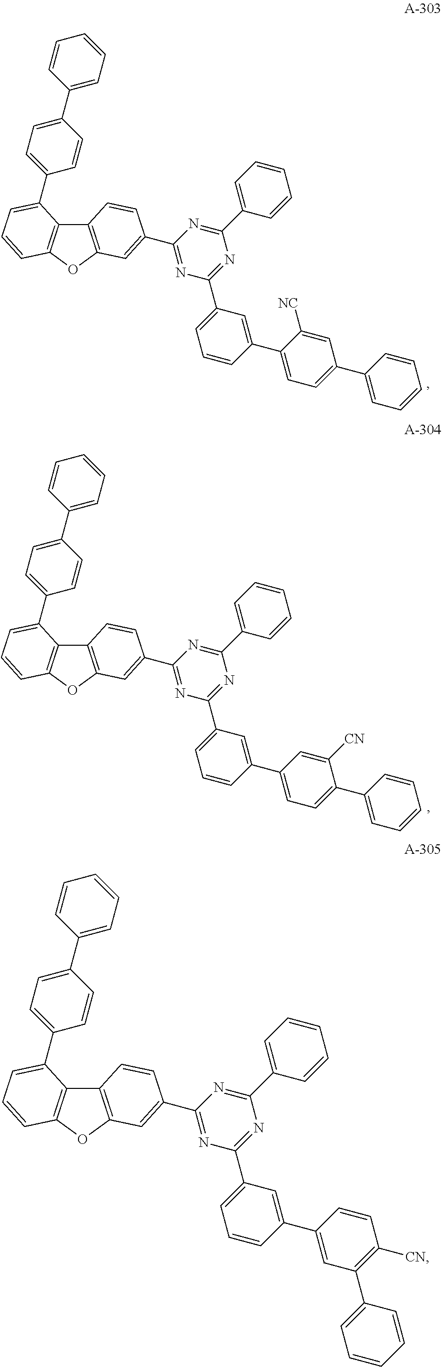 [SMILES: N#Cc1cc(-c2ccccc2)ccc1-c1cccc(-c2nc(-c3ccccc3)nc(-c3ccc4c(c3)oc3cccc(-c5ccc(-c6ccccc6)cc5)c34)n2)c1 ; N#Cc1cc(-c2cccc(-c3nc(-c4ccccc4)nc(-c4ccc5c(c4)oc4cccc(-c6ccc(-c7ccccc7)cc6)c45)n3)c2)ccc1-c1ccccc1 ; N#Cc1ccc(-c2cccc(-c3nc(-c4ccccc4)nc(-c4ccc5c(c4)oc4cccc(-c6ccc(-c7ccccc7)cc6)c45)n3)c2)cc1-c1ccccc1] 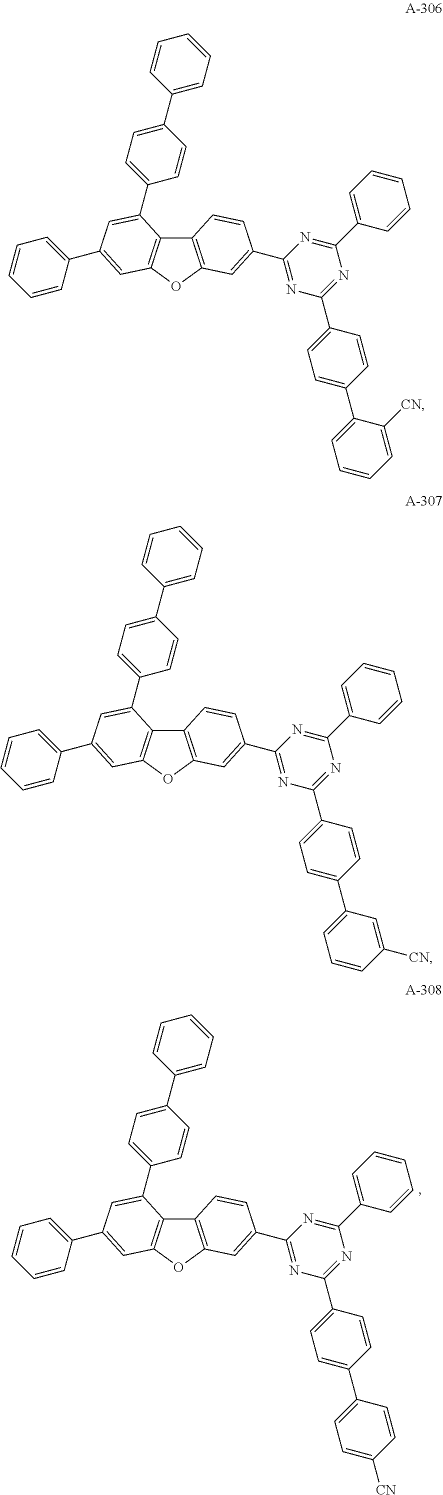 [SMILES: N#Cc1ccccc1-c1ccc(-c2nc(-c3ccccc3)nc(-c3ccc4c(c3)oc3cc(-c5ccccc5)cc(-c5ccc(-c6ccccc6)cc5)c34)n2)cc1 ; N#Cc1cccc(-c2ccc(-c3nc(-c4ccccc4)nc(-c4ccc5c(c4)oc4cc(-c6ccccc6)cc(-c6ccc(-c7ccccc7)cc6)c45)n3)cc2)c1 ; N#Cc1ccc(-c2ccc(-c3nc(-c4ccccc4)nc(-c4ccc5c(c4)oc4cc(-c6ccccc6)cc(-c6ccc(-c7ccccc7)cc6)c45)n3)cc2)cc1] 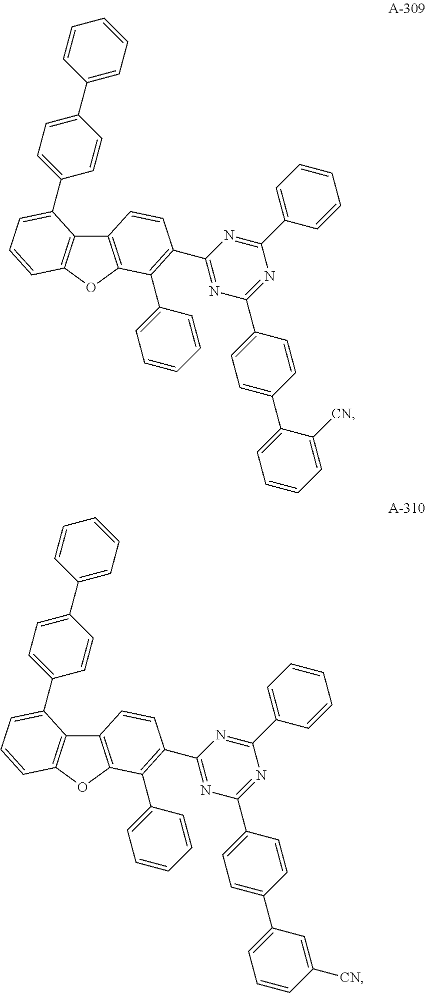 [SMILES: N#Cc1ccccc1-c1ccc(-c2nc(-c3ccccc3)nc(-c3ccc4c(oc5cccc(-c6ccc(-c7ccccc7)cc6)c54)c3-c3ccccc3)n2)cc1 ; N#Cc1cccc(-c2ccc(-c3nc(-c4ccccc4)nc(-c4ccc5c(oc6cccc(-c7ccc(-c8ccccc8)cc7)c65)c4-c4ccccc4)n3)cc2)c1] 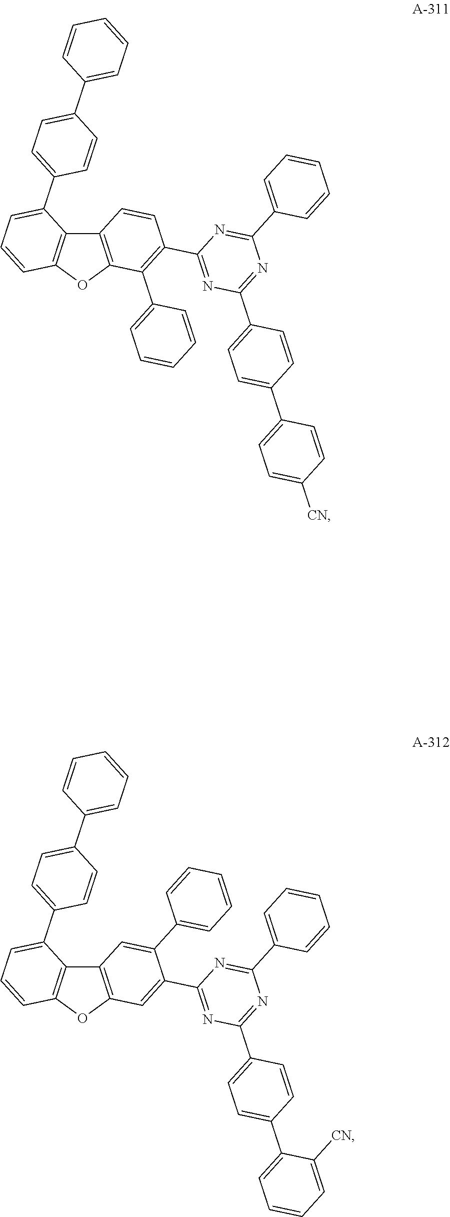 [SMILES: N#Cc1ccc(-c2ccc(-c3nc(-c4ccccc4)nc(-c4ccc5c(oc6cccc(-c7ccc(-c8ccccc8)cc7)c65)c4-c4ccccc4)n3)cc2)cc1 ; N#Cc1ccccc1-c1ccc(-c2nc(-c3ccccc3)nc(-c3cc4oc5cccc(-c6ccc(-c7ccccc7)cc6)c5c4cc3-c3ccccc3)n2)cc1] 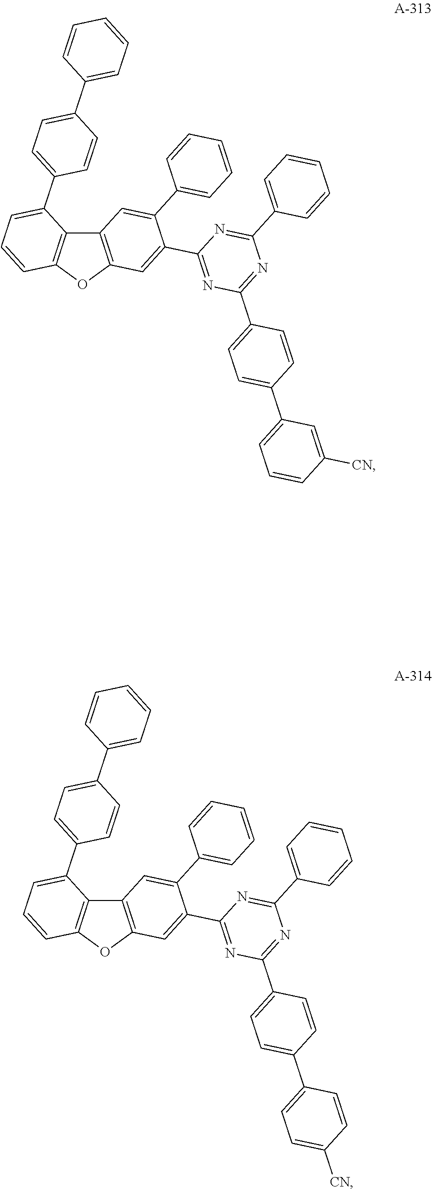 [SMILES: N#Cc1cccc(-c2ccc(-c3nc(-c4ccccc4)nc(-c4cc5oc6cccc(-c7ccc(-c8ccccc8)cc7)c6c5cc4-c4ccccc4)n3)cc2)c1 ; N#Cc1ccc(-c2ccc(-c3nc(-c4ccccc4)nc(-c4cc5oc6cccc(-c7ccc(-c8ccccc8)cc7)c6c5cc4-c4ccccc4)n3)cc2)cc1] 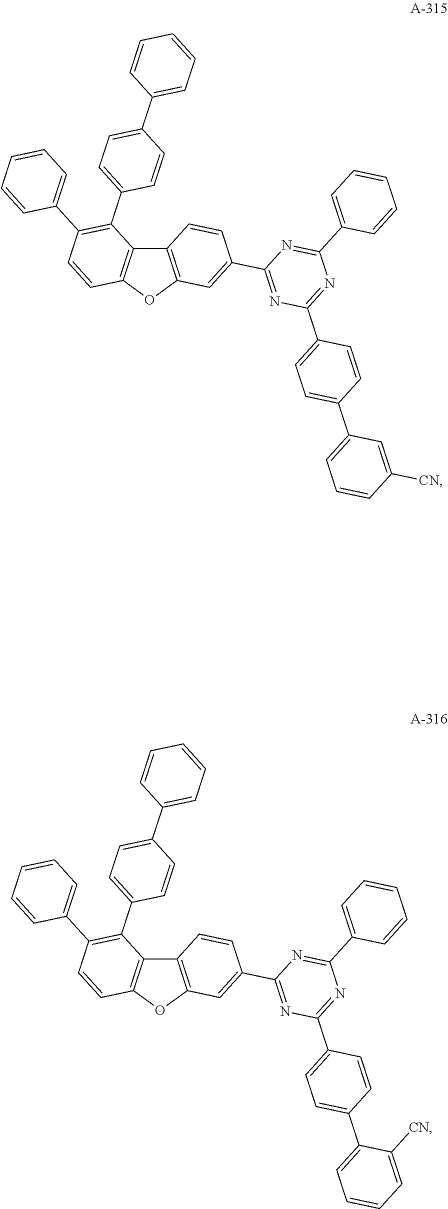 [SMILES: N#Cc1cccc(-c2ccc(-c3nc(-c4ccccc4)nc(-c4ccc5c(c4)oc4ccc(-c6ccccc6)c(-c6ccc(-c7ccccc7)cc6)c45)n3)cc2)c1 ; N#Cc1ccccc1-c1ccc(-c2nc(-c3ccccc3)nc(-c3ccc4c(c3)oc3ccc(-c5ccccc5)c(-c5ccc(-c6ccccc6)cc5)c34)n2)cc1] 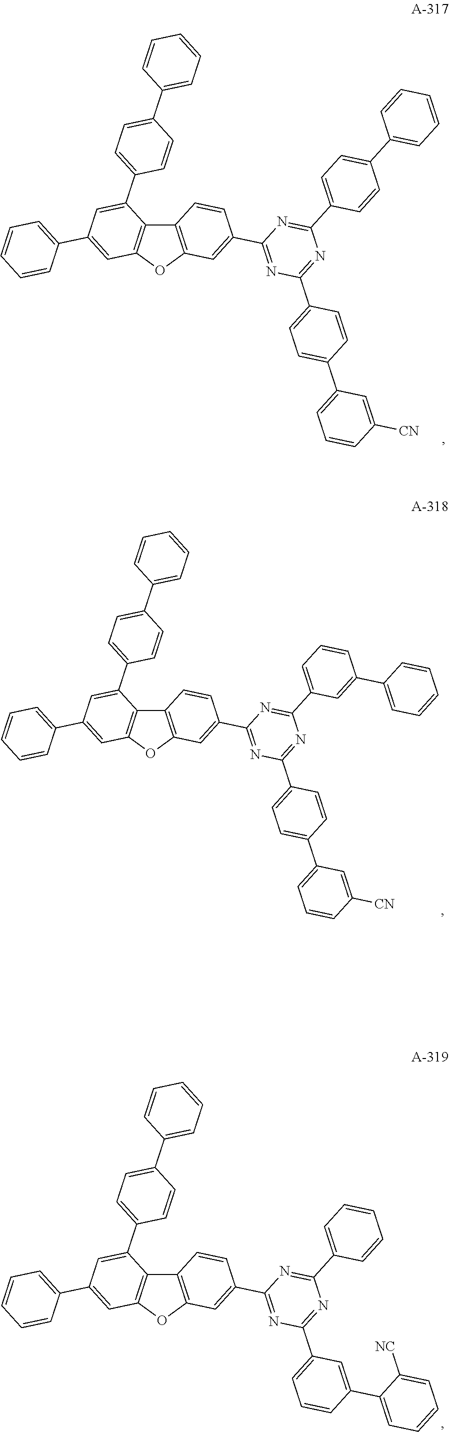 [SMILES: N#Cc1cccc(-c2ccc(-c3nc(-c4ccc(-c5ccccc5)cc4)nc(-c4ccc5c(c4)oc4cc(-c6ccccc6)cc(-c6ccc(-c7ccccc7)cc6)c45)n3)cc2)c1 ; N#Cc1cccc(-c2ccc(-c3nc(-c4cccc(-c5ccccc5)c4)nc(-c4ccc5c(c4)oc4cc(-c6ccccc6)cc(-c6ccc(-c7ccccc7)cc6)c45)n3)cc2)c1 ; N#Cc1ccccc1-c1cccc(-c2nc(-c3ccccc3)nc(-c3ccc4c(c3)oc3cc(-c5ccccc5)cc(-c5ccc(-c6ccccc6)cc5)c34)n2)c1] 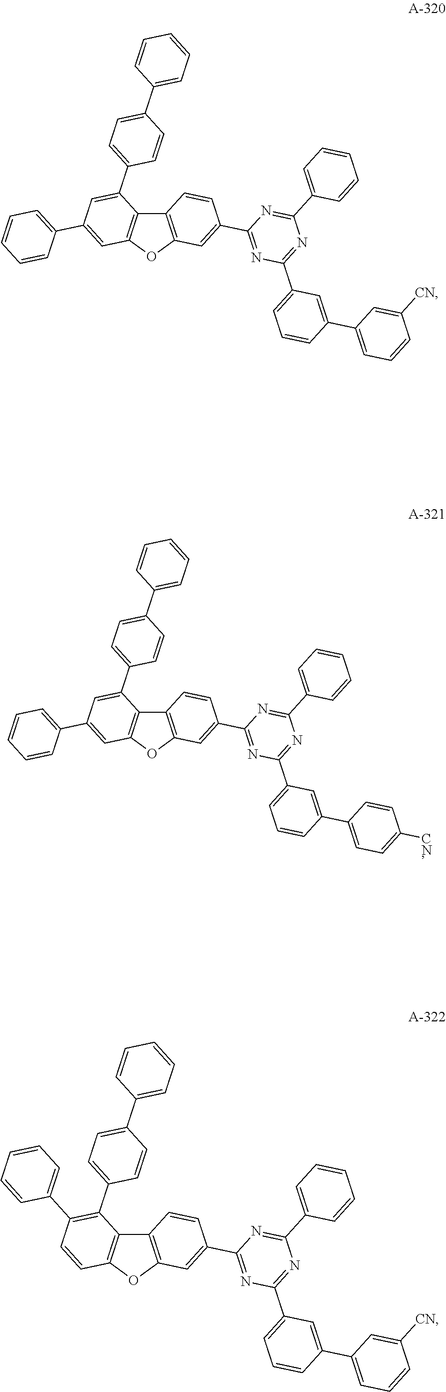 [SMILES: N#Cc1cccc(-c2cccc(-c3nc(-c4ccccc4)nc(-c4ccc5c(c4)oc4cc(-c6ccccc6)cc(-c6ccc(-c7ccccc7)cc6)c45)n3)c2)c1 ; N#Cc1ccc(-c2cccc(-c3nc(-c4ccccc4)nc(-c4ccc5c(c4)oc4cc(-c6ccccc6)cc(-c6ccc(-c7ccccc7)cc6)c45)n3)c2)cc1 ; N#Cc1cccc(-c2cccc(-c3nc(-c4ccccc4)nc(-c4ccc5c(c4)oc4ccc(-c6ccccc6)c(-c6ccc(-c7ccccc7)cc6)c45)n3)c2)c1] 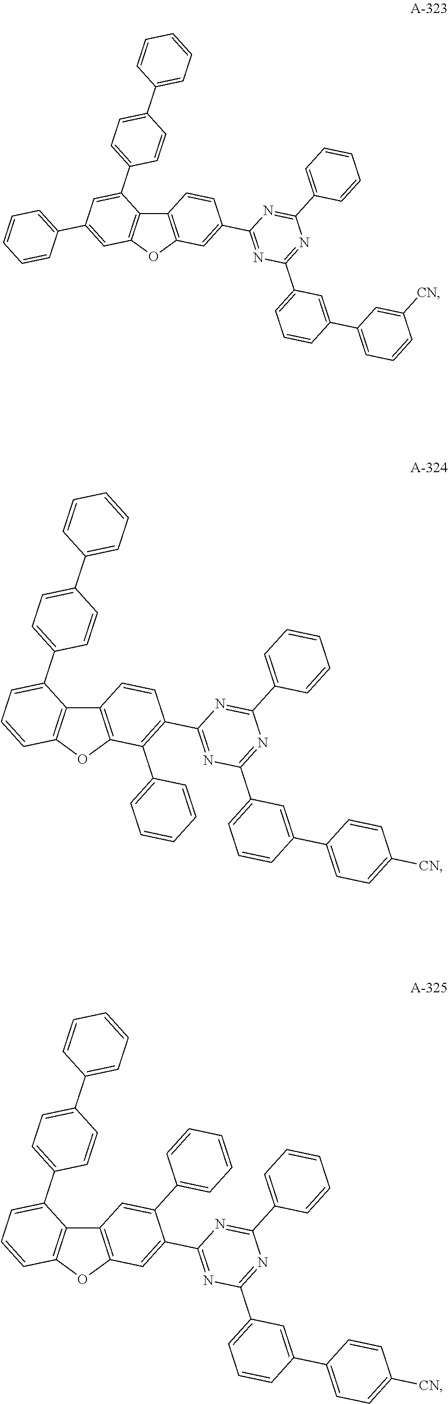 [SMILES: N#Cc1cccc(-c2cccc(-c3nc(-c4ccccc4)nc(-c4ccc5c(c4)oc4cc(-c6ccccc6)cc(-c6ccc(-c7ccccc7)cc6)c45)n3)c2)c1 ; N#Cc1ccc(-c2cccc(-c3nc(-c4ccccc4)nc(-c4ccc5c(oc6cccc(-c7ccc(-c8ccccc8)cc7)c65)c4-c4ccccc4)n3)c2)cc1 ; N#Cc1ccc(-c2cccc(-c3nc(-c4ccccc4)nc(-c4cc5oc6cccc(-c7ccc(-c8ccccc8)cc7)c6c5cc4-c4ccccc4)n3)c2)cc1] 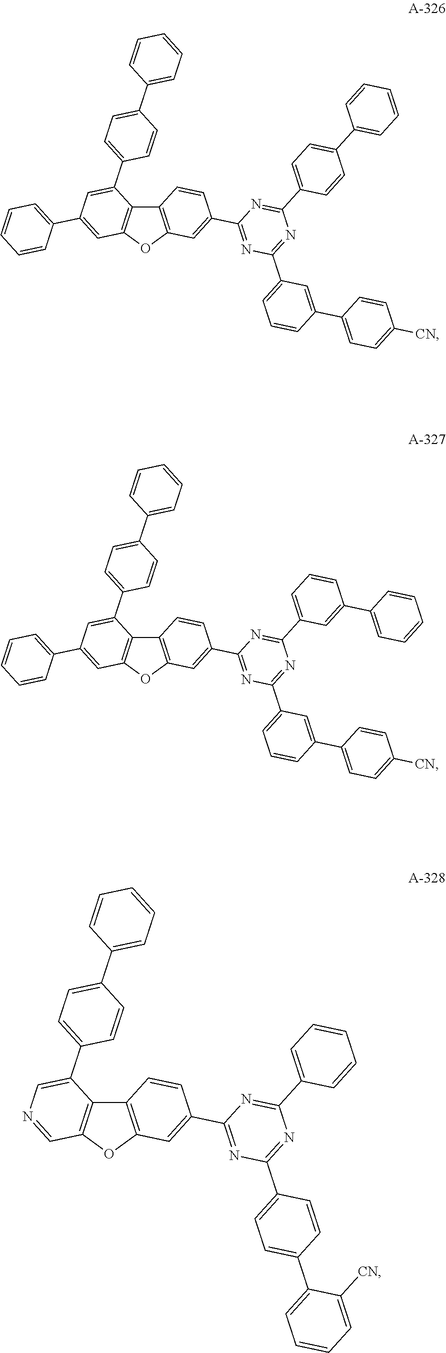 [SMILES: N#Cc1ccc(-c2cccc(-c3nc(-c4ccc(-c5ccccc5)cc4)nc(-c4ccc5c(c4)oc4cc(-c6ccccc6)cc(-c6ccc(-c7ccccc7)cc6)c45)n3)c2)cc1 ; N#Cc1ccc(-c2cccc(-c3nc(-c4cccc(-c5ccccc5)c4)nc(-c4ccc5c(c4)oc4cc(-c6ccccc6)cc(-c6ccc(-c7ccccc7)cc6)c45)n3)c2)cc1 ; N#Cc1ccccc1-c1ccc(-c2nc(-c3ccccc3)nc(-c3ccc4c(c3)oc3cncc(-c5ccc(-c6ccccc6)cc5)c34)n2)cc1] 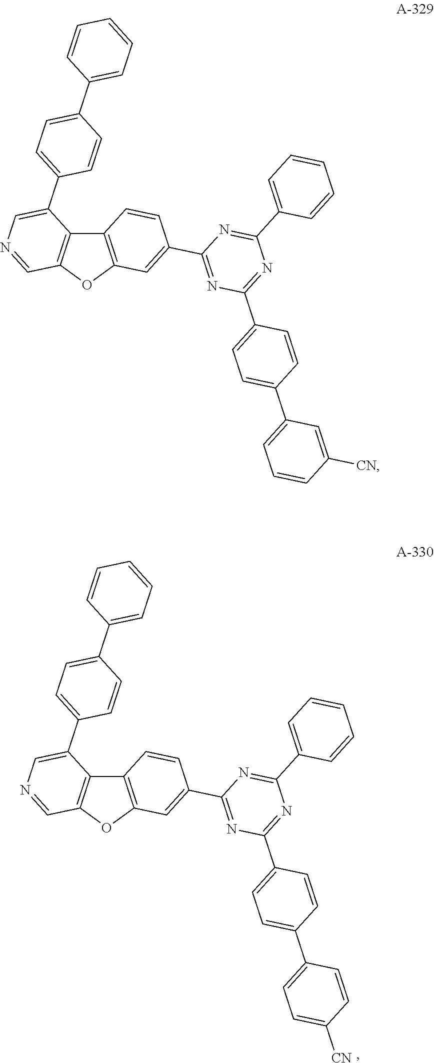 [SMILES: N#Cc1cccc(-c2ccc(-c3nc(-c4ccccc4)nc(-c4ccc5c(c4)oc4cncc(-c6ccc(-c7ccccc7)cc6)c45)n3)cc2)c1 ; N#Cc1ccc(-c2ccc(-c3nc(-c4ccccc4)nc(-c4ccc5c(c4)oc4cncc(-c6ccc(-c7ccccc7)cc6)c45)n3)cc2)cc1] 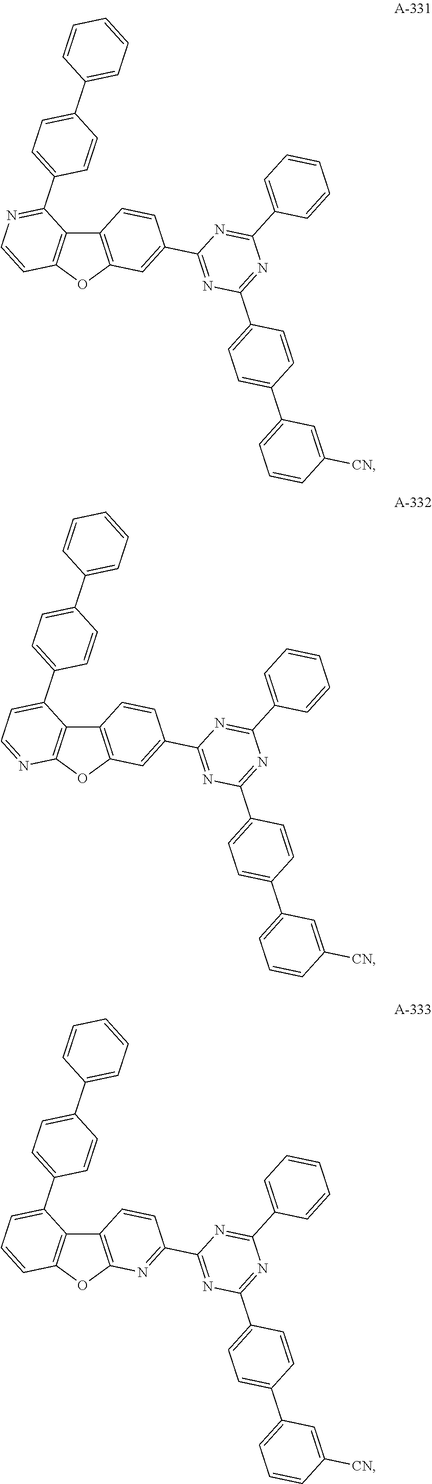 [SMILES: N#Cc1cccc(-c2ccc(-c3nc(-c4ccccc4)nc(-c4ccc5c(c4)oc4ccnc(-c6ccc(-c7ccccc7)cc6)c45)n3)cc2)c1 ; N#Cc1cccc(-c2ccc(-c3nc(-c4ccccc4)nc(-c4ccc5c(c4)oc4nccc(-c6ccc(-c7ccccc7)cc6)c45)n3)cc2)c1 ; N#Cc1cccc(-c2ccc(-c3nc(-c4ccccc4)nc(-c4ccc5c(n4)oc4cccc(-c6ccc(-c7ccccc7)cc6)c45)n3)cc2)c1] 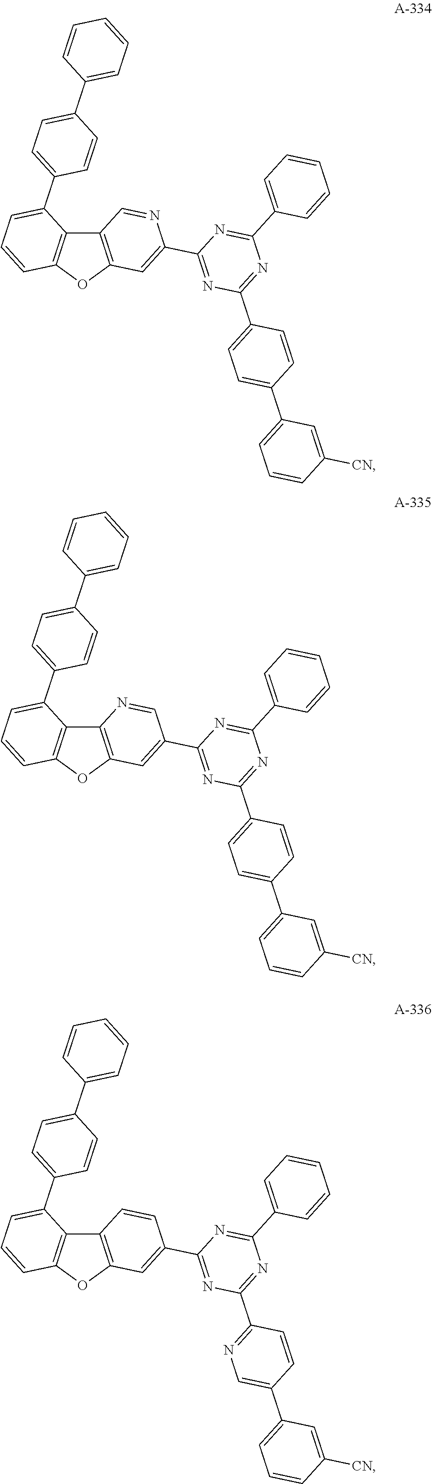 [SMILES: N#Cc1cccc(-c2ccc(-c3nc(-c4ccccc4)nc(-c4cc5oc6cccc(-c7ccc(-c8ccccc8)cc7)c6c5cn4)n3)cc2)c1 ; N#Cc1cccc(-c2ccc(-c3nc(-c4ccccc4)nc(-c4cnc5c(c4)oc4cccc(-c6ccc(-c7ccccc7)cc6)c45)n3)cc2)c1 ; N#Cc1cccc(-c2ccc(-c3nc(-c4ccccc4)nc(-c4ccc5c(c4)oc4cccc(-c6ccc(-c7ccccc7)cc6)c45)n3)nc2)c1] 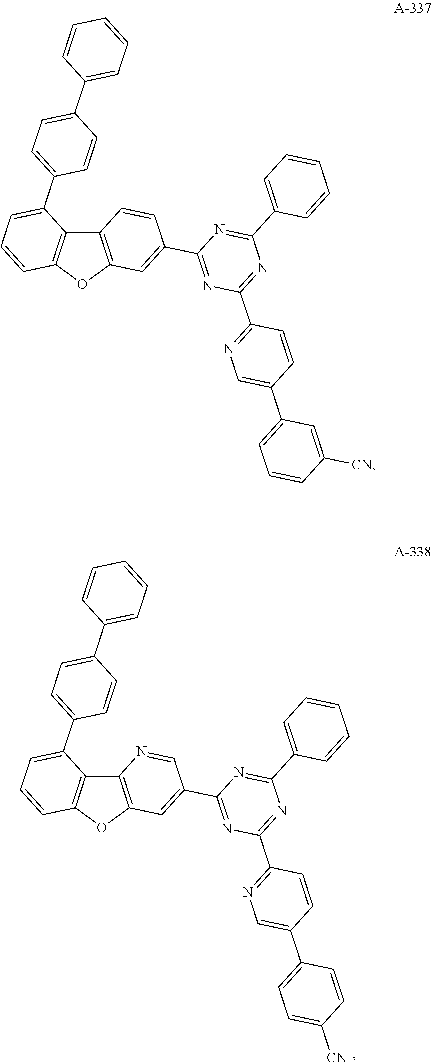 [SMILES: N#Cc1cccc(-c2ccc(-c3nc(-c4ccccc4)nc(-c4ccc5c(c4)oc4cccc(-c6ccc(-c7ccccc7)cc6)c45)n3)nc2)c1 ; N#Cc1ccc(-c2ccc(-c3nc(-c4ccccc4)nc(-c4cnc5c(c4)oc4cccc(-c6ccc(-c7ccccc7)cc6)c45)n3)nc2)cc1] 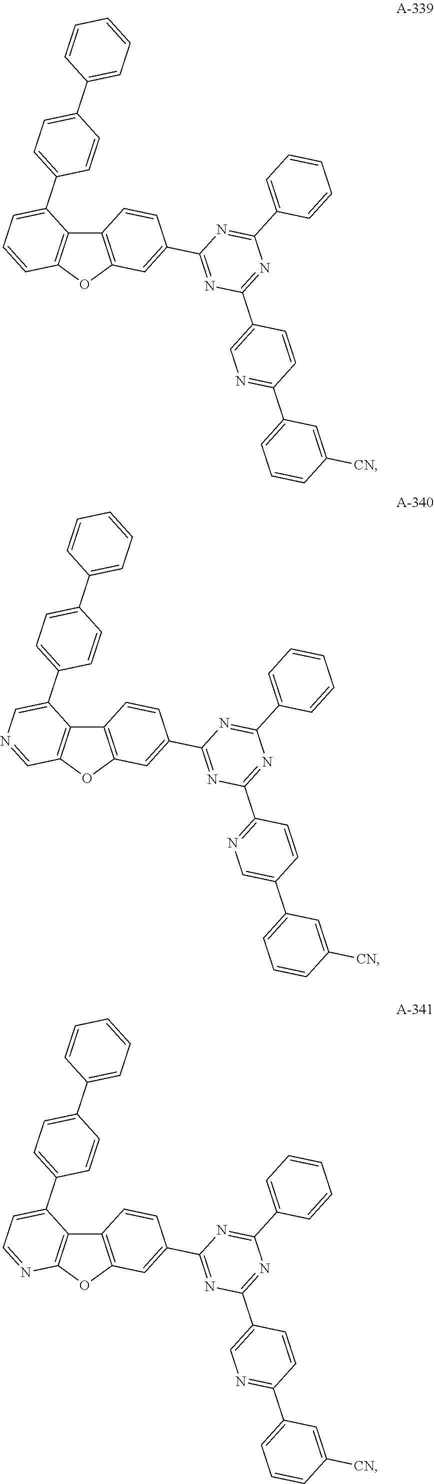 [SMILES: N#Cc1cccc(-c2ccc(-c3nc(-c4ccccc4)nc(-c4ccc5c(c4)oc4cccc(-c6ccc(-c7ccccc7)cc6)c45)n3)cn2)c1 ; N#Cc1cccc(-c2ccc(-c3nc(-c4ccccc4)nc(-c4ccc5c(c4)oc4cncc(-c6ccc(-c7ccccc7)cc6)c45)n3)nc2)c1 ; N#Cc1cccc(-c2ccc(-c3nc(-c4ccccc4)nc(-c4ccc5c(c4)oc4nccc(-c6ccc(-c7ccccc7)cc6)c45)n3)cn2)c1] 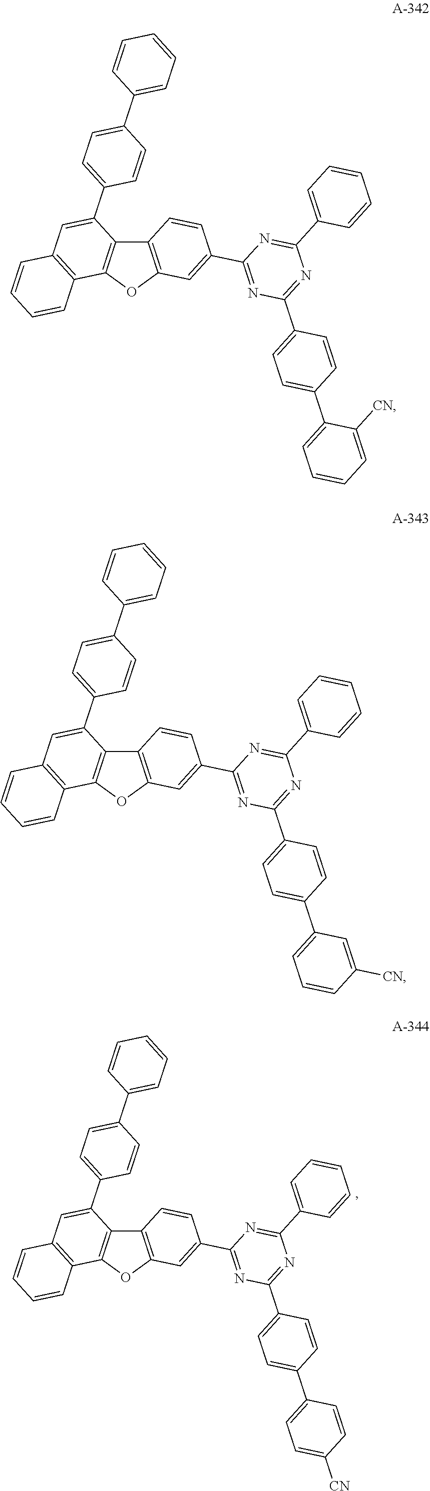 [SMILES: N#Cc1ccccc1-c1ccc(-c2nc(-c3ccccc3)nc(-c3ccc4c(c3)oc3c5ccccc5cc(-c5ccc(-c6ccccc6)cc5)c43)n2)cc1 ; N#Cc1cccc(-c2ccc(-c3nc(-c4ccccc4)nc(-c4ccc5c(c4)oc4c6ccccc6cc(-c6ccc(-c7ccccc7)cc6)c54)n3)cc2)c1 ; N#Cc1ccc(-c2ccc(-c3nc(-c4ccccc4)nc(-c4ccc5c(c4)oc4c6ccccc6cc(-c6ccc(-c7ccccc7)cc6)c54)n3)cc2)cc1] 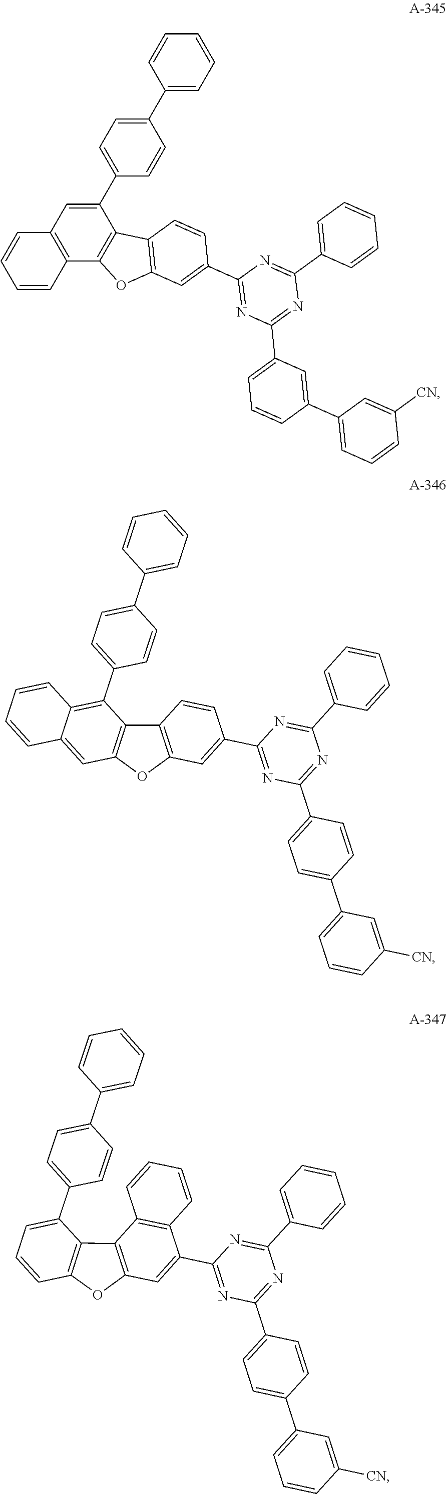 [SMILES: N#Cc1cccc(-c2cccc(-c3nc(-c4ccccc4)nc(-c4ccc5c(c4)oc4c6ccccc6cc(-c6ccc(-c7ccccc7)cc6)c54)n3)c2)c1 ; N#Cc1cccc(-c2ccc(-c3nc(-c4ccccc4)nc(-c4ccc5c(c4)oc4cc6ccccc6c(-c6ccc(-c7ccccc7)cc6)c45)n3)cc2)c1 ; N#Cc1cccc(-c2ccc(-c3nc(-c4ccccc4)nc(-c4cc5oc6cccc(-c7ccc(-c8ccccc8)cc7)c6c5c5ccccc45)n3)cc2)c1] 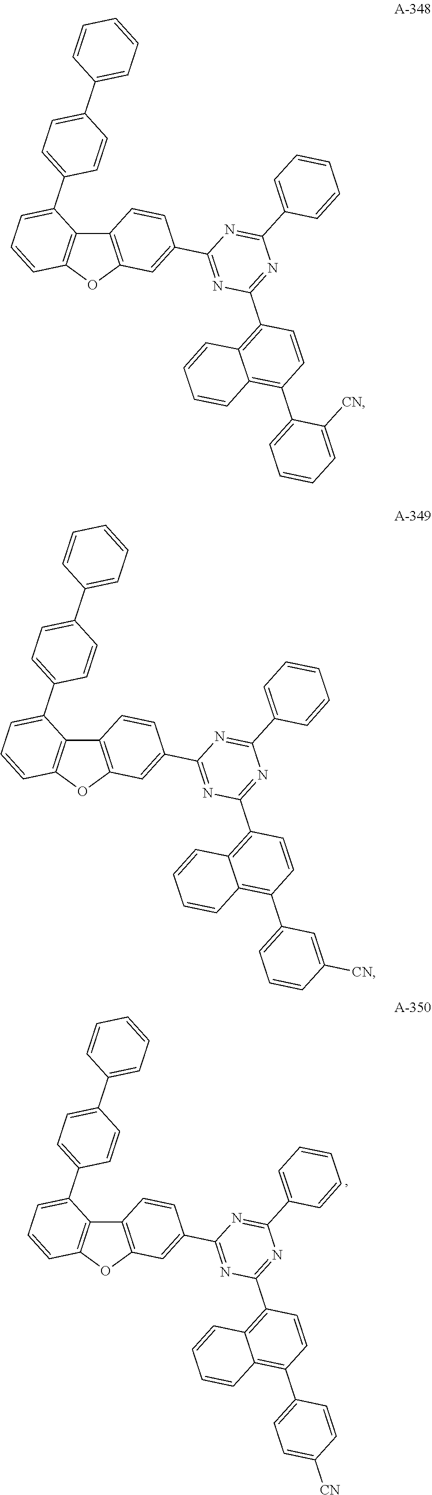 [SMILES: N#Cc1ccccc1-c1ccc(-c2nc(-c3ccccc3)nc(-c3ccc4c(c3)oc3cccc(-c5ccc(-c6ccccc6)cc5)c34)n2)c2ccccc12 ; N#Cc1cccc(-c2ccc(-c3nc(-c4ccccc4)nc(-c4ccc5c(c4)oc4cccc(-c6ccc(-c7ccccc7)cc6)c45)n3)c3ccccc23)c1 ; N#Cc1ccc(-c2ccc(-c3nc(-c4ccccc4)nc(-c4ccc5c(c4)oc4cccc(-c6ccc(-c7ccccc7)cc6)c45)n3)c3ccccc23)cc1] 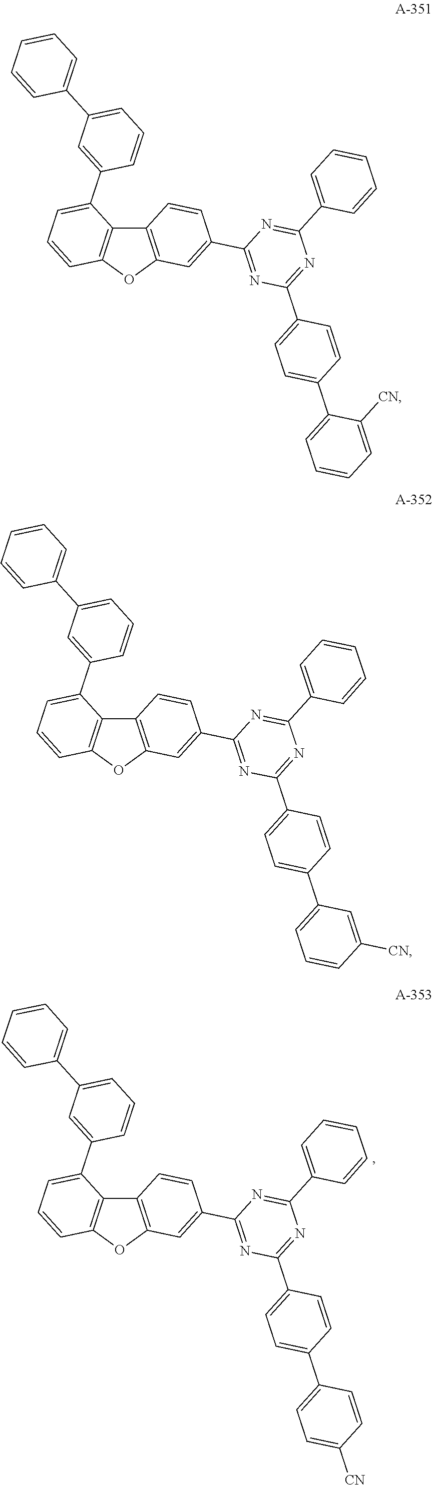 [SMILES: N#Cc1ccccc1-c1ccc(-c2nc(-c3ccccc3)nc(-c3ccc4c(c3)oc3cccc(-c5cccc(-c6ccccc6)c5)c34)n2)cc1 ; N#Cc1cccc(-c2ccc(-c3nc(-c4ccccc4)nc(-c4ccc5c(c4)oc4cccc(-c6cccc(-c7ccccc7)c6)c45)n3)cc2)c1 ; N#Cc1ccc(-c2ccc(-c3nc(-c4ccccc4)nc(-c4ccc5c(c4)oc4cccc(-c6cccc(-c7ccccc7)c6)c45)n3)cc2)cc1] 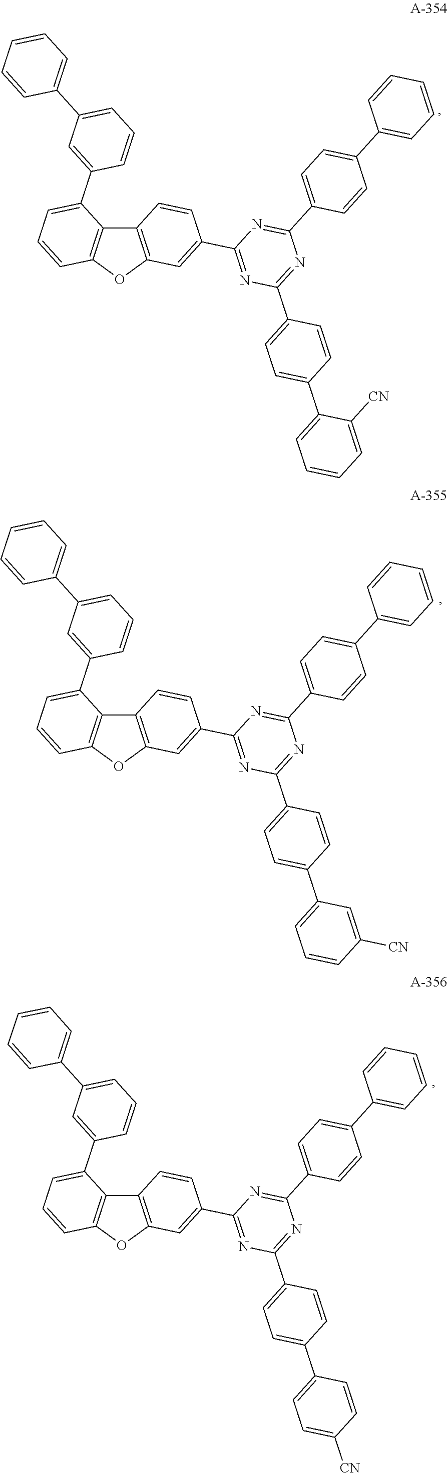 [SMILES: N#Cc1ccccc1-c1ccc(-c2nc(-c3ccc(-c4ccccc4)cc3)nc(-c3ccc4c(c3)oc3cccc(-c5cccc(-c6ccccc6)c5)c34)n2)cc1 ; N#Cc1cccc(-c2ccc(-c3nc(-c4ccc(-c5ccccc5)cc4)nc(-c4ccc5c(c4)oc4cccc(-c6cccc(-c7ccccc7)c6)c45)n3)cc2)c1 ; N#Cc1ccc(-c2ccc(-c3nc(-c4ccc(-c5ccccc5)cc4)nc(-c4ccc5c(c4)oc4cccc(-c6cccc(-c7ccccc7)c6)c45)n3)cc2)cc1] 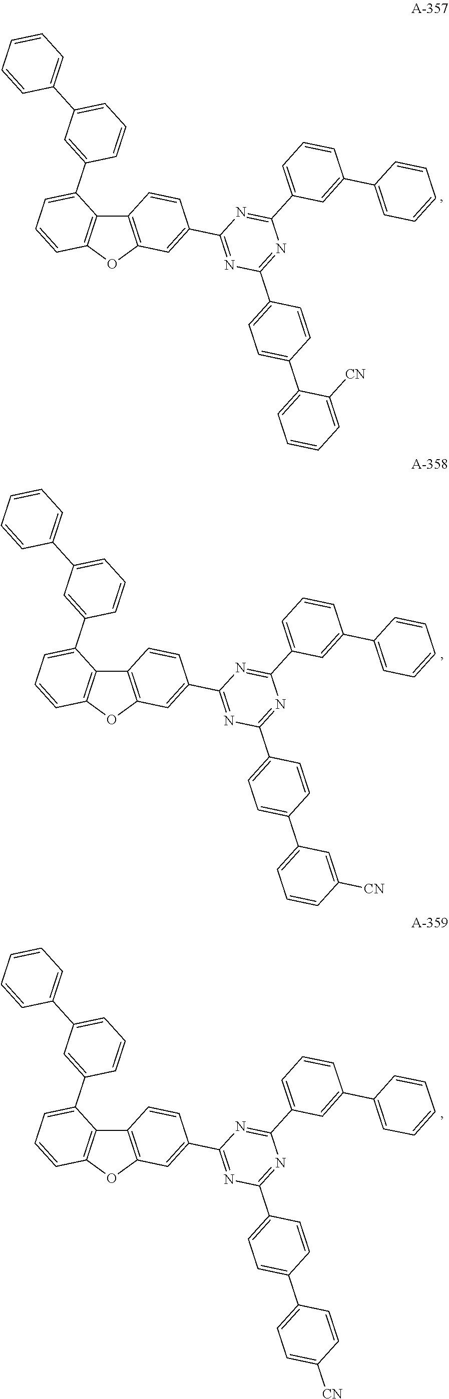 [SMILES: N#Cc1ccccc1-c1ccc(-c2nc(-c3cccc(-c4ccccc4)c3)nc(-c3ccc4c(c3)oc3cccc(-c5cccc(-c6ccccc6)c5)c34)n2)cc1 ; N#Cc1cccc(-c2ccc(-c3nc(-c4cccc(-c5ccccc5)c4)nc(-c4ccc5c(c4)oc4cccc(-c6cccc(-c7ccccc7)c6)c45)n3)cc2)c1 ; N#Cc1ccc(-c2ccc(-c3nc(-c4cccc(-c5ccccc5)c4)nc(-c4ccc5c(c4)oc4cccc(-c6cccc(-c7ccccc7)c6)c45)n3)cc2)cc1] 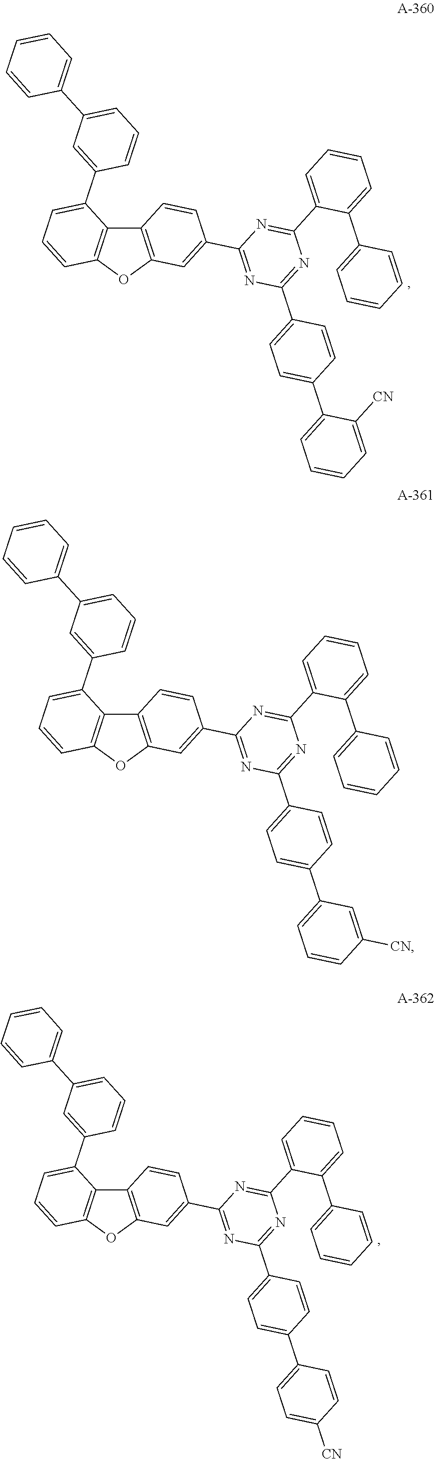 [SMILES: N#Cc1ccccc1-c1ccc(-c2nc(-c3ccc4c(c3)oc3cccc(-c5cccc(-c6ccccc6)c5)c34)nc(-c3ccccc3-c3ccccc3)n2)cc1 ; N#Cc1cccc(-c2ccc(-c3nc(-c4ccc5c(c4)oc4cccc(-c6cccc(-c7ccccc7)c6)c45)nc(-c4ccccc4-c4ccccc4)n3)cc2)c1 ; N#Cc1ccc(-c2ccc(-c3nc(-c4ccc5c(c4)oc4cccc(-c6cccc(-c7ccccc7)c6)c45)nc(-c4ccccc4-c4ccccc4)n3)cc2)cc1] 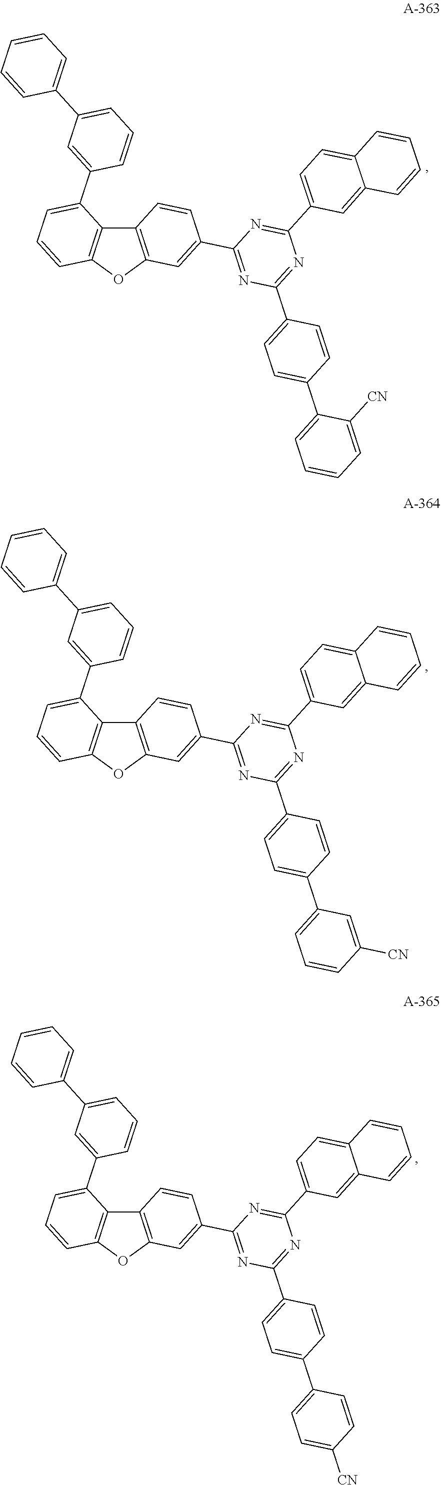 [SMILES: N#Cc1ccccc1-c1ccc(-c2nc(-c3ccc4ccccc4c3)nc(-c3ccc4c(c3)oc3cccc(-c5cccc(-c6ccccc6)c5)c34)n2)cc1 ; N#Cc1cccc(-c2ccc(-c3nc(-c4ccc5ccccc5c4)nc(-c4ccc5c(c4)oc4cccc(-c6cccc(-c7ccccc7)c6)c45)n3)cc2)c1 ; N#Cc1ccc(-c2ccc(-c3nc(-c4ccc5ccccc5c4)nc(-c4ccc5c(c4)oc4cccc(-c6cccc(-c7ccccc7)c6)c45)n3)cc2)cc1] 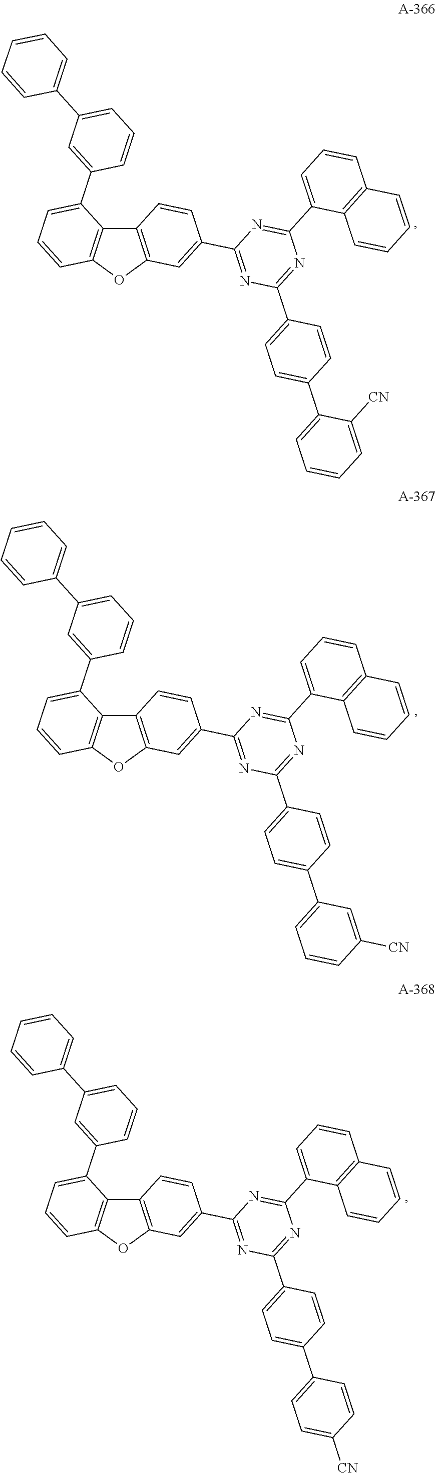 [SMILES: N#Cc1ccccc1-c1ccc(-c2nc(-c3ccc4c(c3)oc3cccc(-c5cccc(-c6ccccc6)c5)c34)nc(-c3cccc4ccccc34)n2)cc1 ; N#Cc1cccc(-c2ccc(-c3nc(-c4ccc5c(c4)oc4cccc(-c6cccc(-c7ccccc7)c6)c45)nc(-c4cccc5ccccc45)n3)cc2)c1 ; N#Cc1ccc(-c2ccc(-c3nc(-c4ccc5c(c4)oc4cccc(-c6cccc(-c7ccccc7)c6)c45)nc(-c4cccc5ccccc45)n3)cc2)cc1] 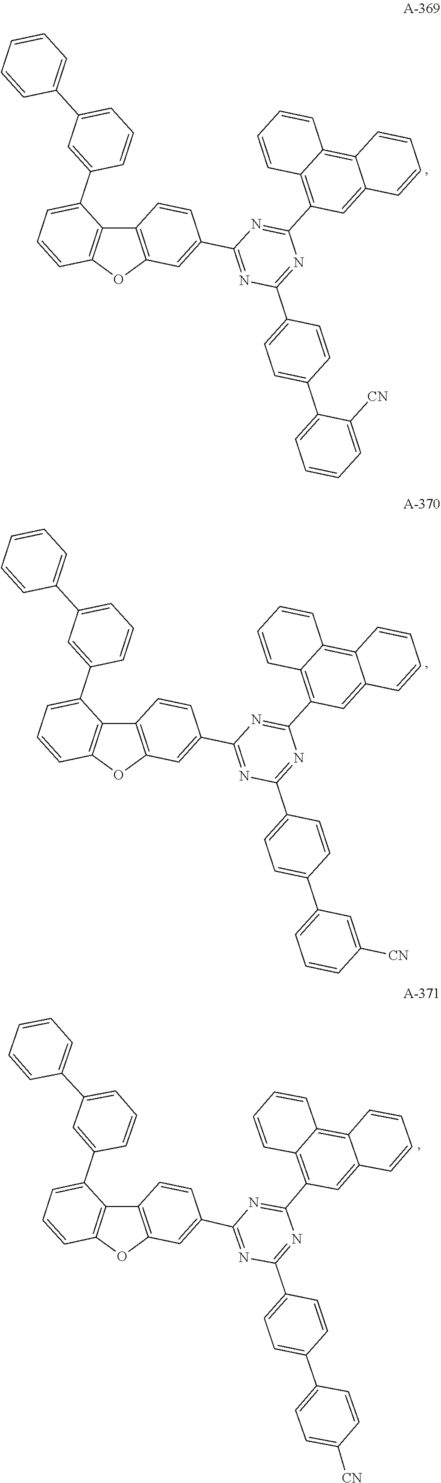 [SMILES: N#Cc1ccccc1-c1ccc(-c2nc(-c3ccc4c(c3)oc3cccc(-c5cccc(-c6ccccc6)c5)c34)nc(-c3cc4ccccc4c4ccccc34)n2)cc1 ; N#Cc1cccc(-c2ccc(-c3nc(-c4ccc5c(c4)oc4cccc(-c6cccc(-c7ccccc7)c6)c45)nc(-c4cc5ccccc5c5ccccc45)n3)cc2)c1 ; N#Cc1ccc(-c2ccc(-c3nc(-c4ccc5c(c4)oc4cccc(-c6cccc(-c7ccccc7)c6)c45)nc(-c4cc5ccccc5c5ccccc45)n3)cc2)cc1] 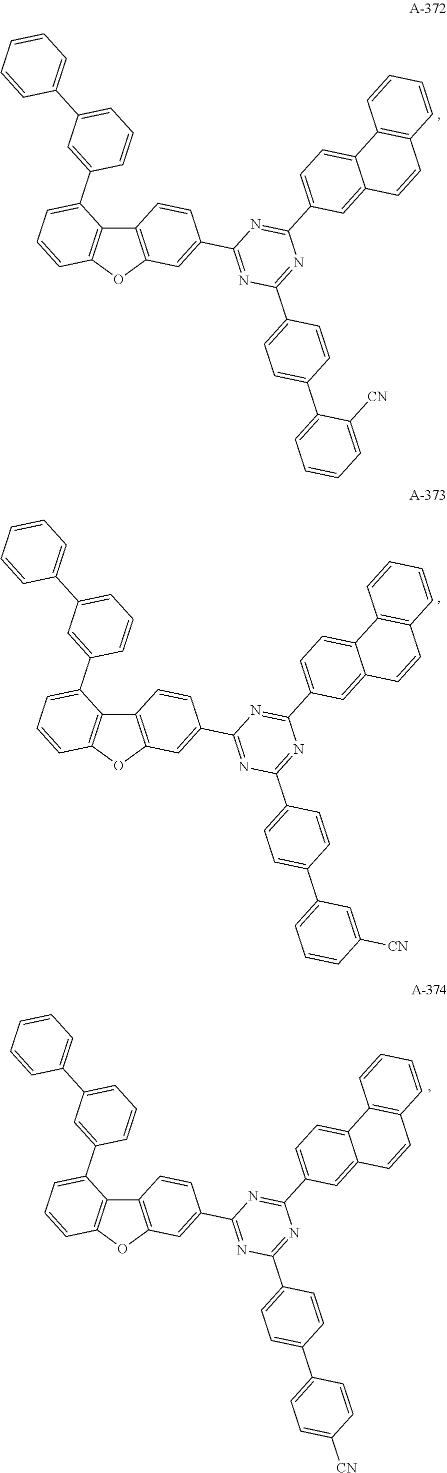 [SMILES: N#Cc1ccccc1-c1ccc(-c2nc(-c3ccc4c(ccc5ccccc54)c3)nc(-c3ccc4c(c3)oc3cccc(-c5cccc(-c6ccccc6)c5)c34)n2)cc1 ; N#Cc1cccc(-c2ccc(-c3nc(-c4ccc5c(ccc6ccccc65)c4)nc(-c4ccc5c(c4)oc4cccc(-c6cccc(-c7ccccc7)c6)c45)n3)cc2)c1 ; N#Cc1ccc(-c2ccc(-c3nc(-c4ccc5c(ccc6ccccc65)c4)nc(-c4ccc5c(c4)oc4cccc(-c6cccc(-c7ccccc7)c6)c45)n3)cc2)cc1] 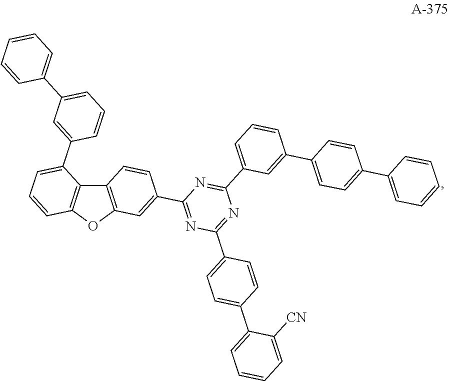 [SMILES: N#Cc1ccccc1-c1ccc(-c2nc(-c3cccc(-c4ccc(-c5ccccc5)cc4)c3)nc(-c3ccc4c(c3)oc3cccc(-c5cccc(-c6ccccc6)c5)c34)n2)cc1] 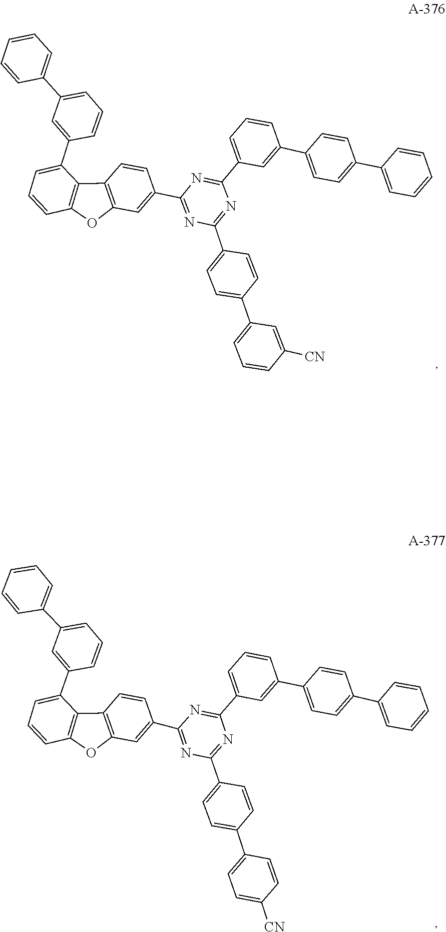 [SMILES: N#Cc1cccc(-c2ccc(-c3nc(-c4cccc(-c5ccc(-c6ccccc6)cc5)c4)nc(-c4ccc5c(c4)oc4cccc(-c6cccc(-c7ccccc7)c6)c45)n3)cc2)c1 ; N#Cc1ccc(-c2ccc(-c3nc(-c4cccc(-c5ccc(-c6ccccc6)cc5)c4)nc(-c4ccc5c(c4)oc4cccc(-c6cccc(-c7ccccc7)c6)c45)n3)cc2)cc1] 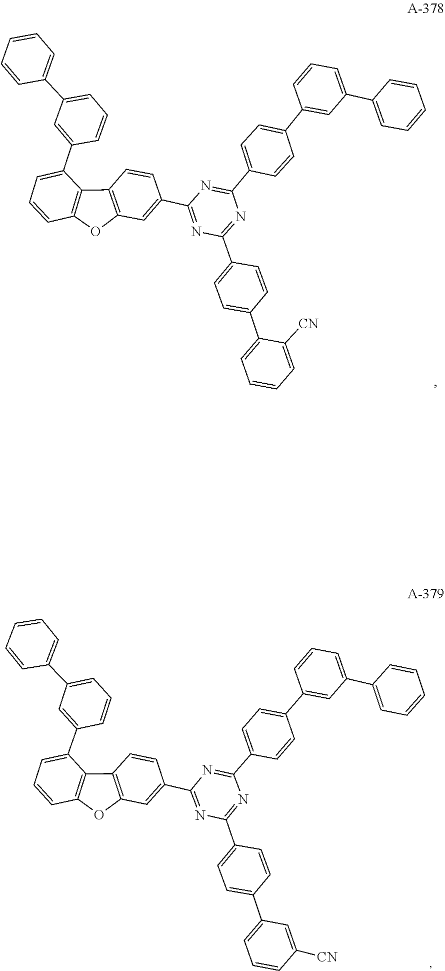 [SMILES: N#Cc1ccccc1-c1ccc(-c2nc(-c3ccc(-c4cccc(-c5ccccc5)c4)cc3)nc(-c3ccc4c(c3)oc3cccc(-c5cccc(-c6ccccc6)c5)c34)n2)cc1 ; N#Cc1cccc(-c2ccc(-c3nc(-c4ccc(-c5cccc(-c6ccccc6)c5)cc4)nc(-c4ccc5c(c4)oc4cccc(-c6cccc(-c7ccccc7)c6)c45)n3)cc2)c1] 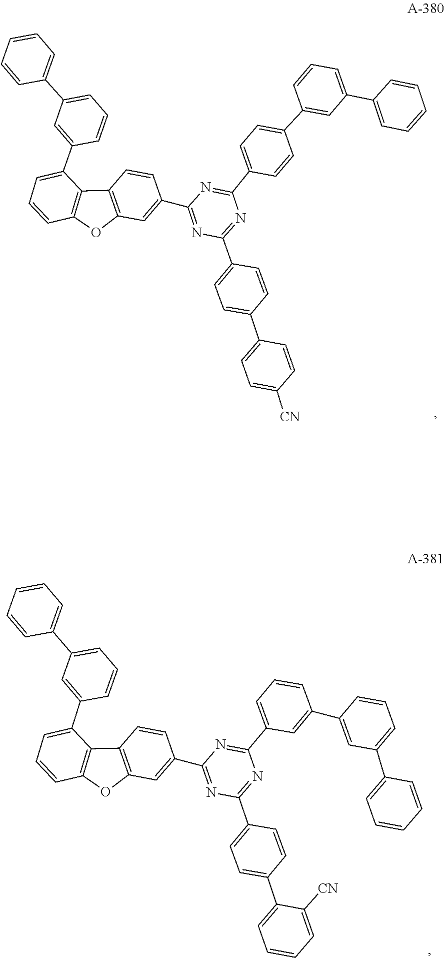 [SMILES: N#Cc1ccc(-c2ccc(-c3nc(-c4ccc(-c5cccc(-c6ccccc6)c5)cc4)nc(-c4ccc5c(c4)oc4cccc(-c6cccc(-c7ccccc7)c6)c45)n3)cc2)cc1 ; N#Cc1ccccc1-c1ccc(-c2nc(-c3cccc(-c4cccc(-c5ccccc5)c4)c3)nc(-c3ccc4c(c3)oc3cccc(-c5cccc(-c6ccccc6)c5)c34)n2)cc1] 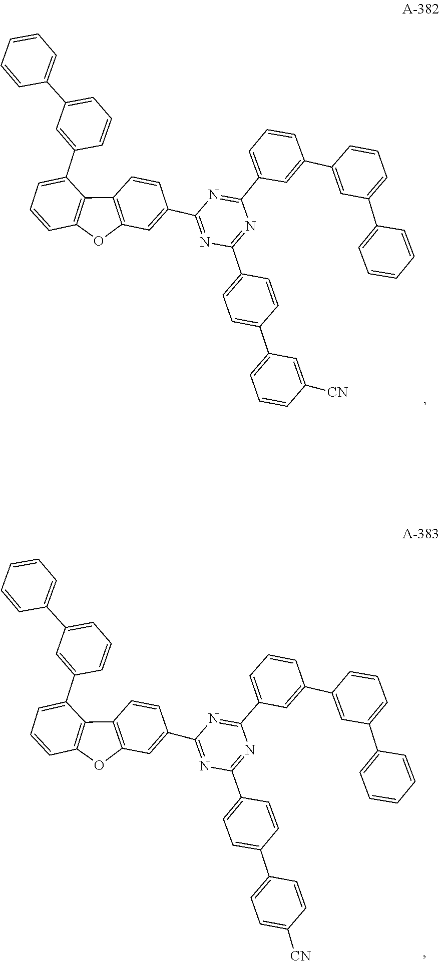 [SMILES: N#Cc1cccc(-c2ccc(-c3nc(-c4cccc(-c5cccc(-c6ccccc6)c5)c4)nc(-c4ccc5c(c4)oc4cccc(-c6cccc(-c7ccccc7)c6)c45)n3)cc2)c1 ; N#Cc1ccc(-c2ccc(-c3nc(-c4cccc(-c5cccc(-c6ccccc6)c5)c4)nc(-c4ccc5c(c4)oc4cccc(-c6cccc(-c7ccccc7)c6)c45)n3)cc2)cc1] 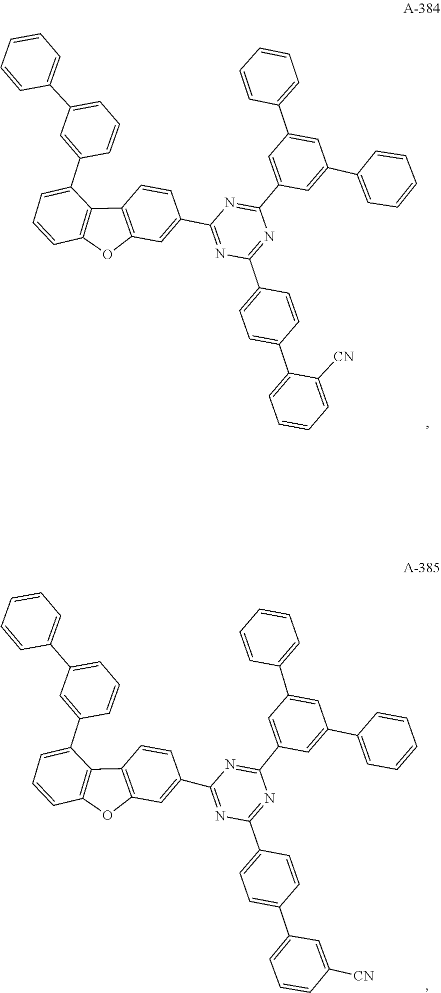 [SMILES: N#Cc1ccccc1-c1ccc(-c2nc(-c3cc(-c4ccccc4)cc(-c4ccccc4)c3)nc(-c3ccc4c(c3)oc3cccc(-c5cccc(-c6ccccc6)c5)c34)n2)cc1 ; N#Cc1cccc(-c2ccc(-c3nc(-c4cc(-c5ccccc5)cc(-c5ccccc5)c4)nc(-c4ccc5c(c4)oc4cccc(-c6cccc(-c7ccccc7)c6)c45)n3)cc2)c1] 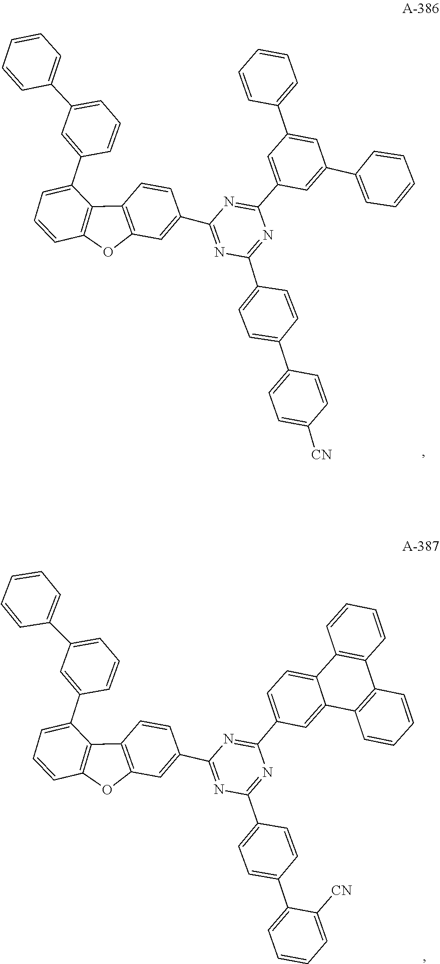 [SMILES: N#Cc1ccc(-c2ccc(-c3nc(-c4cc(-c5ccccc5)cc(-c5ccccc5)c4)nc(-c4ccc5c(c4)oc4cccc(-c6cccc(-c7ccccc7)c6)c45)n3)cc2)cc1 ; N#Cc1ccccc1-c1ccc(-c2nc(-c3ccc4c(c3)oc3cccc(-c5cccc(-c6ccccc6)c5)c34)nc(-c3ccc4c5ccccc5c5ccccc5c4c3)n2)cc1] 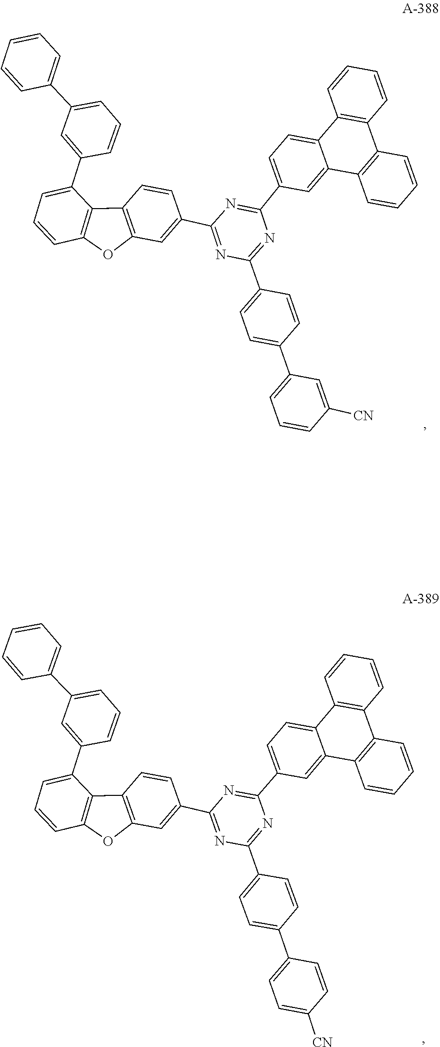 [SMILES: N#Cc1cccc(-c2ccc(-c3nc(-c4ccc5c(c4)oc4cccc(-c6cccc(-c7ccccc7)c6)c45)nc(-c4ccc5c6ccccc6c6ccccc6c5c4)n3)cc2)c1 ; N#Cc1ccc(-c2ccc(-c3nc(-c4ccc5c(c4)oc4cccc(-c6cccc(-c7ccccc7)c6)c45)nc(-c4ccc5c6ccccc6c6ccccc6c5c4)n3)cc2)cc1] 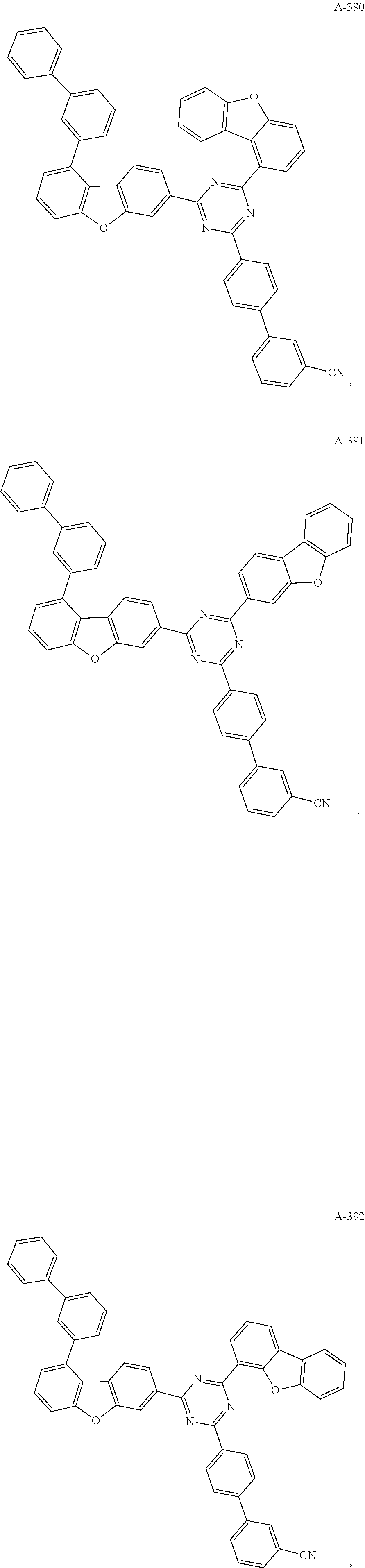 [SMILES: N#Cc1cccc(-c2ccc(-c3nc(-c4ccc5c(c4)oc4cccc(-c6cccc(-c7ccccc7)c6)c45)nc(-c4cccc5oc6ccccc6c45)n3)cc2)c1 ; N#Cc1cccc(-c2ccc(-c3nc(-c4ccc5c(c4)oc4ccccc45)nc(-c4ccc5c(c4)oc4cccc(-c6cccc(-c7ccccc7)c6)c45)n3)cc2)c1 ; N#Cc1cccc(-c2ccc(-c3nc(-c4ccc5c(c4)oc4cccc(-c6cccc(-c7ccccc7)c6)c45)nc(-c4cccc5c4oc4ccccc45)n3)cc2)c1] 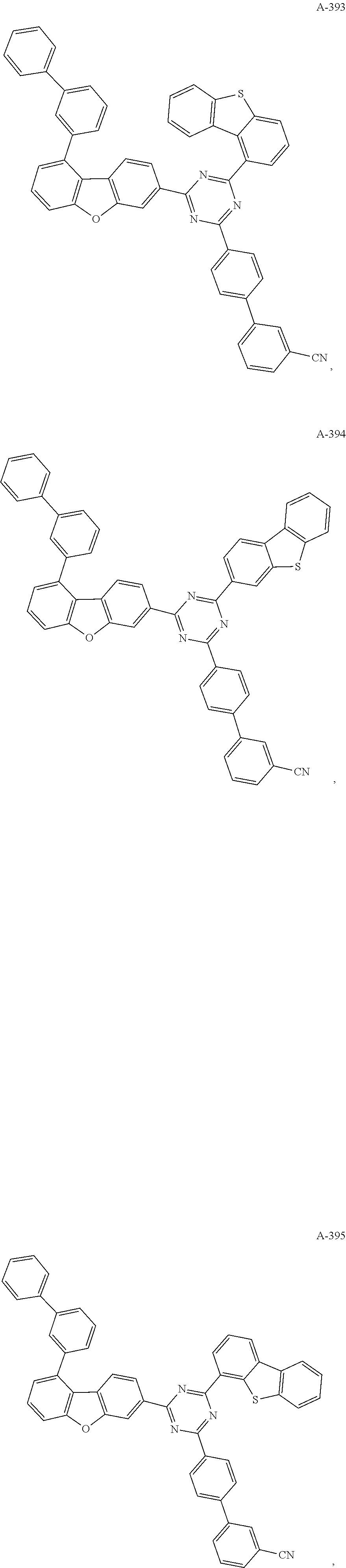 [SMILES: N#Cc1cccc(-c2ccc(-c3nc(-c4ccc5c(c4)oc4cccc(-c6cccc(-c7ccccc7)c6)c45)nc(-c4cccc5sc6ccccc6c45)n3)cc2)c1 ; N#Cc1cccc(-c2ccc(-c3nc(-c4ccc5c(c4)oc4cccc(-c6cccc(-c7ccccc7)c6)c45)nc(-c4ccc5c(c4)sc4ccccc45)n3)cc2)c1 ; N#Cc1cccc(-c2ccc(-c3nc(-c4ccc5c(c4)oc4cccc(-c6cccc(-c7ccccc7)c6)c45)nc(-c4cccc5c4sc4ccccc45)n3)cc2)c1] 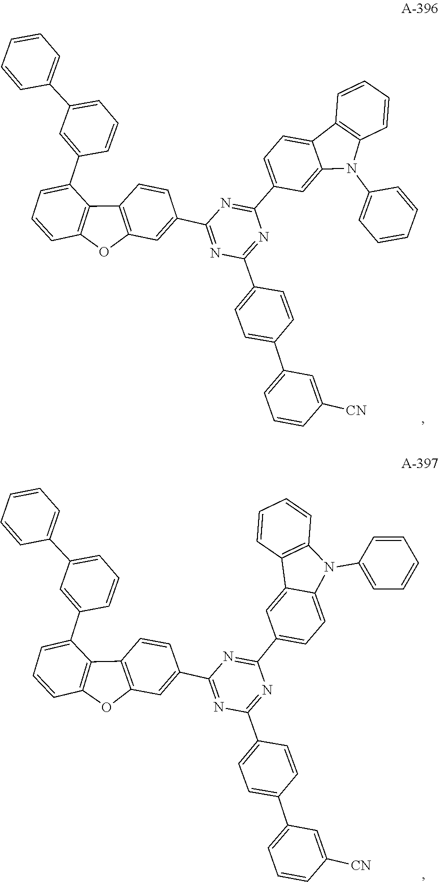 [SMILES: N#Cc1cccc(-c2ccc(-c3nc(-c4ccc5c(c4)oc4cccc(-c6cccc(-c7ccccc7)c6)c45)nc(-c4ccc5c6ccccc6n(-c6ccccc6)c5c4)n3)cc2)c1 ; N#Cc1cccc(-c2ccc(-c3nc(-c4ccc5c(c4)oc4cccc(-c6cccc(-c7ccccc7)c6)c45)nc(-c4ccc5c(c4)c4ccccc4n5-c4ccccc4)n3)cc2)c1] 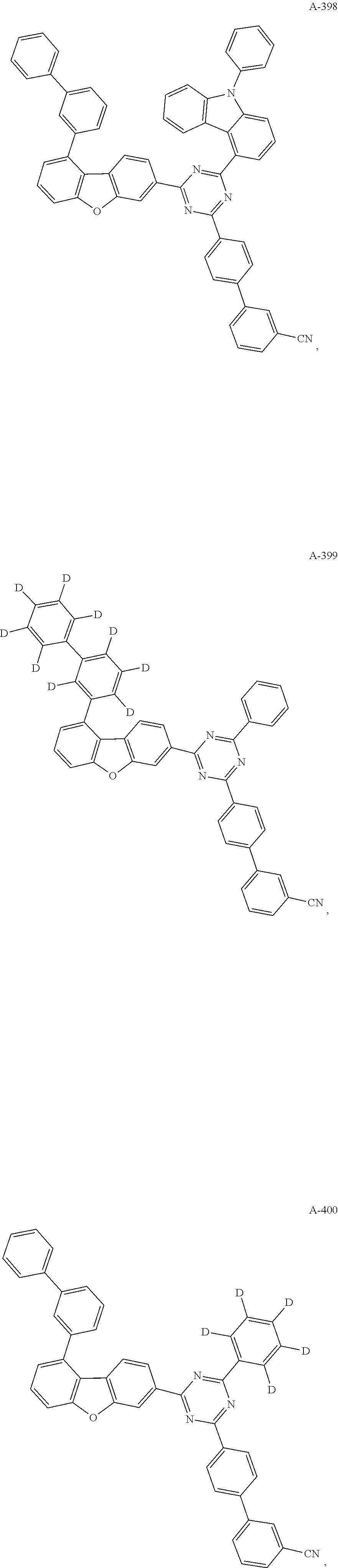 [SMILES: N#Cc1cccc(-c2ccc(-c3nc(-c4ccc5c(c4)oc4cccc(-c6cccc(-c7ccccc7)c6)c45)nc(-c4cccc5c4c4ccccc4n5-c4ccccc4)n3)cc2)c1 ; [2H]c1c([2H])c([2H])c(-c2c([2H])c([2H])c([2H])c(-c3cccc4oc5cc(-c6nc(-c7ccccc7)nc(-c7ccc(-c8cccc(C#N)c8)cc7)n6)ccc5c34)c2[2H])c([2H])c1[2H] ; [2H]c1c([2H])c([2H])c(-c2nc(-c3ccc(-c4cccc(C#N)c4)cc3)nc(-c3ccc4c(c3)oc3cccc(-c5cccc(-c6ccccc6)c5)c34)n2)c([2H])c1[2H]] 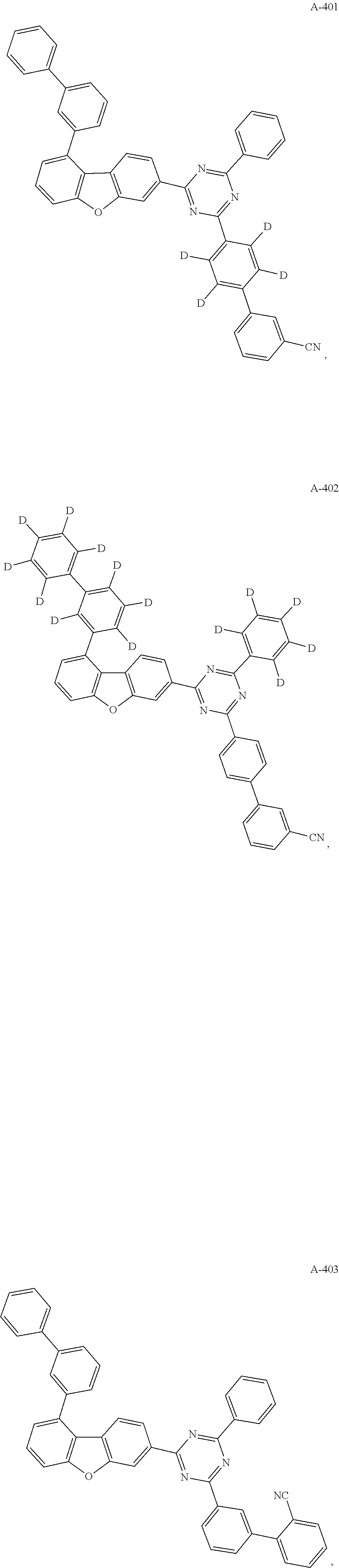 [SMILES: [2H]c1c([2H])c(-c2nc(-c3ccccc3)nc(-c3ccc4c(c3)oc3cccc(-c5cccc(-c6ccccc6)c5)c34)n2)c([2H])c([2H])c1-c1cccc(C#N)c1 ; [2H]c1c([2H])c([2H])c(-c2nc(-c3ccc(-c4cccc(C#N)c4)cc3)nc(-c3ccc4c(c3)oc3cccc(-c5c([2H])c([2H])c([2H])c(-c6c([2H])c([2H])c([2H])c([2H])c6[2H])c5[2H])c34)n2)c([2H])c1[2H] ; N#Cc1ccccc1-c1cccc(-c2nc(-c3ccccc3)nc(-c3ccc4c(c3)oc3cccc(-c5cccc(-c6ccccc6)c5)c34)n2)c1] 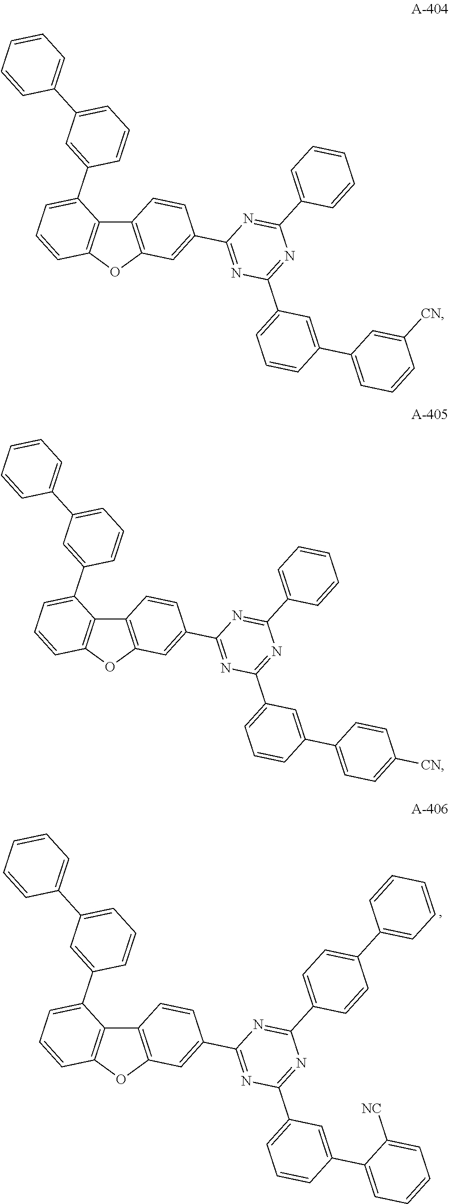 [SMILES: N#Cc1cccc(-c2cccc(-c3nc(-c4ccccc4)nc(-c4ccc5c(c4)oc4cccc(-c6cccc(-c7ccccc7)c6)c45)n3)c2)c1 ; N#Cc1ccc(-c2cccc(-c3nc(-c4ccccc4)nc(-c4ccc5c(c4)oc4cccc(-c6cccc(-c7ccccc7)c6)c45)n3)c2)cc1 ; N#Cc1ccccc1-c1cccc(-c2nc(-c3ccc(-c4ccccc4)cc3)nc(-c3ccc4c(c3)oc3cccc(-c5cccc(-c6ccccc6)c5)c34)n2)c1] 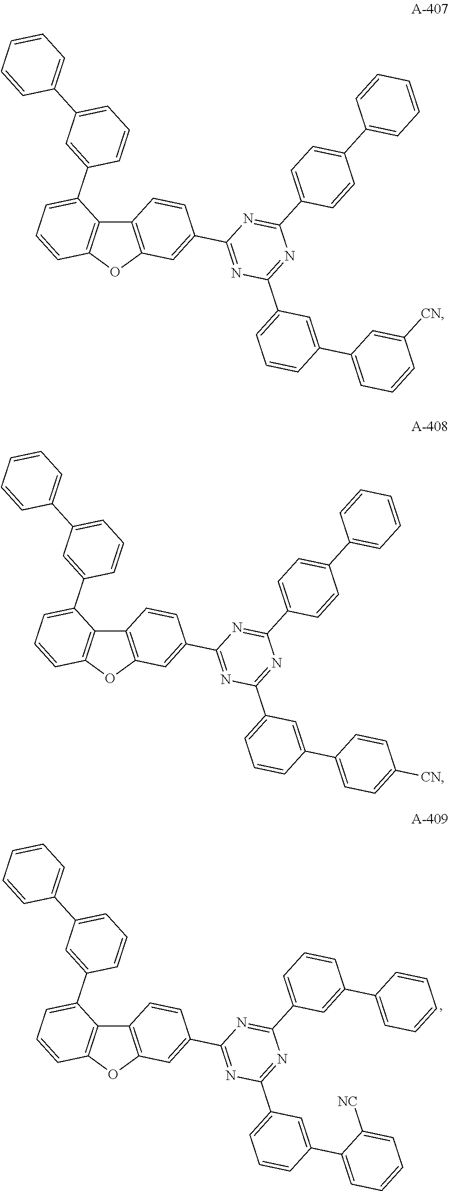 [SMILES: N#Cc1cccc(-c2cccc(-c3nc(-c4ccc(-c5ccccc5)cc4)nc(-c4ccc5c(c4)oc4cccc(-c6cccc(-c7ccccc7)c6)c45)n3)c2)c1 ; N#Cc1ccc(-c2cccc(-c3nc(-c4ccc(-c5ccccc5)cc4)nc(-c4ccc5c(c4)oc4cccc(-c6cccc(-c7ccccc7)c6)c45)n3)c2)cc1 ; N#Cc1ccccc1-c1cccc(-c2nc(-c3cccc(-c4ccccc4)c3)nc(-c3ccc4c(c3)oc3cccc(-c5cccc(-c6ccccc6)c5)c34)n2)c1] 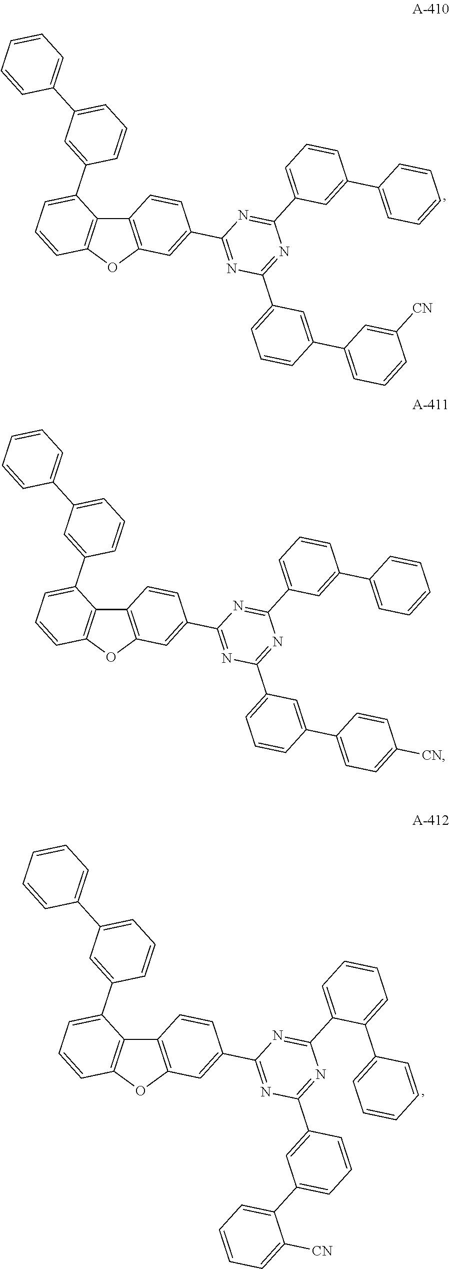 [SMILES: N#Cc1cccc(-c2cccc(-c3nc(-c4cccc(-c5ccccc5)c4)nc(-c4ccc5c(c4)oc4cccc(-c6cccc(-c7ccccc7)c6)c45)n3)c2)c1 ; N#Cc1ccc(-c2cccc(-c3nc(-c4cccc(-c5ccccc5)c4)nc(-c4ccc5c(c4)oc4cccc(-c6cccc(-c7ccccc7)c6)c45)n3)c2)cc1 ; N#Cc1ccccc1-c1cccc(-c2nc(-c3ccc4c(c3)oc3cccc(-c5cccc(-c6ccccc6)c5)c34)nc(-c3ccccc3-c3ccccc3)n2)c1] 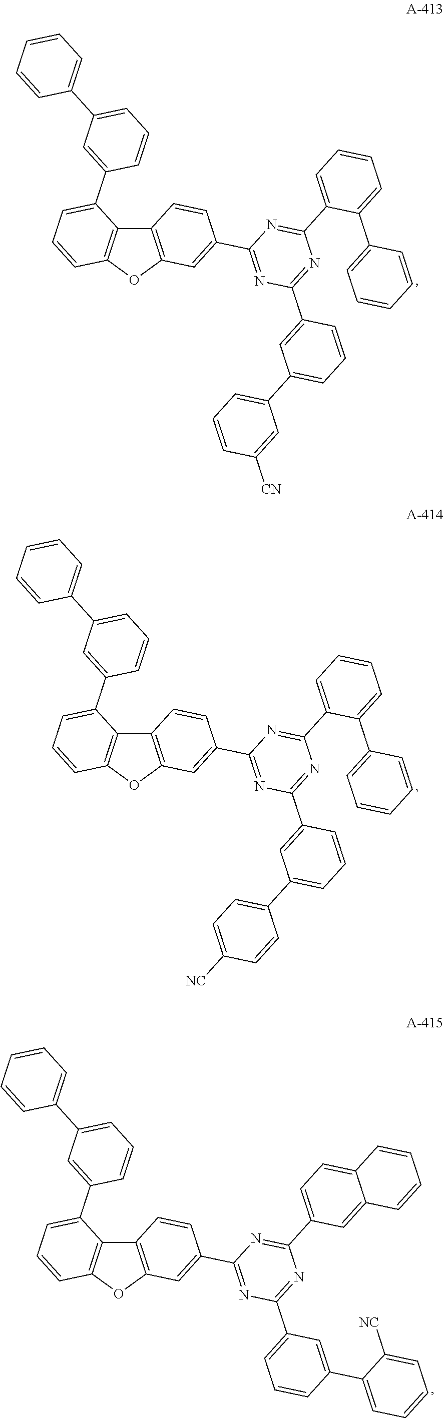 [SMILES: N#Cc1cccc(-c2cccc(-c3nc(-c4ccc5c(c4)oc4cccc(-c6cccc(-c7ccccc7)c6)c45)nc(-c4ccccc4-c4ccccc4)n3)c2)c1 ; N#Cc1ccc(-c2cccc(-c3nc(-c4ccc5c(c4)oc4cccc(-c6cccc(-c7ccccc7)c6)c45)nc(-c4ccccc4-c4ccccc4)n3)c2)cc1 ; N#Cc1ccccc1-c1cccc(-c2nc(-c3ccc4ccccc4c3)nc(-c3ccc4c(c3)oc3cccc(-c5cccc(-c6ccccc6)c5)c34)n2)c1] 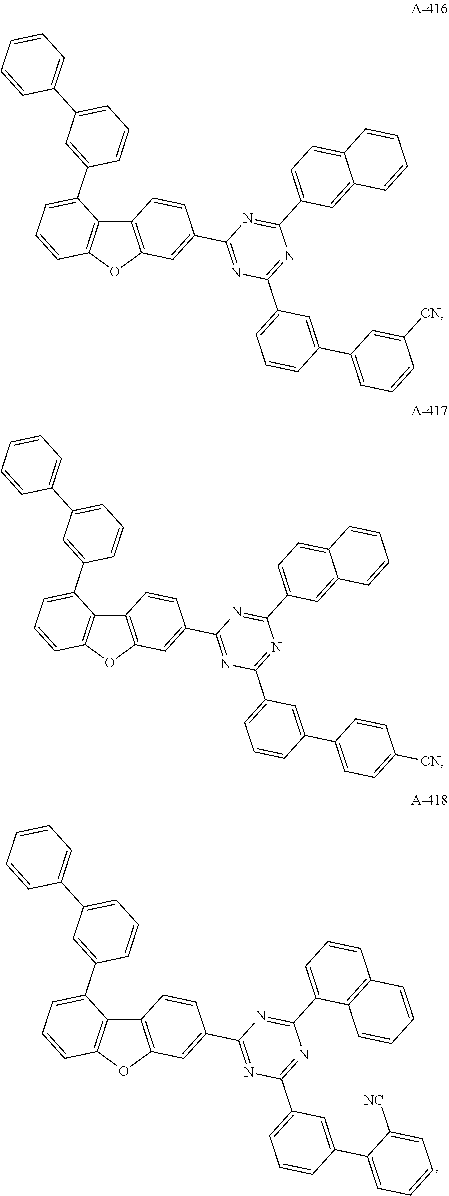 [SMILES: N#Cc1cccc(-c2cccc(-c3nc(-c4ccc5ccccc5c4)nc(-c4ccc5c(c4)oc4cccc(-c6cccc(-c7ccccc7)c6)c45)n3)c2)c1 ; N#Cc1ccc(-c2cccc(-c3nc(-c4ccc5ccccc5c4)nc(-c4ccc5c(c4)oc4cccc(-c6cccc(-c7ccccc7)c6)c45)n3)c2)cc1 ; N#Cc1ccccc1-c1cccc(-c2nc(-c3ccc4c(c3)oc3cccc(-c5cccc(-c6ccccc6)c5)c34)nc(-c3cccc4ccccc34)n2)c1] 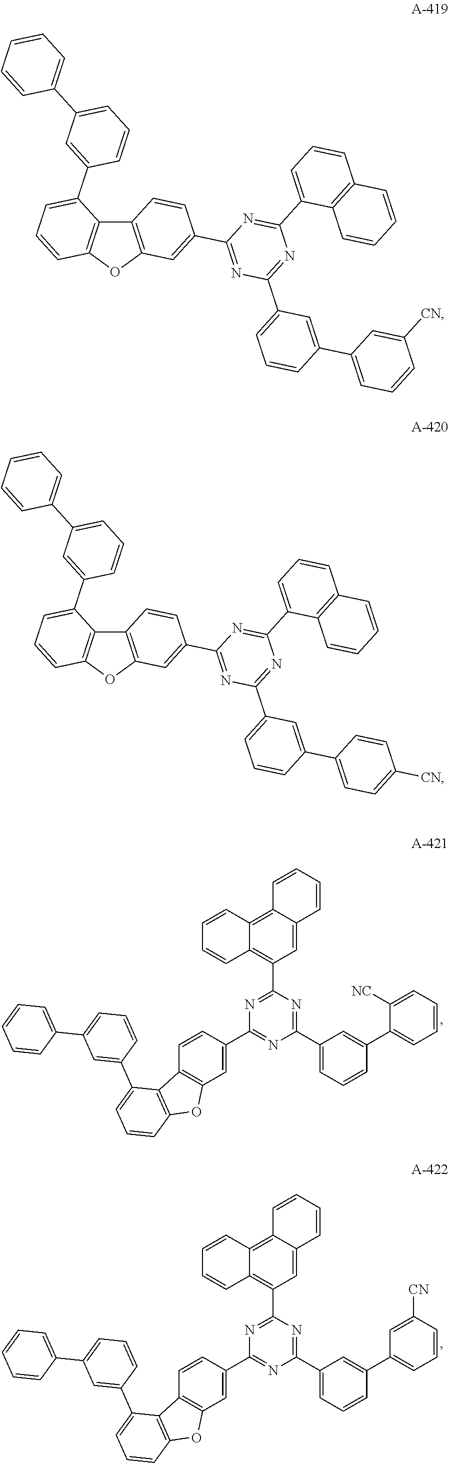 [SMILES: N#Cc1cccc(-c2cccc(-c3nc(-c4ccc5c(c4)oc4cccc(-c6cccc(-c7ccccc7)c6)c45)nc(-c4cccc5ccccc45)n3)c2)c1 ; N#Cc1ccc(-c2cccc(-c3nc(-c4ccc5c(c4)oc4cccc(-c6cccc(-c7ccccc7)c6)c45)nc(-c4cccc5ccccc45)n3)c2)cc1 ; N#Cc1ccccc1-c1cccc(-c2nc(-c3ccc4c(c3)oc3cccc(-c5cccc(-c6ccccc6)c5)c34)nc(-c3cc4ccccc4c4ccccc34)n2)c1 ; N#Cc1cccc(-c2cccc(-c3nc(-c4ccc5c(c4)oc4cccc(-c6cccc(-c7ccccc7)c6)c45)nc(-c4cc5ccccc5c5ccccc45)n3)c2)c1] 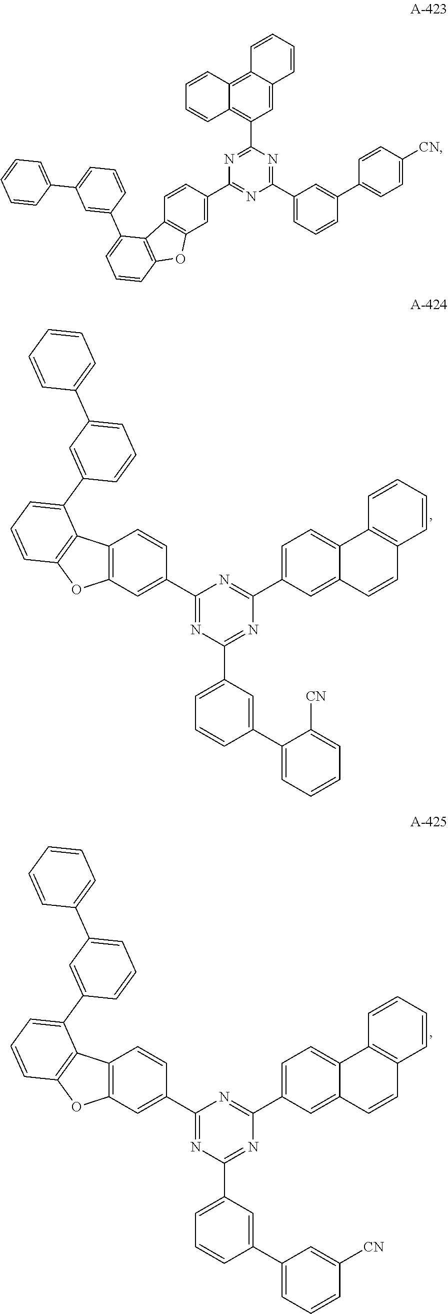 [SMILES: N#Cc1ccc(-c2cccc(-c3nc(-c4ccc5c(c4)oc4cccc(-c6cccc(-c7ccccc7)c6)c45)nc(-c4cc5ccccc5c5ccccc45)n3)c2)cc1 ; N#Cc1ccccc1-c1cccc(-c2nc(-c3ccc4c(ccc5ccccc54)c3)nc(-c3ccc4c(c3)oc3cccc(-c5cccc(-c6ccccc6)c5)c34)n2)c1 ; N#Cc1cccc(-c2cccc(-c3nc(-c4ccc5c(ccc6ccccc65)c4)nc(-c4ccc5c(c4)oc4cccc(-c6cccc(-c7ccccc7)c6)c45)n3)c2)c1] 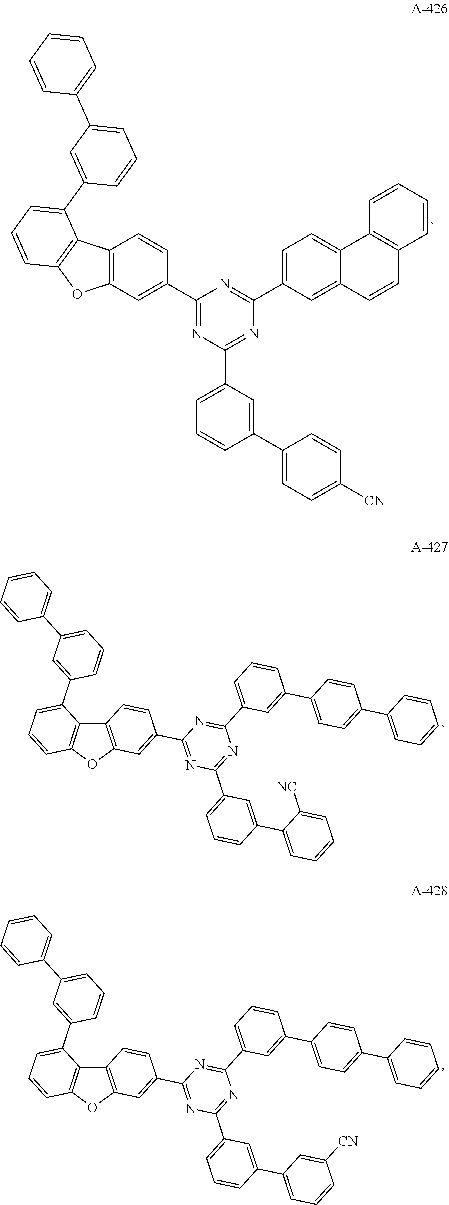 [SMILES: N#Cc1ccc(-c2cccc(-c3nc(-c4ccc5c(ccc6ccccc65)c4)nc(-c4ccc5c(c4)oc4cccc(-c6cccc(-c7ccccc7)c6)c45)n3)c2)cc1 ; N#Cc1ccccc1-c1cccc(-c2nc(-c3cccc(-c4ccc(-c5ccccc5)cc4)c3)nc(-c3ccc4c(c3)oc3cccc(-c5cccc(-c6ccccc6)c5)c34)n2)c1 ; N#Cc1cccc(-c2cccc(-c3nc(-c4cccc(-c5ccc(-c6ccccc6)cc5)c4)nc(-c4ccc5c(c4)oc4cccc(-c6cccc(-c7ccccc7)c6)c45)n3)c2)c1] 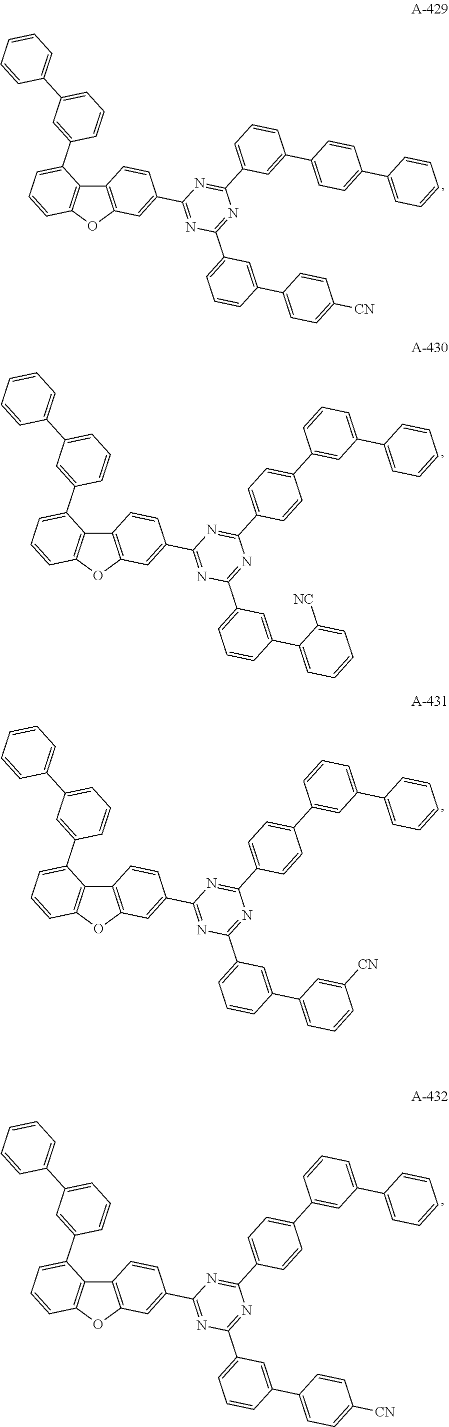 [SMILES: N#Cc1ccc(-c2cccc(-c3nc(-c4cccc(-c5ccc(-c6ccccc6)cc5)c4)nc(-c4ccc5c(c4)oc4cccc(-c6cccc(-c7ccccc7)c6)c45)n3)c2)cc1 ; N#Cc1ccccc1-c1cccc(-c2nc(-c3ccc(-c4cccc(-c5ccccc5)c4)cc3)nc(-c3ccc4c(c3)oc3cccc(-c5cccc(-c6ccccc6)c5)c34)n2)c1 ; N#Cc1cccc(-c2cccc(-c3nc(-c4ccc(-c5cccc(-c6ccccc6)c5)cc4)nc(-c4ccc5c(c4)oc4cccc(-c6cccc(-c7ccccc7)c6)c45)n3)c2)c1 ; N#Cc1ccc(-c2cccc(-c3nc(-c4ccc(-c5cccc(-c6ccccc6)c5)cc4)nc(-c4ccc5c(c4)oc4cccc(-c6cccc(-c7ccccc7)c6)c45)n3)c2)cc1] 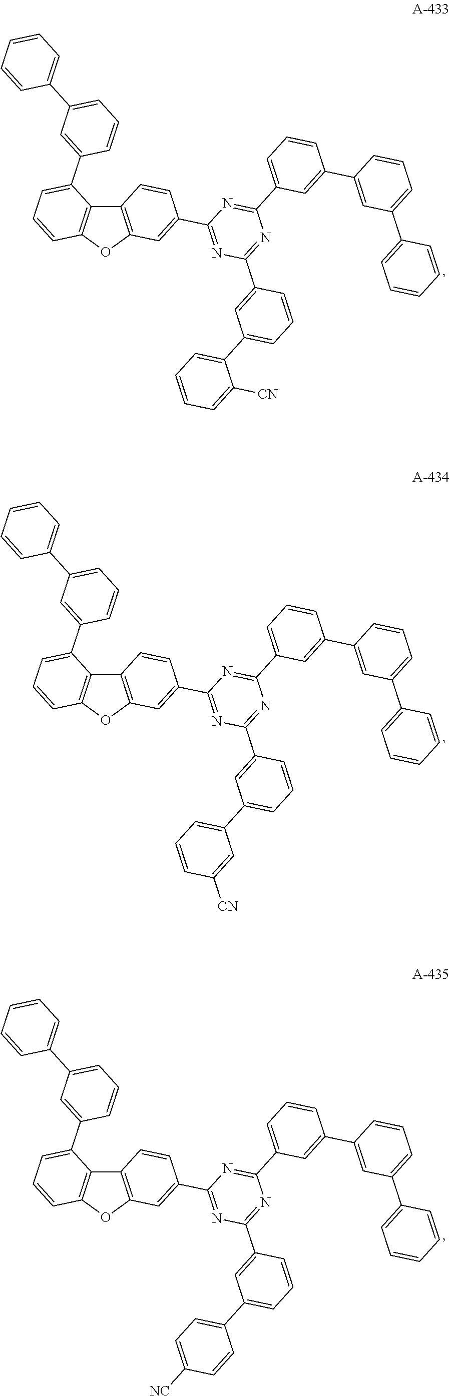 [SMILES: N#Cc1ccccc1-c1cccc(-c2nc(-c3cccc(-c4cccc(-c5ccccc5)c4)c3)nc(-c3ccc4c(c3)oc3cccc(-c5cccc(-c6ccccc6)c5)c34)n2)c1 ; N#Cc1cccc(-c2cccc(-c3nc(-c4cccc(-c5cccc(-c6ccccc6)c5)c4)nc(-c4ccc5c(c4)oc4cccc(-c6cccc(-c7ccccc7)c6)c45)n3)c2)c1 ; N#Cc1ccc(-c2cccc(-c3nc(-c4cccc(-c5cccc(-c6ccccc6)c5)c4)nc(-c4ccc5c(c4)oc4cccc(-c6cccc(-c7ccccc7)c6)c45)n3)c2)cc1] 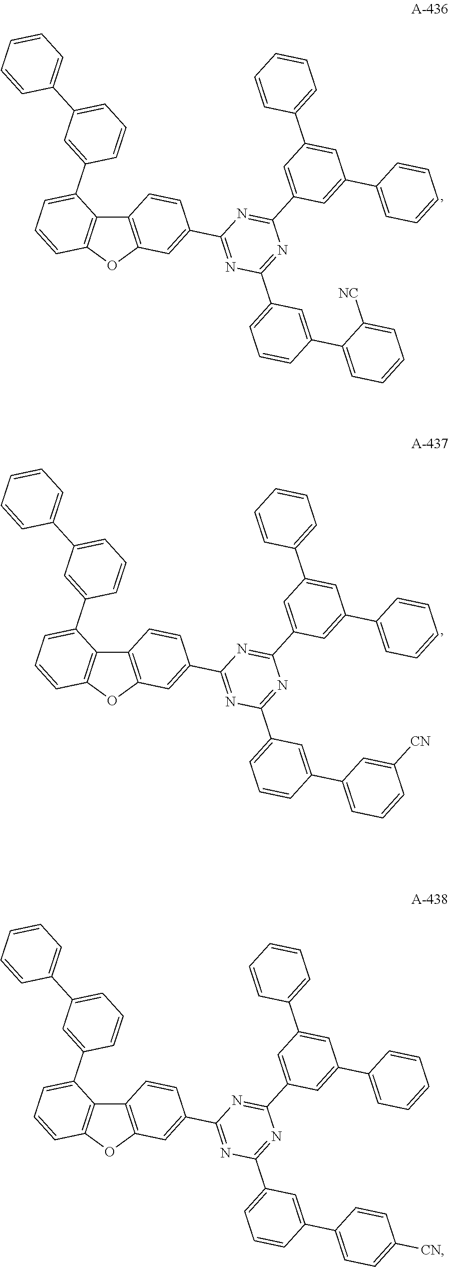 [SMILES: N#Cc1ccccc1-c1cccc(-c2nc(-c3cc(-c4ccccc4)cc(-c4ccccc4)c3)nc(-c3ccc4c(c3)oc3cccc(-c5cccc(-c6ccccc6)c5)c34)n2)c1 ; N#Cc1cccc(-c2cccc(-c3nc(-c4cc(-c5ccccc5)cc(-c5ccccc5)c4)nc(-c4ccc5c(c4)oc4cccc(-c6cccc(-c7ccccc7)c6)c45)n3)c2)c1 ; N#Cc1ccc(-c2cccc(-c3nc(-c4cc(-c5ccccc5)cc(-c5ccccc5)c4)nc(-c4ccc5c(c4)oc4cccc(-c6cccc(-c7ccccc7)c6)c45)n3)c2)cc1] 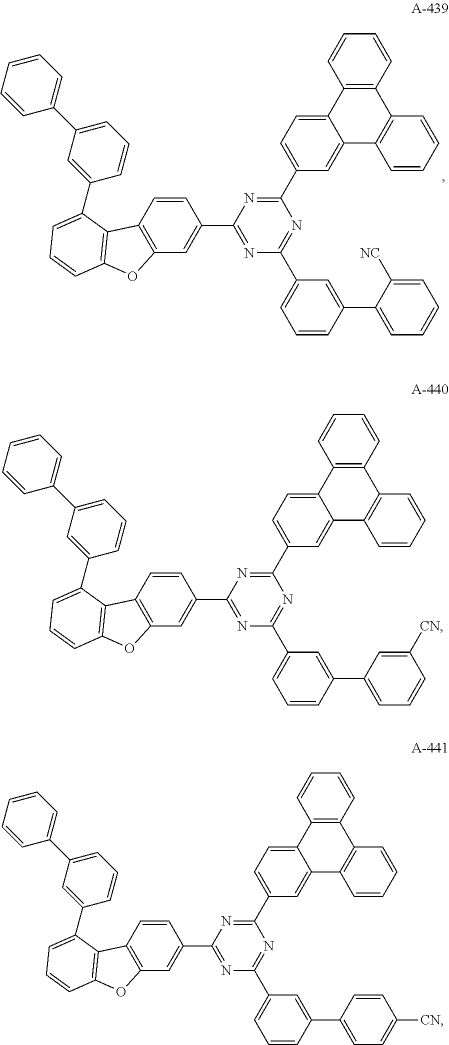 [SMILES: N#Cc1ccccc1-c1cccc(-c2nc(-c3ccc4c(c3)oc3cccc(-c5cccc(-c6ccccc6)c5)c34)nc(-c3ccc4c5ccccc5c5ccccc5c4c3)n2)c1 ; N#Cc1cccc(-c2cccc(-c3nc(-c4ccc5c(c4)oc4cccc(-c6cccc(-c7ccccc7)c6)c45)nc(-c4ccc5c6ccccc6c6ccccc6c5c4)n3)c2)c1 ; N#Cc1ccc(-c2cccc(-c3nc(-c4ccc5c(c4)oc4cccc(-c6cccc(-c7ccccc7)c6)c45)nc(-c4ccc5c6ccccc6c6ccccc6c5c4)n3)c2)cc1] 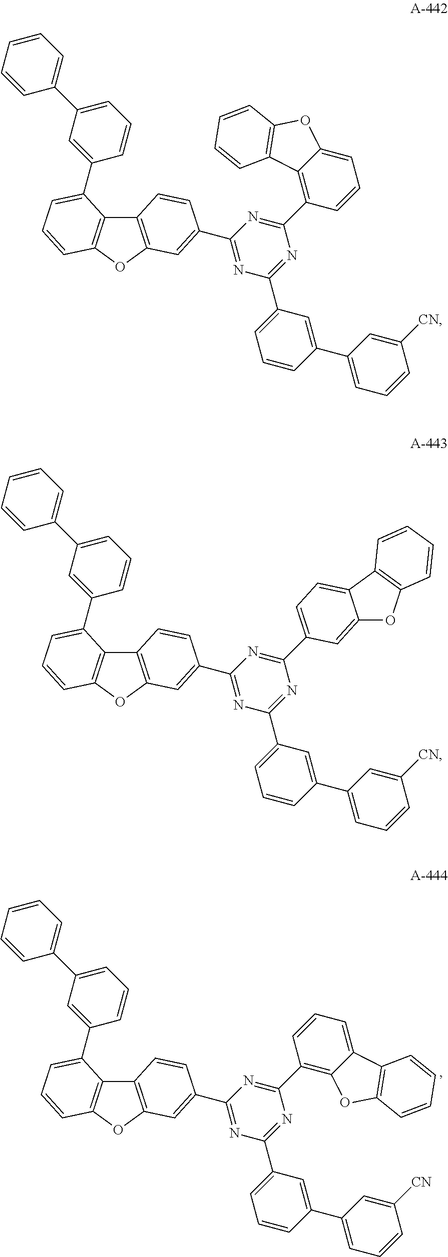 [SMILES: N#Cc1cccc(-c2cccc(-c3nc(-c4ccc5c(c4)oc4cccc(-c6cccc(-c7ccccc7)c6)c45)nc(-c4cccc5oc6ccccc6c45)n3)c2)c1 ; N#Cc1cccc(-c2cccc(-c3nc(-c4ccc5c(c4)oc4ccccc45)nc(-c4ccc5c(c4)oc4cccc(-c6cccc(-c7ccccc7)c6)c45)n3)c2)c1 ; N#Cc1cccc(-c2cccc(-c3nc(-c4ccc5c(c4)oc4cccc(-c6cccc(-c7ccccc7)c6)c45)nc(-c4cccc5c4oc4ccccc45)n3)c2)c1] 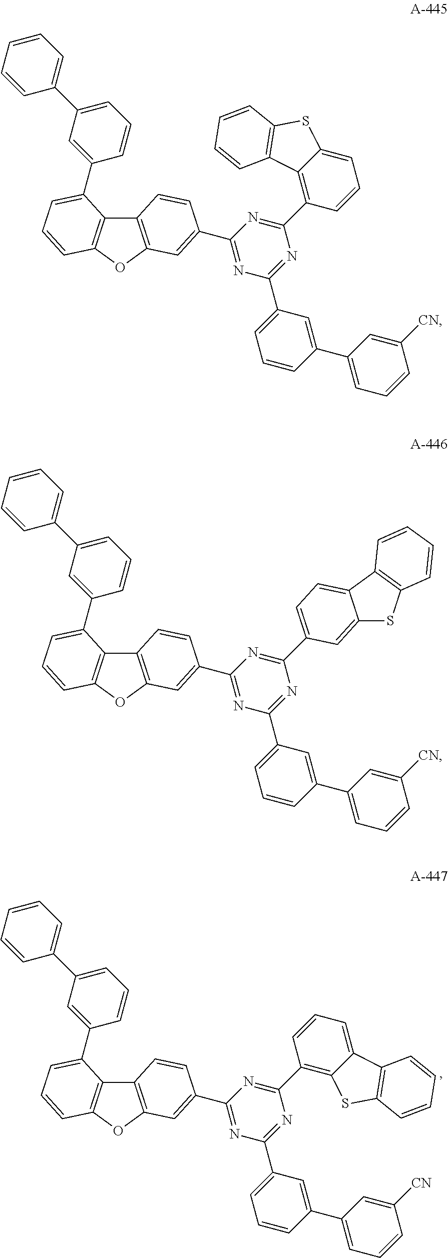 [SMILES: N#Cc1cccc(-c2cccc(-c3nc(-c4ccc5c(c4)oc4cccc(-c6cccc(-c7ccccc7)c6)c45)nc(-c4cccc5sc6ccccc6c45)n3)c2)c1 ; N#Cc1cccc(-c2cccc(-c3nc(-c4ccc5c(c4)oc4cccc(-c6cccc(-c7ccccc7)c6)c45)nc(-c4ccc5c(c4)sc4ccccc45)n3)c2)c1 ; N#Cc1cccc(-c2cccc(-c3nc(-c4ccc5c(c4)oc4cccc(-c6cccc(-c7ccccc7)c6)c45)nc(-c4cccc5c4sc4ccccc45)n3)c2)c1] 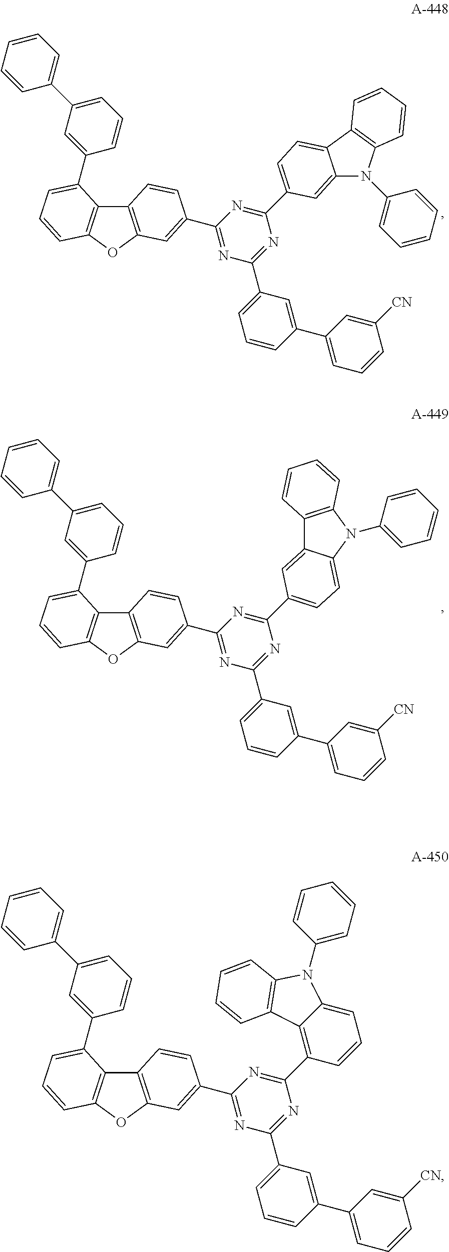 [SMILES: N#Cc1cccc(-c2cccc(-c3nc(-c4ccc5c(c4)oc4cccc(-c6cccc(-c7ccccc7)c6)c45)nc(-c4ccc5c6ccccc6n(-c6ccccc6)c5c4)n3)c2)c1 ; N#Cc1cccc(-c2cccc(-c3nc(-c4ccc5c(c4)oc4cccc(-c6cccc(-c7ccccc7)c6)c45)nc(-c4ccc5c(c4)c4ccccc4n5-c4ccccc4)n3)c2)c1 ; N#Cc1cccc(-c2cccc(-c3nc(-c4ccc5c(c4)oc4cccc(-c6cccc(-c7ccccc7)c6)c45)nc(-c4cccc5c4c4ccccc4n5-c4ccccc4)n3)c2)c1] 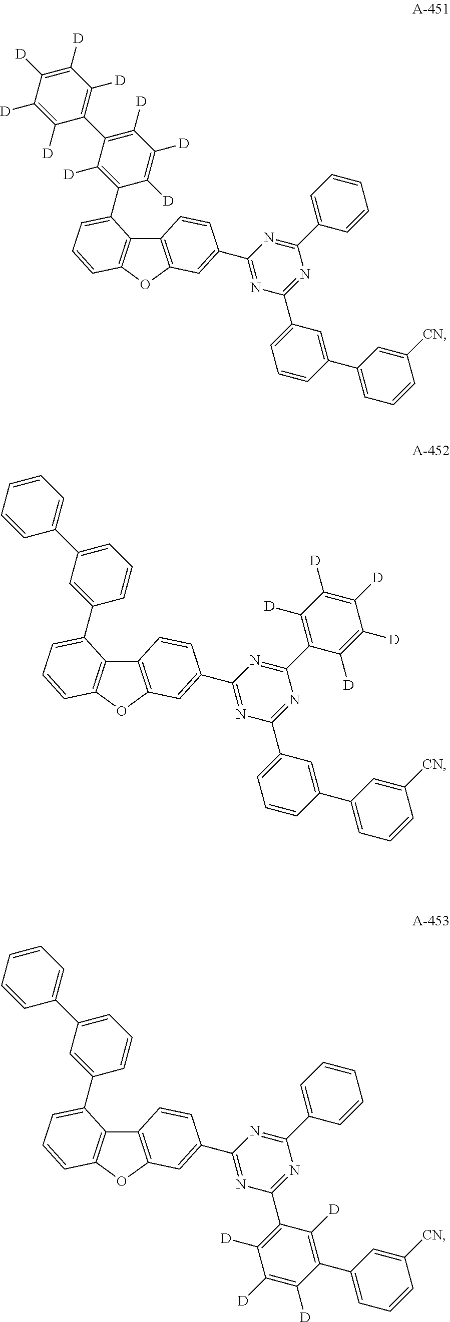 [SMILES: [2H]c1c([2H])c([2H])c(-c2c([2H])c([2H])c([2H])c(-c3cccc4oc5cc(-c6nc(-c7ccccc7)nc(-c7cccc(-c8cccc(C#N)c8)c7)n6)ccc5c34)c2[2H])c([2H])c1[2H] ; [2H]c1c([2H])c([2H])c(-c2nc(-c3cccc(-c4cccc(C#N)c4)c3)nc(-c3ccc4c(c3)oc3cccc(-c5cccc(-c6ccccc6)c5)c34)n2)c([2H])c1[2H] ; [2H]c1c([2H])c(-c2cccc(C#N)c2)c([2H])c(-c2nc(-c3ccccc3)nc(-c3ccc4c(c3)oc3cccc(-c5cccc(-c6ccccc6)c5)c34)n2)c1[2H]] 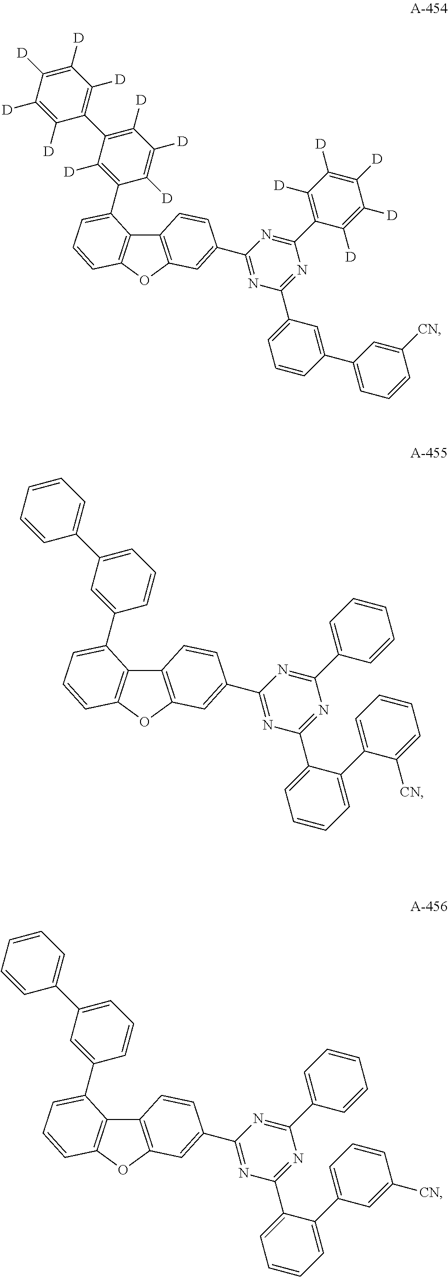 [SMILES: [2H]c1c([2H])c([2H])c(-c2nc(-c3cccc(-c4cccc(C#N)c4)c3)nc(-c3ccc4c(c3)oc3cccc(-c5c([2H])c([2H])c([2H])c(-c6c([2H])c([2H])c([2H])c([2H])c6[2H])c5[2H])c34)n2)c([2H])c1[2H] ; N#Cc1ccccc1-c1ccccc1-c1nc(-c2ccccc2)nc(-c2ccc3c(c2)oc2cccc(-c4cccc(-c5ccccc5)c4)c23)n1 ; N#Cc1cccc(-c2ccccc2-c2nc(-c3ccccc3)nc(-c3ccc4c(c3)oc3cccc(-c5cccc(-c6ccccc6)c5)c34)n2)c1] 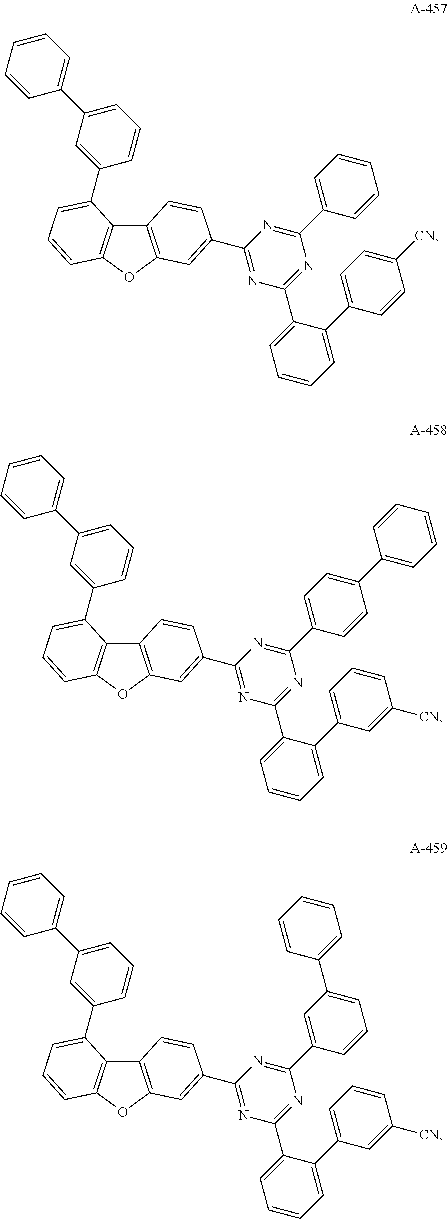 [SMILES: N#Cc1ccc(-c2ccccc2-c2nc(-c3ccccc3)nc(-c3ccc4c(c3)oc3cccc(-c5cccc(-c6ccccc6)c5)c34)n2)cc1 ; N#Cc1cccc(-c2ccccc2-c2nc(-c3ccc(-c4ccccc4)cc3)nc(-c3ccc4c(c3)oc3cccc(-c5cccc(-c6ccccc6)c5)c34)n2)c1 ; N#Cc1cccc(-c2ccccc2-c2nc(-c3cccc(-c4ccccc4)c3)nc(-c3ccc4c(c3)oc3cccc(-c5cccc(-c6ccccc6)c5)c34)n2)c1] 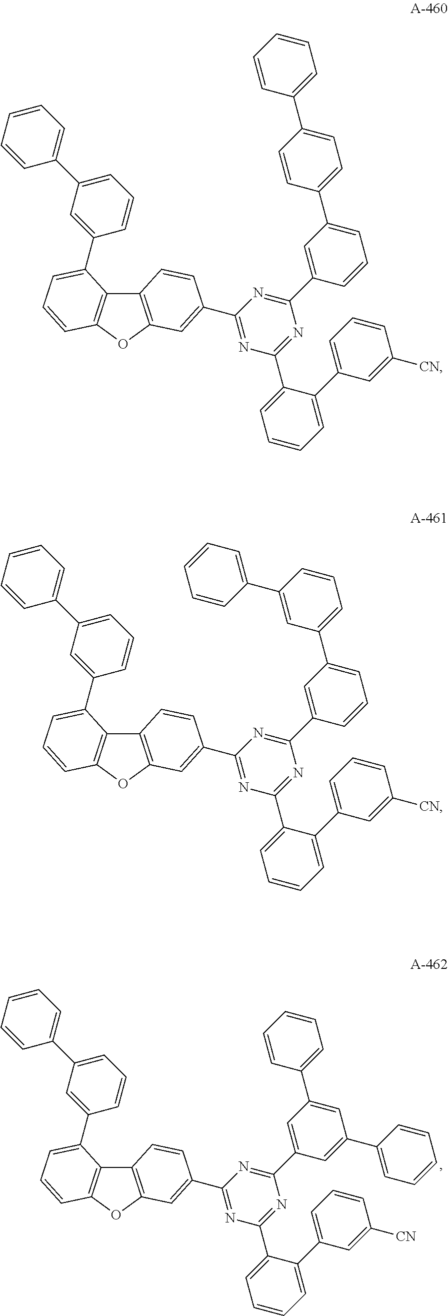 [SMILES: N#Cc1cccc(-c2ccccc2-c2nc(-c3cccc(-c4ccc(-c5ccccc5)cc4)c3)nc(-c3ccc4c(c3)oc3cccc(-c5cccc(-c6ccccc6)c5)c34)n2)c1 ; N#Cc1cccc(-c2ccccc2-c2nc(-c3cccc(-c4cccc(-c5ccccc5)c4)c3)nc(-c3ccc4c(c3)oc3cccc(-c5cccc(-c6ccccc6)c5)c34)n2)c1 ; N#Cc1cccc(-c2ccccc2-c2nc(-c3cc(-c4ccccc4)cc(-c4ccccc4)c3)nc(-c3ccc4c(c3)oc3cccc(-c5cccc(-c6ccccc6)c5)c34)n2)c1] 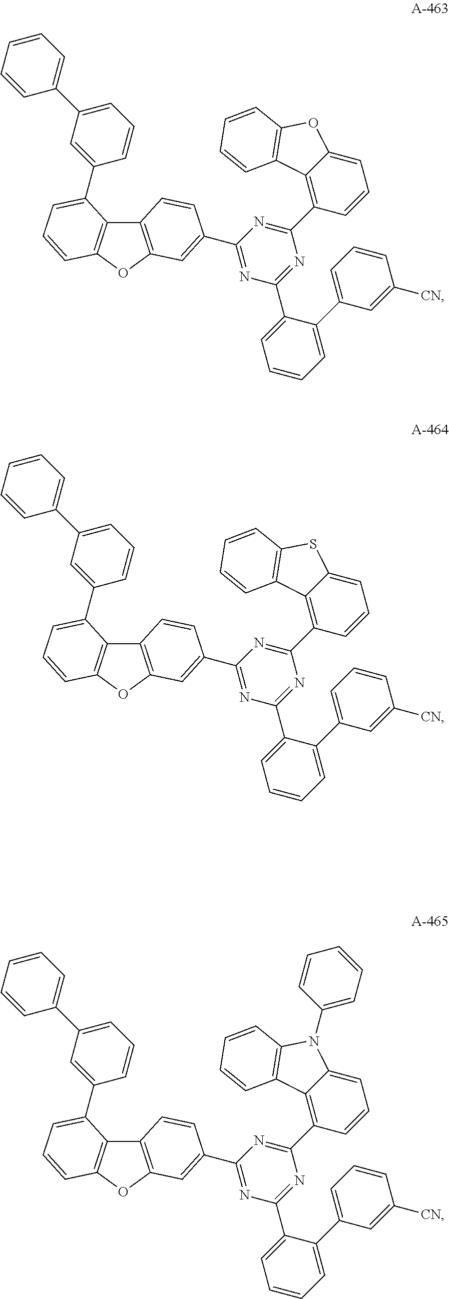 [SMILES: N#Cc1cccc(-c2ccccc2-c2nc(-c3ccc4c(c3)oc3cccc(-c5cccc(-c6ccccc6)c5)c34)nc(-c3cccc4oc5ccccc5c34)n2)c1 ; N#Cc1cccc(-c2ccccc2-c2nc(-c3ccc4c(c3)oc3cccc(-c5cccc(-c6ccccc6)c5)c34)nc(-c3cccc4sc5ccccc5c34)n2)c1 ; N#Cc1cccc(-c2ccccc2-c2nc(-c3ccc4c(c3)oc3cccc(-c5cccc(-c6ccccc6)c5)c34)nc(-c3cccc4c3c3ccccc3n4-c3ccccc3)n2)c1] 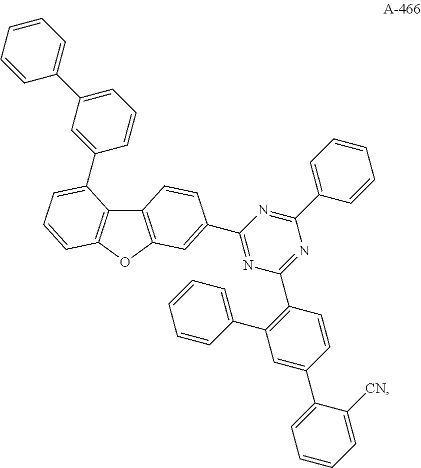 [SMILES: N#Cc1ccccc1-c1ccc(-c2nc(-c3ccccc3)nc(-c3ccc4c(c3)oc3cccc(-c5cccc(-c6ccccc6)c5)c34)n2)c(-c2ccccc2)c1] 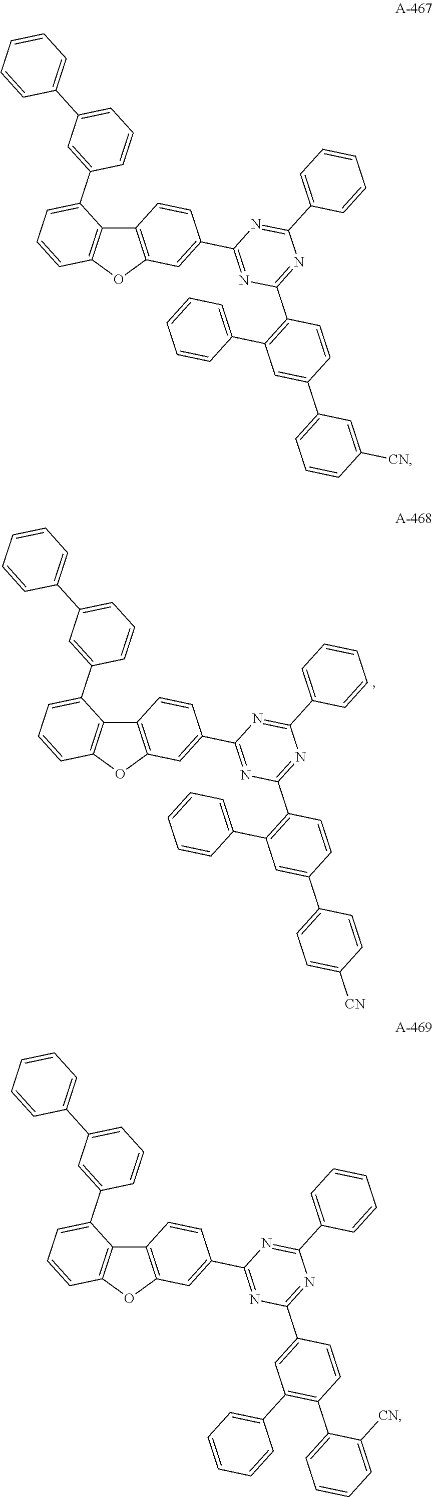 [SMILES: N#Cc1cccc(-c2ccc(-c3nc(-c4ccccc4)nc(-c4ccc5c(c4)oc4cccc(-c6cccc(-c7ccccc7)c6)c45)n3)c(-c3ccccc3)c2)c1 ; N#Cc1ccc(-c2ccc(-c3nc(-c4ccccc4)nc(-c4ccc5c(c4)oc4cccc(-c6cccc(-c7ccccc7)c6)c45)n3)c(-c3ccccc3)c2)cc1 ; N#Cc1ccccc1-c1ccc(-c2nc(-c3ccccc3)nc(-c3ccc4c(c3)oc3cccc(-c5cccc(-c6ccccc6)c5)c34)n2)cc1-c1ccccc1] 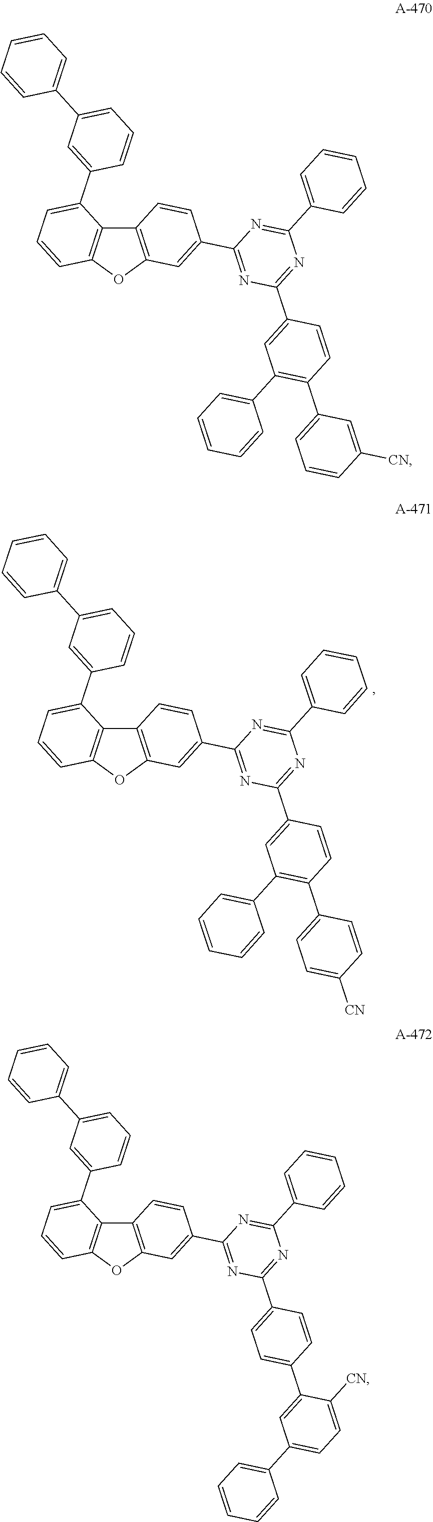 [SMILES: N#Cc1cccc(-c2ccc(-c3nc(-c4ccccc4)nc(-c4ccc5c(c4)oc4cccc(-c6cccc(-c7ccccc7)c6)c45)n3)cc2-c2ccccc2)c1 ; N#Cc1ccc(-c2ccc(-c3nc(-c4ccccc4)nc(-c4ccc5c(c4)oc4cccc(-c6cccc(-c7ccccc7)c6)c45)n3)cc2-c2ccccc2)cc1 ; N#Cc1ccc(-c2ccccc2)cc1-c1ccc(-c2nc(-c3ccccc3)nc(-c3ccc4c(c3)oc3cccc(-c5cccc(-c6ccccc6)c5)c34)n2)cc1] 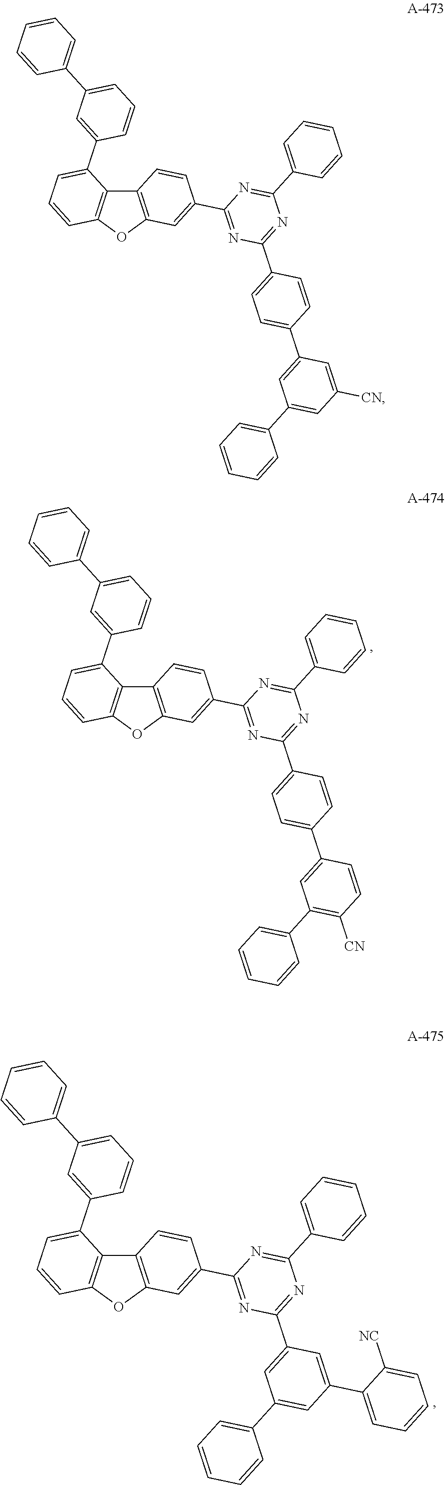 [SMILES: N#Cc1cc(-c2ccccc2)cc(-c2ccc(-c3nc(-c4ccccc4)nc(-c4ccc5c(c4)oc4cccc(-c6cccc(-c7ccccc7)c6)c45)n3)cc2)c1 ; N#Cc1ccc(-c2ccc(-c3nc(-c4ccccc4)nc(-c4ccc5c(c4)oc4cccc(-c6cccc(-c7ccccc7)c6)c45)n3)cc2)cc1-c1ccccc1 ; N#Cc1ccccc1-c1cc(-c2ccccc2)cc(-c2nc(-c3ccccc3)nc(-c3ccc4c(c3)oc3cccc(-c5cccc(-c6ccccc6)c5)c34)n2)c1] 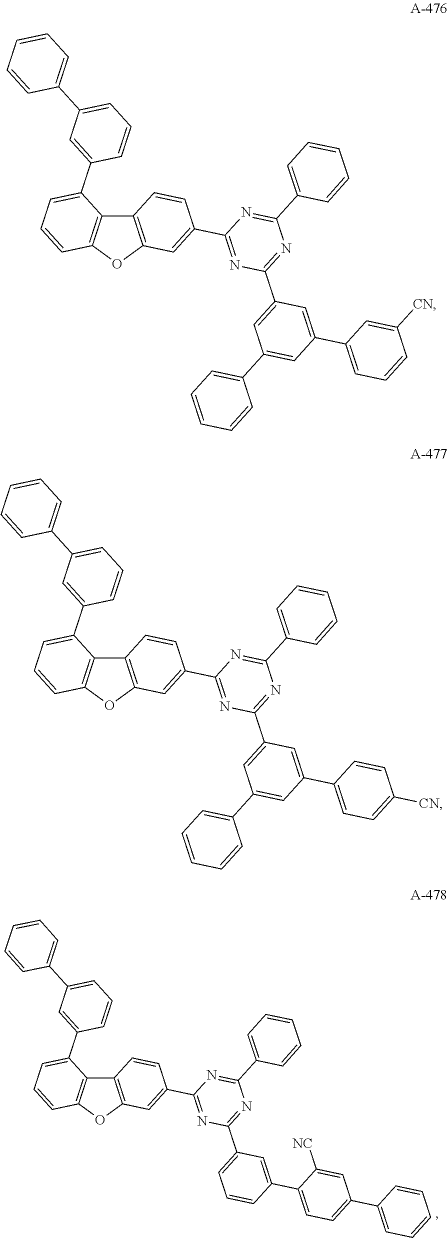 [SMILES: N#Cc1cccc(-c2cc(-c3ccccc3)cc(-c3nc(-c4ccccc4)nc(-c4ccc5c(c4)oc4cccc(-c6cccc(-c7ccccc7)c6)c45)n3)c2)c1 ; N#Cc1ccc(-c2cc(-c3ccccc3)cc(-c3nc(-c4ccccc4)nc(-c4ccc5c(c4)oc4cccc(-c6cccc(-c7ccccc7)c6)c45)n3)c2)cc1 ; N#Cc1cc(-c2ccccc2)ccc1-c1cccc(-c2nc(-c3ccccc3)nc(-c3ccc4c(c3)oc3cccc(-c5cccc(-c6ccccc6)c5)c34)n2)c1] 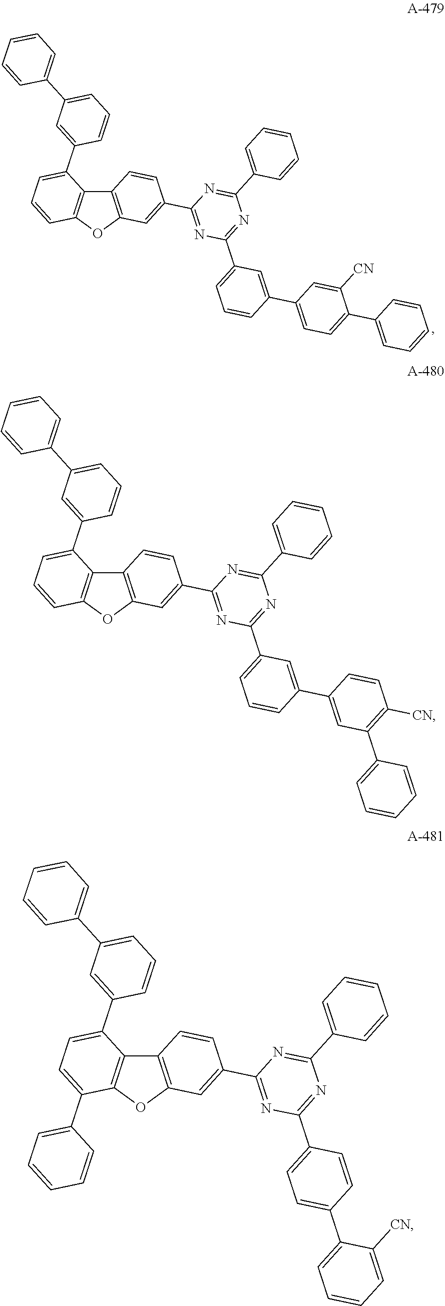 [SMILES: N#Cc1cc(-c2cccc(-c3nc(-c4ccccc4)nc(-c4ccc5c(c4)oc4cccc(-c6cccc(-c7ccccc7)c6)c45)n3)c2)ccc1-c1ccccc1 ; N#Cc1ccc(-c2cccc(-c3nc(-c4ccccc4)nc(-c4ccc5c(c4)oc4cccc(-c6cccc(-c7ccccc7)c6)c45)n3)c2)cc1-c1ccccc1 ; N#Cc1ccccc1-c1ccc(-c2nc(-c3ccccc3)nc(-c3ccc4c(c3)oc3c(-c5ccccc5)ccc(-c5cccc(-c6ccccc6)c5)c34)n2)cc1] 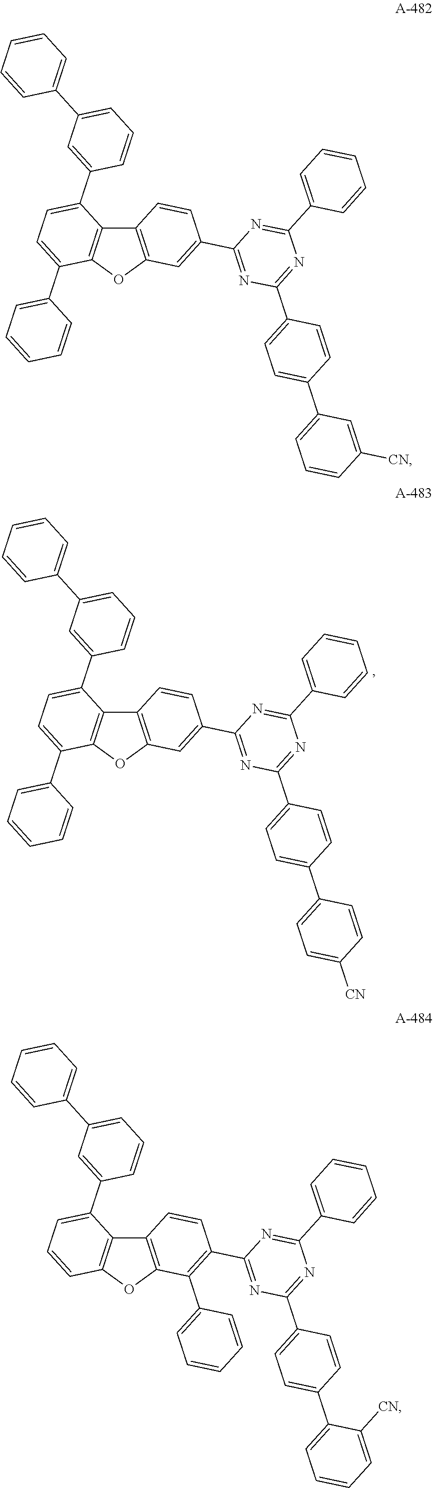 [SMILES: N#Cc1cccc(-c2ccc(-c3nc(-c4ccccc4)nc(-c4ccc5c(c4)oc4c(-c6ccccc6)ccc(-c6cccc(-c7ccccc7)c6)c45)n3)cc2)c1 ; N#Cc1ccc(-c2ccc(-c3nc(-c4ccccc4)nc(-c4ccc5c(c4)oc4c(-c6ccccc6)ccc(-c6cccc(-c7ccccc7)c6)c45)n3)cc2)cc1 ; N#Cc1ccccc1-c1ccc(-c2nc(-c3ccccc3)nc(-c3ccc4c(oc5cccc(-c6cccc(-c7ccccc7)c6)c54)c3-c3ccccc3)n2)cc1] 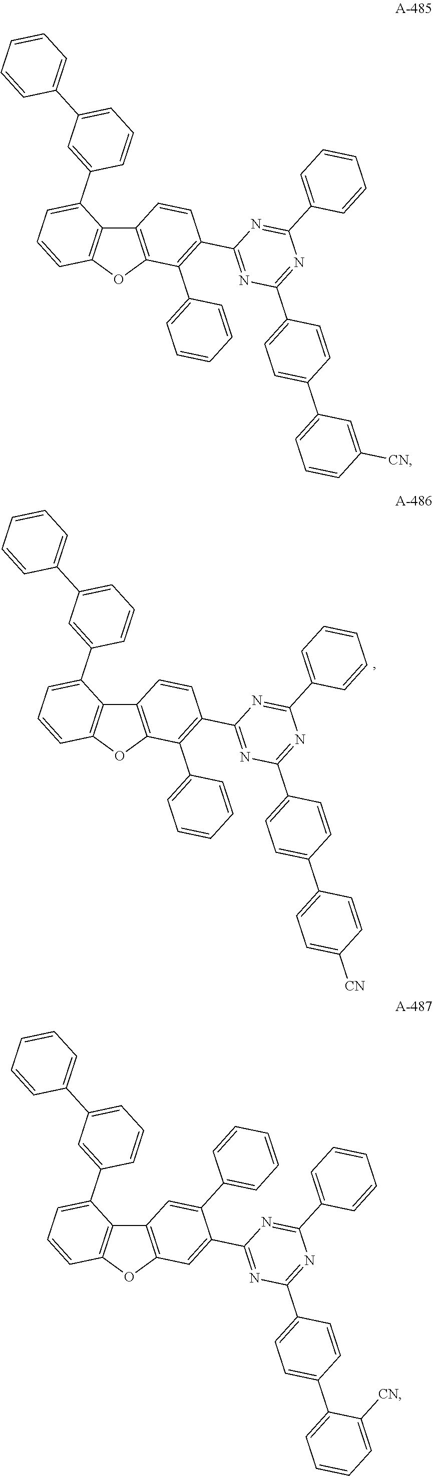 [SMILES: N#Cc1cccc(-c2ccc(-c3nc(-c4ccccc4)nc(-c4ccc5c(oc6cccc(-c7cccc(-c8ccccc8)c7)c65)c4-c4ccccc4)n3)cc2)c1 ; N#Cc1ccc(-c2ccc(-c3nc(-c4ccccc4)nc(-c4ccc5c(oc6cccc(-c7cccc(-c8ccccc8)c7)c65)c4-c4ccccc4)n3)cc2)cc1 ; N#Cc1ccccc1-c1ccc(-c2nc(-c3ccccc3)nc(-c3cc4oc5cccc(-c6cccc(-c7ccccc7)c6)c5c4cc3-c3ccccc3)n2)cc1] 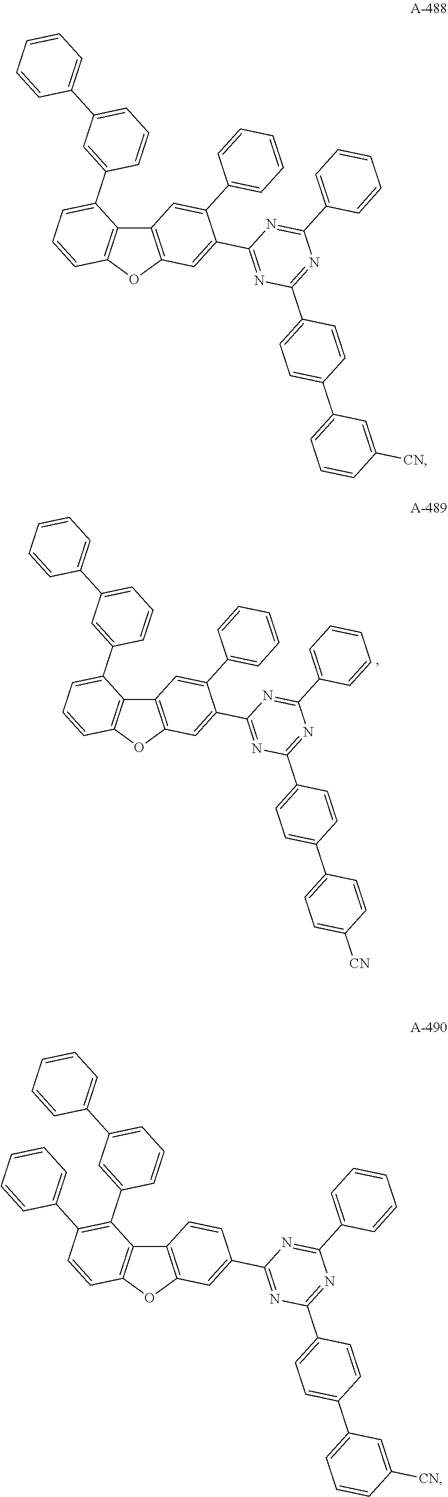 [SMILES: N#Cc1cccc(-c2ccc(-c3nc(-c4ccccc4)nc(-c4cc5oc6cccc(-c7cccc(-c8ccccc8)c7)c6c5cc4-c4ccccc4)n3)cc2)c1 ; N#Cc1ccc(-c2ccc(-c3nc(-c4ccccc4)nc(-c4cc5oc6cccc(-c7cccc(-c8ccccc8)c7)c6c5cc4-c4ccccc4)n3)cc2)cc1 ; N#Cc1cccc(-c2ccc(-c3nc(-c4ccccc4)nc(-c4ccc5c(c4)oc4ccc(-c6ccccc6)c(-c6cccc(-c7ccccc7)c6)c45)n3)cc2)c1] 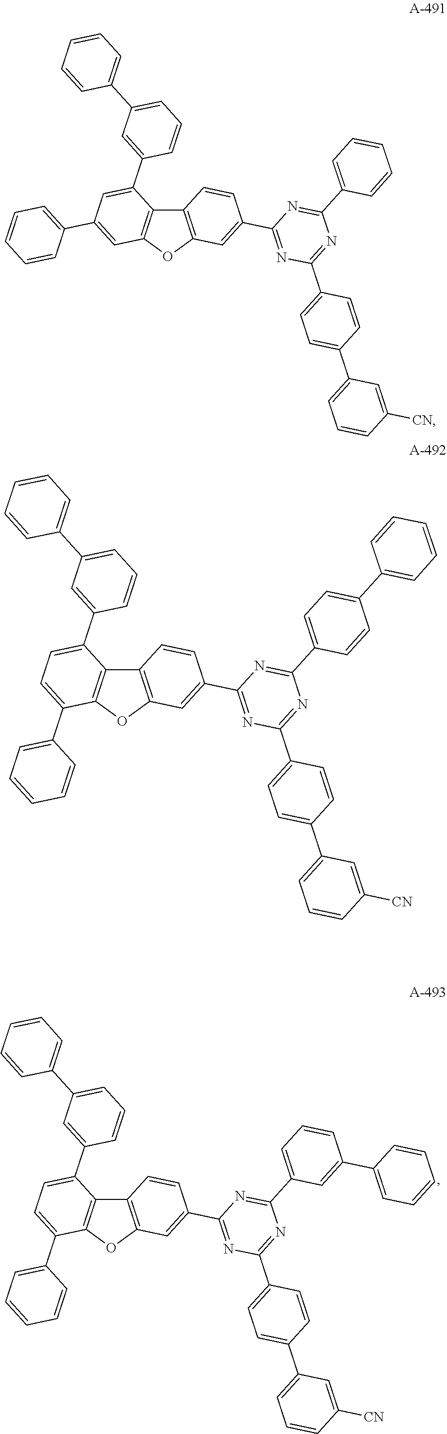 [SMILES: N#Cc1cccc(-c2ccc(-c3nc(-c4ccccc4)nc(-c4ccc5c(c4)oc4cc(-c6ccccc6)cc(-c6cccc(-c7ccccc7)c6)c45)n3)cc2)c1 ; N#Cc1cccc(-c2ccc(-c3nc(-c4ccc(-c5ccccc5)cc4)nc(-c4ccc5c(c4)oc4c(-c6ccccc6)ccc(-c6cccc(-c7ccccc7)c6)c45)n3)cc2)c1 ; N#Cc1cccc(-c2ccc(-c3nc(-c4cccc(-c5ccccc5)c4)nc(-c4ccc5c(c4)oc4c(-c6ccccc6)ccc(-c6cccc(-c7ccccc7)c6)c45)n3)cc2)c1] 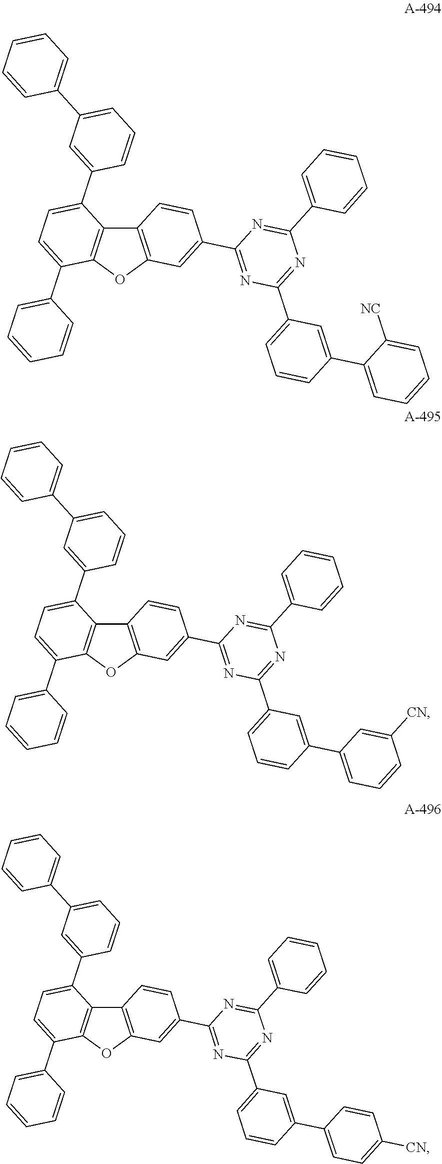 [SMILES: N#Cc1ccccc1-c1cccc(-c2nc(-c3ccccc3)nc(-c3ccc4c(c3)oc3c(-c5ccccc5)ccc(-c5cccc(-c6ccccc6)c5)c34)n2)c1 ; N#Cc1cccc(-c2cccc(-c3nc(-c4ccccc4)nc(-c4ccc5c(c4)oc4c(-c6ccccc6)ccc(-c6cccc(-c7ccccc7)c6)c45)n3)c2)c1 ; N#Cc1ccc(-c2cccc(-c3nc(-c4ccccc4)nc(-c4ccc5c(c4)oc4c(-c6ccccc6)ccc(-c6cccc(-c7ccccc7)c6)c45)n3)c2)cc1] 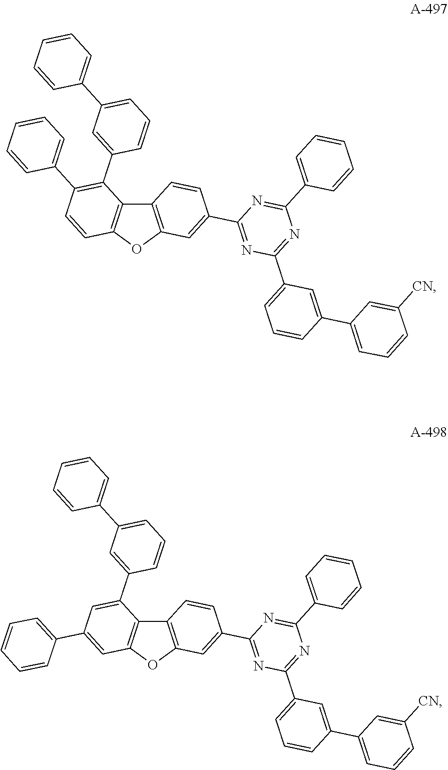 [SMILES: N#Cc1cccc(-c2cccc(-c3nc(-c4ccccc4)nc(-c4ccc5c(c4)oc4ccc(-c6ccccc6)c(-c6cccc(-c7ccccc7)c6)c45)n3)c2)c1 ; N#Cc1cccc(-c2cccc(-c3nc(-c4ccccc4)nc(-c4ccc5c(c4)oc4cc(-c6ccccc6)cc(-c6cccc(-c7ccccc7)c6)c45)n3)c2)c1] 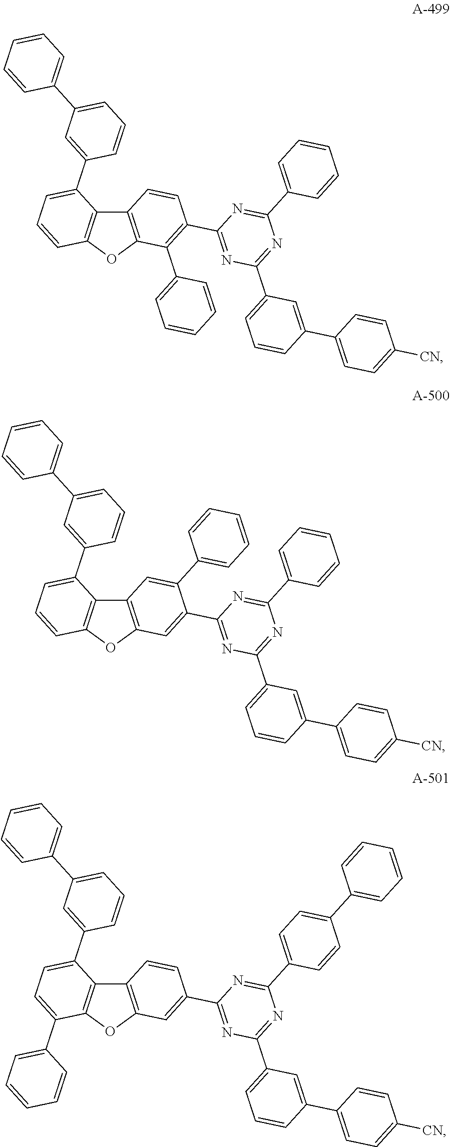 [SMILES: N#Cc1ccc(-c2cccc(-c3nc(-c4ccccc4)nc(-c4ccc5c(oc6cccc(-c7cccc(-c8ccccc8)c7)c65)c4-c4ccccc4)n3)c2)cc1 ; N#Cc1ccc(-c2cccc(-c3nc(-c4ccccc4)nc(-c4cc5oc6cccc(-c7cccc(-c8ccccc8)c7)c6c5cc4-c4ccccc4)n3)c2)cc1 ; N#Cc1ccc(-c2cccc(-c3nc(-c4ccc(-c5ccccc5)cc4)nc(-c4ccc5c(c4)oc4c(-c6ccccc6)ccc(-c6cccc(-c7ccccc7)c6)c45)n3)c2)cc1] 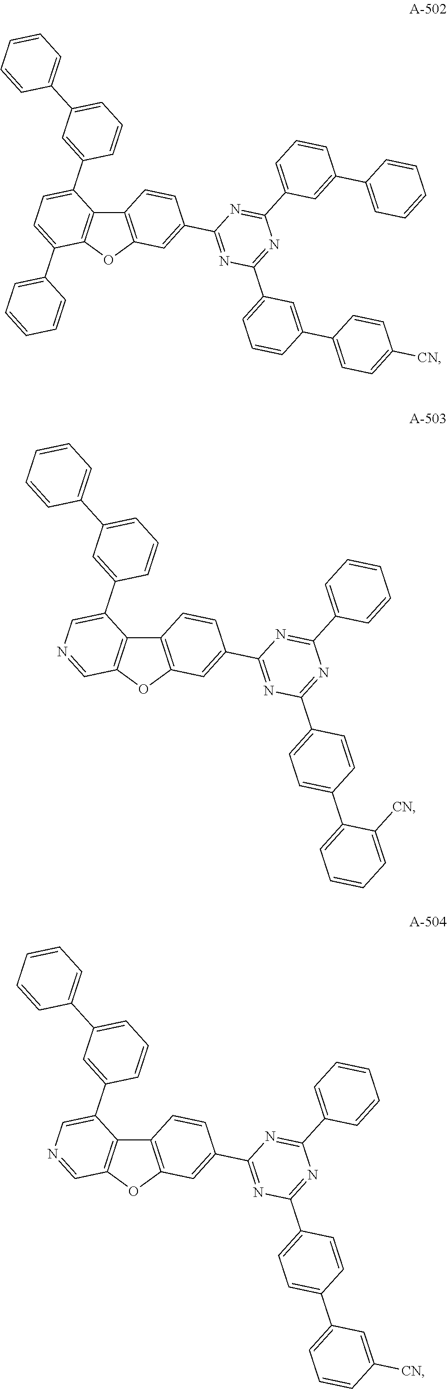 [SMILES: N#Cc1ccc(-c2cccc(-c3nc(-c4cccc(-c5ccccc5)c4)nc(-c4ccc5c(c4)oc4c(-c6ccccc6)ccc(-c6cccc(-c7ccccc7)c6)c45)n3)c2)cc1 ; N#Cc1ccccc1-c1ccc(-c2nc(-c3ccccc3)nc(-c3ccc4c(c3)oc3cncc(-c5cccc(-c6ccccc6)c5)c34)n2)cc1 ; N#Cc1cccc(-c2ccc(-c3nc(-c4ccccc4)nc(-c4ccc5c(c4)oc4cncc(-c6cccc(-c7ccccc7)c6)c45)n3)cc2)c1] 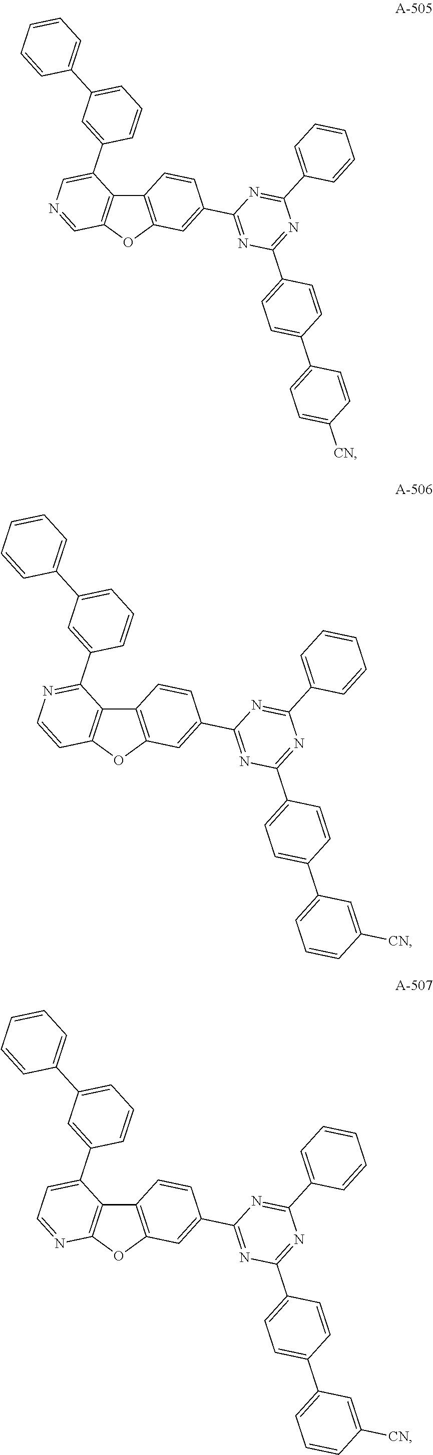 [SMILES: N#Cc1ccc(-c2ccc(-c3nc(-c4ccccc4)nc(-c4ccc5c(c4)oc4cncc(-c6cccc(-c7ccccc7)c6)c45)n3)cc2)cc1 ; N#Cc1cccc(-c2ccc(-c3nc(-c4ccccc4)nc(-c4ccc5c(c4)oc4ccnc(-c6cccc(-c7ccccc7)c6)c45)n3)cc2)c1 ; N#Cc1cccc(-c2ccc(-c3nc(-c4ccccc4)nc(-c4ccc5c(c4)oc4nccc(-c6cccc(-c7ccccc7)c6)c45)n3)cc2)c1] 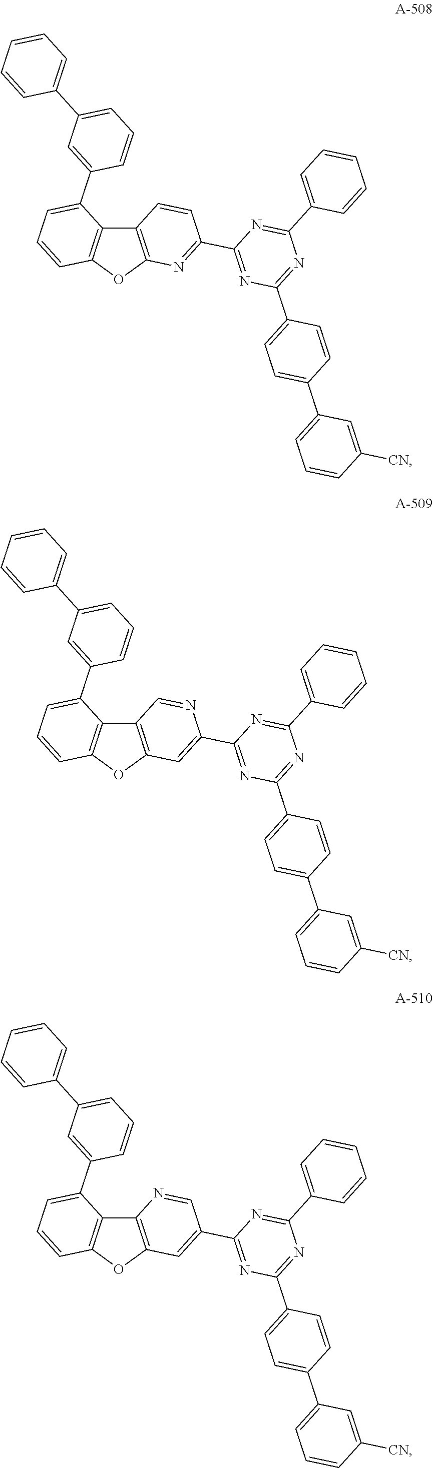 [SMILES: N#Cc1cccc(-c2ccc(-c3nc(-c4ccccc4)nc(-c4ccc5c(n4)oc4cccc(-c6cccc(-c7ccccc7)c6)c45)n3)cc2)c1 ; N#Cc1cccc(-c2ccc(-c3nc(-c4ccccc4)nc(-c4cc5oc6cccc(-c7cccc(-c8ccccc8)c7)c6c5cn4)n3)cc2)c1 ; N#Cc1cccc(-c2ccc(-c3nc(-c4ccccc4)nc(-c4cnc5c(c4)oc4cccc(-c6cccc(-c7ccccc7)c6)c45)n3)cc2)c1] 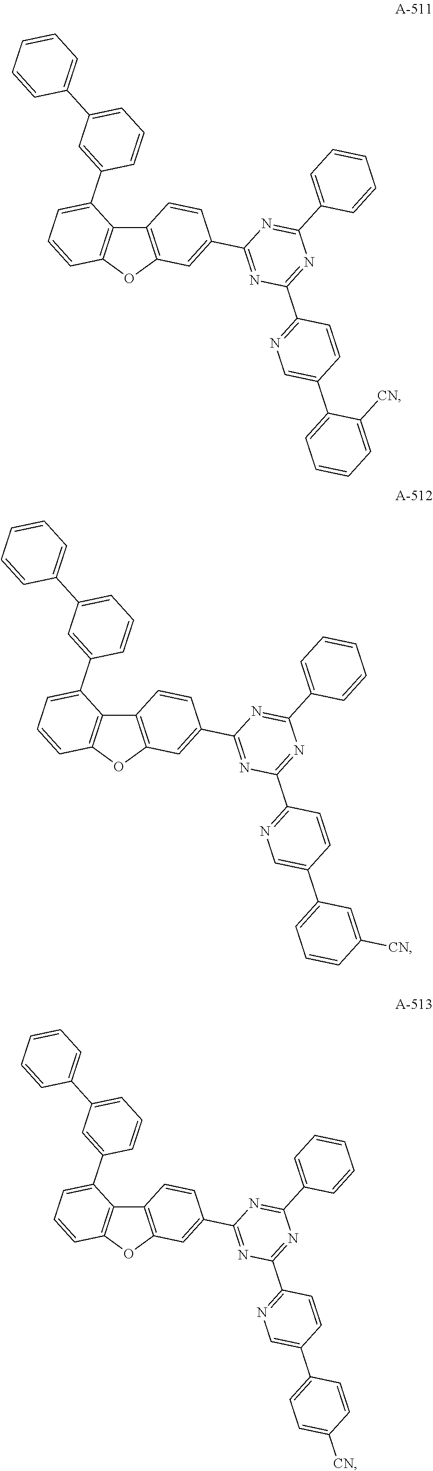 [SMILES: N#Cc1ccccc1-c1ccc(-c2nc(-c3ccccc3)nc(-c3ccc4c(c3)oc3cccc(-c5cccc(-c6ccccc6)c5)c34)n2)nc1 ; N#Cc1cccc(-c2ccc(-c3nc(-c4ccccc4)nc(-c4ccc5c(c4)oc4cccc(-c6cccc(-c7ccccc7)c6)c45)n3)nc2)c1 ; N#Cc1ccc(-c2ccc(-c3nc(-c4ccccc4)nc(-c4ccc5c(c4)oc4cccc(-c6cccc(-c7ccccc7)c6)c45)n3)nc2)cc1] 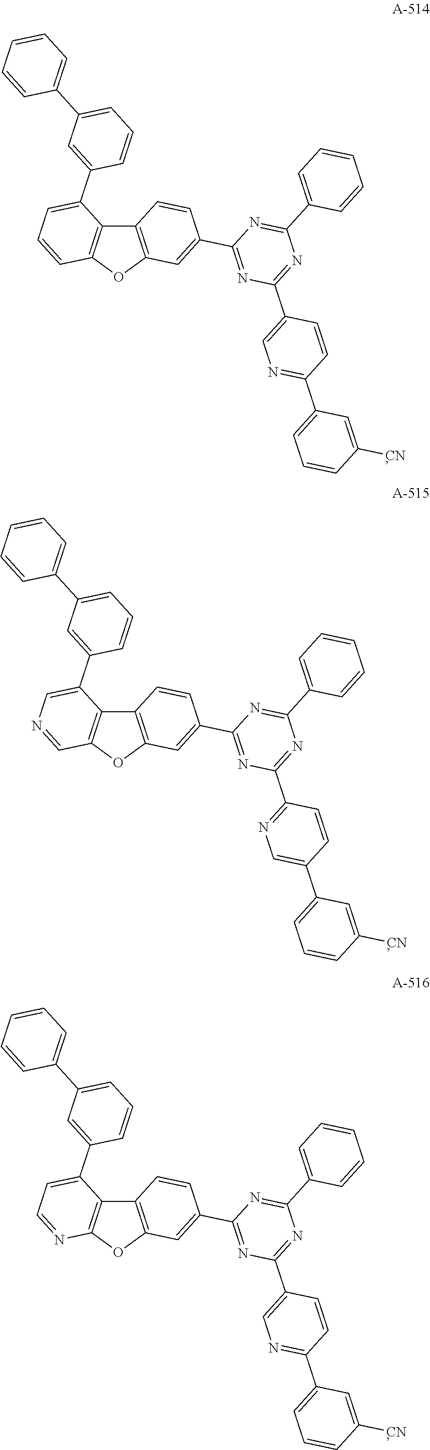 [SMILES: N#Cc1cccc(-c2ccc(-c3nc(-c4ccccc4)nc(-c4ccc5c(c4)oc4cccc(-c6cccc(-c7ccccc7)c6)c45)n3)cn2)c1 ; N#Cc1cccc(-c2ccc(-c3nc(-c4ccccc4)nc(-c4ccc5c(c4)oc4cncc(-c6cccc(-c7ccccc7)c6)c45)n3)nc2)c1 ; N#Cc1cccc(-c2ccc(-c3nc(-c4ccccc4)nc(-c4ccc5c(c4)oc4nccc(-c6cccc(-c7ccccc7)c6)c45)n3)cn2)c1] 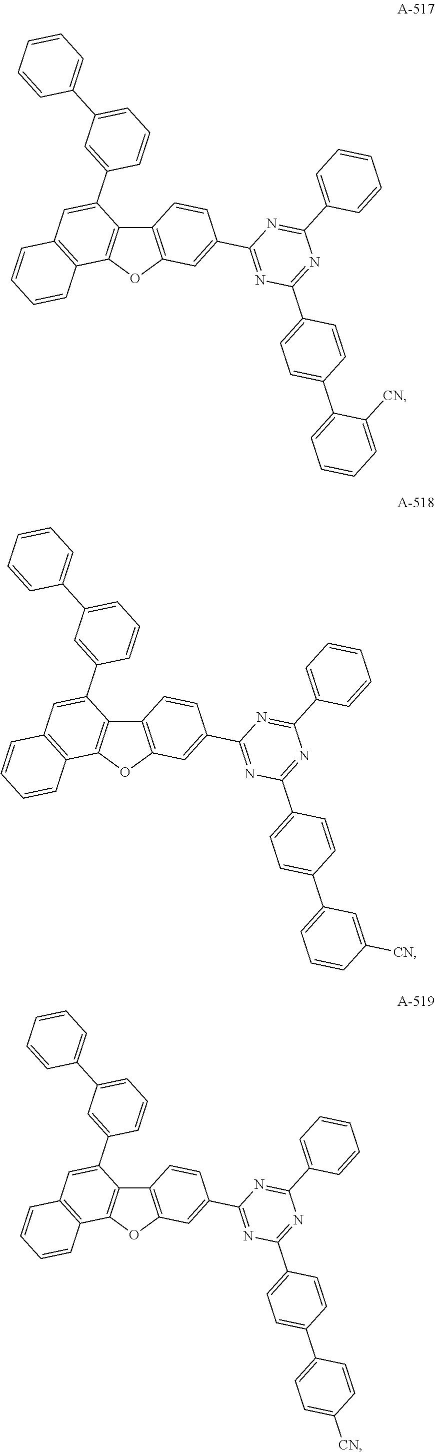 [SMILES: N#Cc1ccccc1-c1ccc(-c2nc(-c3ccccc3)nc(-c3ccc4c(c3)oc3c5ccccc5cc(-c5cccc(-c6ccccc6)c5)c43)n2)cc1 ; N#Cc1cccc(-c2ccc(-c3nc(-c4ccccc4)nc(-c4ccc5c(c4)oc4c6ccccc6cc(-c6cccc(-c7ccccc7)c6)c54)n3)cc2)c1 ; N#Cc1ccc(-c2ccc(-c3nc(-c4ccccc4)nc(-c4ccc5c(c4)oc4c6ccccc6cc(-c6cccc(-c7ccccc7)c6)c54)n3)cc2)cc1] 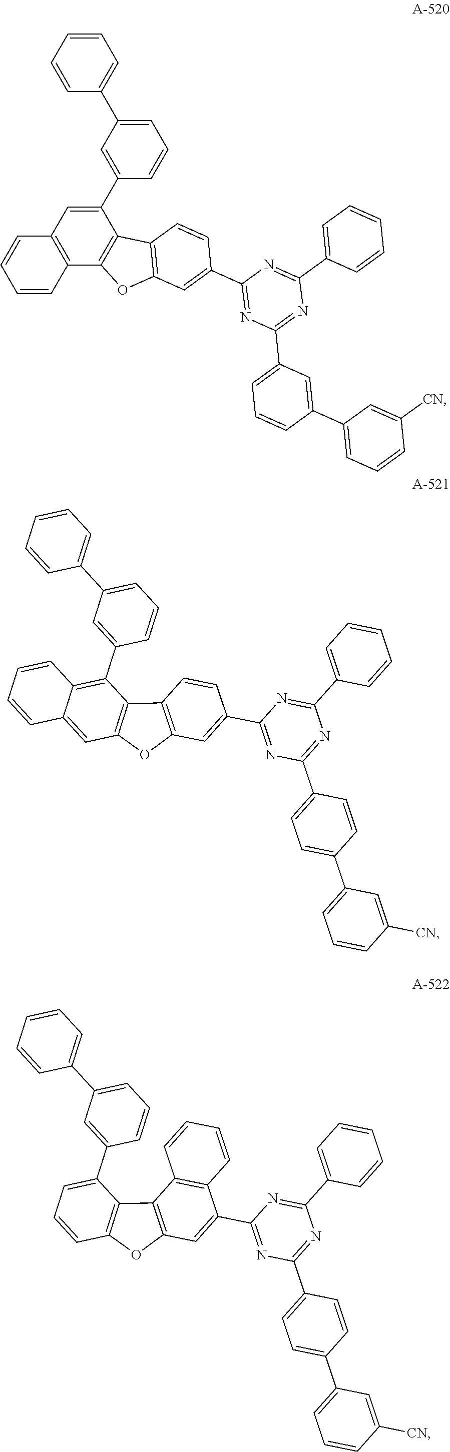 [SMILES: N#Cc1cccc(-c2cccc(-c3nc(-c4ccccc4)nc(-c4ccc5c(c4)oc4c6ccccc6cc(-c6cccc(-c7ccccc7)c6)c54)n3)c2)c1 ; N#Cc1cccc(-c2ccc(-c3nc(-c4ccccc4)nc(-c4ccc5c(c4)oc4cc6ccccc6c(-c6cccc(-c7ccccc7)c6)c45)n3)cc2)c1 ; N#Cc1cccc(-c2ccc(-c3nc(-c4ccccc4)nc(-c4cc5oc6cccc(-c7cccc(-c8ccccc8)c7)c6c5c5ccccc45)n3)cc2)c1] 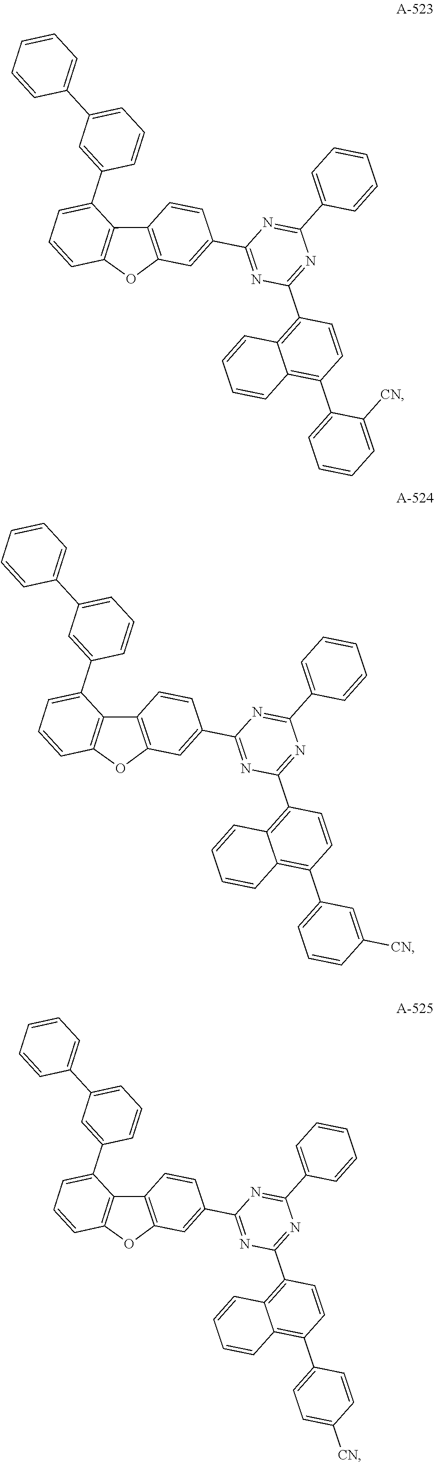 [SMILES: N#Cc1ccccc1-c1ccc(-c2nc(-c3ccccc3)nc(-c3ccc4c(c3)oc3cccc(-c5cccc(-c6ccccc6)c5)c34)n2)c2ccccc12 ; N#Cc1cccc(-c2ccc(-c3nc(-c4ccccc4)nc(-c4ccc5c(c4)oc4cccc(-c6cccc(-c7ccccc7)c6)c45)n3)c3ccccc23)c1 ; N#Cc1ccc(-c2ccc(-c3nc(-c4ccccc4)nc(-c4ccc5c(c4)oc4cccc(-c6cccc(-c7ccccc7)c6)c45)n3)c3ccccc23)cc1] 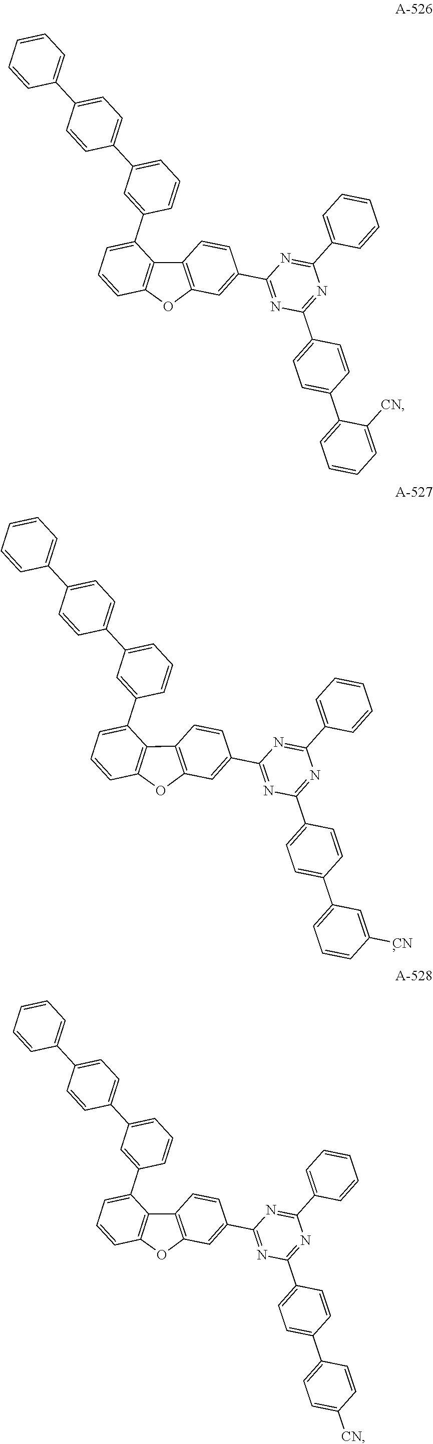 [SMILES: N#Cc1ccccc1-c1ccc(-c2nc(-c3ccccc3)nc(-c3ccc4c(c3)oc3cccc(-c5cccc(-c6ccc(-c7ccccc7)cc6)c5)c34)n2)cc1 ; N#Cc1cccc(-c2ccc(-c3nc(-c4ccccc4)nc(-c4ccc5c(c4)oc4cccc(-c6cccc(-c7ccc(-c8ccccc8)cc7)c6)c45)n3)cc2)c1 ; N#Cc1ccc(-c2ccc(-c3nc(-c4ccccc4)nc(-c4ccc5c(c4)oc4cccc(-c6cccc(-c7ccc(-c8ccccc8)cc7)c6)c45)n3)cc2)cc1] 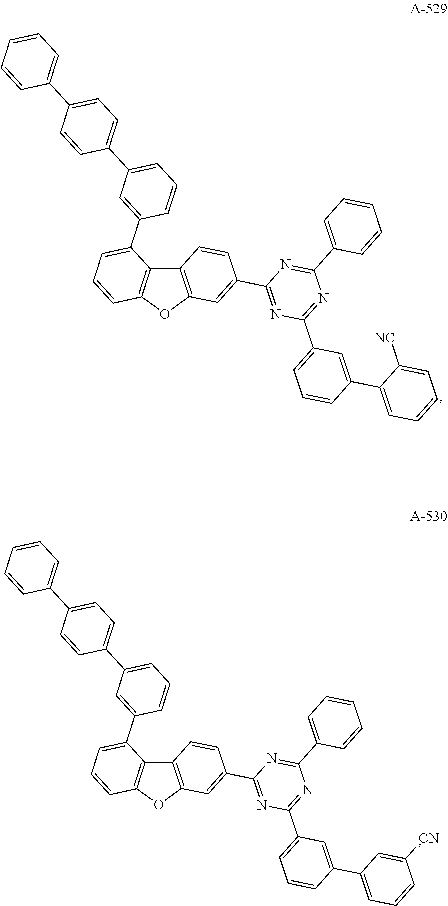 [SMILES: N#Cc1ccccc1-c1cccc(-c2nc(-c3ccccc3)nc(-c3ccc4c(c3)oc3cccc(-c5cccc(-c6ccc(-c7ccccc7)cc6)c5)c34)n2)c1 ; N#Cc1cccc(-c2cccc(-c3nc(-c4ccccc4)nc(-c4ccc5c(c4)oc4cccc(-c6cccc(-c7ccc(-c8ccccc8)cc7)c6)c45)n3)c2)c1] 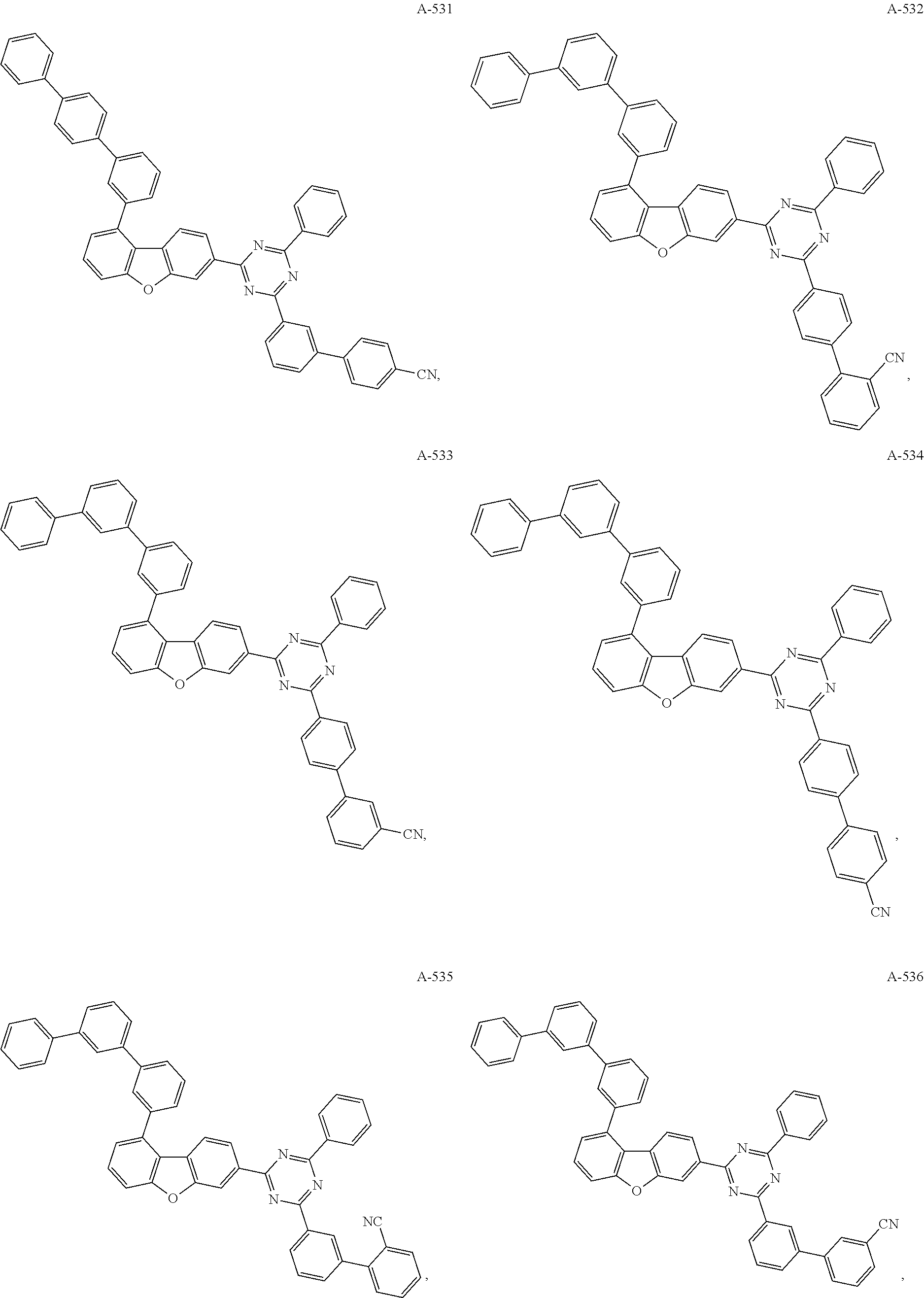 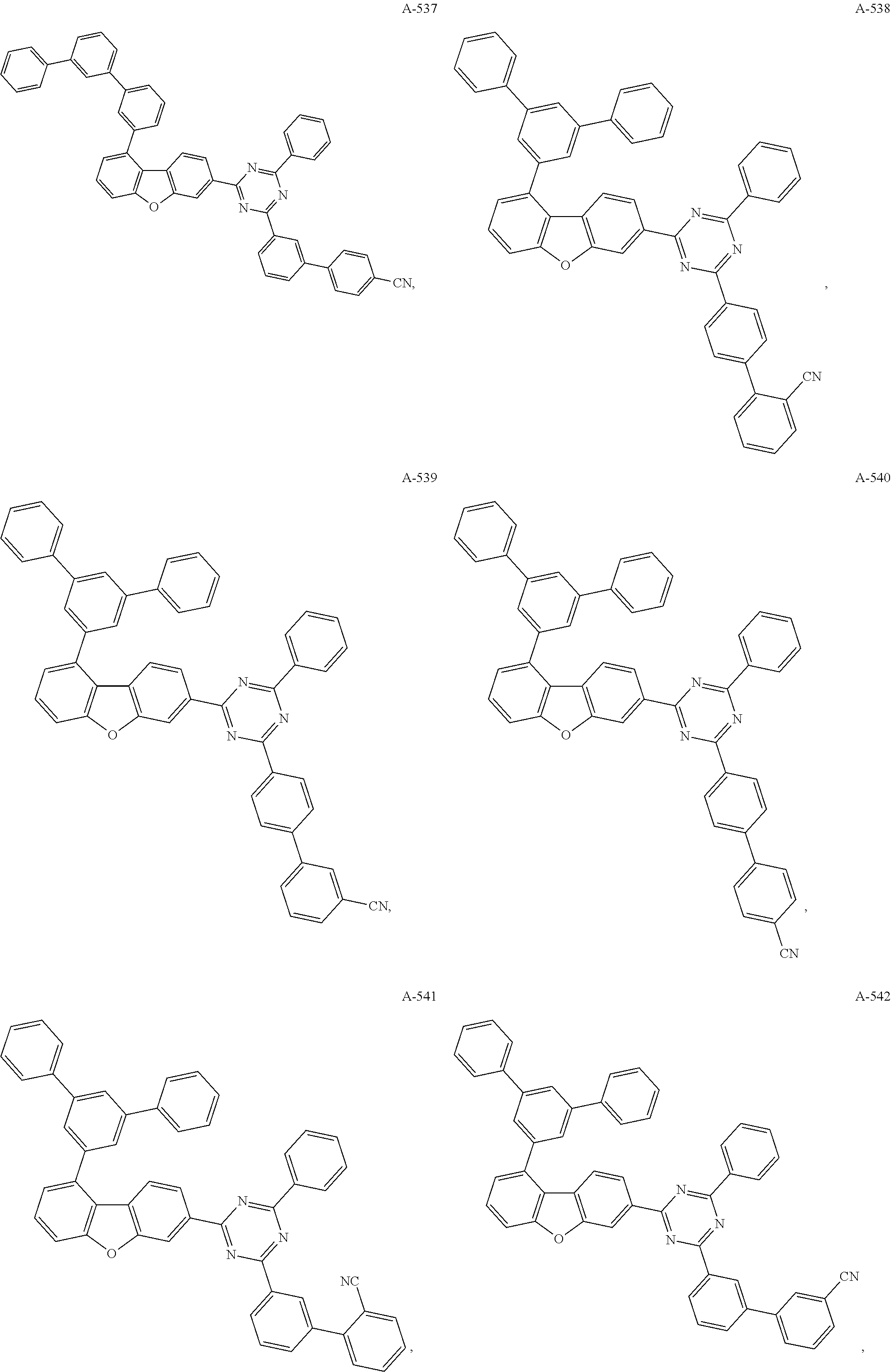 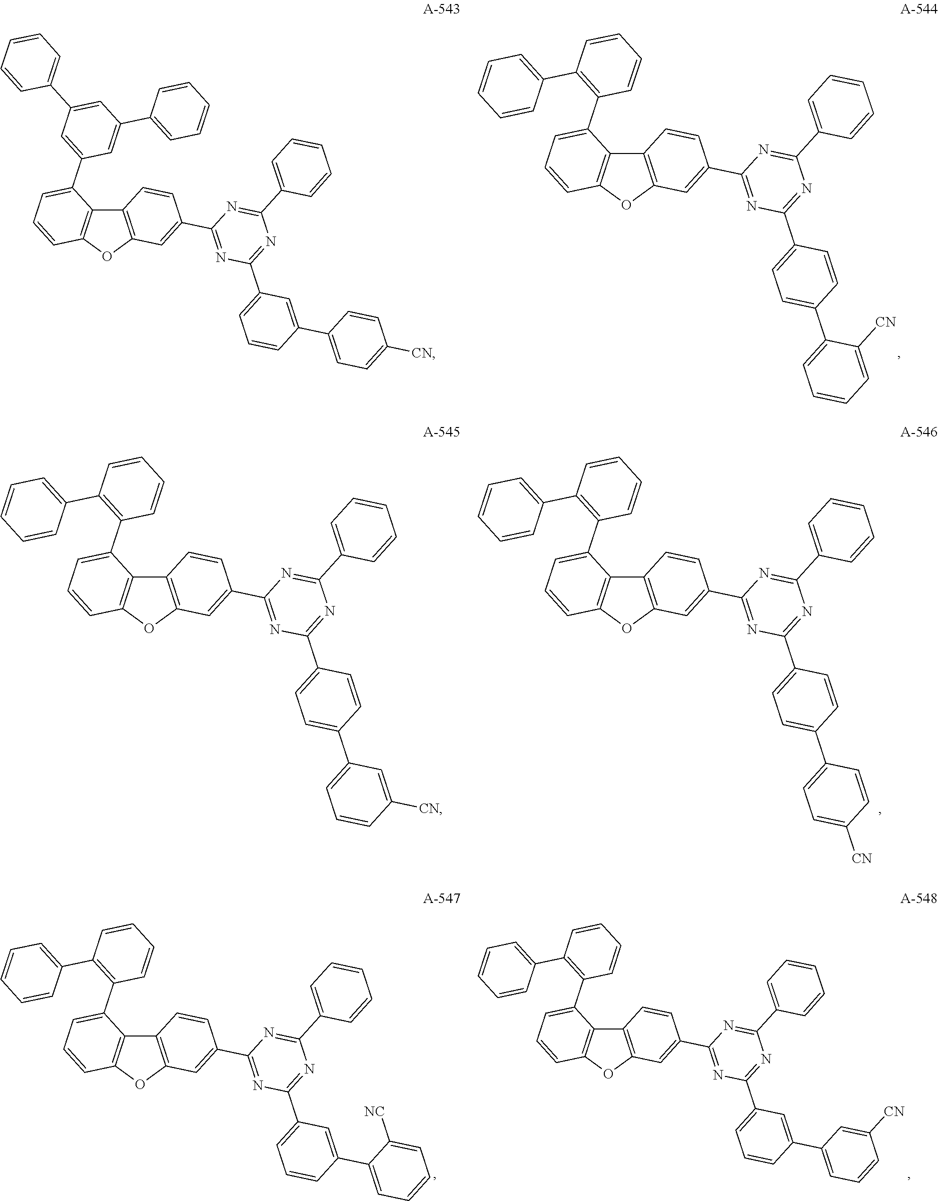 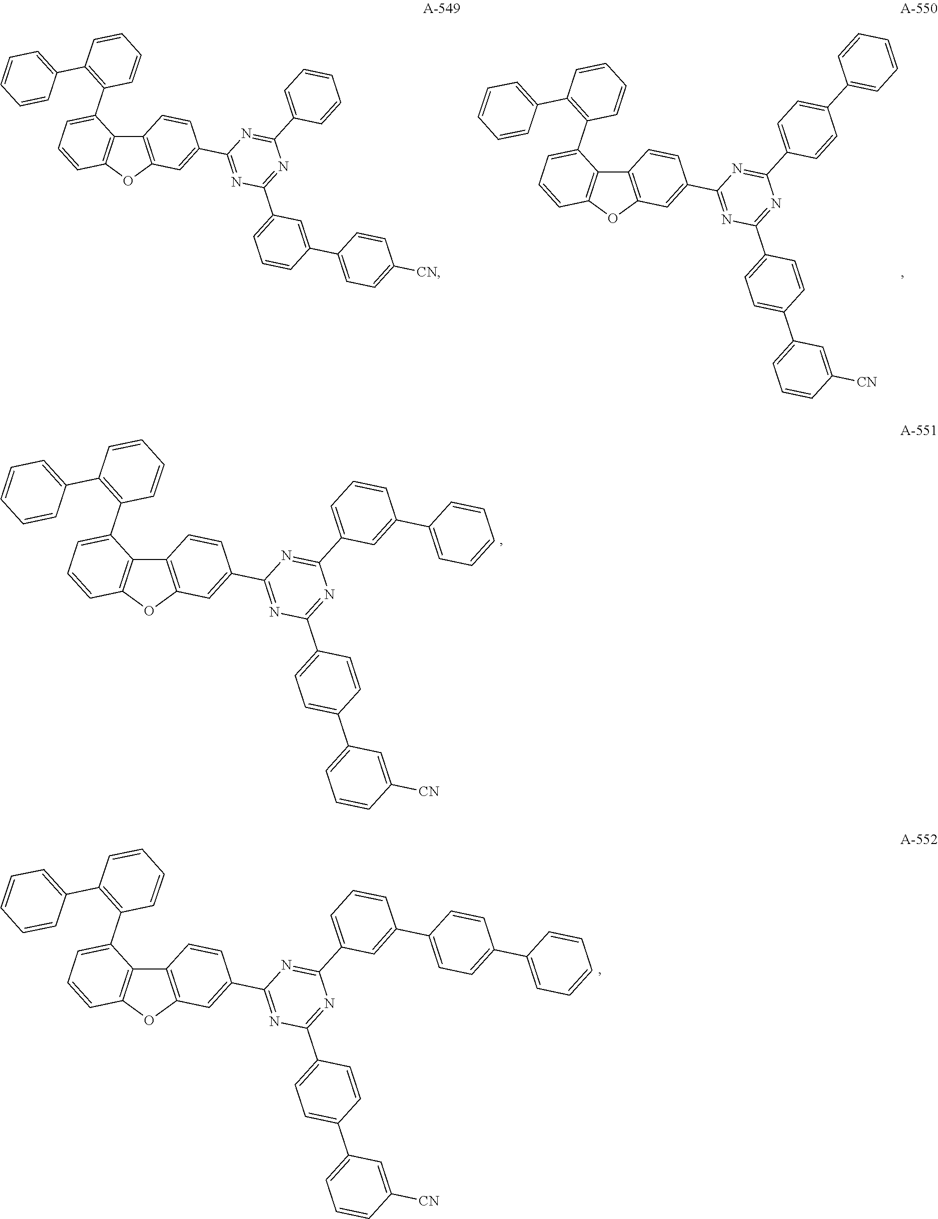 [SMILES: N#Cc1ccc(-c2cccc(-c3nc(-c4ccccc4)nc(-c4ccc5c(c4)oc4cccc(-c6ccccc6-c6ccccc6)c45)n3)c2)cc1 ; N#Cc1cccc(-c2ccc(-c3nc(-c4ccc(-c5ccccc5)cc4)nc(-c4ccc5c(c4)oc4cccc(-c6ccccc6-c6ccccc6)c45)n3)cc2)c1 ; N#Cc1cccc(-c2ccc(-c3nc(-c4cccc(-c5ccccc5)c4)nc(-c4ccc5c(c4)oc4cccc(-c6ccccc6-c6ccccc6)c45)n3)cc2)c1 ; N#Cc1cccc(-c2ccc(-c3nc(-c4cccc(-c5ccc(-c6ccccc6)cc5)c4)nc(-c4ccc5c(c4)oc4cccc(-c6ccccc6-c6ccccc6)c45)n3)cc2)c1] 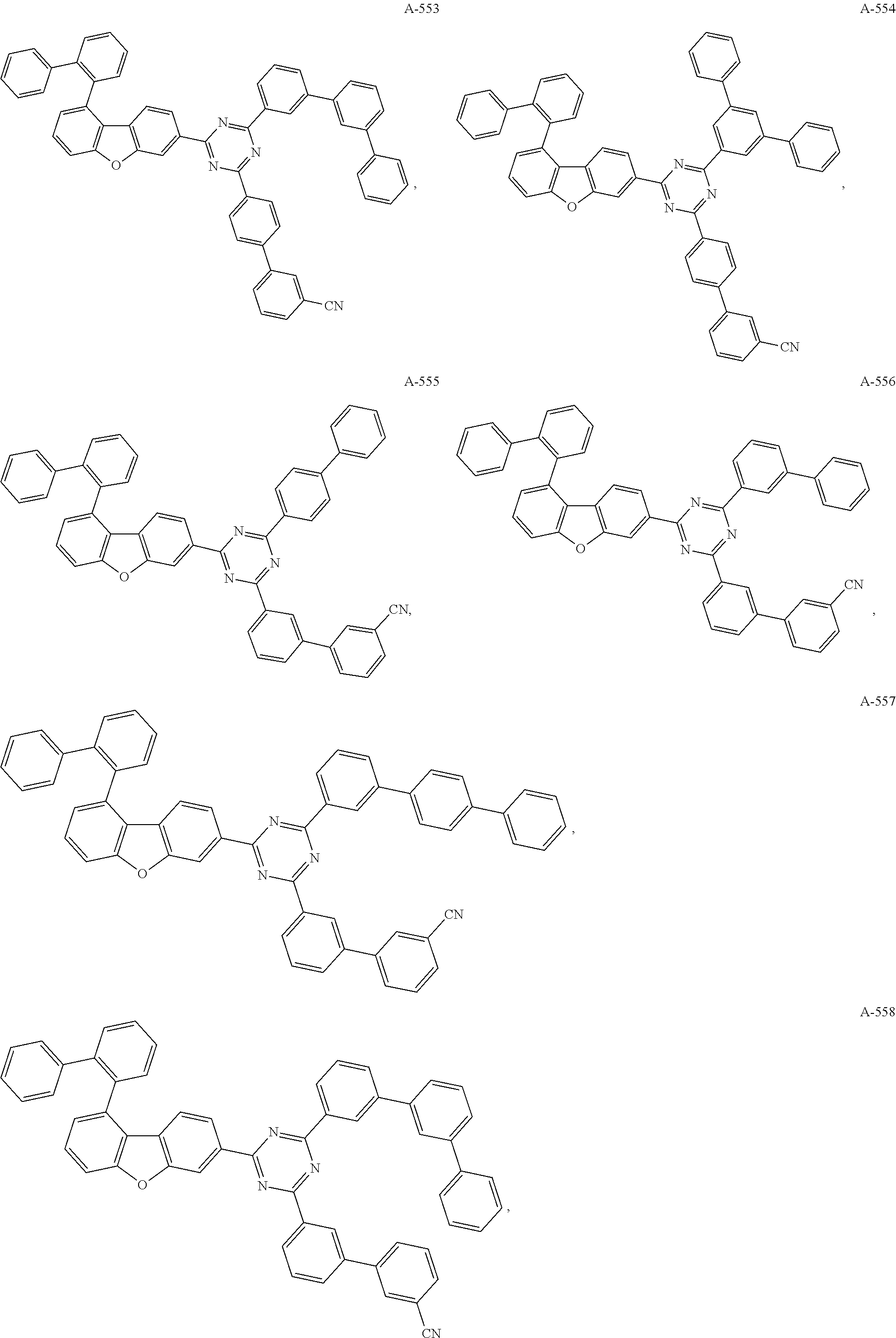 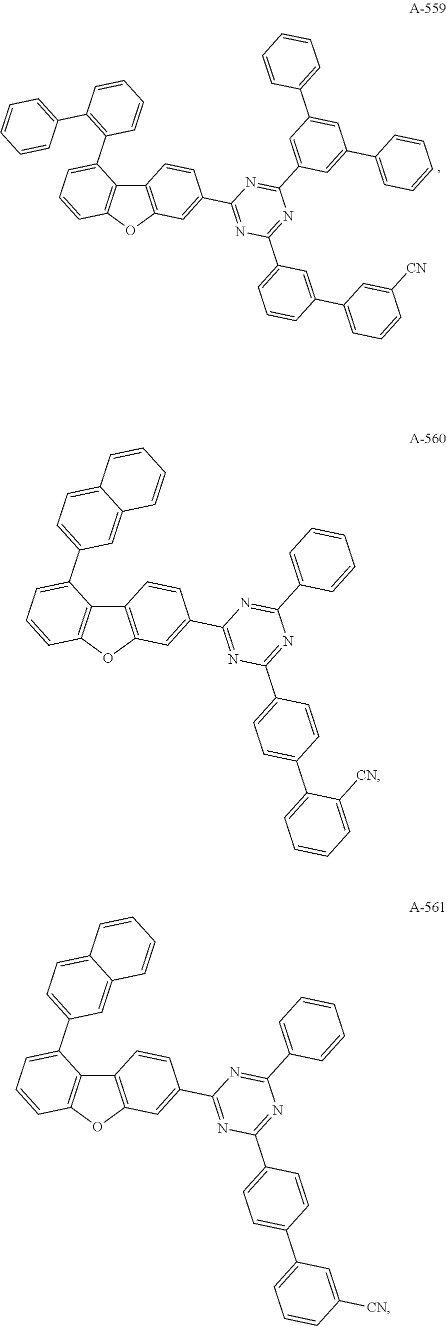 [SMILES: N#Cc1cccc(-c2cccc(-c3nc(-c4cc(-c5ccccc5)cc(-c5ccccc5)c4)nc(-c4ccc5c(c4)oc4cccc(-c6ccccc6-c6ccccc6)c45)n3)c2)c1 ; N#Cc1ccccc1-c1ccc(-c2nc(-c3ccccc3)nc(-c3ccc4c(c3)oc3cccc(-c5ccc6ccccc6c5)c34)n2)cc1 ; N#Cc1cccc(-c2ccc(-c3nc(-c4ccccc4)nc(-c4ccc5c(c4)oc4cccc(-c6ccc7ccccc7c6)c45)n3)cc2)c1] 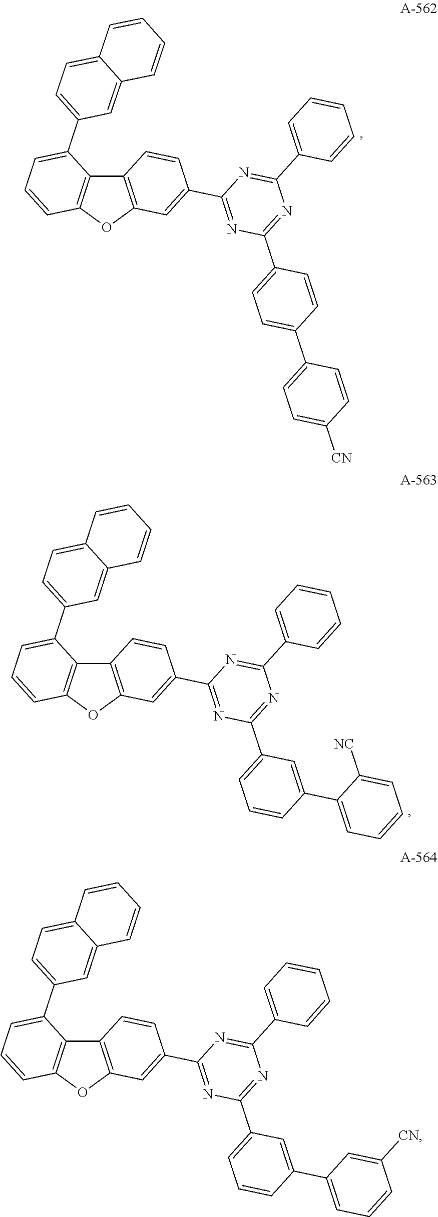 [SMILES: N#Cc1ccc(-c2ccc(-c3nc(-c4ccccc4)nc(-c4ccc5c(c4)oc4cccc(-c6ccc7ccccc7c6)c45)n3)cc2)cc1 ; N#Cc1ccccc1-c1cccc(-c2nc(-c3ccccc3)nc(-c3ccc4c(c3)oc3cccc(-c5ccc6ccccc6c5)c34)n2)c1 ; N#Cc1cccc(-c2cccc(-c3nc(-c4ccccc4)nc(-c4ccc5c(c4)oc4cccc(-c6ccc7ccccc7c6)c45)n3)c2)c1] 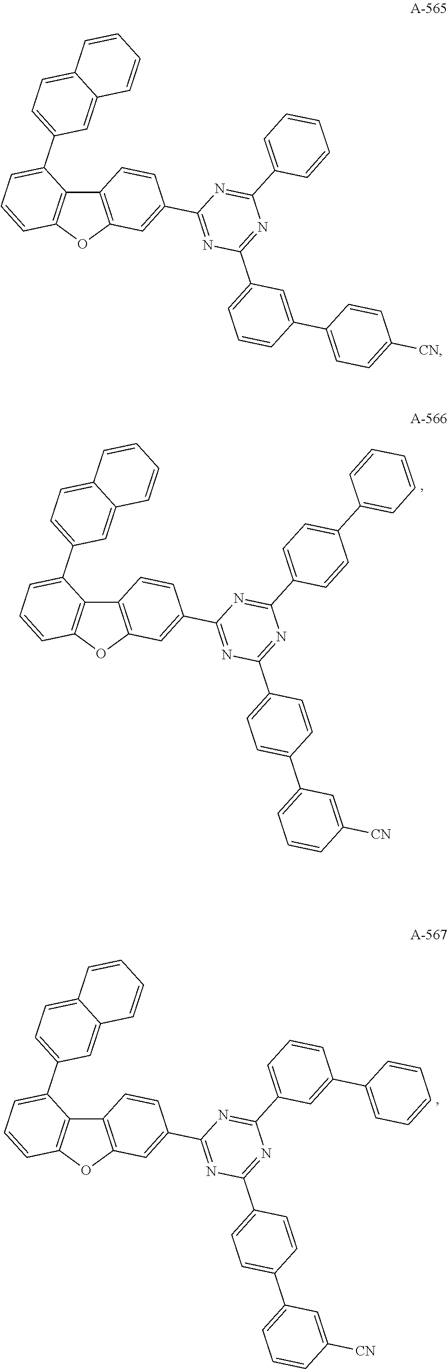 [SMILES: N#Cc1ccc(-c2cccc(-c3nc(-c4ccccc4)nc(-c4ccc5c(c4)oc4cccc(-c6ccc7ccccc7c6)c45)n3)c2)cc1 ; N#Cc1cccc(-c2ccc(-c3nc(-c4ccc(-c5ccccc5)cc4)nc(-c4ccc5c(c4)oc4cccc(-c6ccc7ccccc7c6)c45)n3)cc2)c1 ; N#Cc1cccc(-c2ccc(-c3nc(-c4cccc(-c5ccccc5)c4)nc(-c4ccc5c(c4)oc4cccc(-c6ccc7ccccc7c6)c45)n3)cc2)c1] 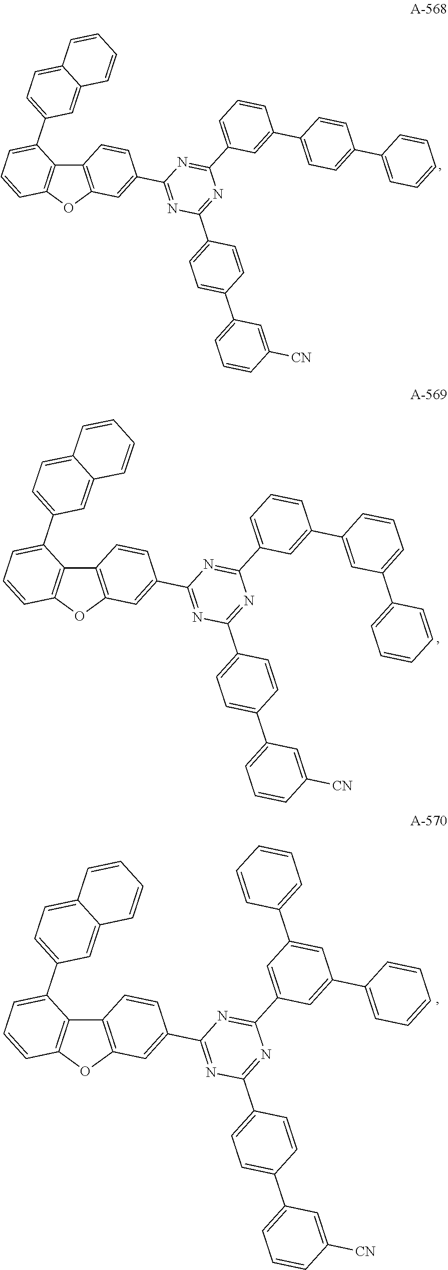 [SMILES: N#Cc1cccc(-c2ccc(-c3nc(-c4cccc(-c5ccc(-c6ccccc6)cc5)c4)nc(-c4ccc5c(c4)oc4cccc(-c6ccc7ccccc7c6)c45)n3)cc2)c1 ; N#Cc1cccc(-c2ccc(-c3nc(-c4cccc(-c5cccc(-c6ccccc6)c5)c4)nc(-c4ccc5c(c4)oc4cccc(-c6ccc7ccccc7c6)c45)n3)cc2)c1 ; N#Cc1cccc(-c2ccc(-c3nc(-c4cc(-c5ccccc5)cc(-c5ccccc5)c4)nc(-c4ccc5c(c4)oc4cccc(-c6ccc7ccccc7c6)c45)n3)cc2)c1] 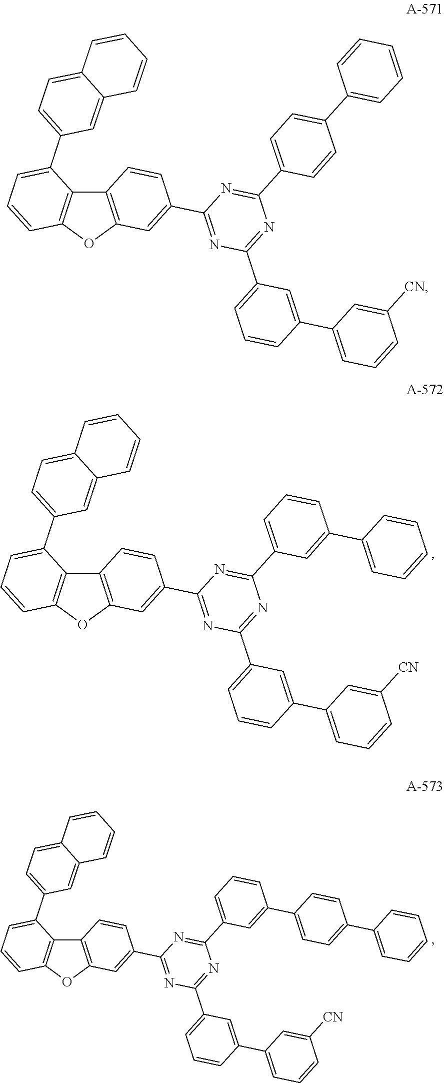 [SMILES: N#Cc1cccc(-c2cccc(-c3nc(-c4ccc(-c5ccccc5)cc4)nc(-c4ccc5c(c4)oc4cccc(-c6ccc7ccccc7c6)c45)n3)c2)c1 ; N#Cc1cccc(-c2cccc(-c3nc(-c4cccc(-c5ccccc5)c4)nc(-c4ccc5c(c4)oc4cccc(-c6ccc7ccccc7c6)c45)n3)c2)c1 ; N#Cc1cccc(-c2cccc(-c3nc(-c4cccc(-c5ccc(-c6ccccc6)cc5)c4)nc(-c4ccc5c(c4)oc4cccc(-c6ccc7ccccc7c6)c45)n3)c2)c1] 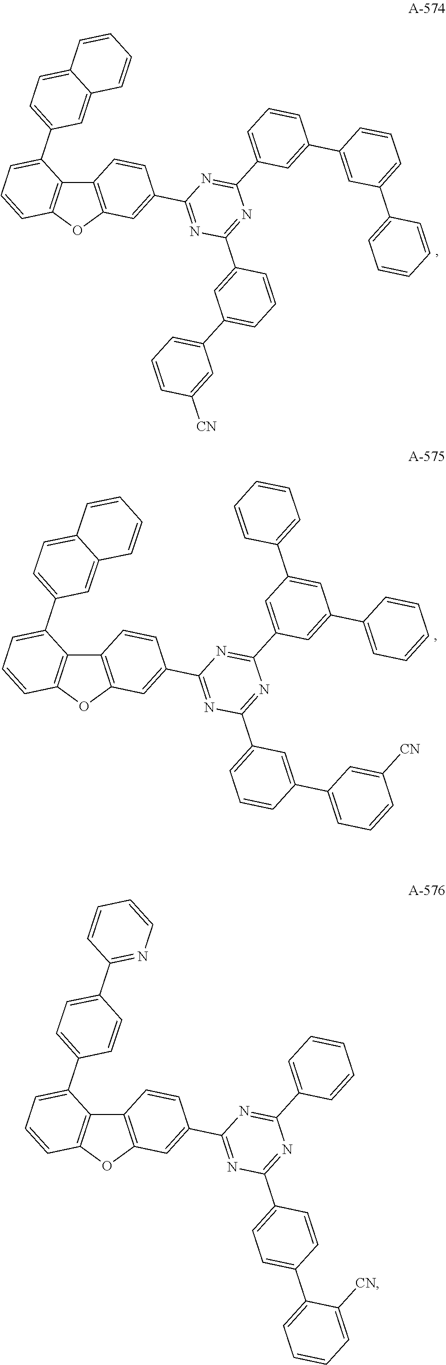 [SMILES: N#Cc1cccc(-c2cccc(-c3nc(-c4cccc(-c5cccc(-c6ccccc6)c5)c4)nc(-c4ccc5c(c4)oc4cccc(-c6ccc7ccccc7c6)c45)n3)c2)c1 ; N#Cc1cccc(-c2cccc(-c3nc(-c4cc(-c5ccccc5)cc(-c5ccccc5)c4)nc(-c4ccc5c(c4)oc4cccc(-c6ccc7ccccc7c6)c45)n3)c2)c1 ; N#Cc1ccccc1-c1ccc(-c2nc(-c3ccccc3)nc(-c3ccc4c(c3)oc3cccc(-c5ccc(-c6ccccn6)cc5)c34)n2)cc1] 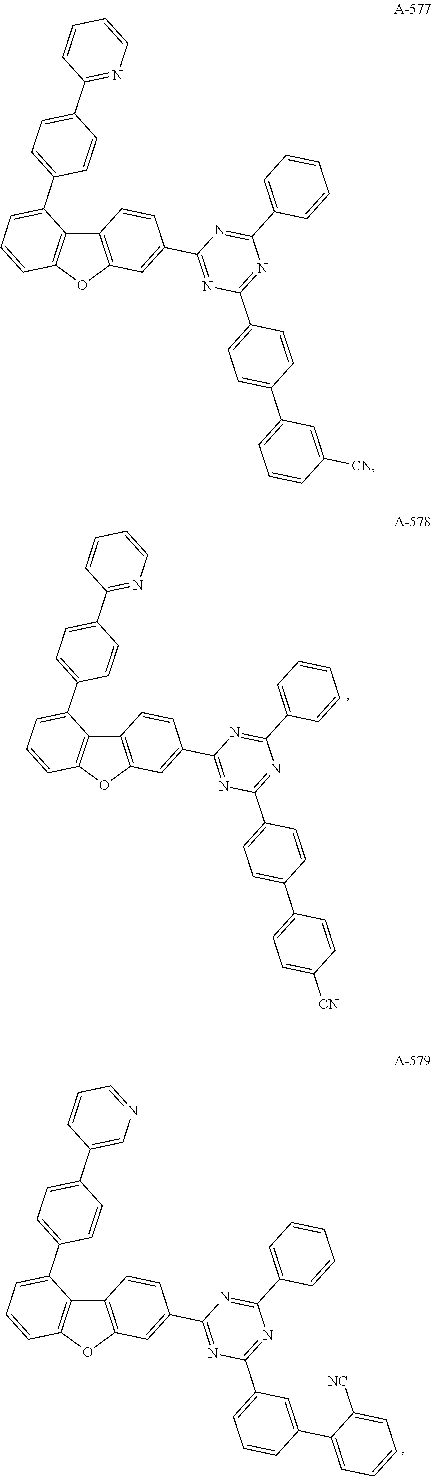 [SMILES: N#Cc1cccc(-c2ccc(-c3nc(-c4ccccc4)nc(-c4ccc5c(c4)oc4cccc(-c6ccc(-c7ccccn7)cc6)c45)n3)cc2)c1 ; N#Cc1ccc(-c2ccc(-c3nc(-c4ccccc4)nc(-c4ccc5c(c4)oc4cccc(-c6ccc(-c7ccccn7)cc6)c45)n3)cc2)cc1 ; N#Cc1ccccc1-c1cccc(-c2nc(-c3ccccc3)nc(-c3ccc4c(c3)oc3cccc(-c5ccc(-c6cccnc6)cc5)c34)n2)c1] 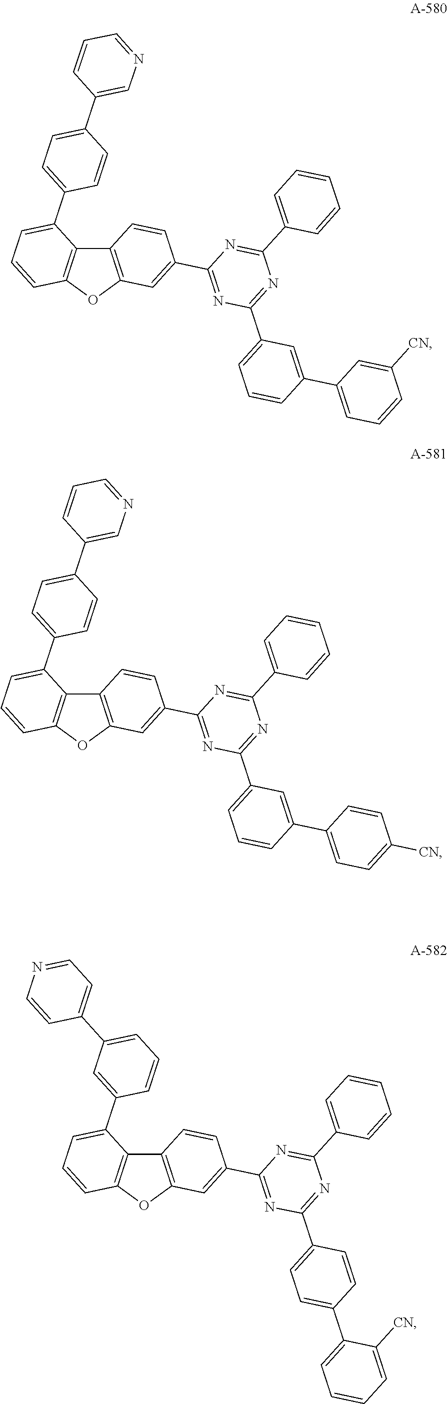 [SMILES: N#Cc1cccc(-c2cccc(-c3nc(-c4ccccc4)nc(-c4ccc5c(c4)oc4cccc(-c6ccc(-c7cccnc7)cc6)c45)n3)c2)c1 ; N#Cc1ccc(-c2cccc(-c3nc(-c4ccccc4)nc(-c4ccc5c(c4)oc4cccc(-c6ccc(-c7cccnc7)cc6)c45)n3)c2)cc1 ; N#Cc1ccccc1-c1ccc(-c2nc(-c3ccccc3)nc(-c3ccc4c(c3)oc3cccc(-c5cccc(-c6ccncc6)c5)c34)n2)cc1] 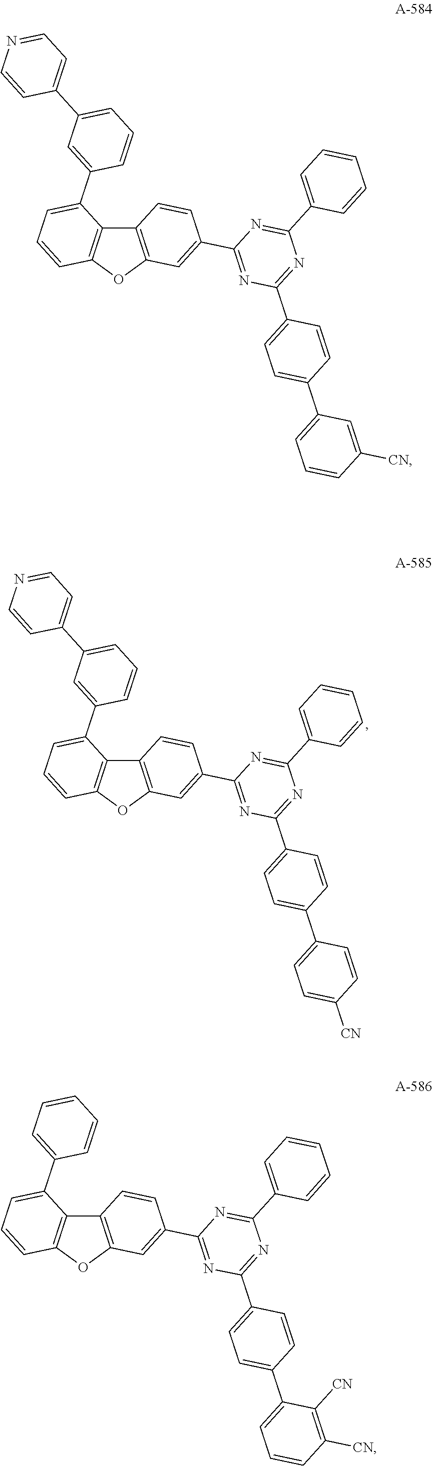 [SMILES: N#Cc1cccc(-c2ccc(-c3nc(-c4ccccc4)nc(-c4ccc5c(c4)oc4cccc(-c6cccc(-c7ccncc7)c6)c45)n3)cc2)c1 ; N#Cc1ccc(-c2ccc(-c3nc(-c4ccccc4)nc(-c4ccc5c(c4)oc4cccc(-c6cccc(-c7ccncc7)c6)c45)n3)cc2)cc1 ; N#Cc1cccc(-c2ccc(-c3nc(-c4ccccc4)nc(-c4ccc5c(c4)oc4cccc(-c6ccccc6)c45)n3)cc2)c1C#N] 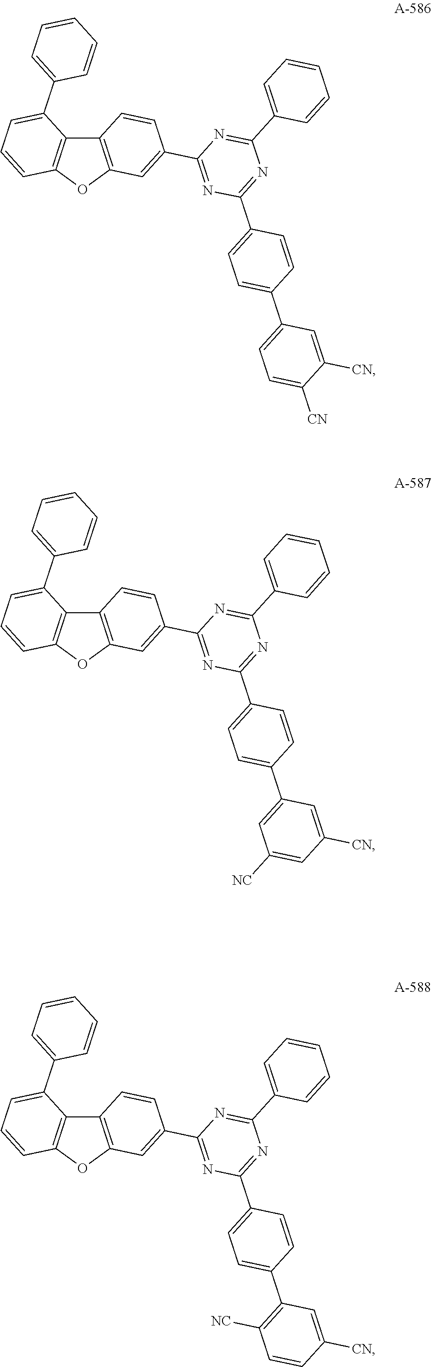 [SMILES: N#Cc1ccc(-c2ccc(-c3nc(-c4ccccc4)nc(-c4ccc5c(c4)oc4cccc(-c6ccccc6)c45)n3)cc2)cc1C#N ; N#Cc1cc(C#N)cc(-c2ccc(-c3nc(-c4ccccc4)nc(-c4ccc5c(c4)oc4cccc(-c6ccccc6)c45)n3)cc2)c1 ; N#Cc1ccc(C#N)c(-c2ccc(-c3nc(-c4ccccc4)nc(-c4ccc5c(c4)oc4cccc(-c6ccccc6)c45)n3)cc2)c1] 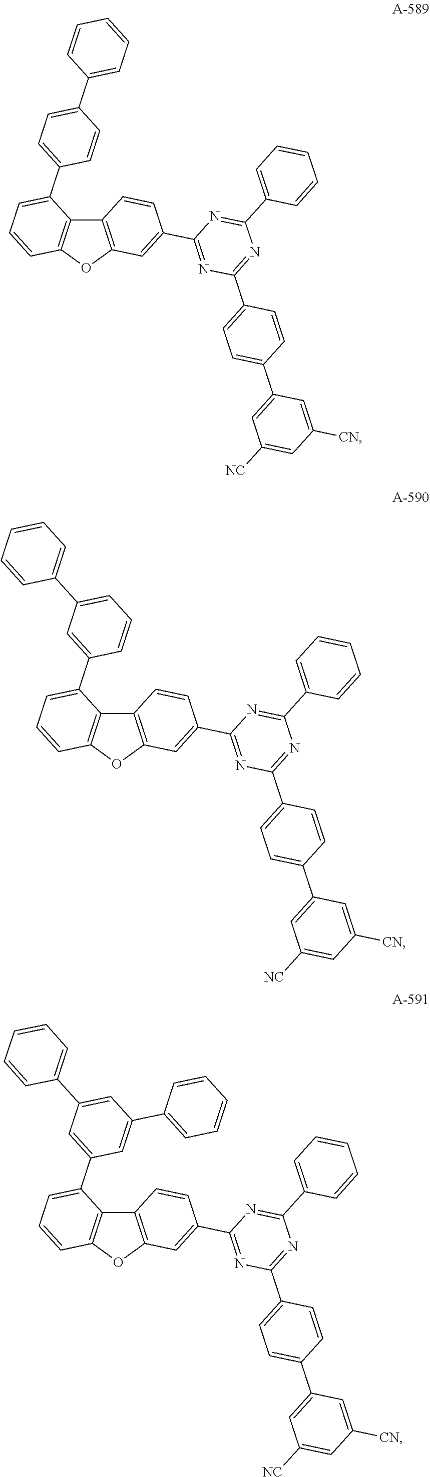 [SMILES: N#Cc1cc(C#N)cc(-c2ccc(-c3nc(-c4ccccc4)nc(-c4ccc5c(c4)oc4cccc(-c6ccc(-c7ccccc7)cc6)c45)n3)cc2)c1 ; N#Cc1cc(C#N)cc(-c2ccc(-c3nc(-c4ccccc4)nc(-c4ccc5c(c4)oc4cccc(-c6cccc(-c7ccccc7)c6)c45)n3)cc2)c1 ; N#Cc1cc(C#N)cc(-c2ccc(-c3nc(-c4ccccc4)nc(-c4ccc5c(c4)oc4cccc(-c6cc(-c7ccccc7)cc(-c7ccccc7)c6)c45)n3)cc2)c1] 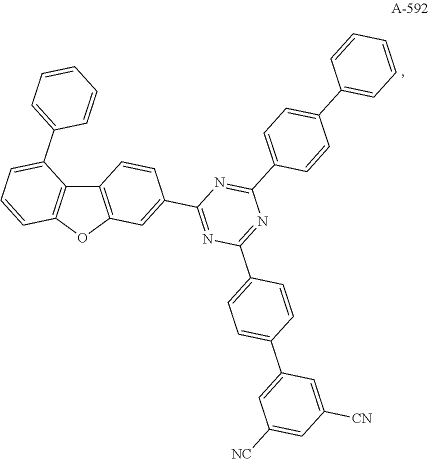 [SMILES: N#Cc1cc(C#N)cc(-c2ccc(-c3nc(-c4ccc(-c5ccccc5)cc4)nc(-c4ccc5c(c4)oc4cccc(-c6ccccc6)c45)n3)cc2)c1] 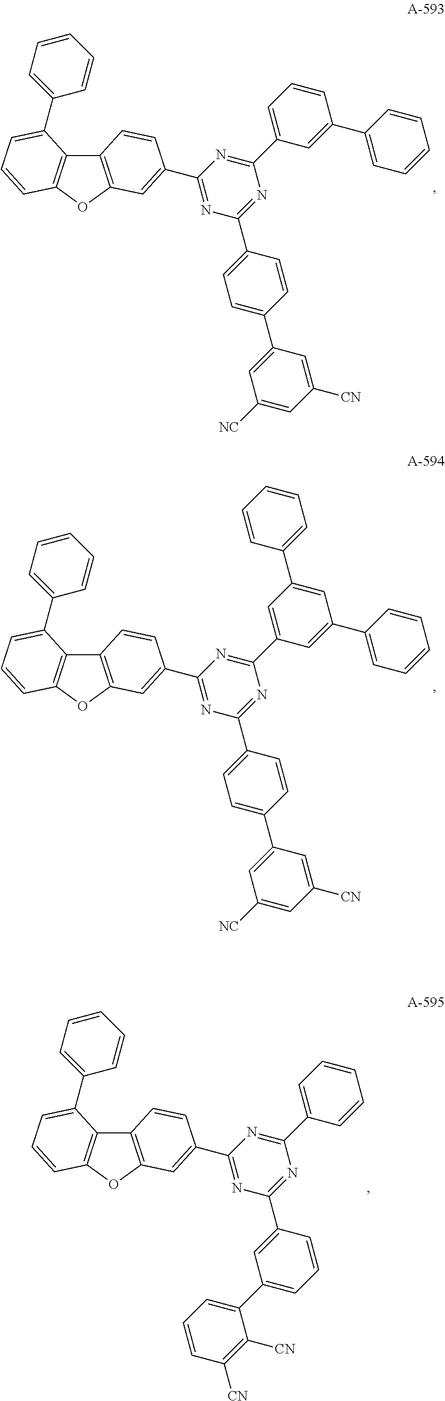 [SMILES: N#Cc1cc(C#N)cc(-c2ccc(-c3nc(-c4cccc(-c5ccccc5)c4)nc(-c4ccc5c(c4)oc4cccc(-c6ccccc6)c45)n3)cc2)c1 ; N#Cc1cc(C#N)cc(-c2ccc(-c3nc(-c4cc(-c5ccccc5)cc(-c5ccccc5)c4)nc(-c4ccc5c(c4)oc4cccc(-c6ccccc6)c45)n3)cc2)c1 ; N#Cc1cccc(-c2cccc(-c3nc(-c4ccccc4)nc(-c4ccc5c(c4)oc4cccc(-c6ccccc6)c45)n3)c2)c1C#N] 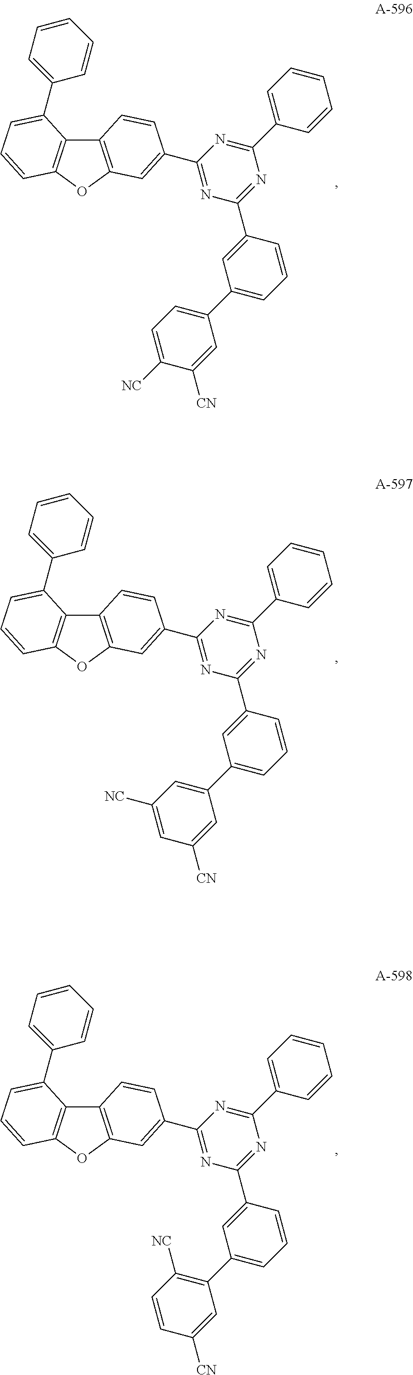 [SMILES: N#Cc1ccc(-c2cccc(-c3nc(-c4ccccc4)nc(-c4ccc5c(c4)oc4cccc(-c6ccccc6)c45)n3)c2)cc1C#N ; N#Cc1cc(C#N)cc(-c2cccc(-c3nc(-c4ccccc4)nc(-c4ccc5c(c4)oc4cccc(-c6ccccc6)c45)n3)c2)c1 ; N#Cc1ccc(C#N)c(-c2cccc(-c3nc(-c4ccccc4)nc(-c4ccc5c(c4)oc4cccc(-c6ccccc6)c45)n3)c2)c1] 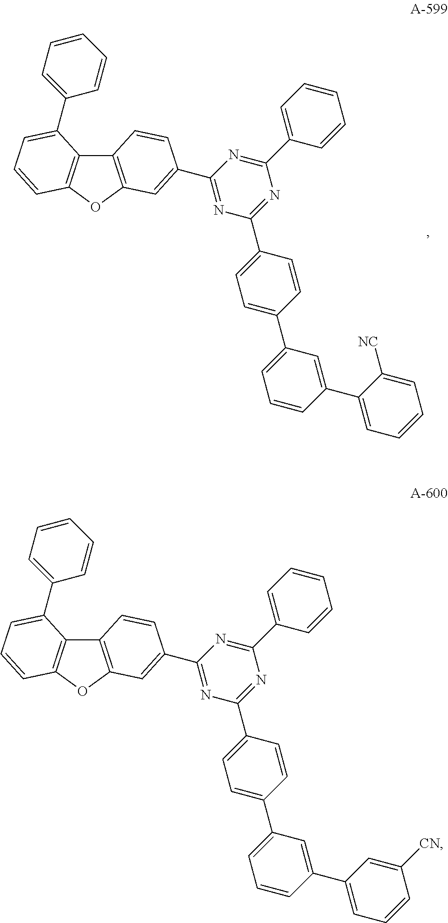 [SMILES: N#Cc1ccccc1-c1cccc(-c2ccc(-c3nc(-c4ccccc4)nc(-c4ccc5c(c4)oc4cccc(-c6ccccc6)c45)n3)cc2)c1 ; N#Cc1cccc(-c2cccc(-c3ccc(-c4nc(-c5ccccc5)nc(-c5ccc6c(c5)oc5cccc(-c7ccccc7)c56)n4)cc3)c2)c1] 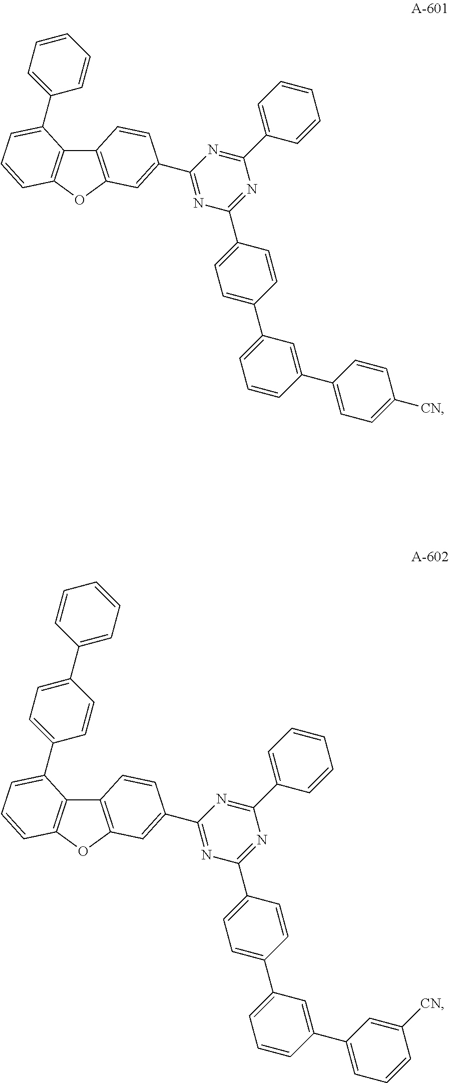 [SMILES: N#Cc1ccc(-c2cccc(-c3ccc(-c4nc(-c5ccccc5)nc(-c5ccc6c(c5)oc5cccc(-c7ccccc7)c56)n4)cc3)c2)cc1 ; N#Cc1cccc(-c2cccc(-c3ccc(-c4nc(-c5ccccc5)nc(-c5ccc6c(c5)oc5cccc(-c7ccc(-c8ccccc8)cc7)c56)n4)cc3)c2)c1] 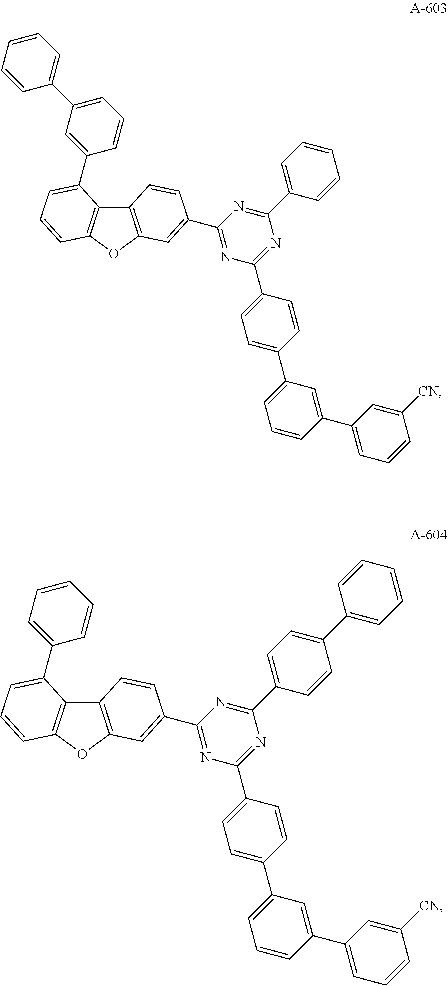 [SMILES: N#Cc1cccc(-c2cccc(-c3ccc(-c4nc(-c5ccccc5)nc(-c5ccc6c(c5)oc5cccc(-c7cccc(-c8ccccc8)c7)c56)n4)cc3)c2)c1 ; N#Cc1cccc(-c2cccc(-c3ccc(-c4nc(-c5ccc(-c6ccccc6)cc5)nc(-c5ccc6c(c5)oc5cccc(-c7ccccc7)c56)n4)cc3)c2)c1] 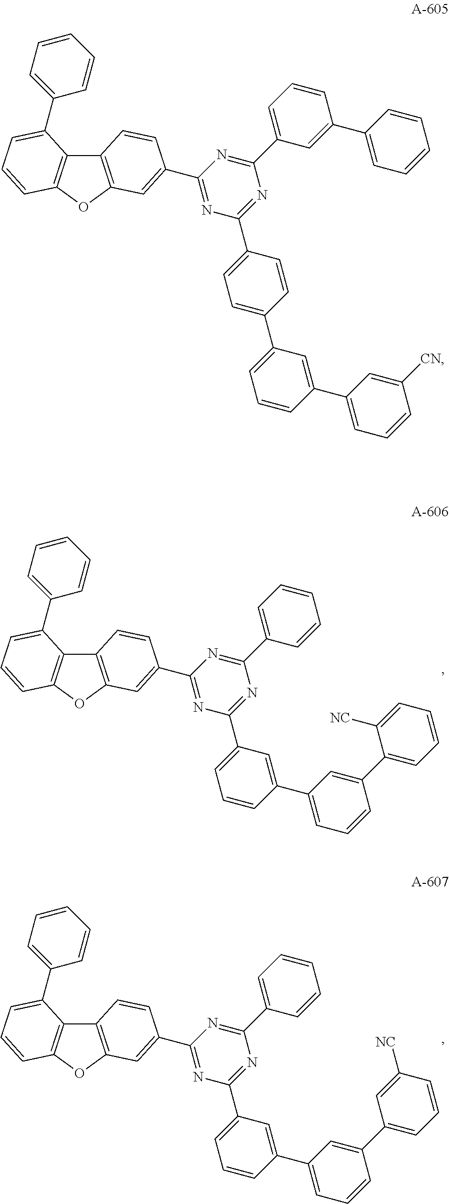 [SMILES: N#Cc1cccc(-c2cccc(-c3ccc(-c4nc(-c5cccc(-c6ccccc6)c5)nc(-c5ccc6c(c5)oc5cccc(-c7ccccc7)c56)n4)cc3)c2)c1 ; N#Cc1ccccc1-c1cccc(-c2cccc(-c3nc(-c4ccccc4)nc(-c4ccc5c(c4)oc4cccc(-c6ccccc6)c45)n3)c2)c1 ; N#Cc1cccc(-c2cccc(-c3cccc(-c4nc(-c5ccccc5)nc(-c5ccc6c(c5)oc5cccc(-c7ccccc7)c56)n4)c3)c2)c1] 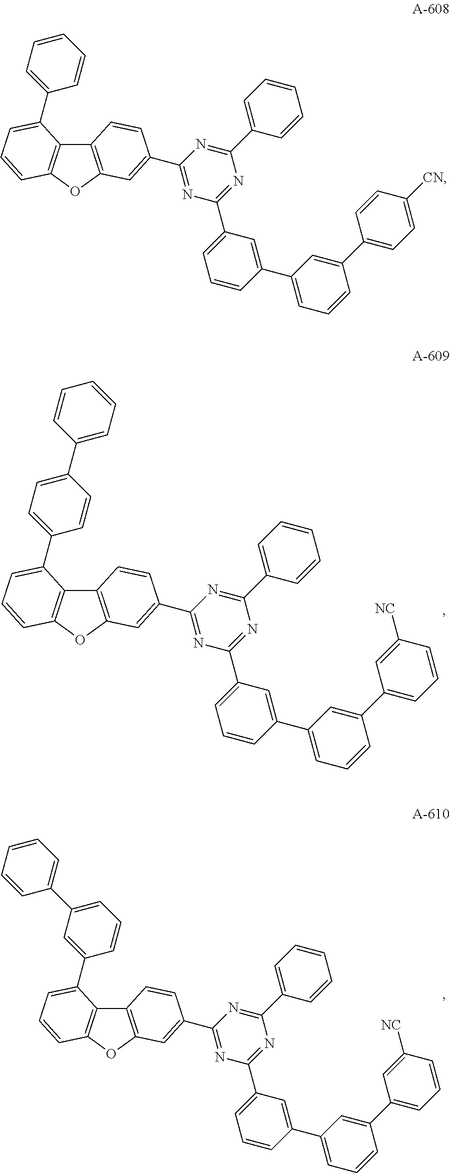 [SMILES: N#Cc1ccc(-c2cccc(-c3cccc(-c4nc(-c5ccccc5)nc(-c5ccc6c(c5)oc5cccc(-c7ccccc7)c56)n4)c3)c2)cc1 ; N#Cc1cccc(-c2cccc(-c3cccc(-c4nc(-c5ccccc5)nc(-c5ccc6c(c5)oc5cccc(-c7ccc(-c8ccccc8)cc7)c56)n4)c3)c2)c1 ; N#Cc1cccc(-c2cccc(-c3cccc(-c4nc(-c5ccccc5)nc(-c5ccc6c(c5)oc5cccc(-c7cccc(-c8ccccc8)c7)c56)n4)c3)c2)c1] 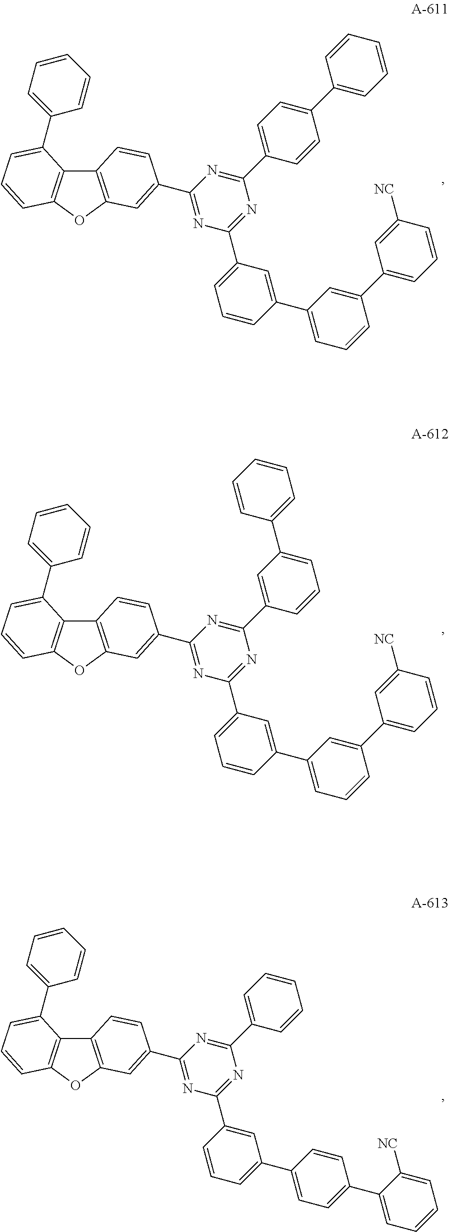 [SMILES: N#Cc1cccc(-c2cccc(-c3cccc(-c4nc(-c5ccc(-c6ccccc6)cc5)nc(-c5ccc6c(c5)oc5cccc(-c7ccccc7)c56)n4)c3)c2)c1 ; N#Cc1cccc(-c2cccc(-c3cccc(-c4nc(-c5cccc(-c6ccccc6)c5)nc(-c5ccc6c(c5)oc5cccc(-c7ccccc7)c56)n4)c3)c2)c1 ; N#Cc1ccccc1-c1ccc(-c2cccc(-c3nc(-c4ccccc4)nc(-c4ccc5c(c4)oc4cccc(-c6ccccc6)c45)n3)c2)cc1] 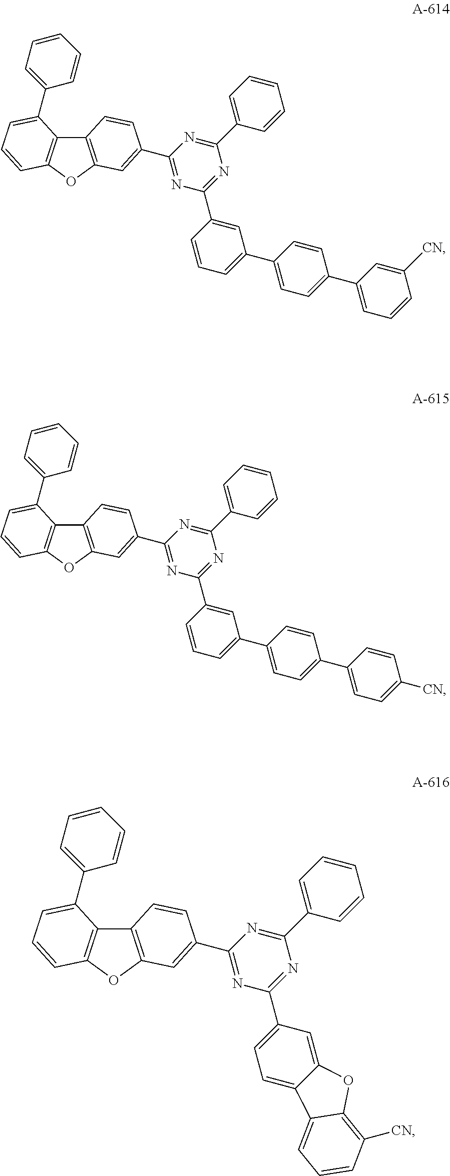 [SMILES: N#Cc1cccc(-c2ccc(-c3cccc(-c4nc(-c5ccccc5)nc(-c5ccc6c(c5)oc5cccc(-c7ccccc7)c56)n4)c3)cc2)c1 ; N#Cc1ccc(-c2ccc(-c3cccc(-c4nc(-c5ccccc5)nc(-c5ccc6c(c5)oc5cccc(-c7ccccc7)c56)n4)c3)cc2)cc1 ; N#Cc1cccc2c1oc1cc(-c3nc(-c4ccccc4)nc(-c4ccc5c(c4)oc4cccc(-c6ccccc6)c45)n3)ccc12] 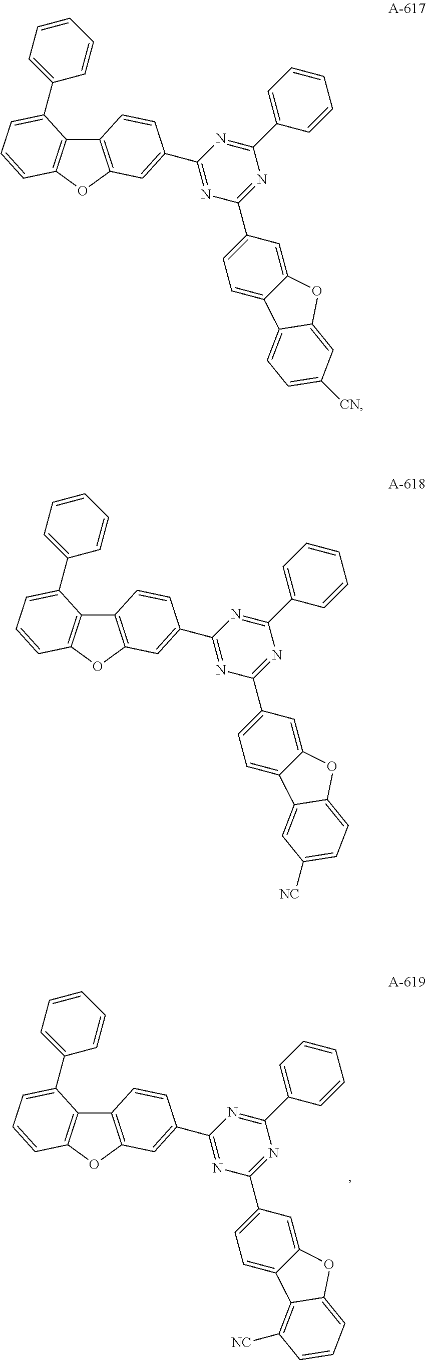 [SMILES: N#Cc1ccc2c(c1)oc1cc(-c3nc(-c4ccccc4)nc(-c4ccc5c(c4)oc4cccc(-c6ccccc6)c45)n3)ccc12 ; N#Cc1ccc2oc3cc(-c4nc(-c5ccccc5)nc(-c5ccc6c(c5)oc5cccc(-c7ccccc7)c56)n4)ccc3c2c1 ; N#Cc1cccc2oc3cc(-c4nc(-c5ccccc5)nc(-c5ccc6c(c5)oc5cccc(-c7ccccc7)c56)n4)ccc3c12] 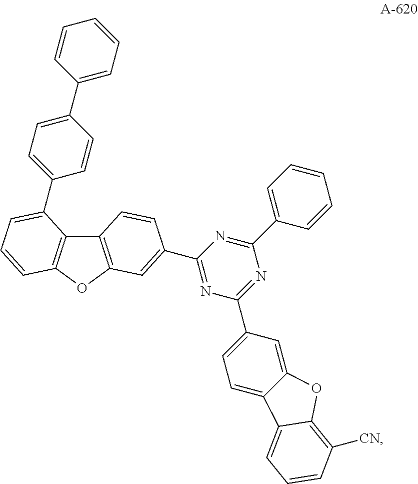 [SMILES: N#Cc1cccc2c1oc1cc(-c3nc(-c4ccccc4)nc(-c4ccc5c(c4)oc4cccc(-c6ccc(-c7ccccc7)cc6)c45)n3)ccc12] 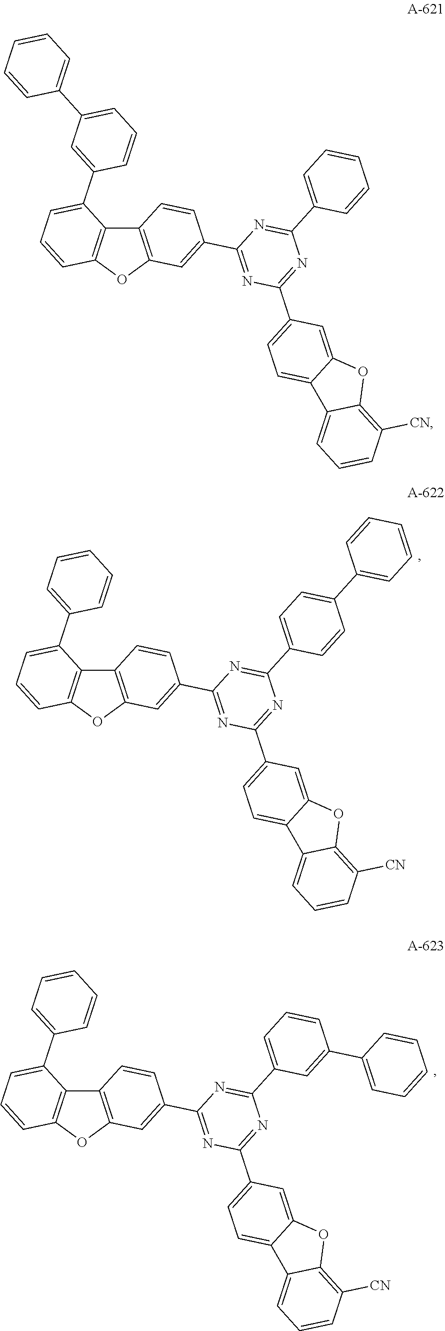 [SMILES: N#Cc1cccc2c1oc1cc(-c3nc(-c4ccccc4)nc(-c4ccc5c(c4)oc4cccc(-c6cccc(-c7ccccc7)c6)c45)n3)ccc12 ; N#Cc1cccc2c1oc1cc(-c3nc(-c4ccc(-c5ccccc5)cc4)nc(-c4ccc5c(c4)oc4cccc(-c6ccccc6)c45)n3)ccc12 ; N#Cc1cccc2c1oc1cc(-c3nc(-c4cccc(-c5ccccc5)c4)nc(-c4ccc5c(c4)oc4cccc(-c6ccccc6)c45)n3)ccc12] 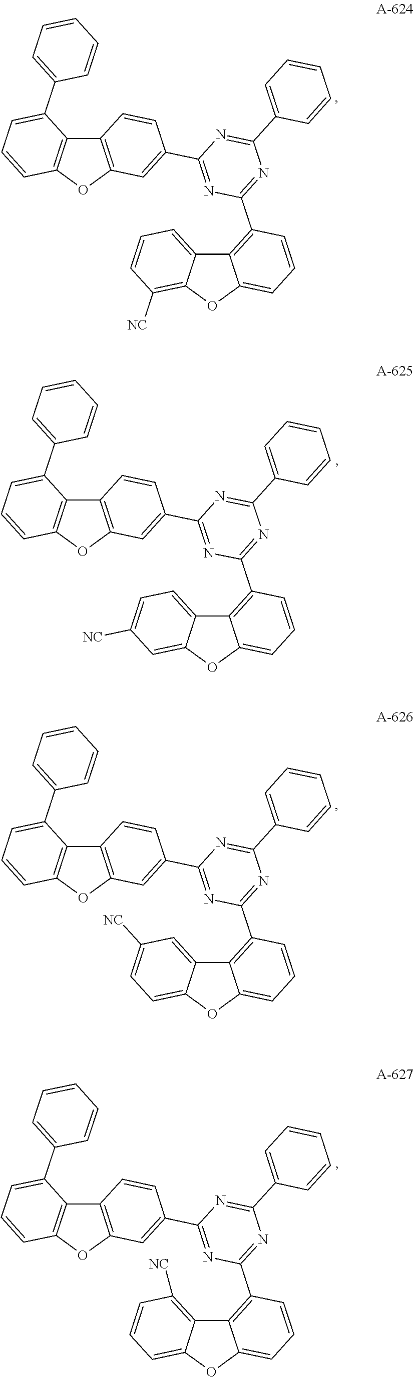 [SMILES: N#Cc1cccc2c1oc1cccc(-c3nc(-c4ccccc4)nc(-c4ccc5c(c4)oc4cccc(-c6ccccc6)c45)n3)c12 ; N#Cc1ccc2c(c1)oc1cccc(-c3nc(-c4ccccc4)nc(-c4ccc5c(c4)oc4cccc(-c6ccccc6)c45)n3)c12 ; N#Cc1ccc2oc3cccc(-c4nc(-c5ccccc5)nc(-c5ccc6c(c5)oc5cccc(-c7ccccc7)c56)n4)c3c2c1 ; N#Cc1cccc2oc3cccc(-c4nc(-c5ccccc5)nc(-c5ccc6c(c5)oc5cccc(-c7ccccc7)c56)n4)c3c12] 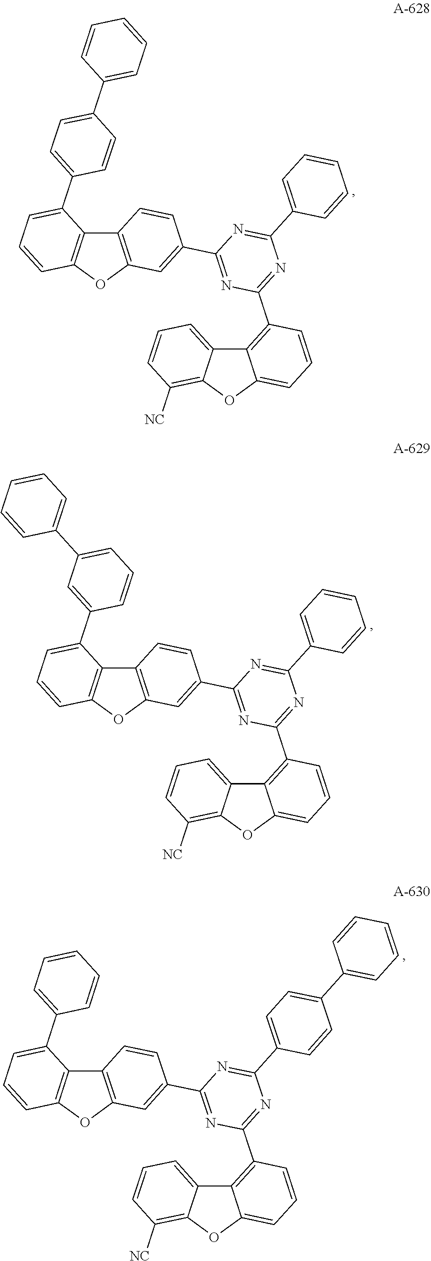 [SMILES: N#Cc1cccc2c1oc1cccc(-c3nc(-c4ccccc4)nc(-c4ccc5c(c4)oc4cccc(-c6ccc(-c7ccccc7)cc6)c45)n3)c12 ; N#Cc1cccc2c1oc1cccc(-c3nc(-c4ccccc4)nc(-c4ccc5c(c4)oc4cccc(-c6cccc(-c7ccccc7)c6)c45)n3)c12 ; N#Cc1cccc2c1oc1cccc(-c3nc(-c4ccc(-c5ccccc5)cc4)nc(-c4ccc5c(c4)oc4cccc(-c6ccccc6)c45)n3)c12] 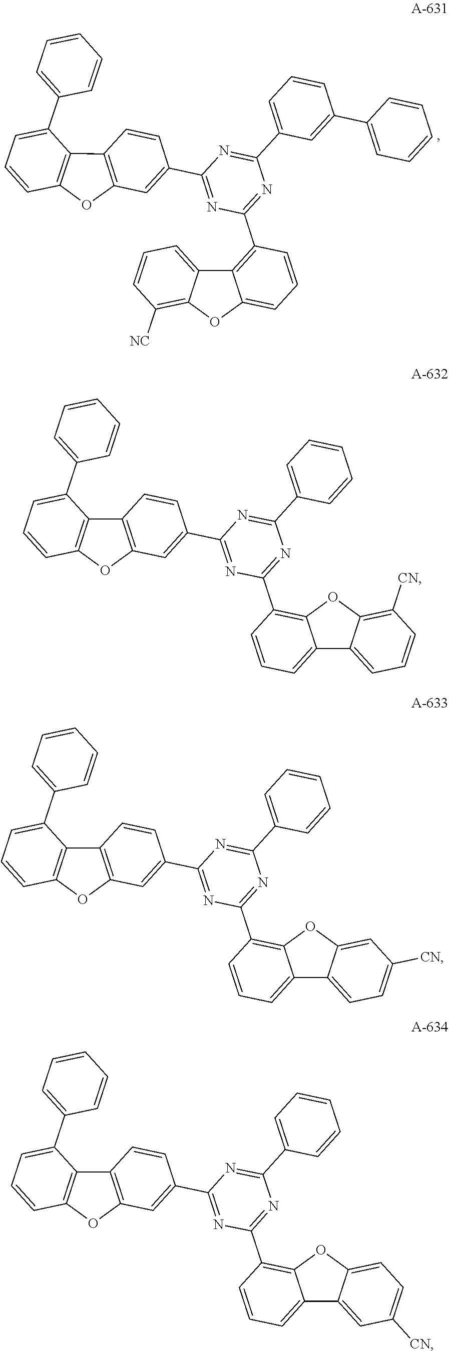 [SMILES: N#Cc1cccc2c1oc1cccc(-c3nc(-c4cccc(-c5ccccc5)c4)nc(-c4ccc5c(c4)oc4cccc(-c6ccccc6)c45)n3)c12 ; N#Cc1cccc2c1oc1c(-c3nc(-c4ccccc4)nc(-c4ccc5c(c4)oc4cccc(-c6ccccc6)c45)n3)cccc12 ; N#Cc1ccc2c(c1)oc1c(-c3nc(-c4ccccc4)nc(-c4ccc5c(c4)oc4cccc(-c6ccccc6)c45)n3)cccc12 ; N#Cc1ccc2oc3c(-c4nc(-c5ccccc5)nc(-c5ccc6c(c5)oc5cccc(-c7ccccc7)c56)n4)cccc3c2c1] 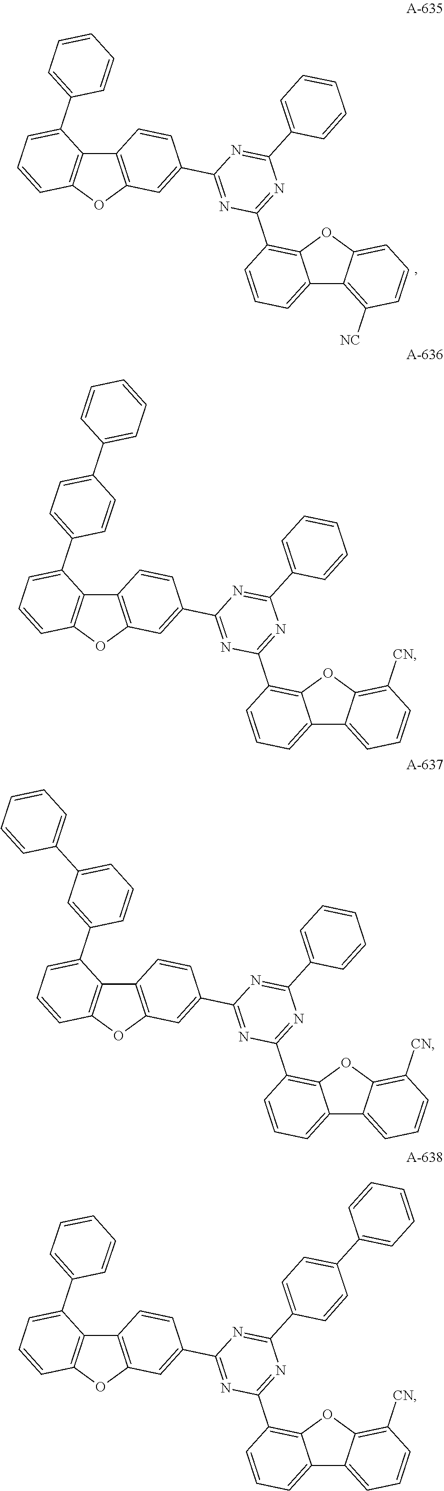 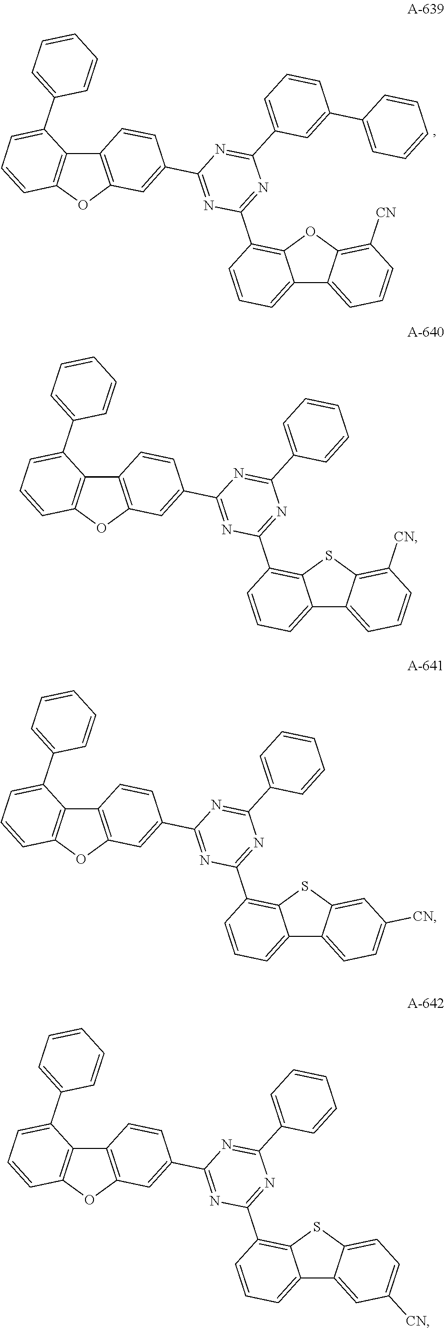 [SMILES: N#Cc1cccc2c1oc1c(-c3nc(-c4cccc(-c5ccccc5)c4)nc(-c4ccc5c(c4)oc4cccc(-c6ccccc6)c45)n3)cccc12 ; N#Cc1cccc2c1sc1c(-c3nc(-c4ccccc4)nc(-c4ccc5c(c4)oc4cccc(-c6ccccc6)c45)n3)cccc12 ; N#Cc1ccc2c(c1)sc1c(-c3nc(-c4ccccc4)nc(-c4ccc5c(c4)oc4cccc(-c6ccccc6)c45)n3)cccc12 ; N#Cc1ccc2sc3c(-c4nc(-c5ccccc5)nc(-c5ccc6c(c5)oc5cccc(-c7ccccc7)c56)n4)cccc3c2c1] 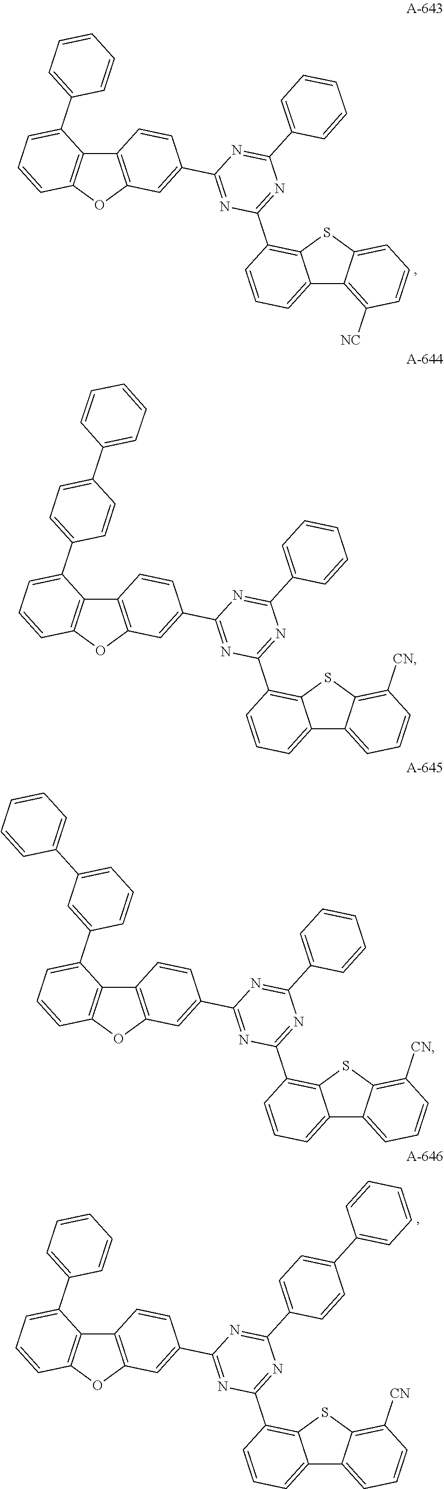 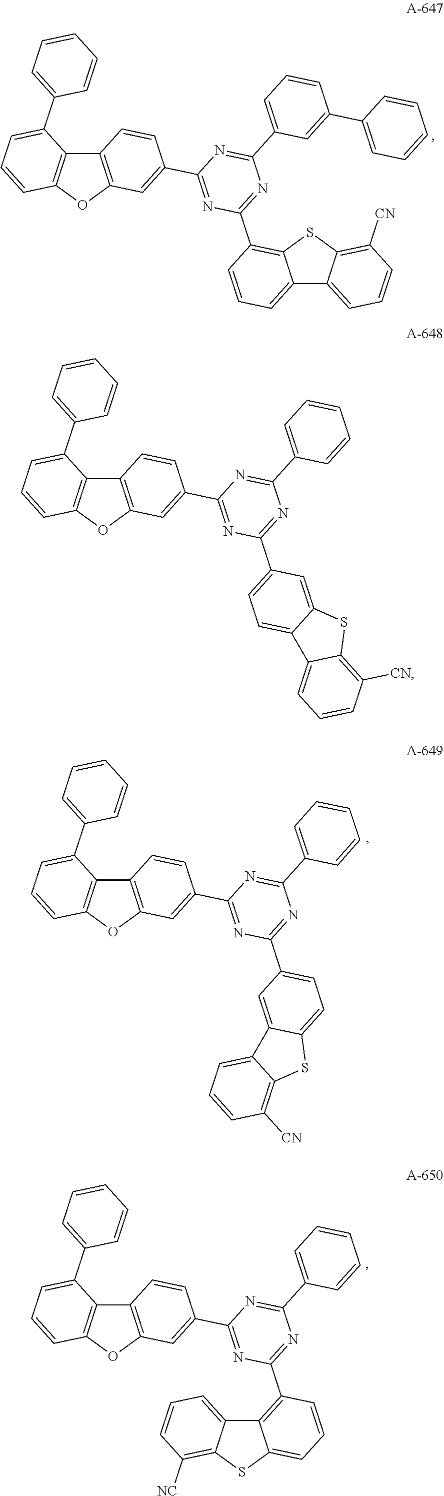 [SMILES: N#Cc1cccc2c1sc1c(-c3nc(-c4cccc(-c5ccccc5)c4)nc(-c4ccc5c(c4)oc4cccc(-c6ccccc6)c45)n3)cccc12 ; N#Cc1cccc2c1sc1cc(-c3nc(-c4ccccc4)nc(-c4ccc5c(c4)oc4cccc(-c6ccccc6)c45)n3)ccc12 ; N#Cc1cccc2c1sc1ccc(-c3nc(-c4ccccc4)nc(-c4ccc5c(c4)oc4cccc(-c6ccccc6)c45)n3)cc12 ; N#Cc1cccc2c1sc1cccc(-c3nc(-c4ccccc4)nc(-c4ccc5c(c4)oc4cccc(-c6ccccc6)c45)n3)c12] 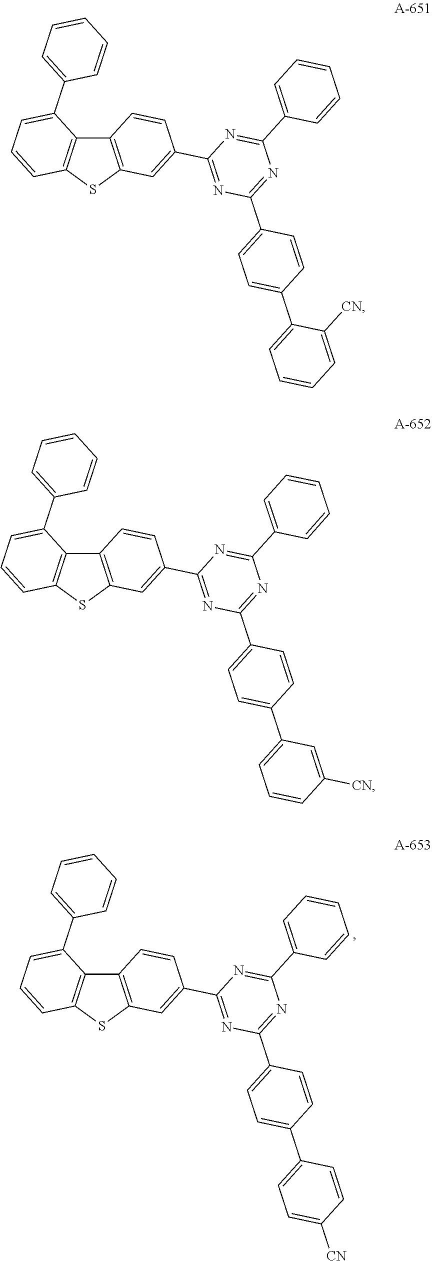 [SMILES: N#Cc1ccccc1-c1ccc(-c2nc(-c3ccccc3)nc(-c3ccc4c(c3)sc3cccc(-c5ccccc5)c34)n2)cc1 ; N#Cc1cccc(-c2ccc(-c3nc(-c4ccccc4)nc(-c4ccc5c(c4)sc4cccc(-c6ccccc6)c45)n3)cc2)c1 ; N#Cc1ccc(-c2ccc(-c3nc(-c4ccccc4)nc(-c4ccc5c(c4)sc4cccc(-c6ccccc6)c45)n3)cc2)cc1] 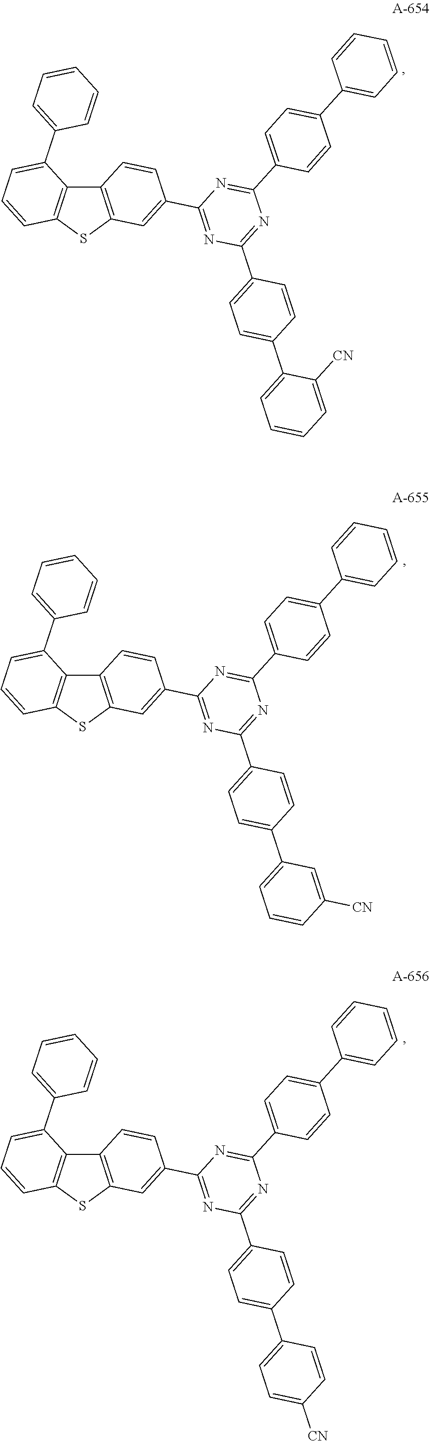 [SMILES: N#Cc1ccccc1-c1ccc(-c2nc(-c3ccc(-c4ccccc4)cc3)nc(-c3ccc4c(c3)sc3cccc(-c5ccccc5)c34)n2)cc1 ; N#Cc1cccc(-c2ccc(-c3nc(-c4ccc(-c5ccccc5)cc4)nc(-c4ccc5c(c4)sc4cccc(-c6ccccc6)c45)n3)cc2)c1 ; N#Cc1ccc(-c2ccc(-c3nc(-c4ccc(-c5ccccc5)cc4)nc(-c4ccc5c(c4)sc4cccc(-c6ccccc6)c45)n3)cc2)cc1] 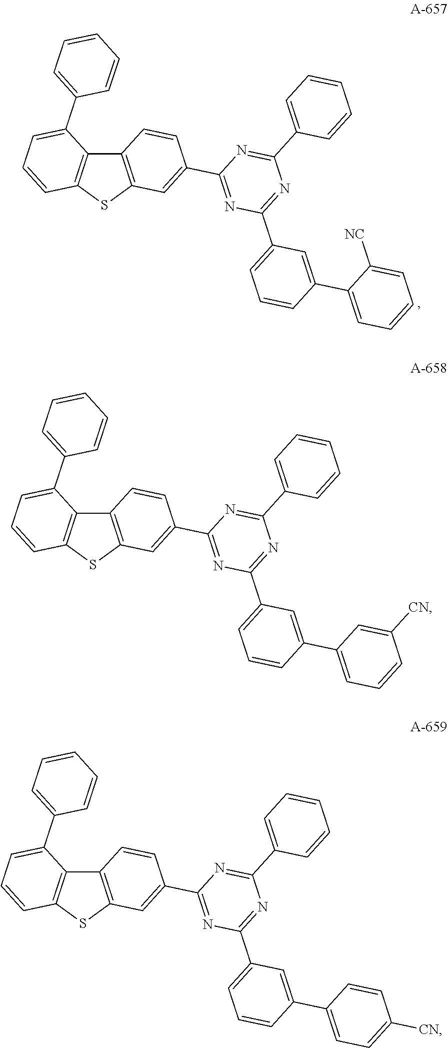 [SMILES: N#Cc1ccccc1-c1cccc(-c2nc(-c3ccccc3)nc(-c3ccc4c(c3)sc3cccc(-c5ccccc5)c34)n2)c1 ; N#Cc1cccc(-c2cccc(-c3nc(-c4ccccc4)nc(-c4ccc5c(c4)sc4cccc(-c6ccccc6)c45)n3)c2)c1 ; N#Cc1ccc(-c2cccc(-c3nc(-c4ccccc4)nc(-c4ccc5c(c4)sc4cccc(-c6ccccc6)c45)n3)c2)cc1] 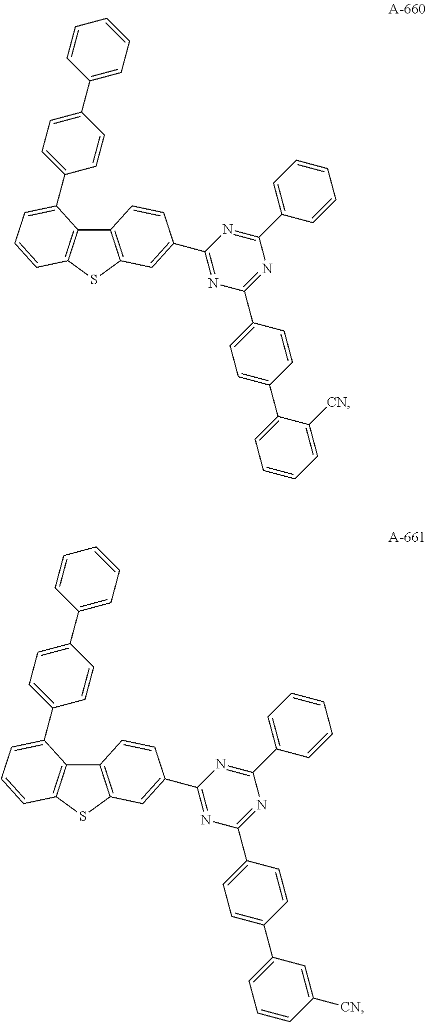 [SMILES: N#Cc1ccccc1-c1ccc(-c2nc(-c3ccccc3)nc(-c3ccc4c(c3)sc3cccc(-c5ccc(-c6ccccc6)cc5)c34)n2)cc1 ; N#Cc1cccc(-c2ccc(-c3nc(-c4ccccc4)nc(-c4ccc5c(c4)sc4cccc(-c6ccc(-c7ccccc7)cc6)c45)n3)cc2)c1] 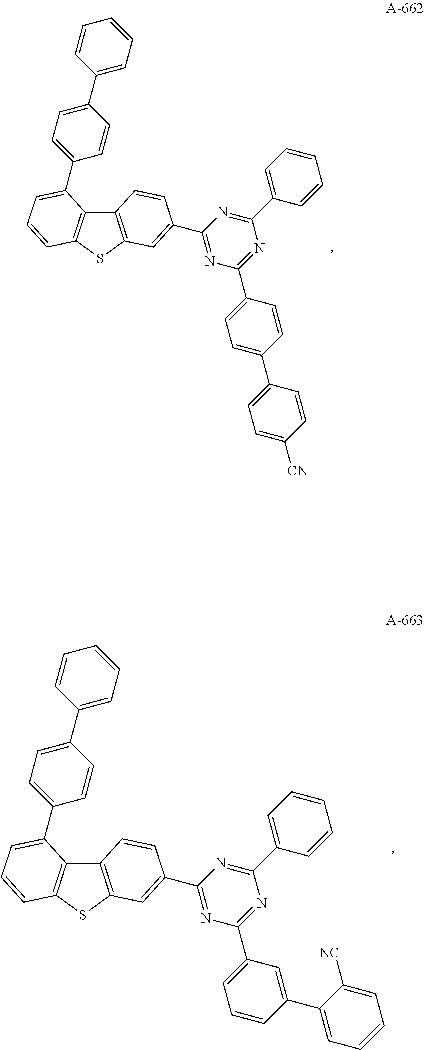 [SMILES: N#Cc1ccc(-c2ccc(-c3nc(-c4ccccc4)nc(-c4ccc5c(c4)sc4cccc(-c6ccc(-c7ccccc7)cc6)c45)n3)cc2)cc1 ; N#Cc1ccccc1-c1cccc(-c2nc(-c3ccccc3)nc(-c3ccc4c(c3)sc3cccc(-c5ccc(-c6ccccc6)cc5)c34)n2)c1] 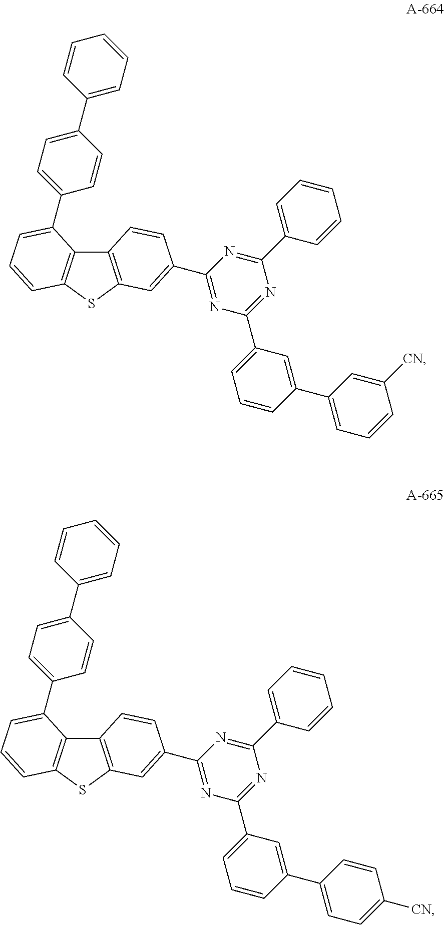 [SMILES: N#Cc1cccc(-c2cccc(-c3nc(-c4ccccc4)nc(-c4ccc5c(c4)sc4cccc(-c6ccc(-c7ccccc7)cc6)c45)n3)c2)c1 ; N#Cc1ccc(-c2cccc(-c3nc(-c4ccccc4)nc(-c4ccc5c(c4)sc4cccc(-c6ccc(-c7ccccc7)cc6)c45)n3)c2)cc1] 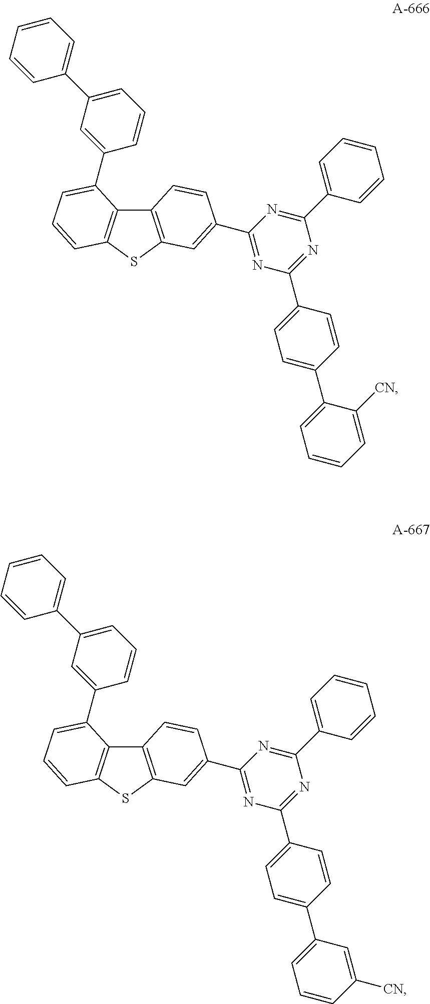 [SMILES: N#Cc1ccccc1-c1ccc(-c2nc(-c3ccccc3)nc(-c3ccc4c(c3)sc3cccc(-c5cccc(-c6ccccc6)c5)c34)n2)cc1 ; N#Cc1cccc(-c2ccc(-c3nc(-c4ccccc4)nc(-c4ccc5c(c4)sc4cccc(-c6cccc(-c7ccccc7)c6)c45)n3)cc2)c1] 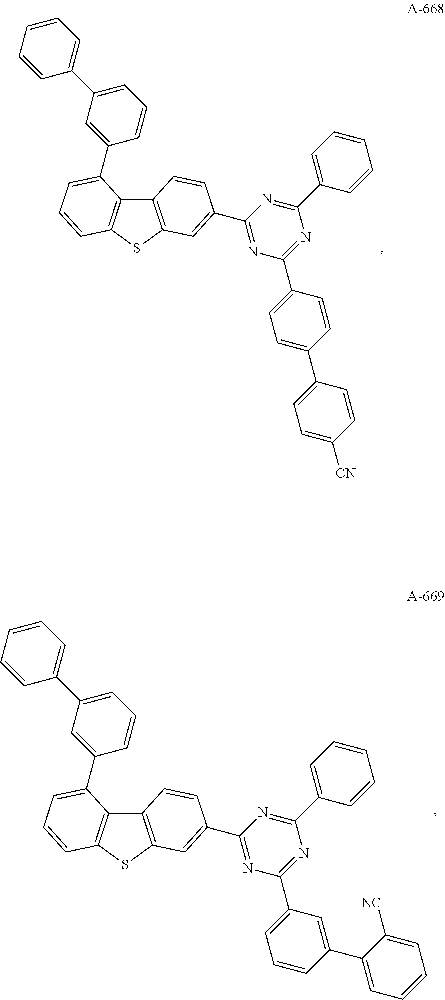 [SMILES: N#Cc1ccc(-c2ccc(-c3nc(-c4ccccc4)nc(-c4ccc5c(c4)sc4cccc(-c6cccc(-c7ccccc7)c6)c45)n3)cc2)cc1 ; N#Cc1ccccc1-c1cccc(-c2nc(-c3ccccc3)nc(-c3ccc4c(c3)sc3cccc(-c5cccc(-c6ccccc6)c5)c34)n2)c1] 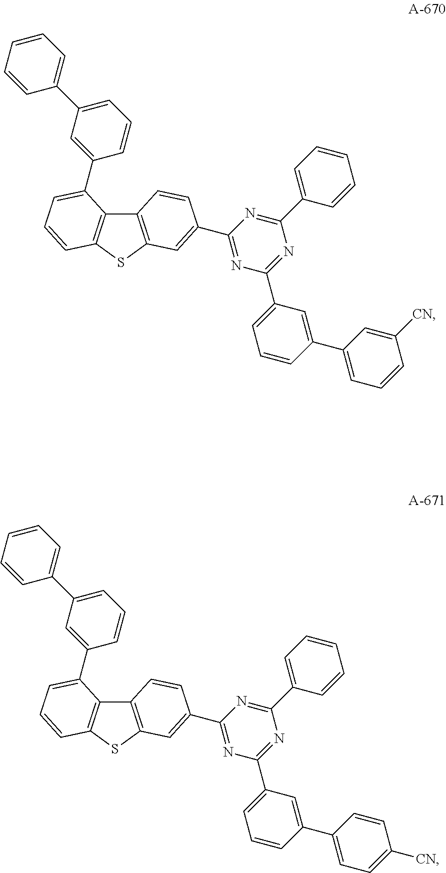 [SMILES: N#Cc1cccc(-c2cccc(-c3nc(-c4ccccc4)nc(-c4ccc5c(c4)sc4cccc(-c6cccc(-c7ccccc7)c6)c45)n3)c2)c1 ; N#Cc1ccc(-c2cccc(-c3nc(-c4ccccc4)nc(-c4ccc5c(c4)sc4cccc(-c6cccc(-c7ccccc7)c6)c45)n3)c2)cc1] 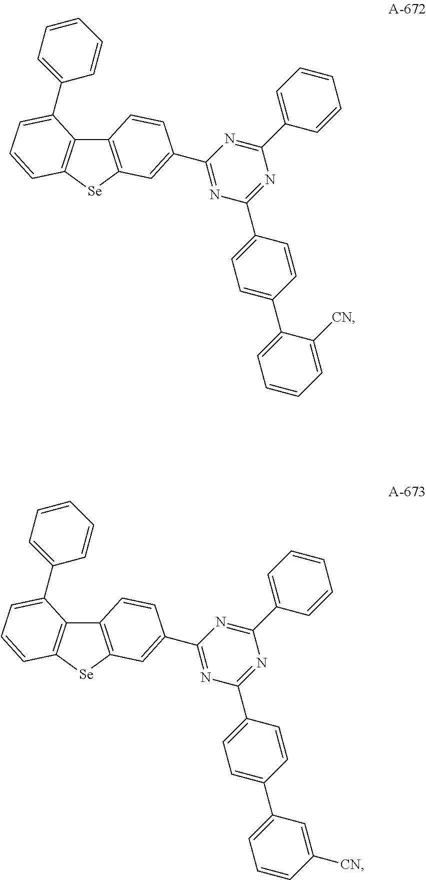 [SMILES: N#Cc1ccccc1-c1ccc(-c2nc(-c3ccccc3)nc(-c3ccc4c(c3)[se]c3cccc(-c5ccccc5)c34)n2)cc1 ; N#Cc1cccc(-c2ccc(-c3nc(-c4ccccc4)nc(-c4ccc5c(c4)[se]c4cccc(-c6ccccc6)c45)n3)cc2)c1] 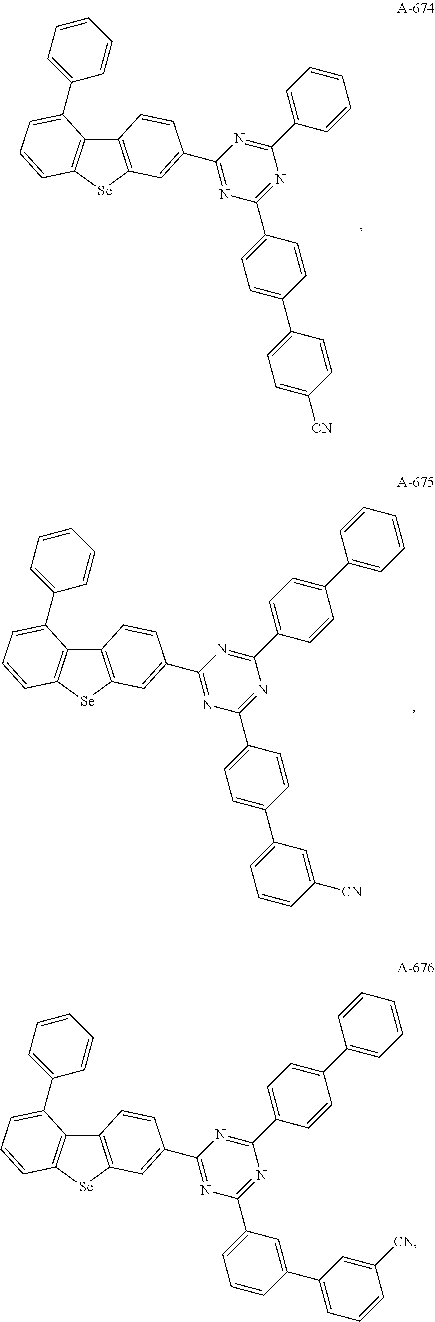 [SMILES: N#Cc1ccc(-c2ccc(-c3nc(-c4ccccc4)nc(-c4ccc5c(c4)[se]c4cccc(-c6ccccc6)c45)n3)cc2)cc1 ; N#Cc1cccc(-c2ccc(-c3nc(-c4ccc(-c5ccccc5)cc4)nc(-c4ccc5c(c4)[se]c4cccc(-c6ccccc6)c45)n3)cc2)c1 ; N#Cc1cccc(-c2cccc(-c3nc(-c4ccc(-c5ccccc5)cc4)nc(-c4ccc5c(c4)[se]c4cccc(-c6ccccc6)c45)n3)c2)c1] 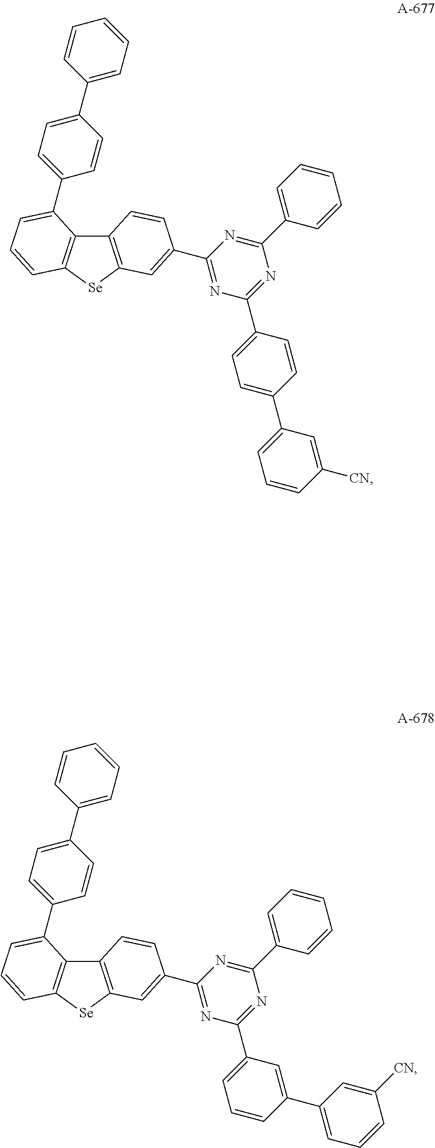 [SMILES: N#Cc1cccc(-c2ccc(-c3nc(-c4ccccc4)nc(-c4ccc5c(c4)[se]c4cccc(-c6ccc(-c7ccccc7)cc6)c45)n3)cc2)c1 ; N#Cc1cccc(-c2cccc(-c3nc(-c4ccccc4)nc(-c4ccc5c(c4)[se]c4cccc(-c6ccc(-c7ccccc7)cc6)c45)n3)c2)c1] 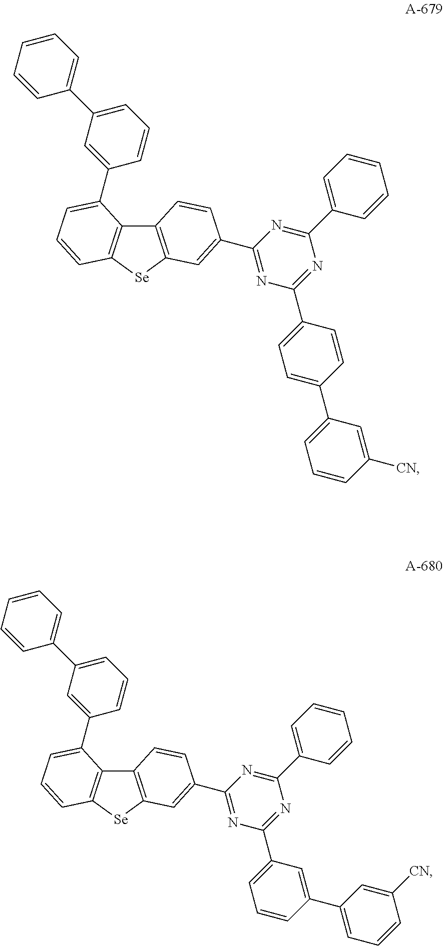 [SMILES: N#Cc1cccc(-c2ccc(-c3nc(-c4ccccc4)nc(-c4ccc5c(c4)[se]c4cccc(-c6cccc(-c7ccccc7)c6)c45)n3)cc2)c1 ; N#Cc1cccc(-c2cccc(-c3nc(-c4ccccc4)nc(-c4ccc5c(c4)[se]c4cccc(-c6cccc(-c7ccccc7)c6)c45)n3)c2)c1] 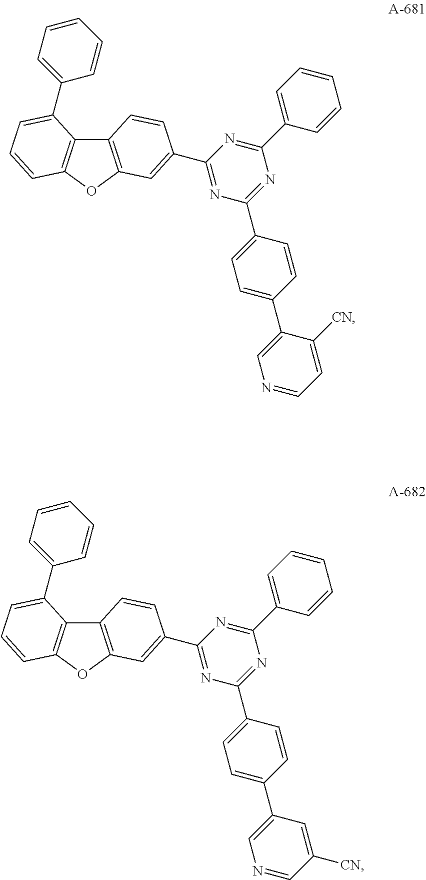 [SMILES: N#Cc1ccncc1-c1ccc(-c2nc(-c3ccccc3)nc(-c3ccc4c(c3)oc3cccc(-c5ccccc5)c34)n2)cc1 ; N#Cc1cncc(-c2ccc(-c3nc(-c4ccccc4)nc(-c4ccc5c(c4)oc4cccc(-c6ccccc6)c45)n3)cc2)c1] 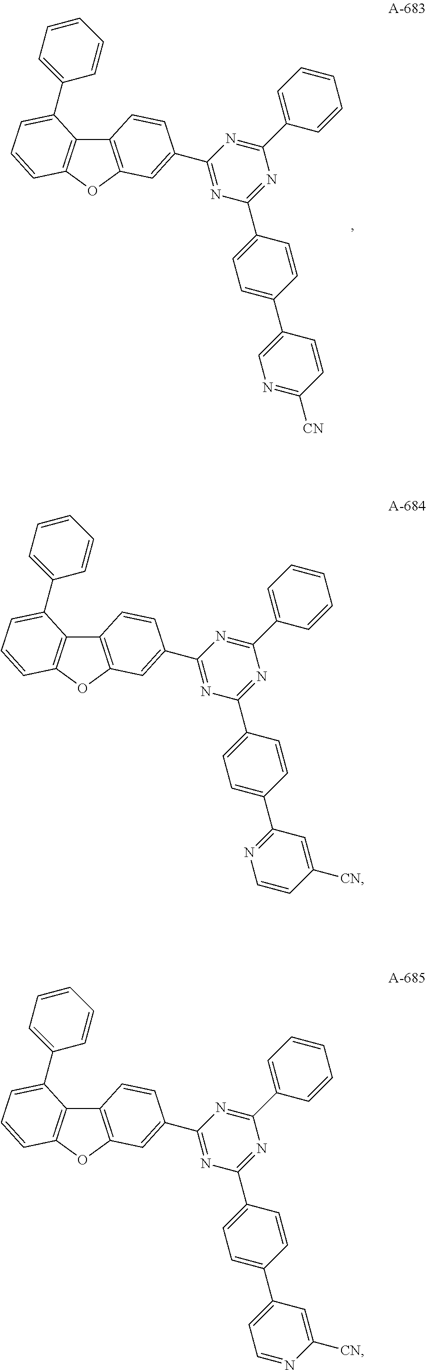 [SMILES: N#Cc1ccc(-c2ccc(-c3nc(-c4ccccc4)nc(-c4ccc5c(c4)oc4cccc(-c6ccccc6)c45)n3)cc2)cn1 ; N#Cc1ccnc(-c2ccc(-c3nc(-c4ccccc4)nc(-c4ccc5c(c4)oc4cccc(-c6ccccc6)c45)n3)cc2)c1 ; N#Cc1cc(-c2ccc(-c3nc(-c4ccccc4)nc(-c4ccc5c(c4)oc4cccc(-c6ccccc6)c45)n3)cc2)ccn1] 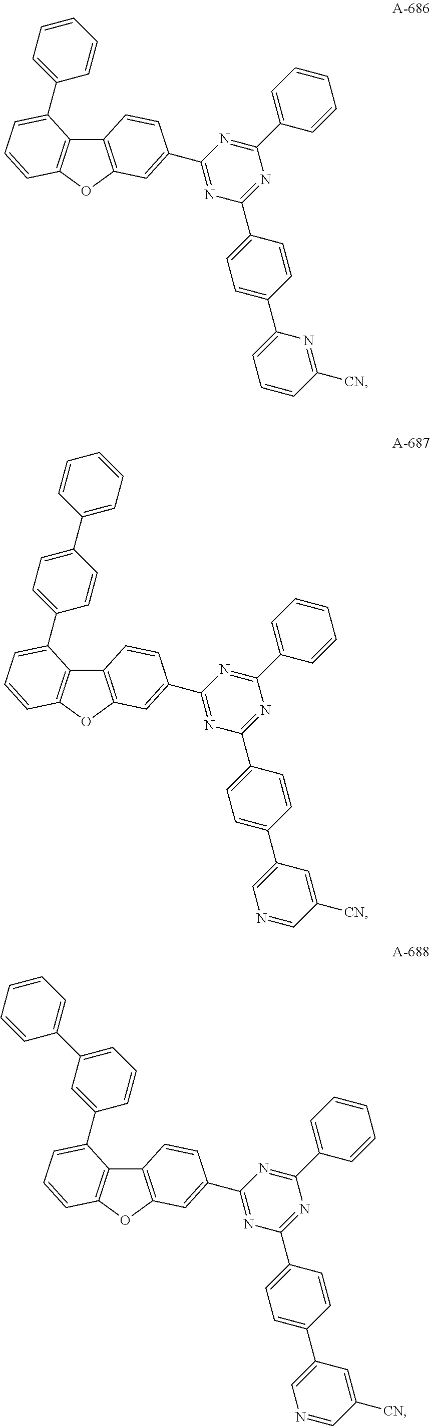 [SMILES: N#Cc1cccc(-c2ccc(-c3nc(-c4ccccc4)nc(-c4ccc5c(c4)oc4cccc(-c6ccccc6)c45)n3)cc2)n1 ; N#Cc1cncc(-c2ccc(-c3nc(-c4ccccc4)nc(-c4ccc5c(c4)oc4cccc(-c6ccc(-c7ccccc7)cc6)c45)n3)cc2)c1 ; N#Cc1cncc(-c2ccc(-c3nc(-c4ccccc4)nc(-c4ccc5c(c4)oc4cccc(-c6cccc(-c7ccccc7)c6)c45)n3)cc2)c1] 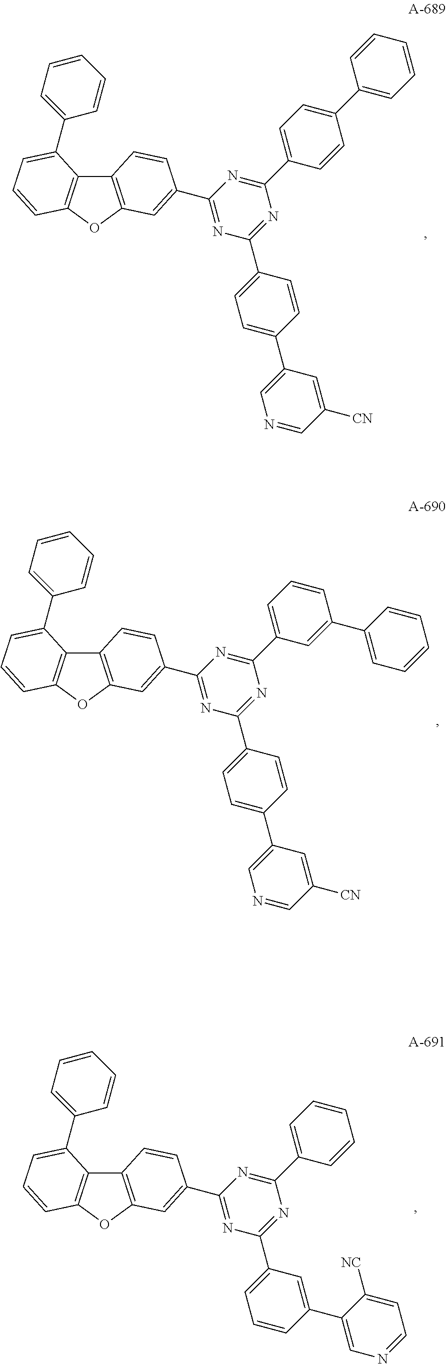 [SMILES: N#Cc1cncc(-c2ccc(-c3nc(-c4ccc(-c5ccccc5)cc4)nc(-c4ccc5c(c4)oc4cccc(-c6ccccc6)c45)n3)cc2)c1 ; N#Cc1cncc(-c2ccc(-c3nc(-c4cccc(-c5ccccc5)c4)nc(-c4ccc5c(c4)oc4cccc(-c6ccccc6)c45)n3)cc2)c1 ; N#Cc1ccncc1-c1cccc(-c2nc(-c3ccccc3)nc(-c3ccc4c(c3)oc3cccc(-c5ccccc5)c34)n2)c1] 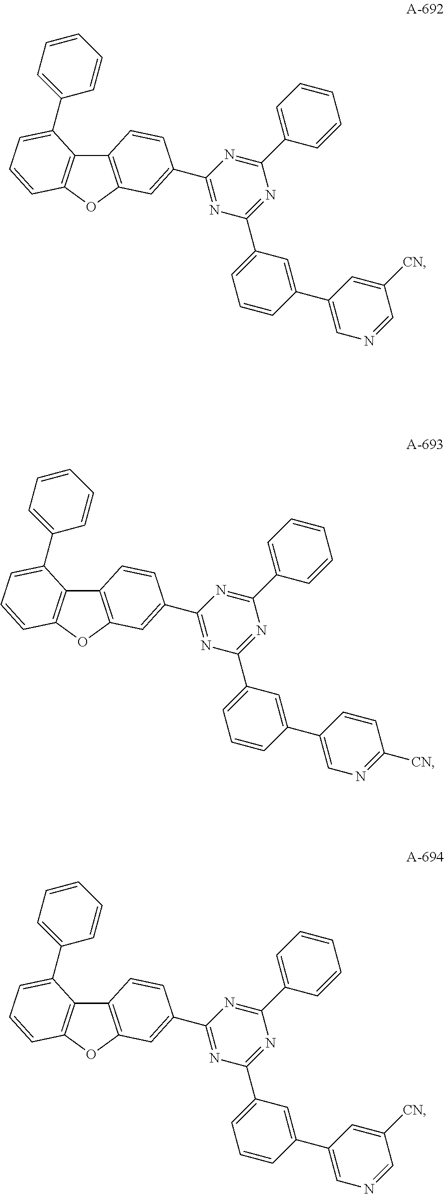 [SMILES: N#Cc1cncc(-c2cccc(-c3nc(-c4ccccc4)nc(-c4ccc5c(c4)oc4cccc(-c6ccccc6)c45)n3)c2)c1 ; N#Cc1ccc(-c2cccc(-c3nc(-c4ccccc4)nc(-c4ccc5c(c4)oc4cccc(-c6ccccc6)c45)n3)c2)cn1 ; N#Cc1cncc(-c2cccc(-c3nc(-c4ccccc4)nc(-c4ccc5c(c4)oc4cccc(-c6ccccc6)c45)n3)c2)c1] 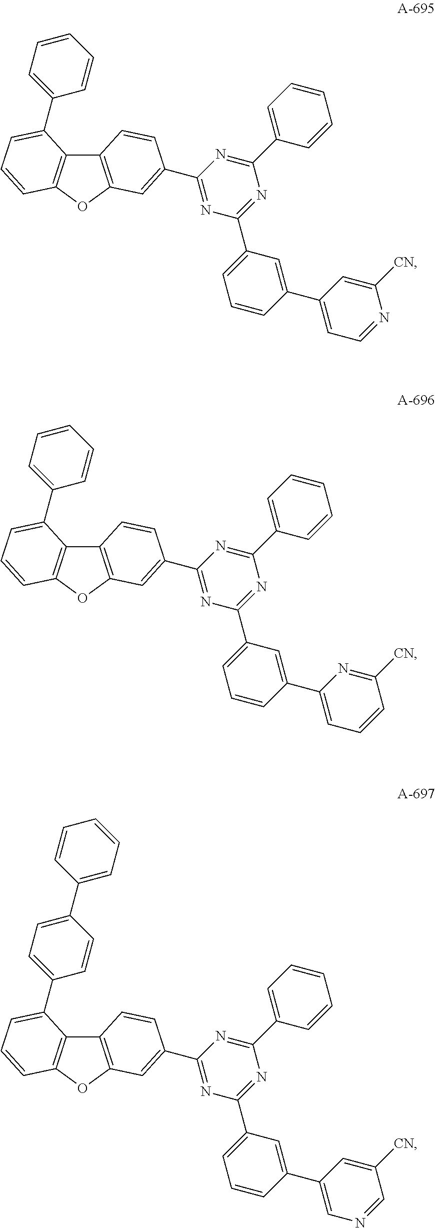 [SMILES: N#Cc1cc(-c2cccc(-c3nc(-c4ccccc4)nc(-c4ccc5c(c4)oc4cccc(-c6ccccc6)c45)n3)c2)ccn1 ; N#Cc1cccc(-c2cccc(-c3nc(-c4ccccc4)nc(-c4ccc5c(c4)oc4cccc(-c6ccccc6)c45)n3)c2)n1 ; N#Cc1cncc(-c2cccc(-c3nc(-c4ccccc4)nc(-c4ccc5c(c4)oc4cccc(-c6ccc(-c7ccccc7)cc6)c45)n3)c2)c1] 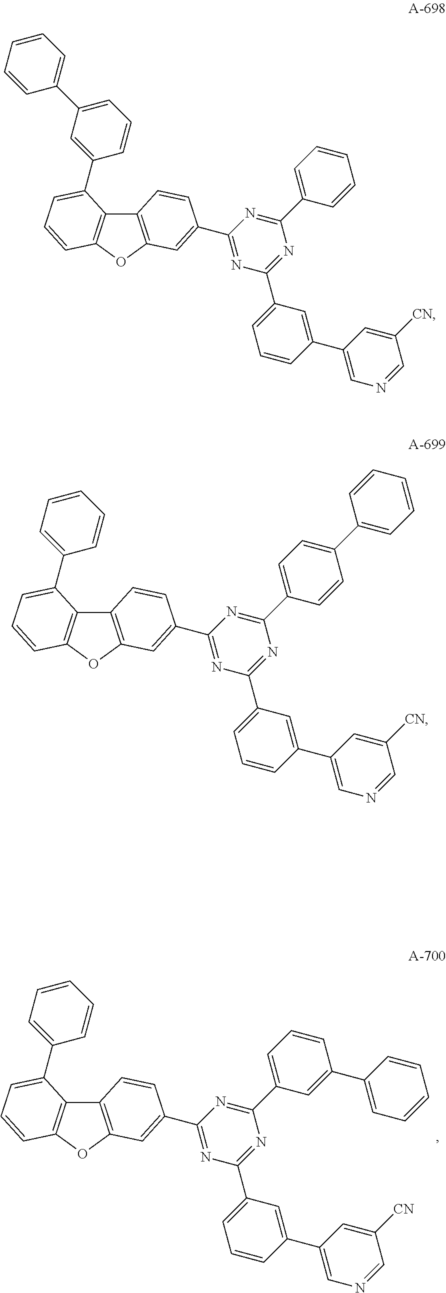 [SMILES: N#Cc1cncc(-c2cccc(-c3nc(-c4ccccc4)nc(-c4ccc5c(c4)oc4cccc(-c6cccc(-c7ccccc7)c6)c45)n3)c2)c1 ; N#Cc1cncc(-c2cccc(-c3nc(-c4ccc(-c5ccccc5)cc4)nc(-c4ccc5c(c4)oc4cccc(-c6ccccc6)c45)n3)c2)c1 ; N#Cc1cncc(-c2cccc(-c3nc(-c4cccc(-c5ccccc5)c4)nc(-c4ccc5c(c4)oc4cccc(-c6ccccc6)c45)n3)c2)c1] 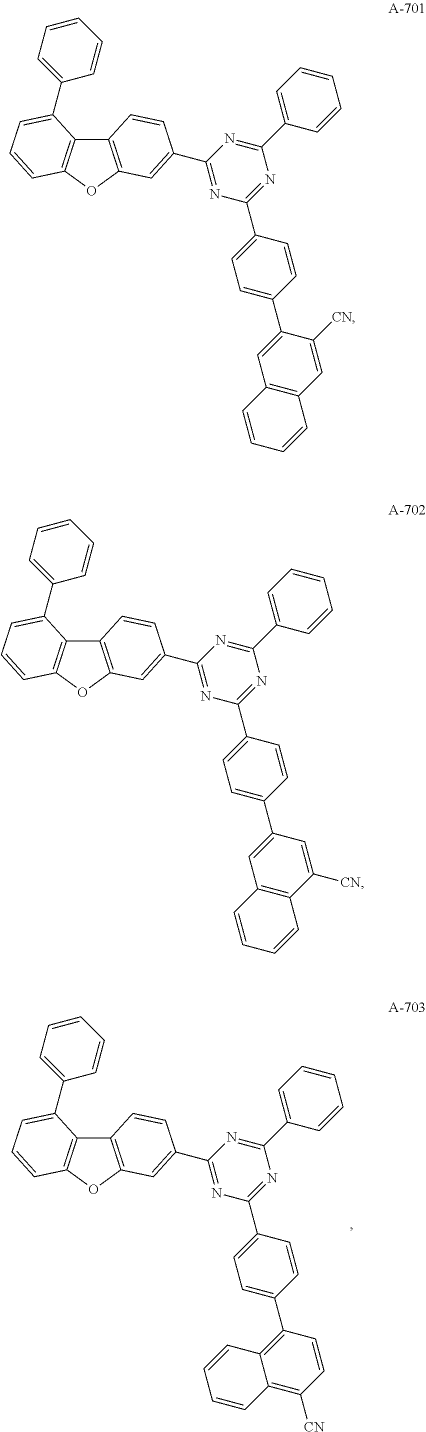 [SMILES: N#Cc1cc2ccccc2cc1-c1ccc(-c2nc(-c3ccccc3)nc(-c3ccc4c(c3)oc3cccc(-c5ccccc5)c34)n2)cc1 ; N#Cc1cc(-c2ccc(-c3nc(-c4ccccc4)nc(-c4ccc5c(c4)oc4cccc(-c6ccccc6)c45)n3)cc2)cc2ccccc12 ; N#Cc1ccc(-c2ccc(-c3nc(-c4ccccc4)nc(-c4ccc5c(c4)oc4cccc(-c6ccccc6)c45)n3)cc2)c2ccccc12] 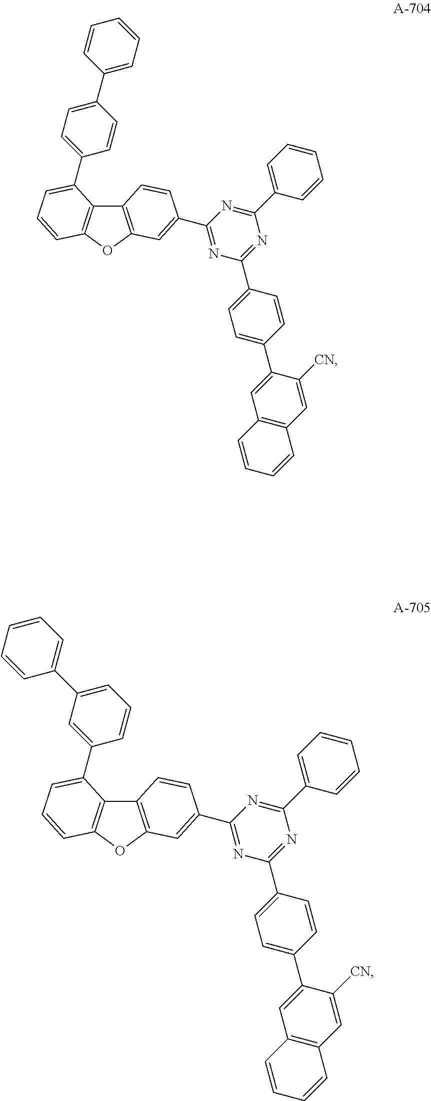 [SMILES: N#Cc1cc2ccccc2cc1-c1ccc(-c2nc(-c3ccccc3)nc(-c3ccc4c(c3)oc3cccc(-c5ccc(-c6ccccc6)cc5)c34)n2)cc1 ; N#Cc1cc2ccccc2cc1-c1ccc(-c2nc(-c3ccccc3)nc(-c3ccc4c(c3)oc3cccc(-c5cccc(-c6ccccc6)c5)c34)n2)cc1] 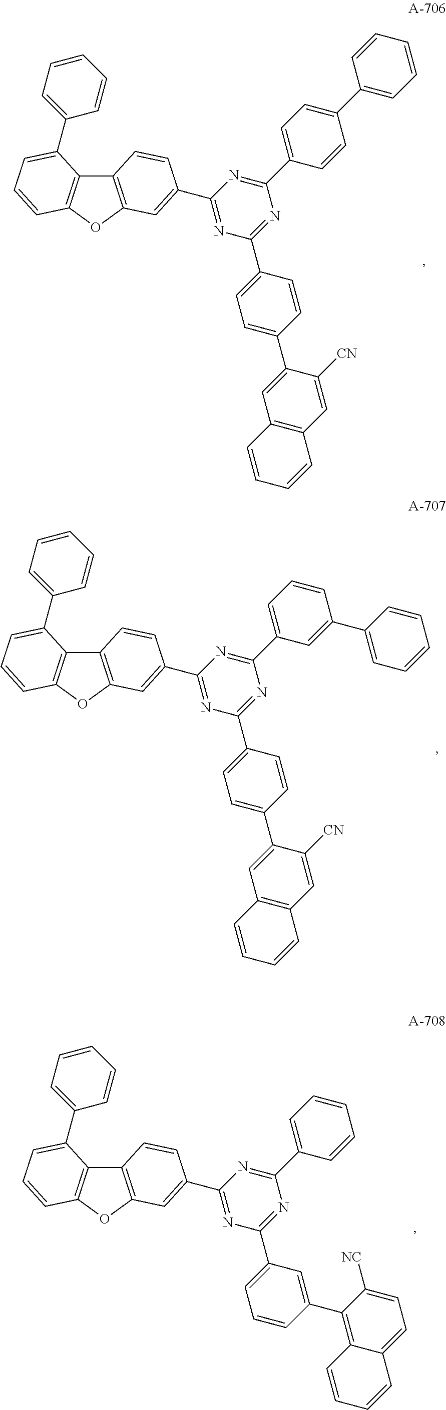 [SMILES: N#Cc1cc2ccccc2cc1-c1ccc(-c2nc(-c3ccc(-c4ccccc4)cc3)nc(-c3ccc4c(c3)oc3cccc(-c5ccccc5)c34)n2)cc1 ; N#Cc1cc2ccccc2cc1-c1ccc(-c2nc(-c3cccc(-c4ccccc4)c3)nc(-c3ccc4c(c3)oc3cccc(-c5ccccc5)c34)n2)cc1 ; N#Cc1ccc2ccccc2c1-c1cccc(-c2nc(-c3ccccc3)nc(-c3ccc4c(c3)oc3cccc(-c5ccccc5)c34)n2)c1] 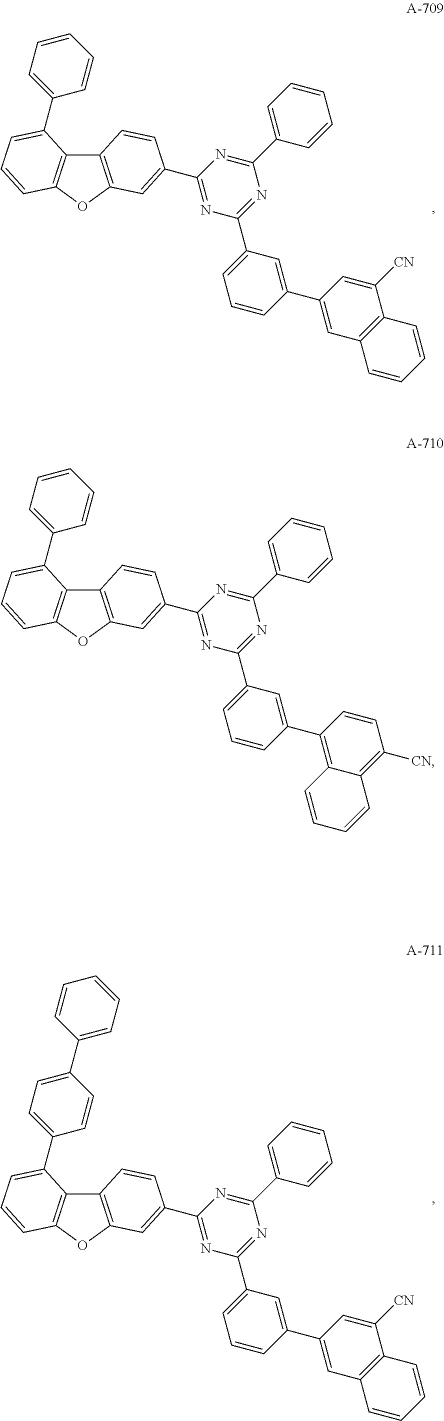 [SMILES: N#Cc1cc(-c2cccc(-c3nc(-c4ccccc4)nc(-c4ccc5c(c4)oc4cccc(-c6ccccc6)c45)n3)c2)cc2ccccc12 ; N#Cc1ccc(-c2cccc(-c3nc(-c4ccccc4)nc(-c4ccc5c(c4)oc4cccc(-c6ccccc6)c45)n3)c2)c2ccccc12 ; N#Cc1cc(-c2cccc(-c3nc(-c4ccccc4)nc(-c4ccc5c(c4)oc4cccc(-c6ccc(-c7ccccc7)cc6)c45)n3)c2)cc2ccccc12] 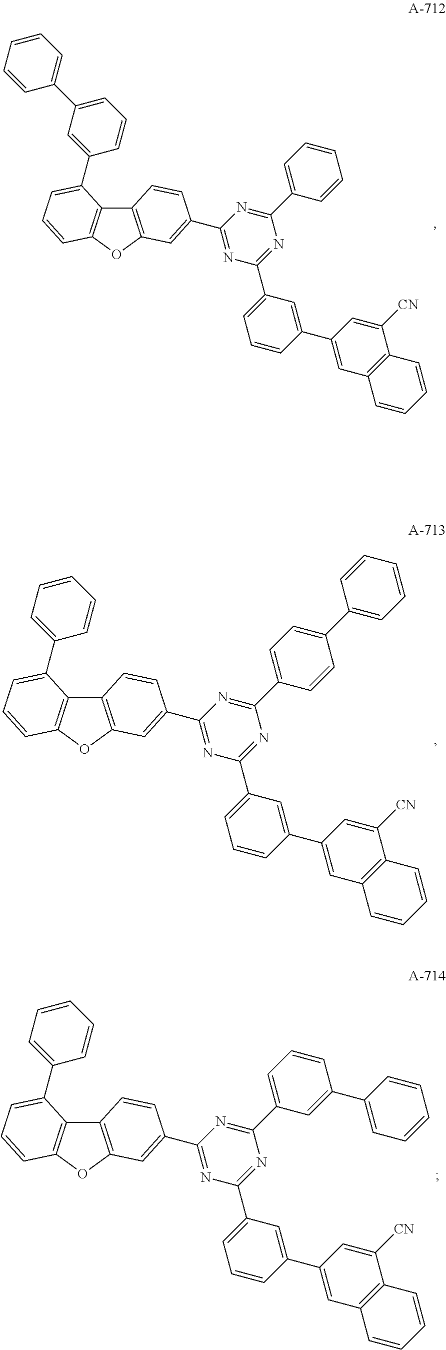 [SMILES: N#Cc1cc(-c2cccc(-c3nc(-c4ccccc4)nc(-c4ccc5c(c4)oc4cccc(-c6cccc(-c7ccccc7)c6)c45)n3)c2)cc2ccccc12 ; N#Cc1cc(-c2cccc(-c3nc(-c4ccc(-c5ccccc5)cc4)nc(-c4ccc5c(c4)oc4cccc(-c6ccccc6)c45)n3)c2)cc2ccccc12 ; N#Cc1cc(-c2cccc(-c3nc(-c4cccc(-c5ccccc5)c4)nc(-c4ccc5c(c4)oc4cccc(-c6ccccc6)c45)n3)c2)cc2ccccc12]
optionally, hydrogens in Compound A-1 to Compound A-714 can be partially or fully substituted with deuterium.

9. An organic electroluminescent device, comprising an anode, a cathode and an organic layer disposed between the anode and the cathode, wherein at least one layer of the organic layer comprises the compound according to claim 1.

10. The organic electroluminescent device according to claim 9, wherein the organic layer is an emissive layer, the compound is a host compound, and the emissive layer comprises at least a first metal complex.

11. The organic electroluminescent device according to claim 10, wherein the first metal complex has a general formula of M(Lₐ)ₘ(L_{b})ₙ(L_{c})_{q};
the metal M is selected from a metal with a relative atomic mass greater than 40;
Lₐ, L_{b} and L_{c} are a first ligand, a second ligand and a third ligand coordinated to the metal M, respectively; Lₐ, L_{b} and L_{c} may be the same or different;
Lₐ, L_{b} and L_{c} can be optionally joined to form a multidentate ligand;
m is 1, 2 or 3, n is 0, 1 or 2, q is 0, 1 or 2, and m+n+q is equal to an oxidation state of the metal M; when m is greater than or equal to 2, a plurality of Lₐ may be the same or different; when n is 2, two L_{b} may be the same or different; when q is 2, two L_{c} may be the same or different;
the ligand Lₐ has a structure represented by Formula 2: 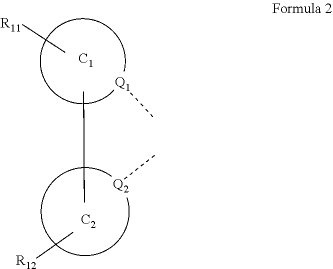
the ring C₁ and the ring C₂ are, at each occurrence identically or differently, selected from an aromatic ring having 5 to 30 ring atoms, a heteroaromatic ring having 5 to 30 ring atoms or a combination thereof;
Q₁ and Q₂ are, at each occurrence identically or differently, selected from C or N;
R₁₁ and R₁₂ represent, at each occurrence identically or differently, mono-substitution, multiple substitutions or non-substitution;
R₁₁ and R₁₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents R₁₁, R₁₂ can be optionally joined to form a ring;
the ligands L_{b} and L_{c} are, at each occurrence identically or differently, selected from a monoanionic bidentate ligand;
preferably, the ligands L_{b} and L_{c} are, at each occurrence identically or differently, selected from any one or two of the following structures: 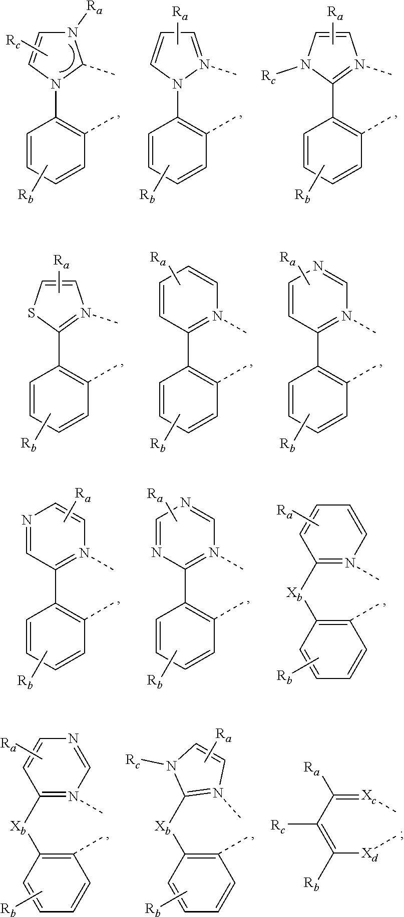
wherein
Rₐ, R_{b} and R_{c} represent, at each occurrence identically or differently, mono-substitution, multiple substitutions or non-substitution;
X_{b} is, at each occurrence identically or differently, selected from the group consisting of: O, S, Se, NR_{N1} and CR_{C1}R_{C2};
X_{c} and X_{d} are, at each occurrence identically or differently, selected from the group consisting of: O, S, Se and NR_{N2};
Rₐ, R_{b}, R_{b}, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents Rₐ, R_{b}, R_{N1}, R_{N2}, R_{C1} and R_{C2} can be optionally joined to form a ring.

12. The organic electroluminescent device according to claim 10, wherein the first metal complex is selected from the group consisting of:

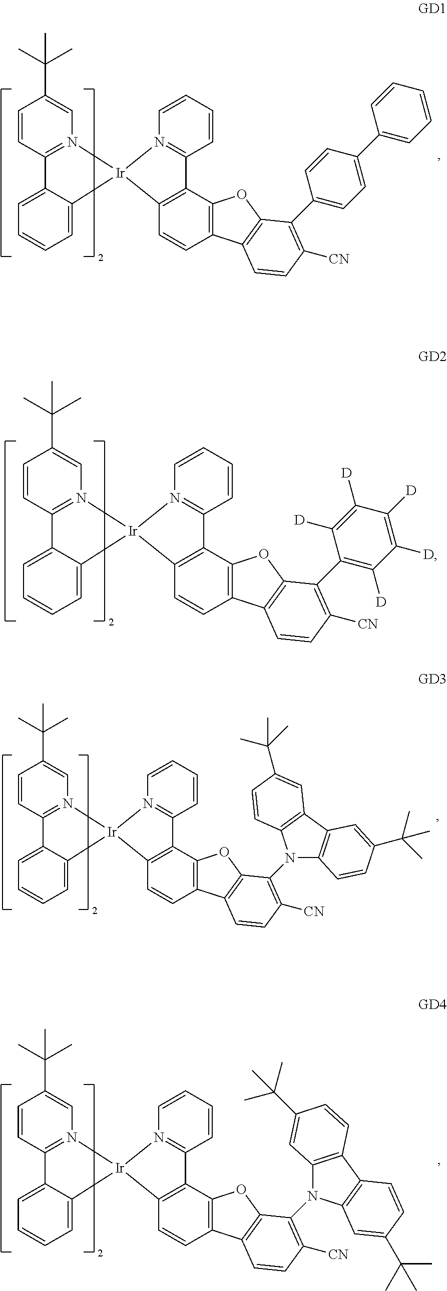

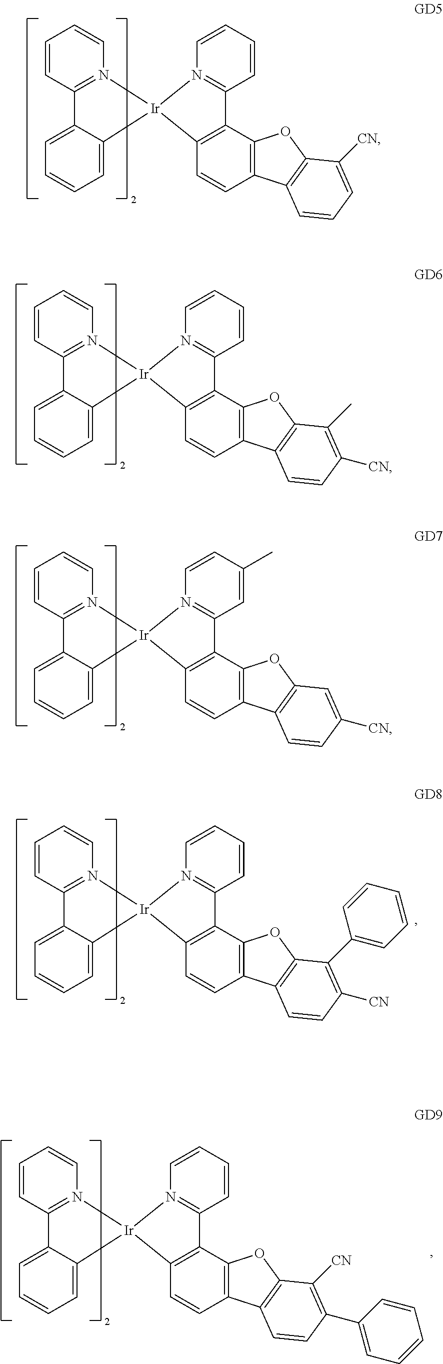

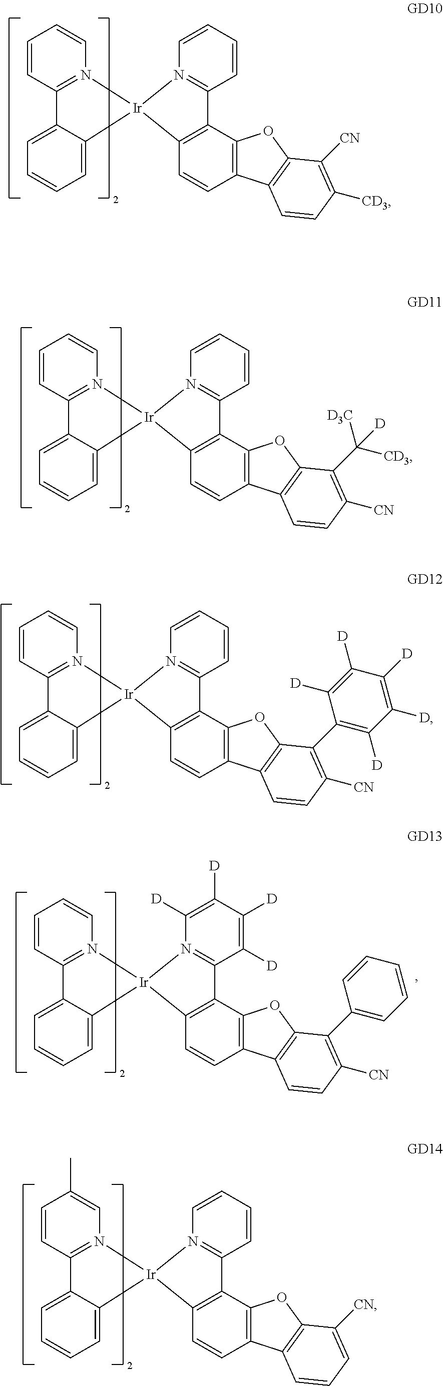

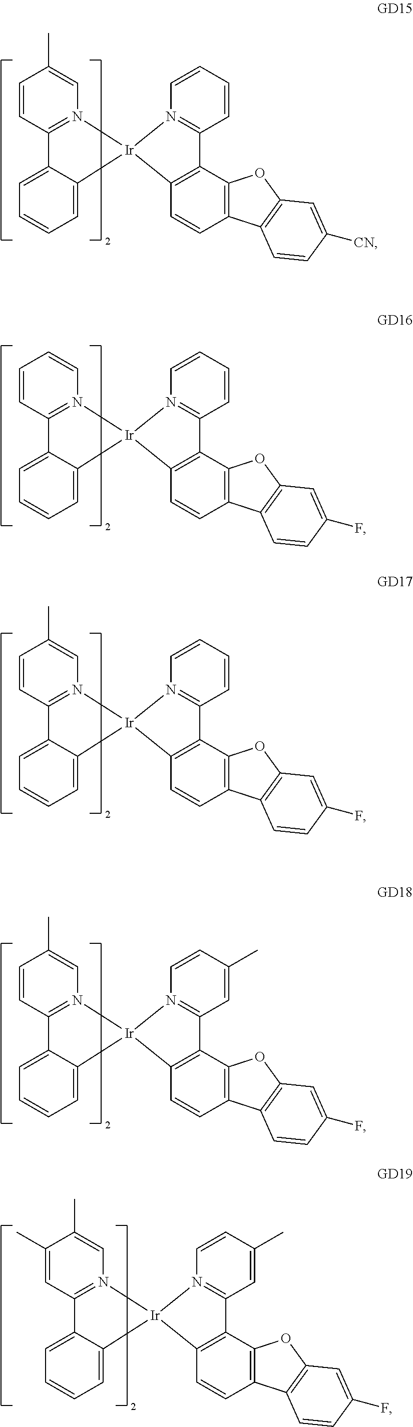

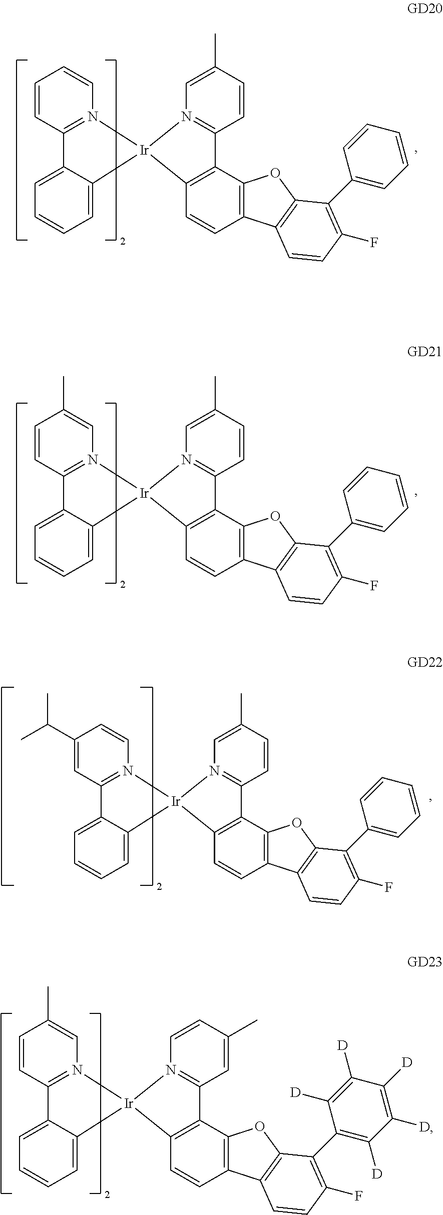

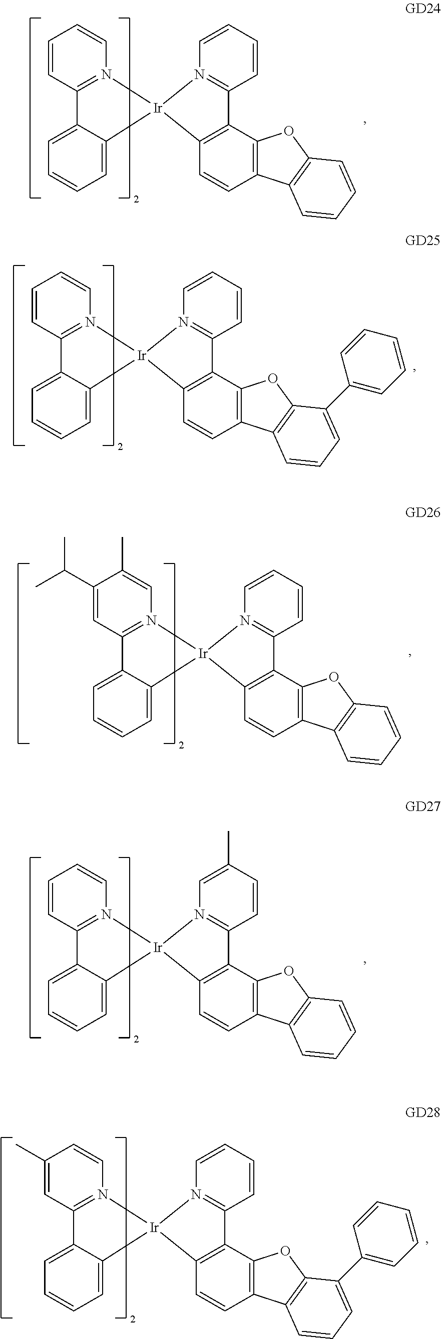

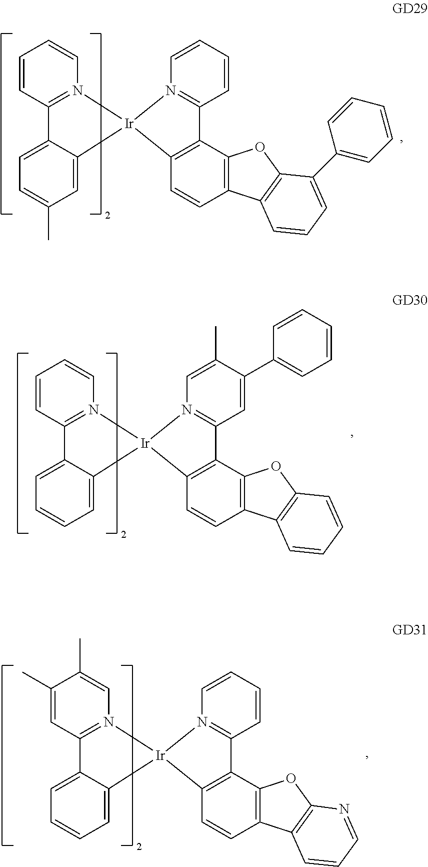

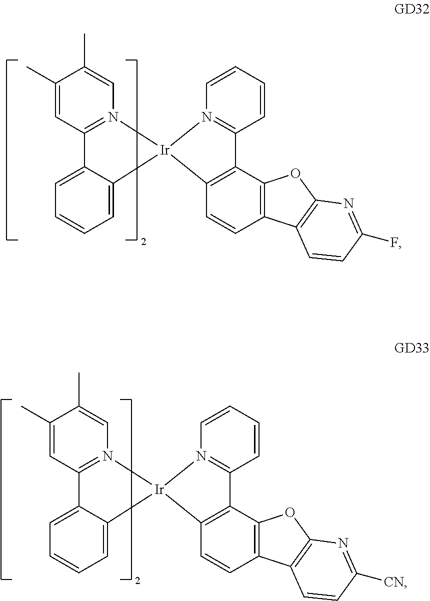

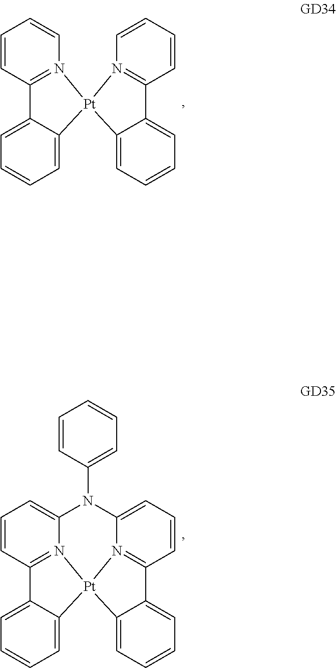

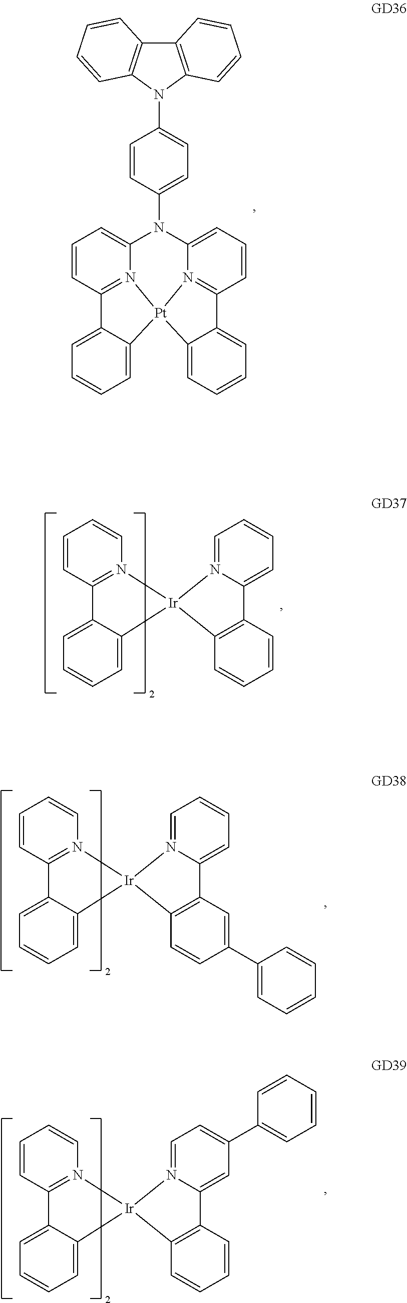

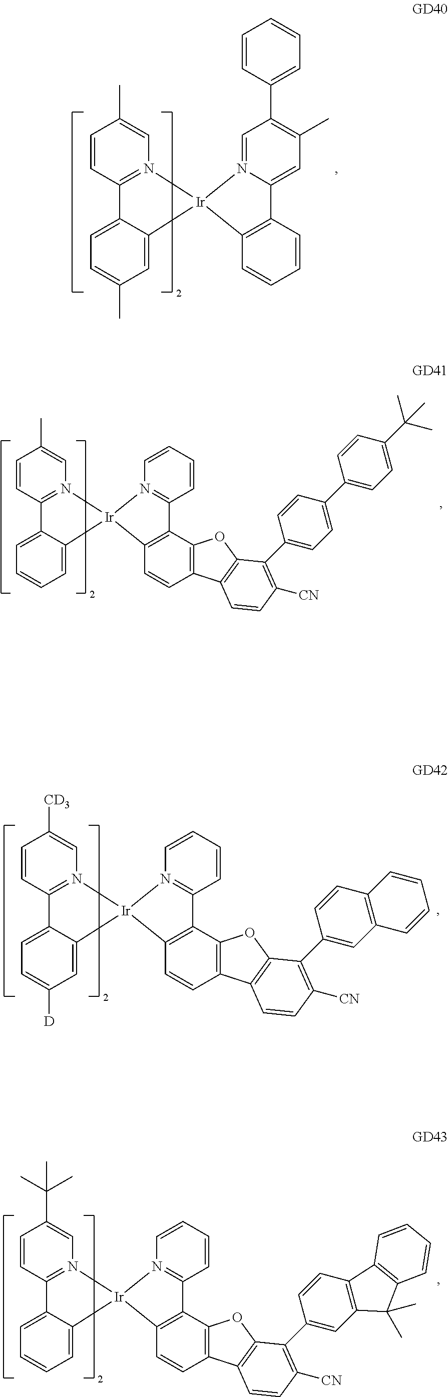

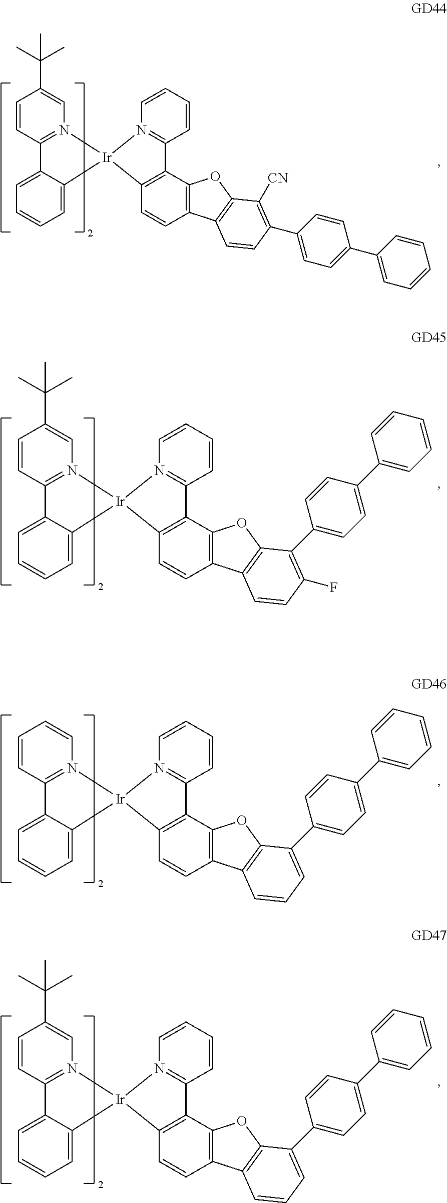

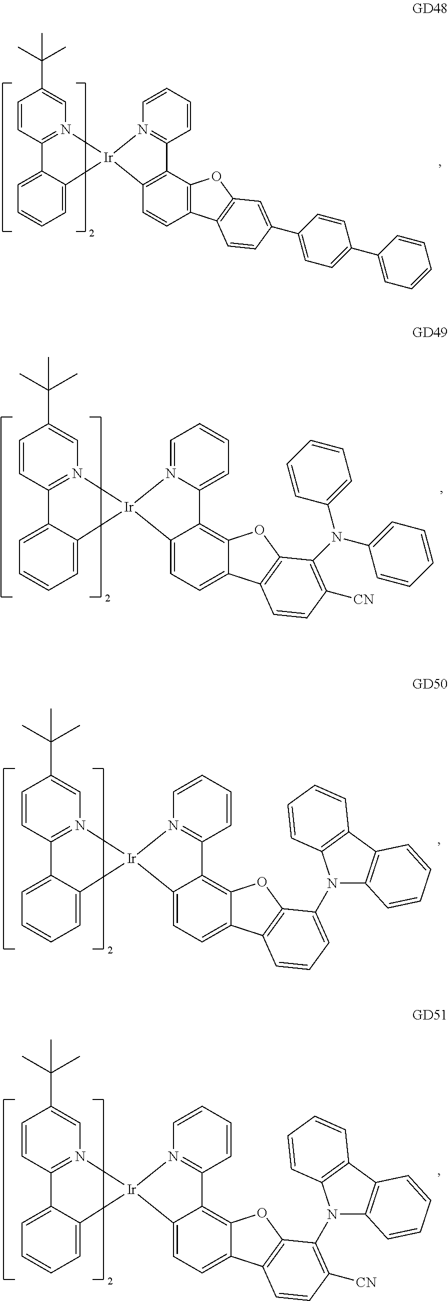

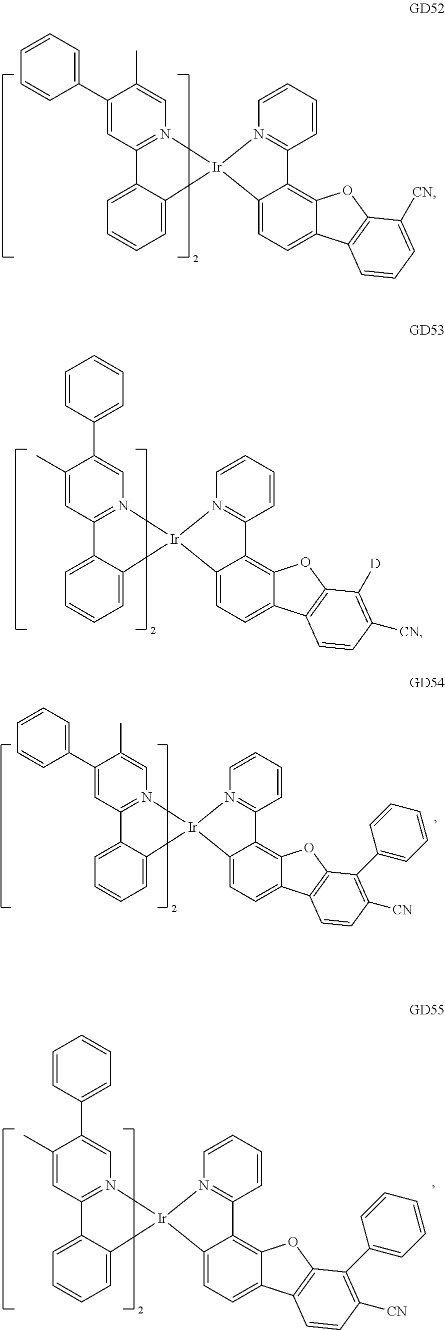

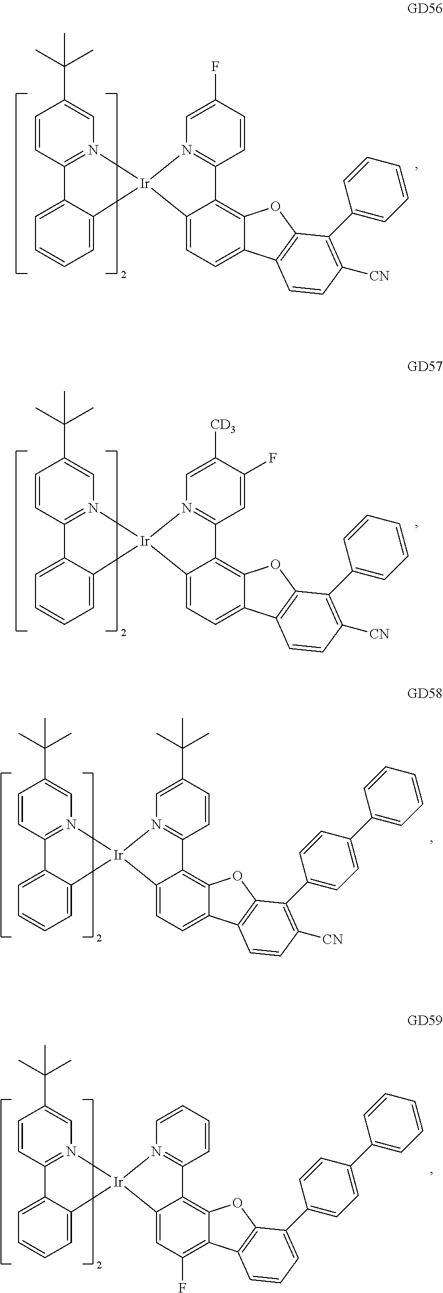

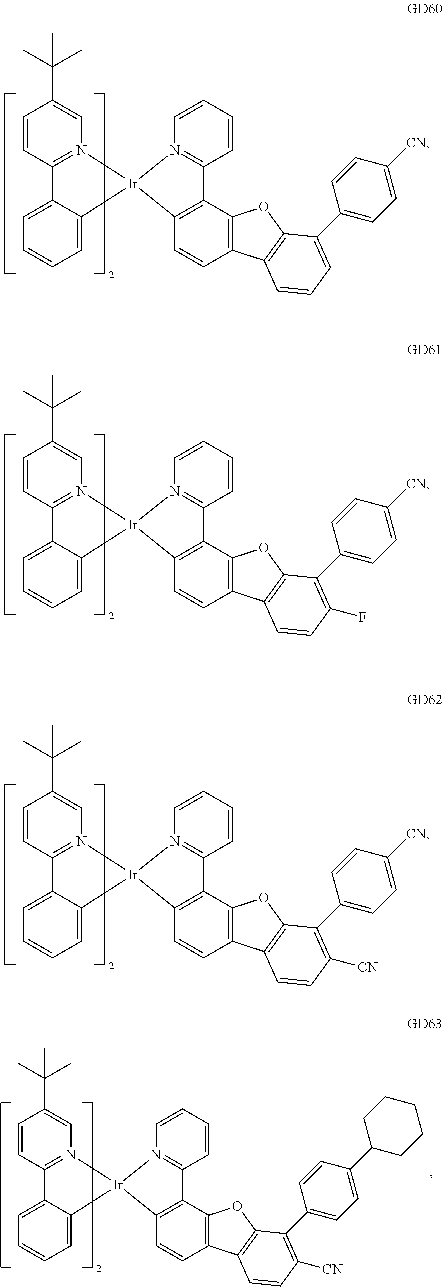

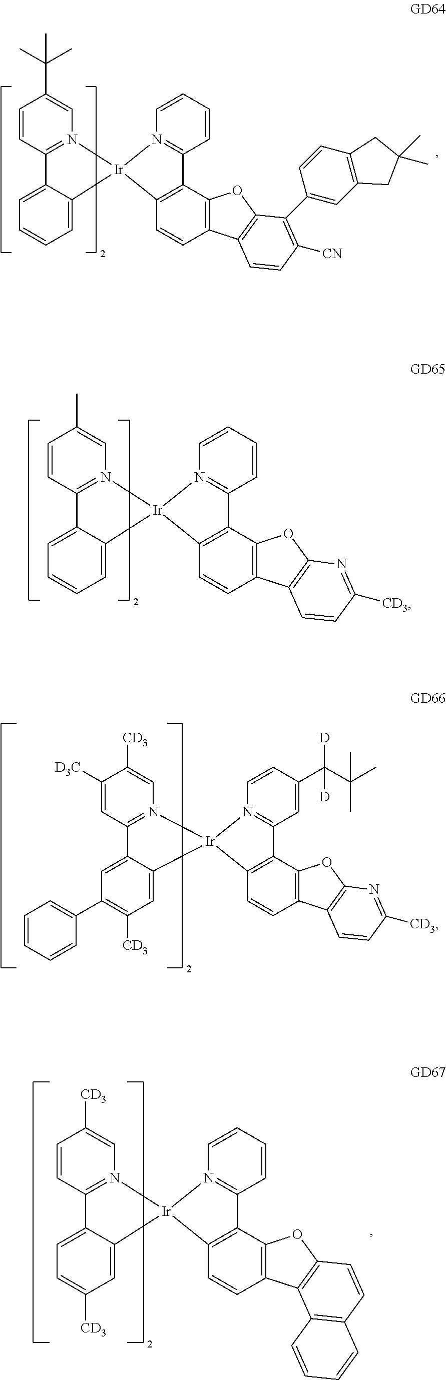

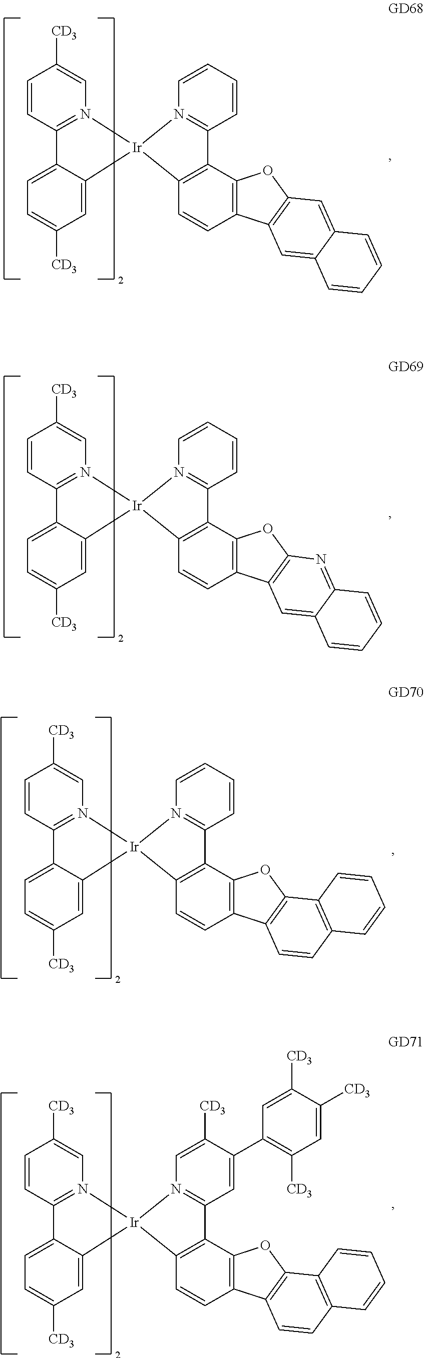

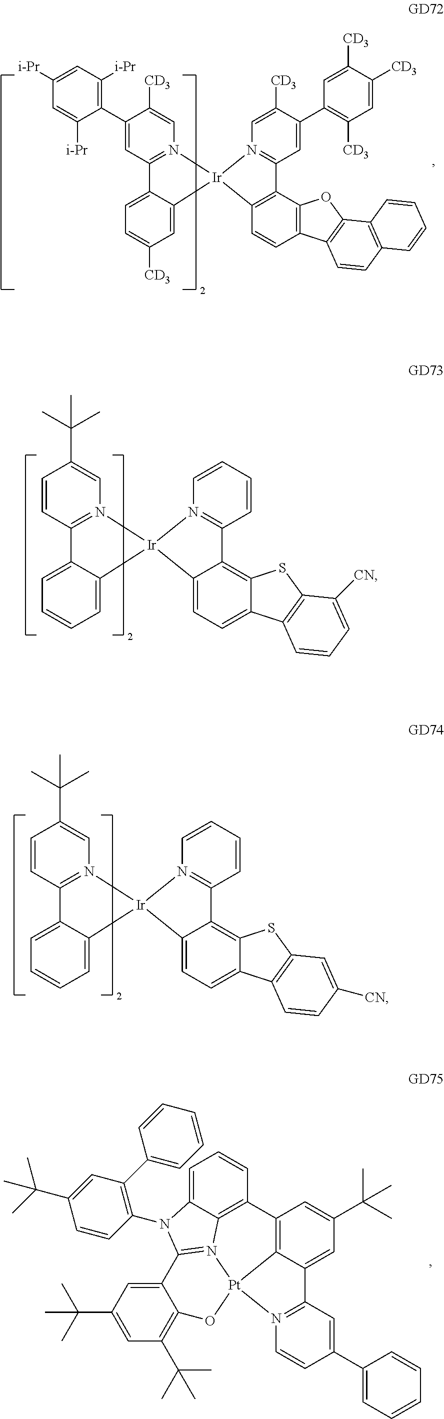

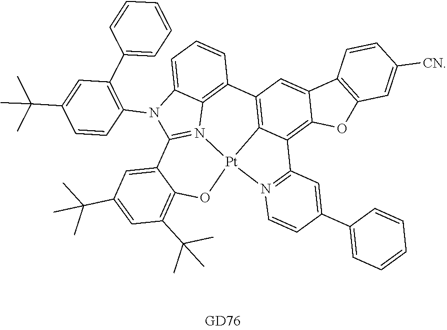

13. The organic electroluminescent device according to claim 10, wherein the emissive layer further comprises a second compound, wherein the second compound comprises at least one chemical group selected from the group consisting of: benzene, pyridine, pyrimidine, triazine, carbazole, azacarbazole, indolocarbazole, dibenzothiophene, aza-dibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene and combinations thereof;
preferably, the second compound comprises at least one chemical group selected from the group consisting of: benzene, carbazole, indolocarbazole, fluorene, silafluorene and combinations thereof.

14. The organic electroluminescent device according to claim 13, wherein the second compound has a structure represented by Formula 3 or Formula 4: 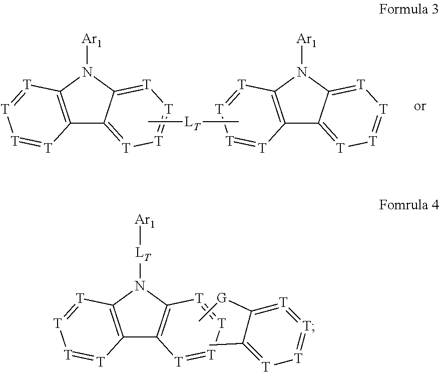
wherein
G is, at each occurrence identically or differently, selected from C(R_{g})₂, NR_{g}, O or S;
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof;
T is, at each occurrence identically or differently, selected from C, CRₜ or N;
Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkenyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar₁ is, at each occurrence identically or differently, selected from substituted or unsubstituted aryl having 6 to 30 carbon atoms, substituted or unsubstituted heteroaryl having 3 to 30 carbon atoms or a combination thereof;
adjacent substituents Rₜ, R_{g} can be optionally joined to form a ring;
preferably, the second compound has a structure represented by one of Formulas 3-a to 3-j and Formulas 4-a to 4-f: 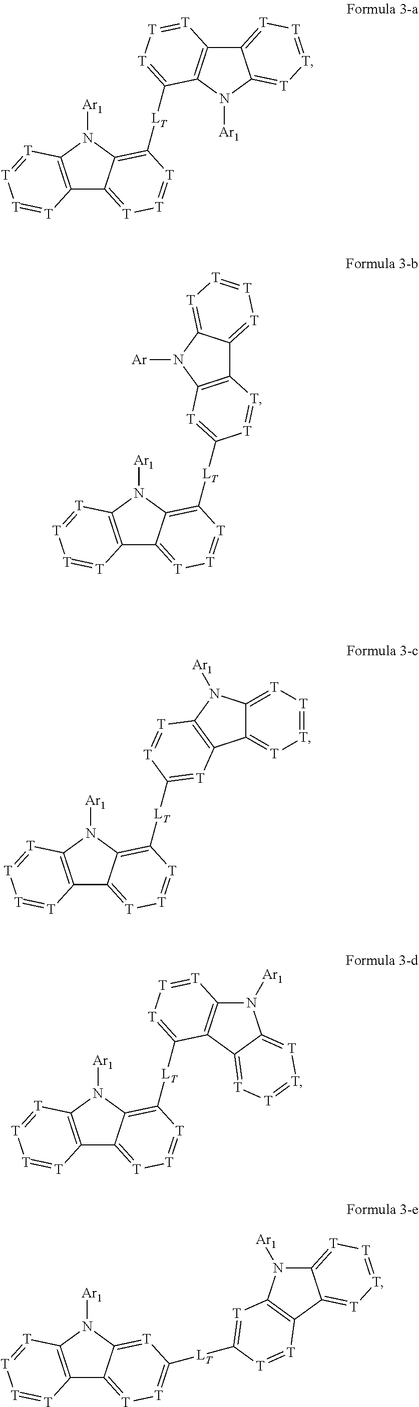 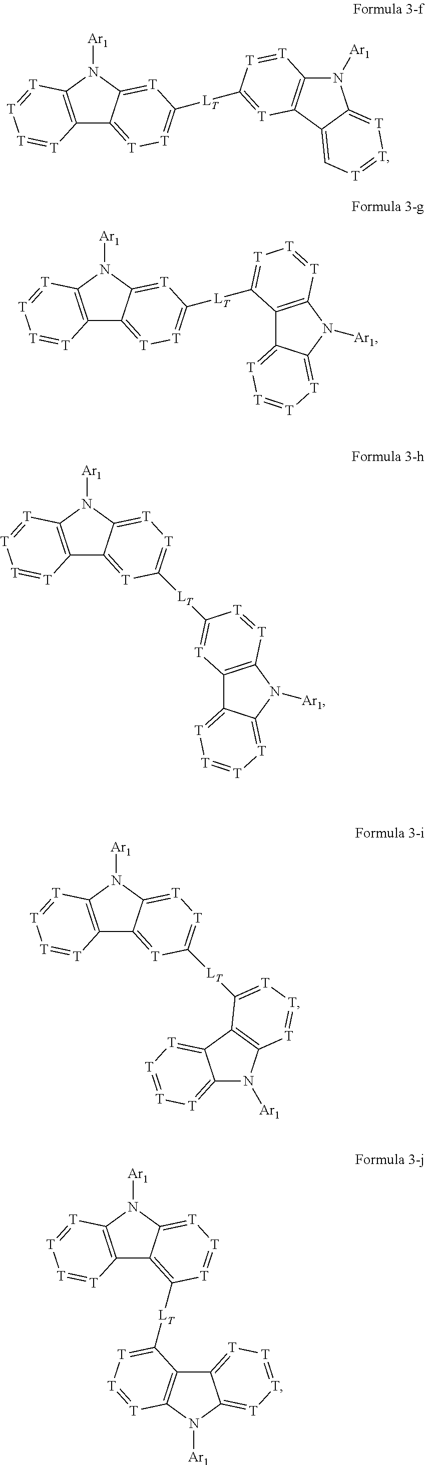 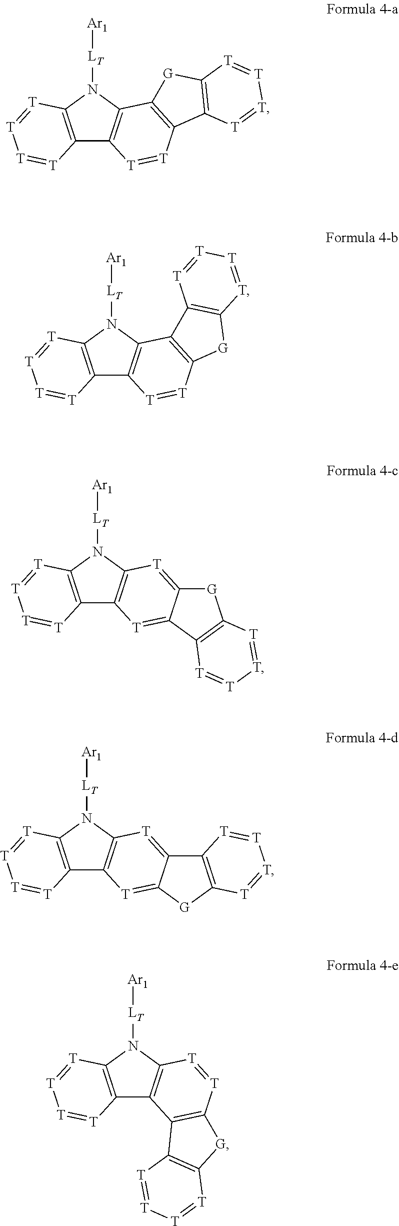 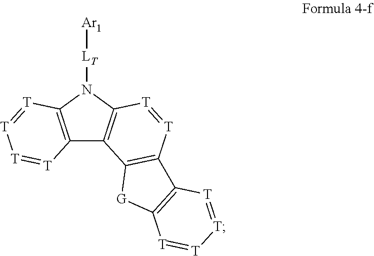
5 wherein in Formulas 3-a to 3-j, T, L_{T} and Ar₁ are defined the same as in Formula 3; and
wherein in Formulas 4-a to 4-f, T, G, L_{T} and Ar₁ are defined the same as in Formula 4.

15. A compound composition, comprising the compound according to claim 1.
